(19) United States
(12) Patent Application Publication
(10) Pub. No.: US 2006/0199821 A1
(43) Pub. Date: Sep. 7, 2006

(54) Heterocyclic amides and sulfonamides

(75) Inventors: Richard Tester, Alameda, CA (US); Xuefei Tan, Union City, CA (US); Kurt Schinzel, San Jose, CA (US); Imad Nashashibi, Milpitas, CA (US); Gregory R. Luedtke, Royal Oaks, CA (US); Weiling Liang, Palo Alto, CA (US); Joon Jung, Fremont, CA (US); R. Richard Goehring, Pipersville, PA (US); Sundeep Dugar, San Jose, CA (US); Steven Do, San Jose, CA (US)

Correspondence Address: MORRISON & FOERSTER LLP, 12531 HIGH BLUFF DRIVE, SUITE 100, SAN DIEGO, CA, 92130-2040, US

(73) Assignee: Scios, Inc., Fremont, CA (US)

(21) Appl. No.: 11/196,650

(22) Filed: Aug. 3, 2005

Related U.S. Application Data
(63) Continuation of application No. 10/957,504, filed on Sep. 30, 2004.
(60) Provisional application No. 60/507,633, filed on Sep. 30, 2003.

(51) Int. Cl. A61K 31/5377; A61K 31/53; A61K 31/513; A61K 31/506; C07D 413/02; C07D 403/02; C07D 405/02

(52) U.S. Cl. 514/235.5; 544/209; 544/314; 544/329; 544/112; 544/122; 514/241; 544/295; 514/252.14; 514/269; 514/275

(57) ABSTRACT

The invention is directed to compounds and methods to inhibit p38 kinase wherein the compounds are a pyrimidine or pyridine coupled to two mandatory substituents.

### CROSS-REFERENCE TO RELATED APPLICATIONS

This application claims benefit of U.S. provisional application 60/507,633 filed Sep. 30, 2003. The contents of this document are incorporated herein by reference.

### FIELD OF THE INVENTION

The invention relates to compounds useful in treating various disorders associated with enhanced activity of kinase p38. More specifically, it concerns compounds that are related to a pyrimidine or a pyridine having a mandatory amide substituent as useful in these methods.

### BACKGROUND OF THE INVENTION

A large number of chronic and acute conditions have been recognized to be associated with perturbation of the inflammatory response. A large number of cytokines participate in this response, including IL-1, IL-6, IL-8 and TNF. It appears that the activity of these cytokines in the regulation of inflammation rely at least in part on the activation of an enzyme on the cell signaling pathway, a member of the MAP kinase family generally known as p38 and alternatively known as CSBP and RK. This kinase is activated by dual phosphorylation after stimulation by physiochemical stress, treatment with lipopolysaccharides or with proinflammatory cytokines such as IL-1 and TNF. Therefore, inhibitors of the kinase activity of p38 are useful anti-inflammatory agents.

PCT applications WO98/06715, WO98/07425, and WO 96/40143, all of which are incorporated herein by reference, describe the relationship of p38 kinase inhibitors with various disease states. As mentioned in these applications, inhibitors of p38 kinase are useful in treating a variety of diseases associated with chronic inflammation. These applications list rheumatoid arthritis, rheumatoid spondylitis, osteoarthritis, gouty arthritis and other arthritic conditions, sepsis, septic shock, endotoxic shock, Gram-negative sepsis, toxic shock syndrome, asthma, adult respiratory distress syndrome, stroke, reperfusion injury, CNS injuries such as neural trauma and ischemia, psoriasis, restenosis, cerebral malaria, chronic pulmonary inflammatory disease, chronic obstructive pulmonary disease, cystic fibrosis, silicosis, pulmonary sarcosis, bone fracture healing, bone resorption diseases such as osteoporosis, soft tissue damage, graft-versus-host reaction, Crohn's Disease, ulcerative colitis including inflammatory bowel disease (IBD) and pyresis.

The above-referenced PCT applications disclose compounds which are p38 kinase inhibitors said to be useful in treating these disease states. These compounds are either imidazoles or are indoles substituted at the 3- or 4-position with a piperazine ring linked through a carboxamide linkage.

Certain aroyl/phenyl-substituted piperazines and piperidines which inhibit p38-α kinase are described in PCT publication WO00/12074 published 9 Mar. 2000. In addition, indolyl substituted piperidines and piperazines which inhibit this enzyme are described in PCT publication No. WO99/61426 published 2 Dec. 1999. Carbolene derivatives of piperidine and piperazine as p38-α inhibitors are described in PCT publication WO 00/59904 published 12 Oct. 2000. Additional substitutions on similar compounds are described in PCT publication WO 00/71535 published 30 Nov. 2000.

### DISCLOSURE OF THE INVENTION

The invention is directed to methods and compounds useful in treating conditions that are characterized by enhanced p38-α activity. These conditions include inflammation, proliferative diseases, and certain cardiovascular disorders as well as Alzheimer's disease as further described below.

Compounds of the invention have been found to inhibit p38 kinase, the α-isoform in particular, and are thus useful in treating diseases mediated by these activities.

The invention is related to compounds of Formula I:

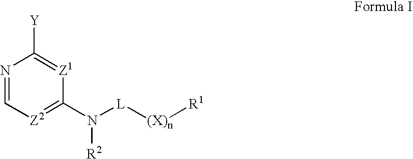

or a pharmaceutically acceptable salt or prodrug thereof, wherein

R¹ is C₁₋₁₀ alkyl, or a C3-12 cyclic hydrocarbyl and which may contain 0, 1, 2, or 3 heteroatoms and which may be optionally substituted by 1-4 groups selected from halo, R³, C₁₋₆ optionally substituted alkenyl, amidine, guanidine, R³CO, COOR³, CONR³₂, OR³, NR³R³, SR³, SO₂R³NHCOR³, CN, and NHCONR³₂, wherein R³ is H, C₁₋₆ alkyl or aryl each of which is optionally substituted with R, OR halo, NR₂, SR, SO₂R, CN, COOR, CONR₂ or CF₃, where each R is independently H or C₁-C₆ alkyl;

L is CO or SO₂;

each X is independently O, CO, CR₂, or NR, where R is lower alkyl and two R groups can be joined to form a 5-7 membered ring, provided that where X is NR or O it is not directly linked to another N or O, and that not more than two X groups are CO;

n=0, 1, 2, or 3;

R² is H, C₁-C₆ alkyl, C₁-C₆ alkenyl, C₁-C₆ heteroalkyl, heterocyclylalkyl, aryl, arylalkyl, heteroaryl, or heteroarylalkyl, each of which is optionally substituted with up to four groups selected from R, halo, CN, OR, ═O, C(NR)NR₂, NR₂, COR, COOR, CONR₂, SR, SOR, SO₂R, SO₂NR₂, NRCOOR, and COCOOR, wherein each R is independently H, alkyl, heteroalkyl, arylalkyl, or diarylalkyl, each of which may be substituted with hydroxy, amino, C1-C6 alkoxy, C₁-C₆-alkyl-COOR, C₁-C₆-alkyl-CONR₂ or halo, and wherein two R groups can cyclize to form a 3 to 8 membered ring, optionally including up to two heteroatoms selected from N, O and S;

Y is NR⁴R⁵ or OR⁵,

wherein R⁴ is H or C₁₋₆ alkyl which is optionally substituted with R, OR, NR₂, SR, SO₂R, halo, COOR, ═O, NRCOOR, COR, NRCOR, aryl, arylalkyl, arylalkoxy, or CONR₂, wherein each R is independently H or C₁-C₆ alkyl;

each R⁵ is independently H, a C₁₋₁₀ alkyl optionally substituted with a hydrocarbyl or heterocyclic ring or ring system which may contain 0, 1, 2, or 3 heteroatoms selected from O, N and S, and which is optionally substituted with R, OR, NR₂, SR, SO₂R, halo, COOR, ═O, NRCOOR, COR, NRCOR, aryl, arylalkyl, arylalkoxy, or CONR₂, wherein each R is independently H or C₁-C₆ alkyl; or a C₃₋₇ cycloalkyl, aryl, arylalkyl, heteroaryl, or a fused or unfused carbocyclic or heterocyclic ring, each of which is optionally substituted with up to four groups selected from R, OR, NR₂, SR, SO₂R, halo, COOR, ═O, and CONR₂, wherein each R is independently H or C₁-C₆ alkyl; and

one of Z¹ and Z² is CH, and the other is either CH or N.

### MODES OF CARRYING OUT THE INVENTION

The compounds of formula (I) are useful in treating conditions which are characterized by overactivity of p38 kinase, in particular the α-isoform. Conditions “characterized by enhanced p38-α activity” include those where this enzyme is present in increased amount or wherein the enzyme has been modified to increase its inherent activity, or both. Thus, “enhanced activity” refers to any condition wherein the effectiveness of these proteins is undesirably high, regardless of the cause.

The compounds of the invention are useful in conditions where p38-α kinase shows enhanced activity. These conditions are those in which fibrosis and organ sclerosis are caused by, or accompanied by, inflammation, oxidation injury, hypoxia, altered temperature or extracellular osmolarity, conditions causing cellular stress, apoptosis or necrosis. These conditions include ischemia-reperfusion injury, congestive heart failure, progressive pulmonary and bronchial fibrosis, hepatitis, arthritis, inflammatory bowel disease, glomerular sclerosis, interstitial renal fibrosis, chronic scarring diseases of the eyes, bladder and reproductive tract, bone marrow dysplasia, chronic infectious or autoimmune states and traumatic or surgical wounds. These conditions, of course, would be benefited by compounds which inhibit p38-α. Methods of treatment with the compounds of the invention are further discussed below.

The Invention Compounds

The compounds useful in the invention are derivatives of pyrimidine or pyridine.

The pyridyl or pyrimidinyl moiety has mandatory substituents at the 2 and 4 positions, and in another separate embodiment, a pyrimidyl moiety may have mandatory substituents at the 4 and 6 positions. Such compound has formula 1:

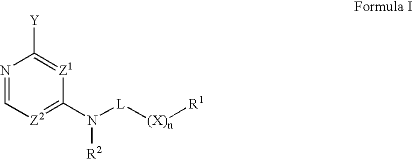

or a pharmaceutically acceptable salt or prodrug thereof, wherein

R¹ is C₁₋₁₀ alkyl, or a C3-12 cyclic hydrocarbyl and which may contain 0, 1, 2, or 3 heteroatoms and which may be optionally substituted by 1-4 groups selected from halo, R³, C₁₋₆ optionally substituted alkenyl, amidine, guanidine, R³CO, COOR³, CONR³₂, OR³, NR³R³, SR³, SO₂R³NHCOR³, CN, and NHCONR³₂, wherein R³ is H, C₁₋₆ alkyl or aryl each of which is optionally substituted with R, OR halo, NR₂, SR, SO₂R, CN, COOR, CONR₂ or CF₃, where each R is independently H or C₁-C₆ alkyl;

L is CO or SO₂;

each X is independently O, CO, CR₂, or NR, where R is lower alkyl and two R groups can be joined to form a 5-7 membered ring, provided that where X is NR or 0 it is not directly linked to another N or O, and that not more than two X groups are CO;

n=0, 1, 2, or 3;

R² is H, C₁-C₆ alkyl, C₁-C₆ alkenyl, C₁-C₆ heteroalkyl, heterocyclylalkyl, aryl, arylalkyl, heteroaryl, or heteroarylalkyl, each of which is optionally substituted with up to four groups selected from R, halo, CN, OR, ═O, C(NR)NR₂, NR₂, COR, COOR, CONR₂, SR, SOR, SO₂R, SO₂NR₂, NRCOOR, and COCOOR, wherein each R is independently H, alkyl, heteroalkyl, arylalkyl, or diarylalkyl, each of which may be substituted with hydroxy, amino, C₁-C₆ alkoxy, C₁-C₆-alkyl-COOR, C₁-C₆-alkyl-CONR₂ or halo, and wherein two R groups can cyclize to form a 3 to 8 membered ring, optionally including up to two heteroatoms selected from N, O and S;

Y is NR⁴R⁵ or OR⁵,

wherein R⁴ is H or C₁₋₆ alkyl which is optionally substituted with R, OR, NR₂, SR, SO₂R, halo, COOR, ═O, NRCOOR, COR, NRCOR, aryl, arylalkyl, arylalkoxy, or CONR₂, wherein each R is independently H or C₁-C₆ alkyl;

each R⁵ is independently H, a C₁₋₁₀ alkyl optionally substituted with a hydrocarbyl or heterocyclic ring or ring system which may contain 0, 1, 2, or 3 heteroatoms selected from O, N and S, and which is optionally substituted with R, OR, NR₂, SR, SO₂R, halo, COOR, ═O, NRCOOR, COR, NRCOR, aryl, arylalkyl, arylalkoxy, or CONR₂, wherein each R is independently H or C₁-C₆ alkyl; or a C₃₋₇ cycloalkyl, aryl, arylalkyl, heteroaryl, or a fused or unfused carbocyclic or heterocyclic ring, each of which is optionally substituted with up to four groups selected from R, OR, NR₂, SR, SO₂R, halo, COOR, ═O, and CONR₂, wherein each R is independently H or C₁-C₆ alkyl; and

one of Z¹ and Z² is CH, and the other is either CH or N.

In one aspect, n=0. In another aspect, L is CO. In one embodiment n=1 and X is O. With respect to the central ring structure, in one embodiment, Z¹ and Z² are both CH. In another embodiment, either Z¹ or Z² is N.

With respect to R¹, in one embodiment R¹ is a C₃-C₁₀ alkyl or a C₃-C₁₂ aromatic or partially aromatic group, each of which may contain 0 to 3 heteroatoms and which may be optionally substituted by 1-4 groups selected from halo, R³, C₁₋₆ optionally substituted alkenyl, amidine, guanidine, R³CO, COOR³, CONR³₂, OR³, NR³R³, SR³, SO₂R³NHCOR³, CN, and NHCONR³₂ wherein R³ is H, C₁₋₆ alkyl or aryl each of which is optionally substituted with R, OR halo, NR₂, SR, SO₂R, CN, COOR, CONR₂ or CF₃, where each R is independently H or C₁-C₆ alkyl.

In another embodiment, R¹ is a aryl(C₂₋₆)alkenyl or a C₃₋₆ cyclic alkyl or aromatic ring or ring system which may contain 0, 1, 2, or 3 heteroatoms and which may be optionally substituted as described above.

In yet another embodiment R¹ is bicyclic, such as naphthyl, benzofuranyl, indanyl, 2,3-dihydrobenzofuranyl, benzothienyl, or 1,2,3,4-tetrahydronaphthyl, each of which is optionally substituted by 1-4 groups selected from halo, R³, C₁₋₆ optionally substituted alkenyl, amidine, guanidine, R³CO, COOR³, CONR³₂, OR³, NR³R³, SR³, SO₂R³NHCOR³, CN, and NHCONR³₂, wherein R³ is H, C₁₋₆ alkyl or aryl each of which is optionally substituted with R, OR halo, NR2, SR, SO2R, CN, or CF3, where each R is independently H or C₁-C₆ alkyl. More preferably, R¹ is naphthyl, indanyl, or 2,3-dihydrobenzofuranyl, each of which may be optionally substituted by 1-4 groups selected from halo, R³, C₁₋₆ optionally substituted alkenyl, amidine, guanidine, R³CO, COOR³, CONR³₂, OR³, NR³R³, SR³, SO₂R³NHCOR³, CN, and NHCONR³₂, wherein R³ is H, C₁₋₆ alkyl or aryl each of which is optionally substituted with R, OR halo, NR₂, SR, SO₂R, CN, or CF₃, where each R is independently H or C₁-C₆ alkyl.

In another embodiment of the compound described above, R¹ is a cyclic hydrocarbyl residue having 0-3 heteroatoms. In another embodiment, R¹ is an optionally substituted furanyl, thienyl, thiazolyl, or phenyl system having 0, 1, or 2 heterocyclic N atoms or naphthyl system having 0, 1, 2, or 3 heterocyclic N atoms, optionally substituted with halo, nitro, optionally substituted C₁₋₆alkyl or C₁₋₆alkenyl, guanidine CF₃, R³CO, COOR³, CONR³₂, SO₂NR³₂, —OOCR³, —NR³OCR³, —NR³OCOR³, NR³₂, OR³, or SR³, wherein R³ is H or C₁₋₆alkyl, phenyl, each optionally substituted with the foregoing substituents. In another embodiment, R¹ is methyl, naphthyl, fluoronaphthyl, 6-methoxynaphthnyl, benzoxy, phenyl, phenylethyl, ethylphenyl, hydroxyphenyl, phenylethenyl, ethenylphenyl, chlorophenylethenyl, bromophenyl, iodophenyl, fluorophenyl, chlorophenyl, dichlorophenyl, difluorophenyl, fluorochlorophenyl, bromofluorophenyl, methoxyphenyl, ethoxyphenyl, methylmethoxyphenyl, methylphenyl, dimethylphenyl, ethylphenyl, methylfluorophenyl, methyldifluorophenyl, dichloromethylphenyl, methylchlorophenyl, methylbromophenyl, cyclopropylphenyl, dimethylfuranyl, difluorothiophenyl, dimethylaminophenyl, quinoxalinyl, 3,4-dihydro-isoquinolinyl, benzodihydrofuranyl, benzofuranyl, benzo-1,2,3-thiadiazolyl, thienyl, benzo-dioxolanyl, benzodioxanyl, benzthiazole, trifluoromethylphenyl, trifluoromethoxyphenyl, di-trifluoromethyl phenyl, benzothienyl, benzochlorothienyl, thiomethylphenyl, thienylthiazolyl, fluorophenoxyisopropyl, N-sulfonyl phenylisoindolyl, benzofuranyl thiazolyl, benzodiazolyl, 4,5,6,7, tetrahydrobenzothienyl, benzocyclopentyl, benzocyclohexyl, N-methylisoindolyl, dimethoxyphenyl, trimethoxyphenyl, phenylthienyl, methylfuranyl, cyanophenyl, 9-oxofluorene, benzodifluorodioxolanyl, piperidinylmethyl, phenyl methylester.

In a more preferred embodiment R¹ is naphthyl, 2-bromonaphthyl, 6-methoxynaphthyl, benzoxy, phenyl, phenylethyl, phenylethenyl, 2-bromophenyl, 2-methylphenyl, 2-fluorophenyl, 3-chlorophenyl, 4-chlorophenyl, quinoxalinyl, 3,4-dihydroisoquinolinyl, or benzodihydrofuranyl.

In yet another embodiment, R¹ is optionally substituted phenyl, thienyl, furanyl, or thiazolyl.

In one aspect, R¹ is selected from the group consisting of

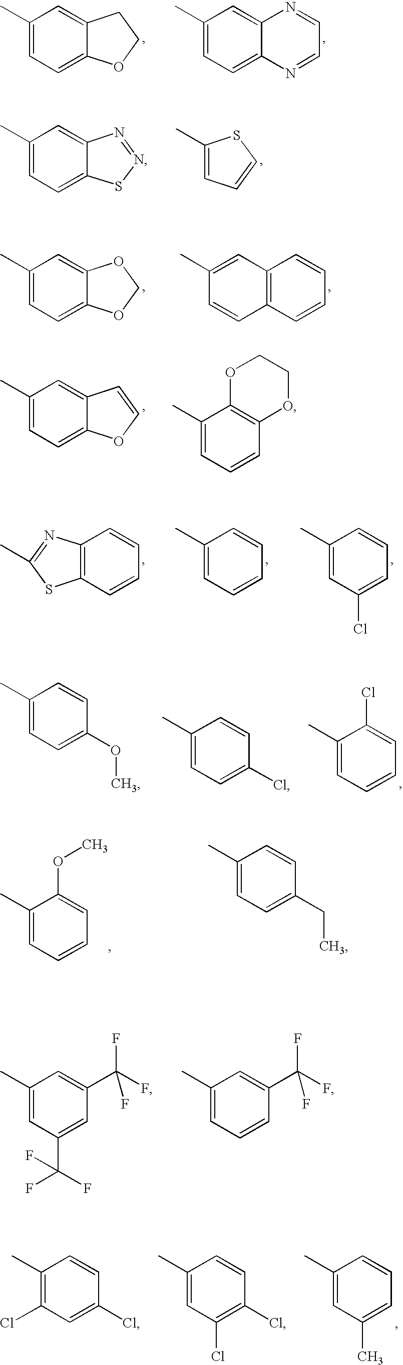

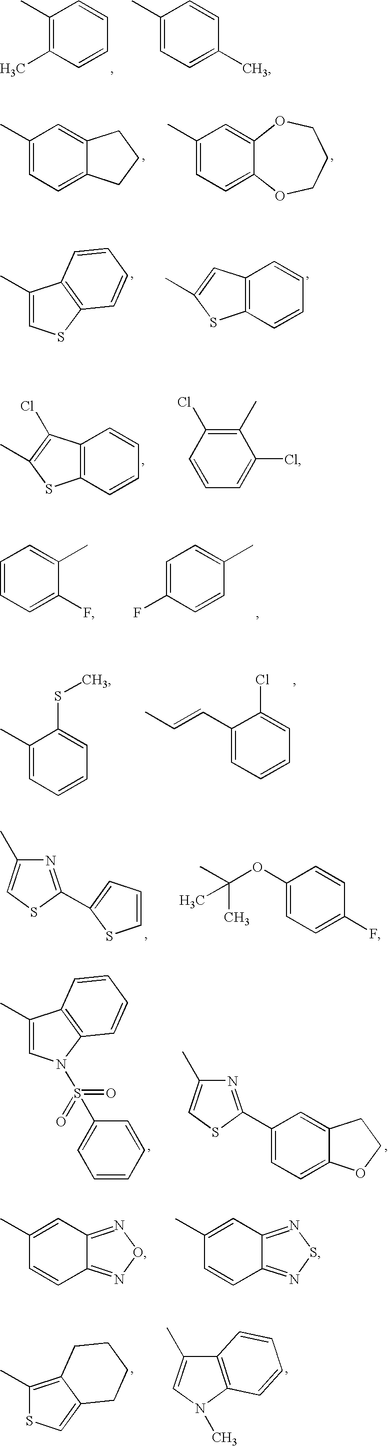

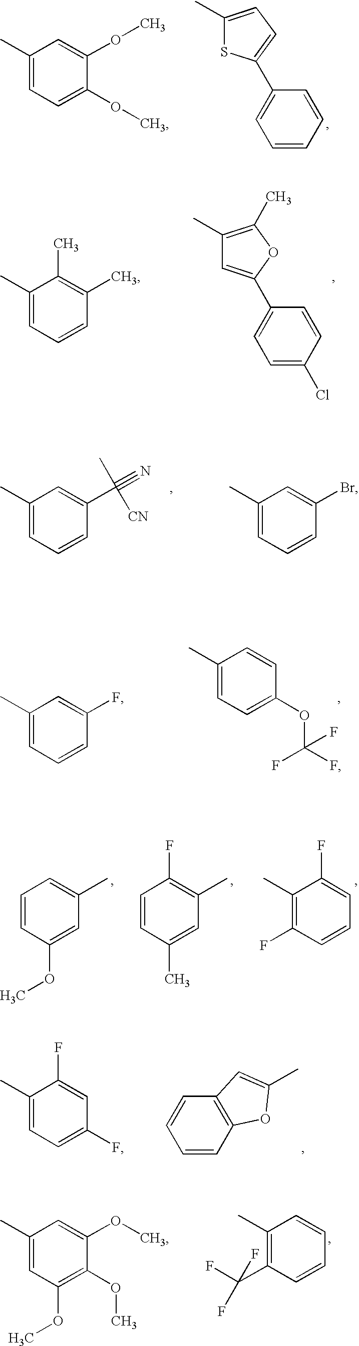

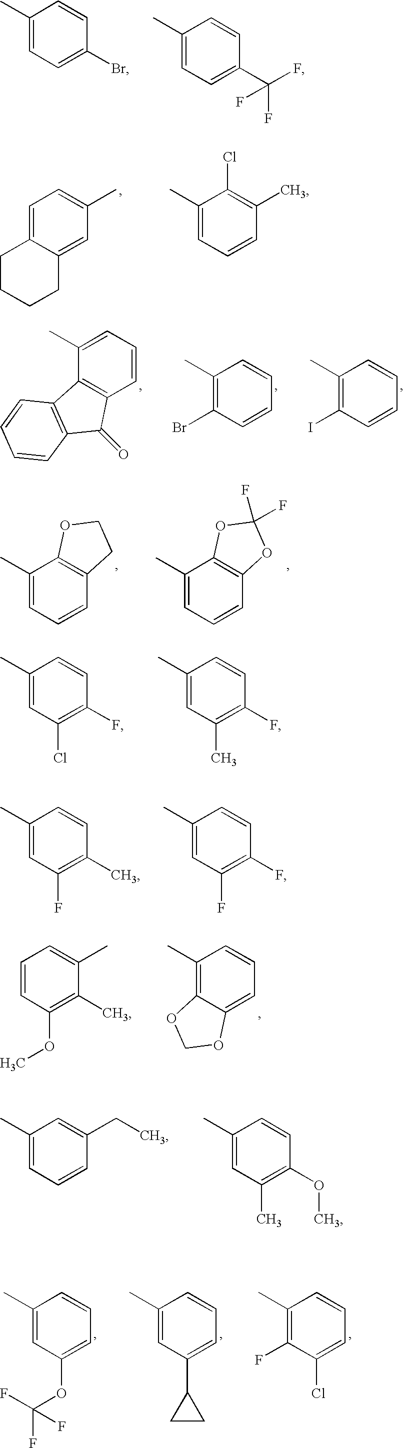

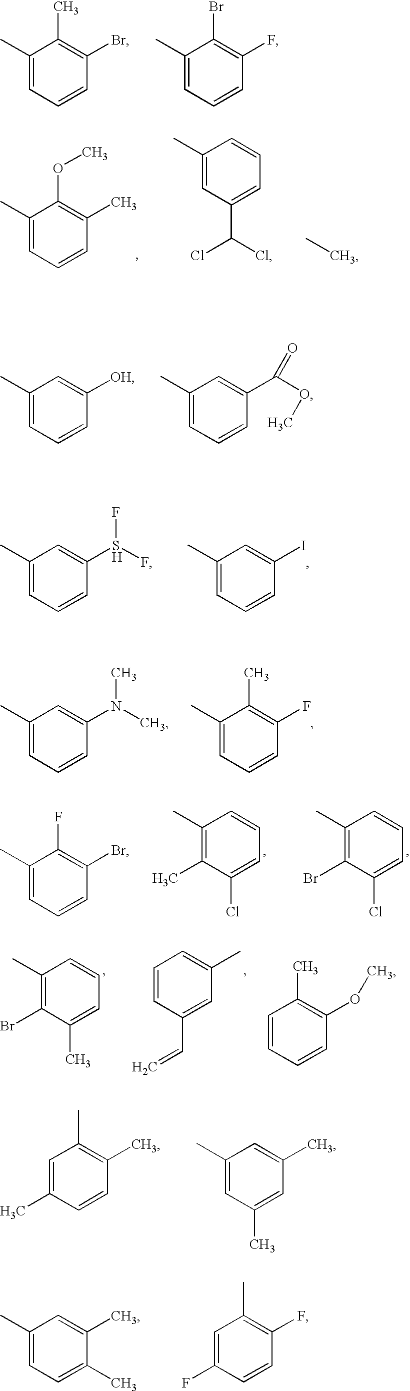

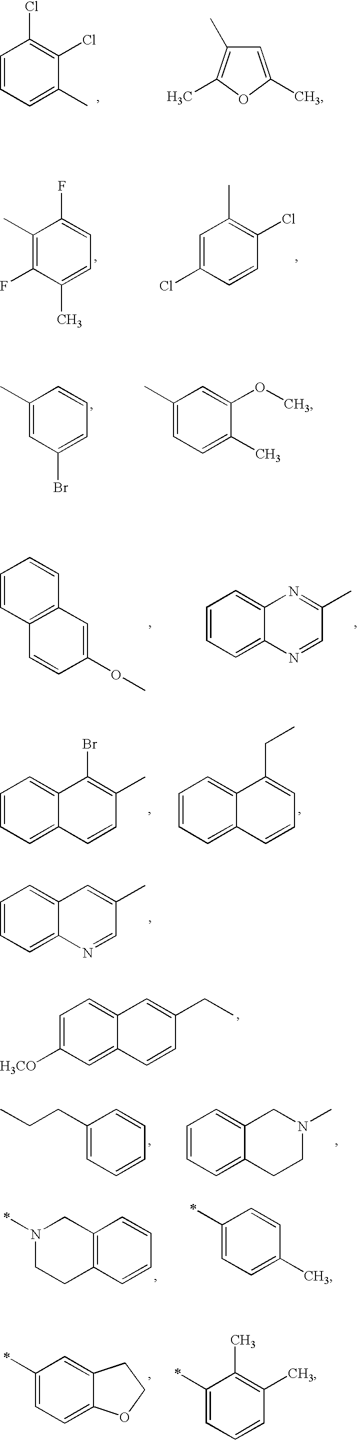

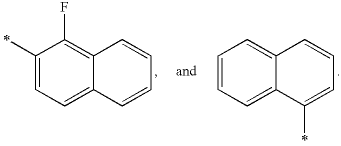

With respect to Y, Y is NH₂ or NR⁴R^{5,} preferably NHR⁵ or OR⁵, more preferably wherein R⁵ is C₁₋₁₀ alkyl, optionally substituted with a heterocyclic or hydrocarbyl ring or ring system. Preferably the hydrocarbyl or heterocyclic ring is cyclopropyl, cyclobutyl, cyclopentyl, cyclohexyl, pyrrolidinyl, piperidinyl, morpholinyl, phenyl, pyridinyl, napthalenyl, tetrahydronapthalenyl, indanyl, tetradrofuranyl, dihydro-furan-2-one, or tetrahydropyranyl. In another aspect R⁵ is C₁₋₁₀ alkyl substituted with a phenyl group. In another aspect of Y, the heterocyclic or hydrocarbyl ring or ring system is cyclopropyl, cyclobutyl, cyclopentyl, cyclohexyl, pyrrolidinyl, piperidinyl, morpholinyl, phenyl, pyridinyl, napthalenyl, tetrahydronapthalenyl, indanyl, tetradrofuranyl, dihydro-furan-2-one, or tetrahydropyranyl.

In another embodiment, Y is arylalkylamine. Preferably, Y is an optionally substituted phenylethylamine, and more preferably, Y is an optionally substituted 1-phenylethylamine. In one aspect, the substituted 1-phenylethylamine is of the S configuration. In another aspect the substituted 1-phenylethylamine is of the R configuration.

In another embodiment, Y is NR⁵R⁶ and more preferably, one of R⁵ or R⁶ is H, and the other of R⁵ or R⁶ is methylbenzyl, isopropyl, 4-hydroxy-cyclohexyl, cyclopropyl, methylcyclopropyl, N-benzyl-pyrrolidinyl, methylpiperidinyl-carbamic acid-tert butyl ester, methylpeperdinyl, pyrrolidinyl, cyclohexyl, cyclohexylamine, trihydropyranyl, methyl-fluorobenzyl, phenoxy, 4-pyridinyl, phenyl, hydroxyl, methoxy, or OR⁴, R⁴ is H or methyl.

In another embodiment, Y is NR⁵R⁶ where one of R⁵ or R⁶ is H and the other is methylbenzyl, isopropyl, or 4-hydroxy-cyclohexyl.

In one aspect, Y is

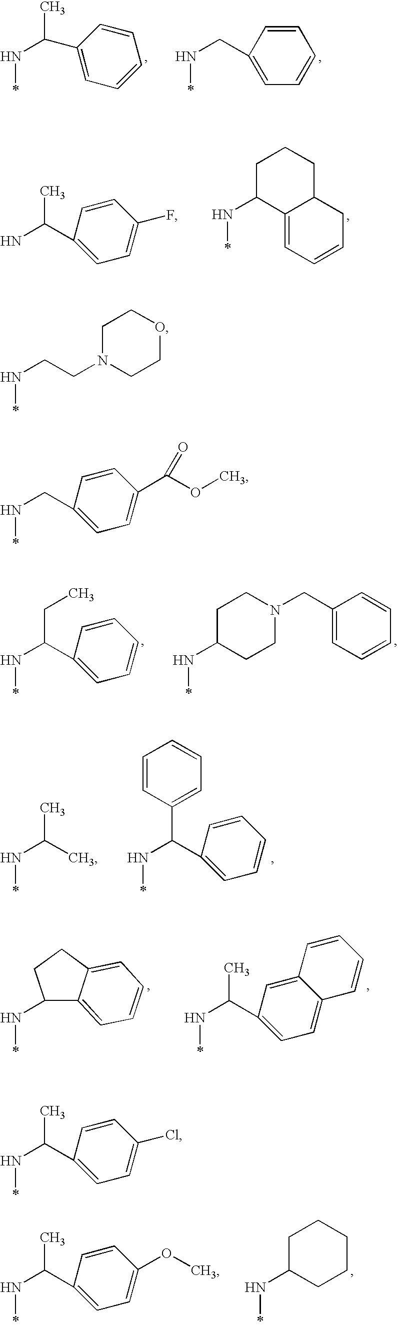

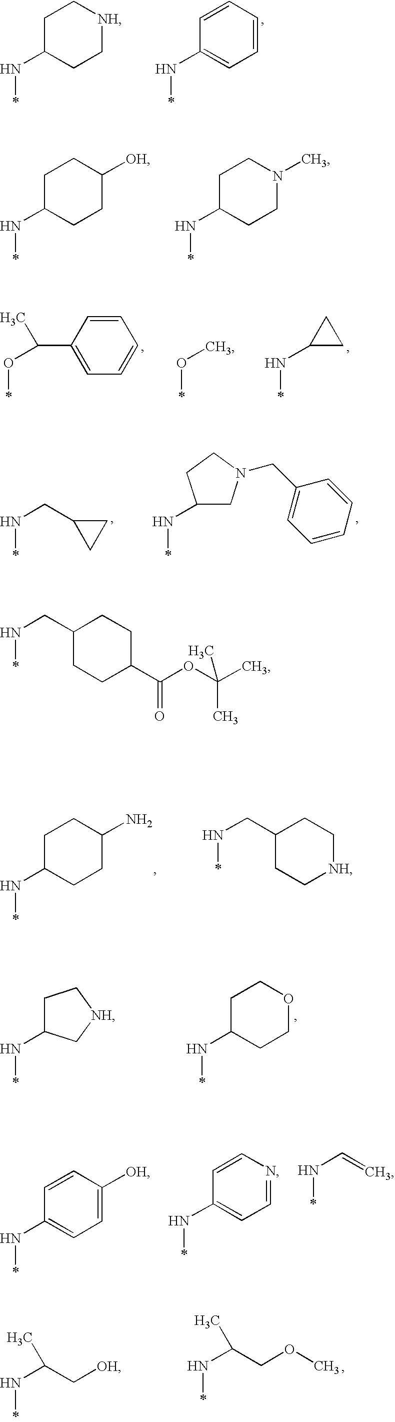

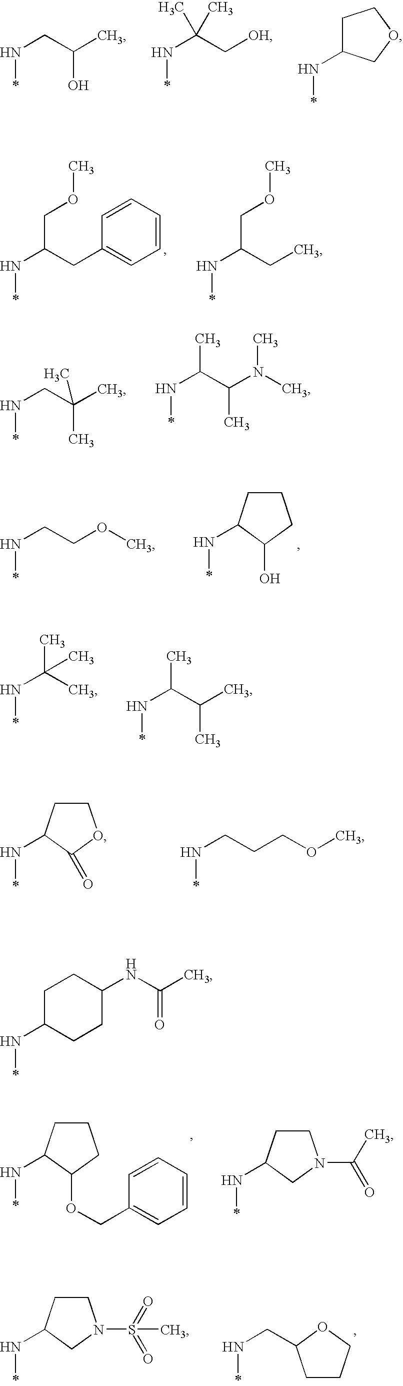

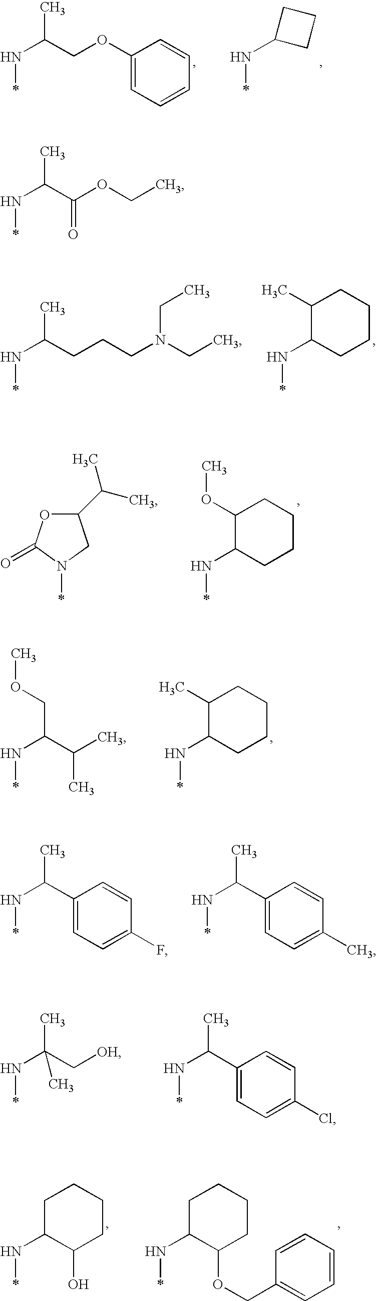

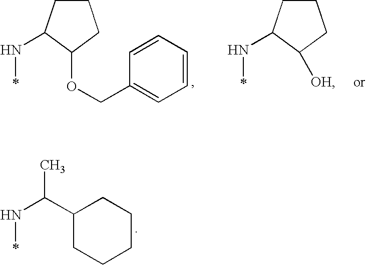

With resepct to R², preferably R² is a non-aromatic, alkyl-containing, group containing at least one N, such as piperidinylmethyl, pyrrolidyinylmethyl, or aminobutyl. Preferably R² is 4-piperidinylmethyl, 3-pyrrolidyinylmethyl, or 4-aminobutyl.

In another embodiment, R² is H, methyl, ethyl, 4-fluoro-benzyl, 4-piperidinyl, piperidinylmethyl, N-isopropylpiperidinylmethyl, N-cyclopentylpiperidinylmethyl, methylsulfanyl-benzyl, methanesulfinyl-benzyl, methanesulfonyl-benzyl, 2-amino-ethyl, 2-hydroxy-ethyl, t-butylamino-ethyl, methylamino-ethyl, isopropylamino-ethyl, or 3-methylazetidinyl. In a more particularly preferred embodiment R² is H, methyl, ethyl, 4-fluoro-benzyl, N-propylmorpholinyl, piperidinyl, methylpiperidinyl, 1-isopropylpiperidinyl, cyclopentylpiperidinylmethyl, methylpiperidinyl-isobutyl ester, methylsulfanyl-benzyl, methanesulfinyl-benzyl, methanesulfonyl-benzyl, amino-ethyl, hydroxyl-ethyl, t-butylamino-ethyl, methylamino-ethyl, isopropylamino-ethyl, 3-methylazetidinyl, ethoxy-glyoxyl peperdinyl.

In one aspect, R² is

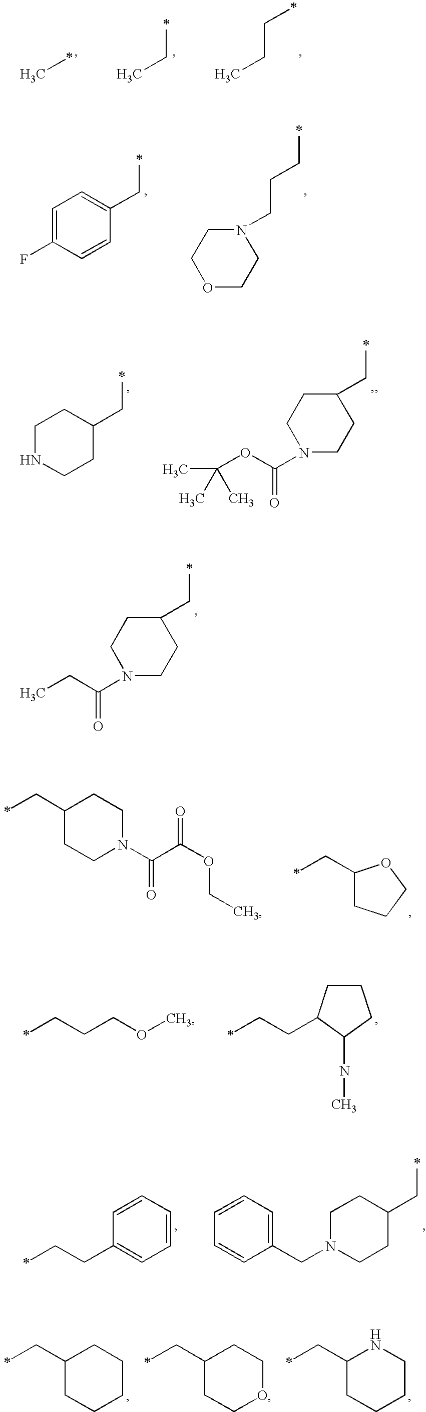

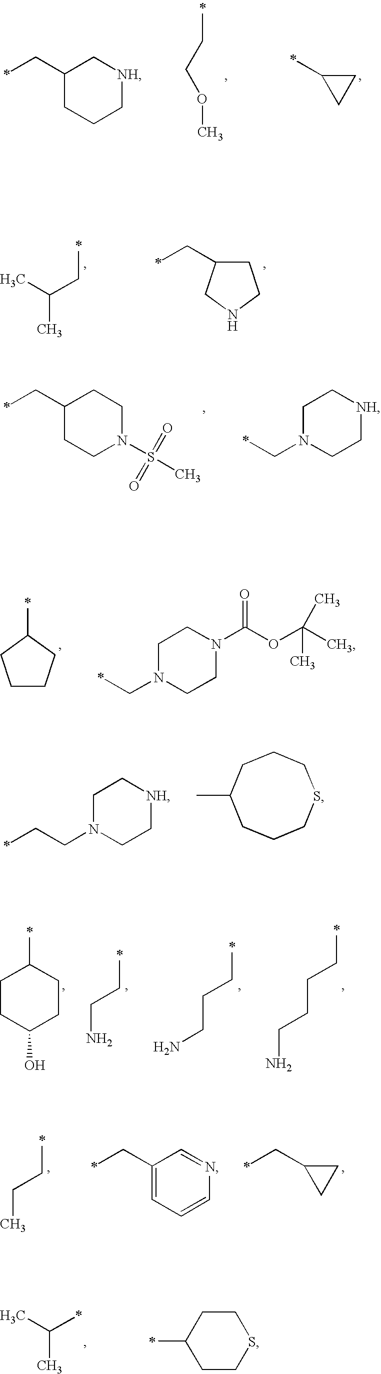

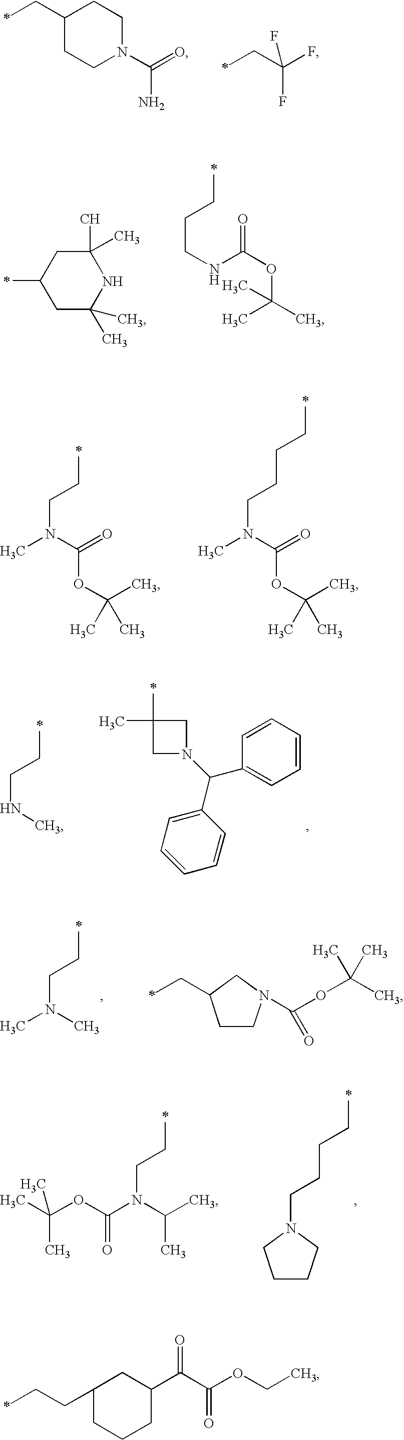

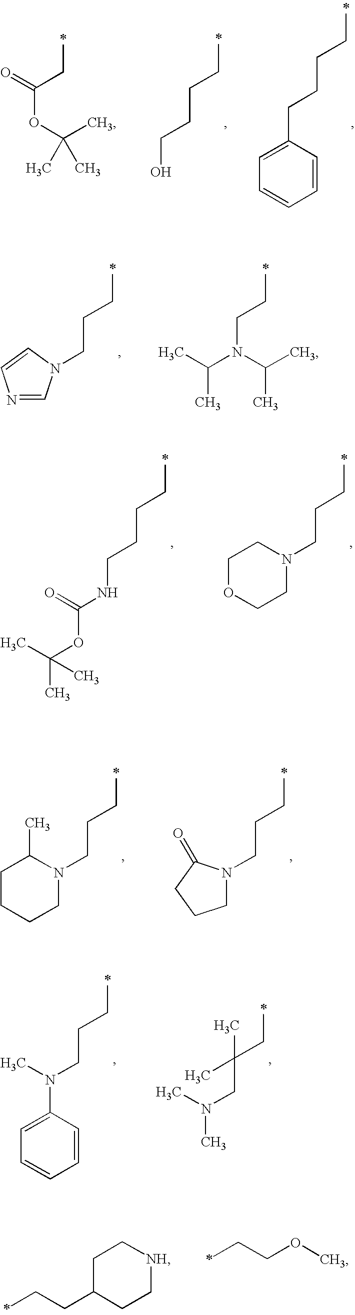

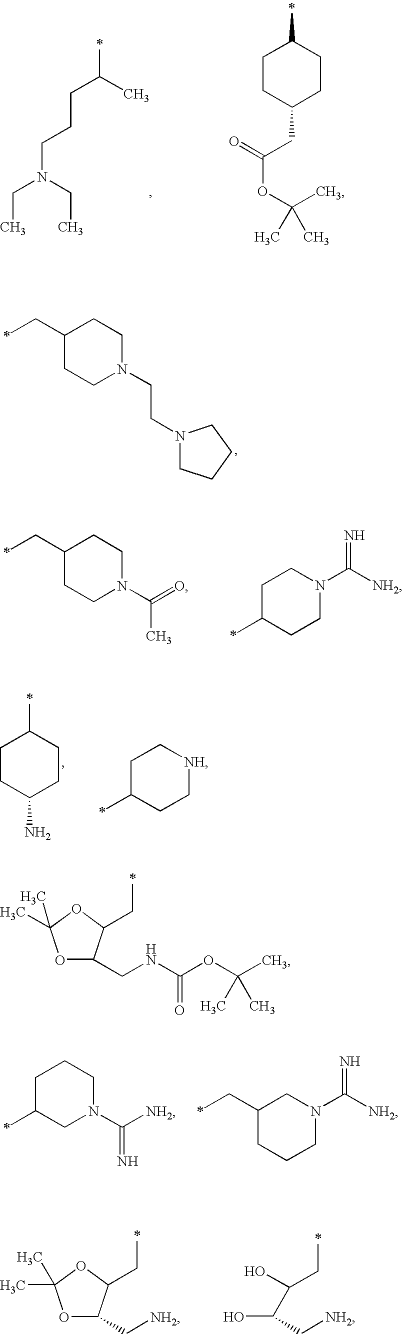

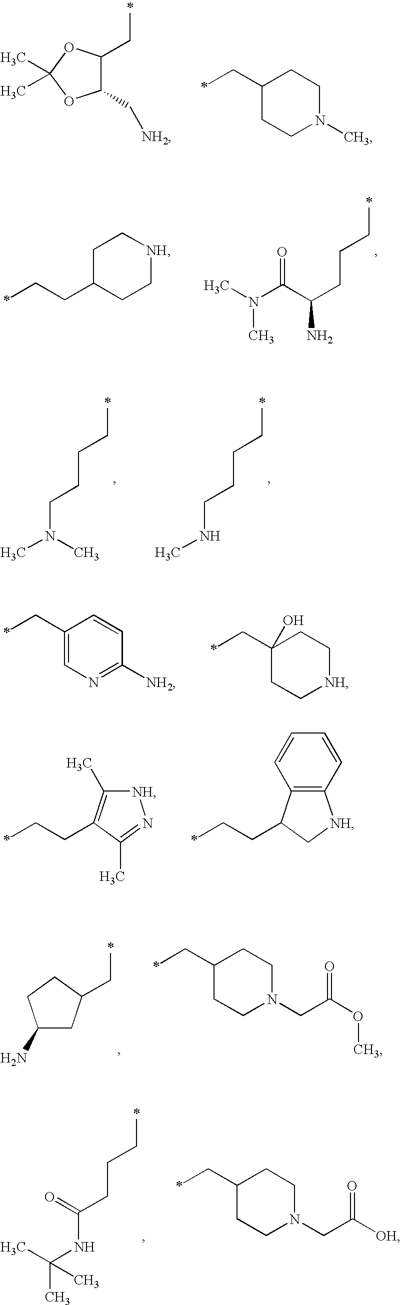

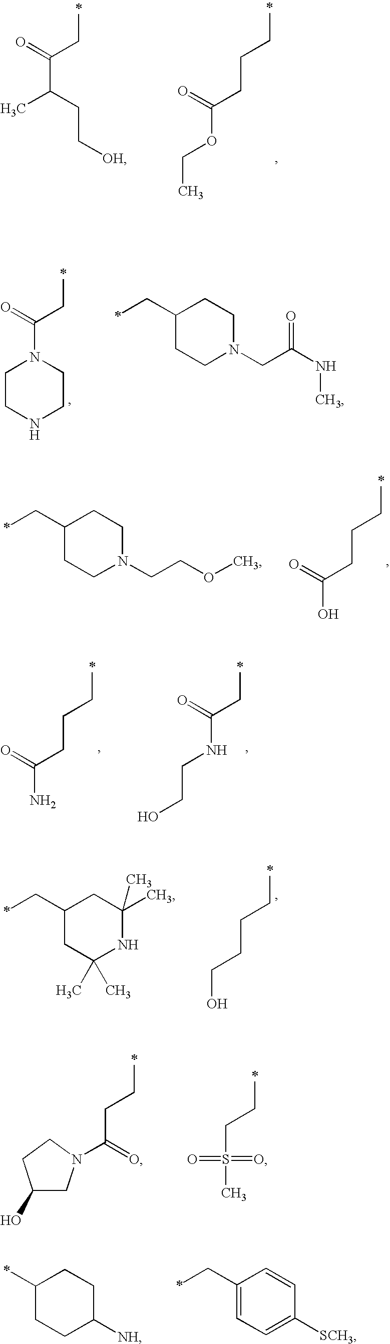

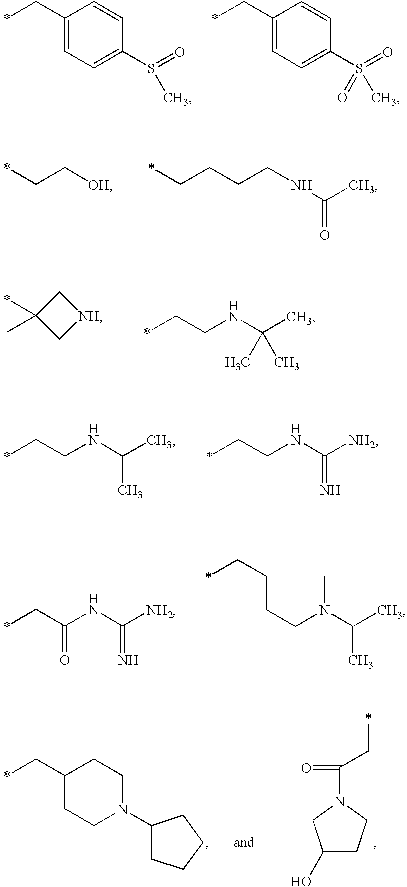

Exemplary substitutions for R¹, R² and Y can be found in Table 1 below.

The invention is also directed to a pharmaceutical composition for treating conditions characterized by enhanced p38-α activity which composition comprises a therapeutically effective amount of at least one compound described above and at least one pharmaceutically acceptable excipient. In one aspect, the composition further contains an additional therapeutic agent, such as a corticosteroid, a monoclonal antibody, or an inhibitor of cell division.

The invention is also directed to a method to treat a condition mediated by p38-α kinase comprising administering to a subject in need of such treatment a compound described above or a pharmaceutical composition thereof. In one aspect, the condition is a proinflammation response, such as multiple sclerosis, IBD, rheumatoid arthritis, rheumatoid spondylitis, osteoarthritis, gouty arthritis, other arthritic conditions, sepsis, septic shock, endotoxic shock, Gram-negative sepsis, toxic shock syndrome, asthma, adult respiratory distress syndrome, stroke, reperfusion injury, CNS injury, psoriasis, restenosis, cerebral malaria, chronic pulmonary inflammatory disease, chronic obstructive pulmonary disease, cystic fibrosis, silicosis, pulmonary sarcosis, bone fracture healing, a bone resorption disease, soft tissue damage, graft-versus-host reaction, Crohn's Disease, ulcerative colitis, Alzheimer's disease or pyresis.

In certain embodiments, L is a carbonyl. In others, it is SO₂. In one embodiment, when L is SO₂, R¹ is a bicyclic ring such as naphthalene.

As used herein, “hydrocarbyl residue” refers to a residue which contains only carbon and hydrogen. The residue may be aliphatic or aromatic, straight-chain, cyclic, branched, saturated or unsaturated or combinations thereof. The hydrocarbyl residue, when so stated however, may contain heteroatoms over and above the carbon and hydrogen members of the substituent residue. Thus, when specifically noted as containing such heteroatoms, the hydrocarbyl residue may contain heteroatoms within the “backbone” of the hydrocarbyl residue.

As used herein, “inorganic residue” refers to a residue that does not contain carbon. Examples include, but are not limited to, halo, hydroxy, NO₂ or NH₂.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-2 O, S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aryl” refers to an aromatic, heteroaromatic or partially aromatic or heteroaromatic ring system. “Aromatic” moiety refers to a monocyclic or fused bicyclic moiety such as phenyl or naphthyl; “heteroaromatic” also refers to monocyclic or fused bicyclic ring systems containing one or more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms. “Partially aromatic or heteroaromatic” refers to a portion of a ring system that has the characteristics of aromaticity in terms of electron distribution throughout at least one ring in a fused ring system, such as indanyl.

Similarly, “arylalkyl,” “arylalkenyl”, “heteroarylalkyl” and “heteroarylalkenyl” and the like refer to aromatic and heteroaromatic systems which are coupled to another residue through a carbon chain, including substituted or unsubstituted, saturated or unsaturated, carbon chains, typically of 1-6C. These carbon chains may also include a carbonyl group, thus making them able to provide substituents as an acyl moiety.

When the compounds of Formula I contain one or more chiral centers, the invention includes optically pure forms as well as mixtures of stereoisomers or enantiomers. For example, in one embodiment the R⁵ group on Y is a 1-phenylethyl amine, and the S enantiomer is preferred. For another embodiment, R⁵ is a 1-phenylethylamine of the R enantiomer.

The compounds of formula (I) may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (I), the compound may also be supplied as a salt with a pharmaceutically acceptable cation.

Synthesis of the Invention Compounds

The compounds of the invention may be synthesized by art-known methods. The following reaction schemes are illustrative:

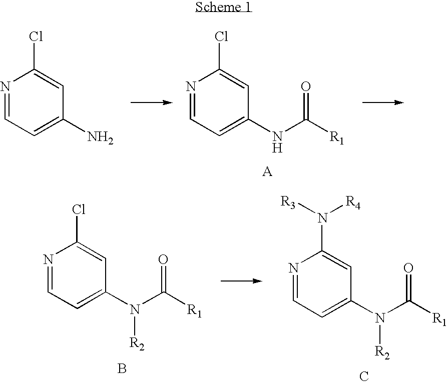

The 4-amino-2-chloropyridine can be converted to amide A by treatment with an appropriately substituted carbonyl chloride or carboxylic acid utilizing an amine base such as triethylamine or an inorganic base such as Na₂CO₃ in CH₂Cl₂ or DMF. A is treated with a base such as NaH in DMF followed by an appropriate alkyl halide to yield B. C is obtained by heating B with a primary or secondary amine in the presence of a palladium catalyst such as Pd(OAc)₂ or Pd₂(dba)₃, an inorganic base such as Cs₂CO₃ or an organic base like Na—O^{t}Bu in a solvent such as toluene or dioxane.

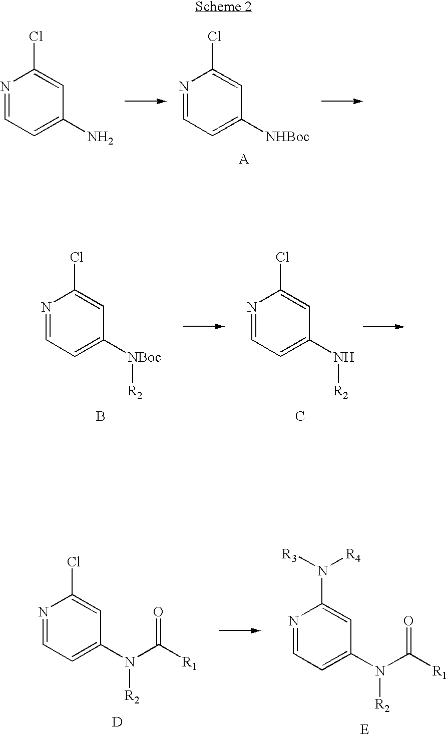

The 4-amino-2-chloropyridine is treated with NaHMDS and BOC₂O in THF to give the corresponding carbamate A. A can then be treated with NaH in DMF followed by the addition of an appropriate alkyl halide to yield B. This is followed by treatment with HCl in dioxane to give C. D is obtained by treating C with an appropriately substituted carbonyl chloride using an amine base such as triethylamine or an inorganic base such as Na₂CO₃ in CH₂Cl₂ or DMF. E is obtained by heating D with a primary or secondary amine in the presence of a palladium catalyst such as Pd(OAc)₂ or Pd₂(dba)₃, an inorganic base such as Cs₂CO₃ or an organic base like Na—O^{t}Bu in a solvent such as toluene or dioxane.

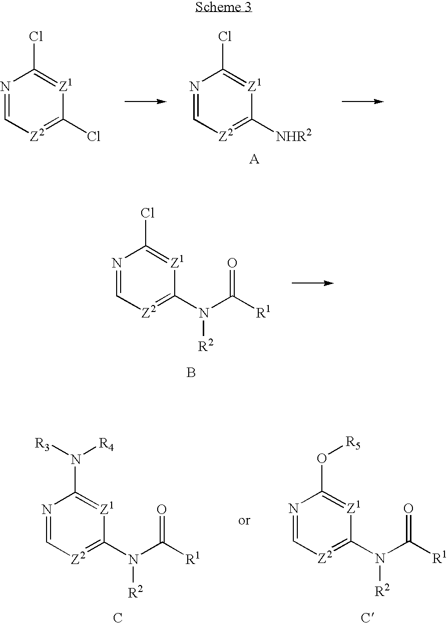

An appropriately substituted primary amine is added to the 2,4-dichloroheterocycle and an inorganic base such as K₂CO₃ in DMF at 60° C. After warming to RT A is obtained. A is treated with a base such as NaH in DMF followed by addition of an appropriately substituted carbonyl chloride to provide B. Compound C is secured by treating B with a primary or secondary amine in the presence of a palladium catalyst such as Pd(OAc)₂ or Pd₂(dba)₃, an inorganic base such as Cs₂CO₃ or an organic base like Na—O^{t}Bu in a solvent such as toluene or dioxane. Alternatively C or C′ can be obtained through heating B with an appropriate amine or alcohol in NMP. 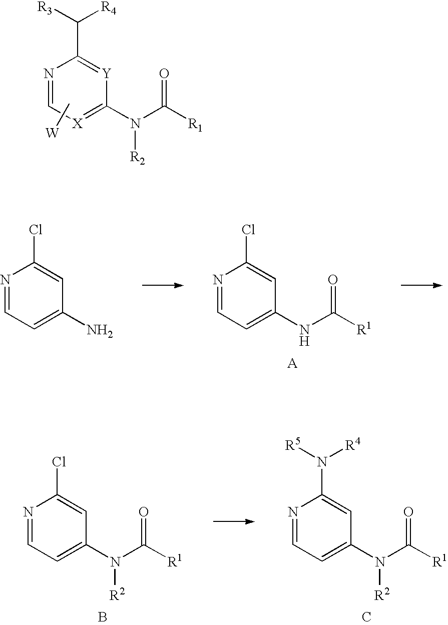
Assays for p38 α Kinase Inhibition

For each of the assay procedures described below, the TNF-α production correlates to the activity of p38-α kinase.

A. Human Whole Blood Assay for p38 Kinase Inhibition

Venous blood is collected from healthy male volunteers into a heparinized syringe and is used within 2 hours of collection. Test compounds are dissolved in 100% DMSO and 1 μl aliquots of drug concentrations ranging from 0 to 1 mM are dispensed into quadruplicate wells of a 24-well microtiter plate (Nunclon Delta SI, Applied Scientific, So. San Francisco, Calif.). Whole blood is added at a volume of 1 ml/well and the mixture is incubated for 15 minutes with constant shaking (Titer Plate Shaker, Lab-Line Instruments, Inc., Melrose Park, Ill.) at a humidified atmosphere of 5% CO₂ at 37° C. Whole blood is cultured either undiluted or at a final dilution of 1:10 with RPMI 1640 (Gibco 31800+NaHCO₃, Life Technologies, Rockville, Md. and Scios, Inc., Sunnyvale, Calif.). At the end of the incubation period, 10 μl of LPS (E. coli 0111:B4, Sigma Chemical Co., St. Louis, Mo.) is added to each well to a final concentration of 1 or 0.1 μg/ml for undiluted or 1:10 diluted whole blood, respectively. The incubation is continued for an additional 2 hours. The reaction is stopped by placing the microtiter plates in an ice bath and plasma or cell-free supernates are collected by centrifugation at 3000 rpm for 10 minutes at 4° C. The plasma samples are stored at −80° C. until assayed for TNF-α levels by ELISA, following the directions supplied by Quantikine Human TNF-α assay kit (R&D Systems, Minneapolis, Minn.).

IC₅₀ values are calculated using the concentration of inhibitor that causes a 50% decrease as compared to a control. IC50 values can be determined with curve-fitting plots available with common software packages. Approximate IC50 values can be calculated using formula:
IC₅₀ (app)=A×i/(1−A)

where A=fractional activity and i=total inhibitor concentration.

B. Enriched Mononuclear Cell Assay for p38 Kinase Inhibition

The enriched mononuclear cell assay, the protocol of which is set forth below, begins with cryopreserved Human Peripheral Blood Mononuclear Cells (HPBMCs) (Clonetics Corp.) that are rinsed and resuspended in a warm mixture of cell growth media. The resuspended cells are then counted and seeded at 1×10⁶ cells/well in a 24-well microtitre plate. The plates are then placed in an incubator for an hour to allow the cells to settle in each well.

After the cells have settled, the media is aspirated and new media containing 100 ng/ml of the cytokine stimulatory factor Lipopolysaccharide (LPS) and a test chemical compound is added to each well of the microtiter plate. Thus, each well contains HPBMCs, LPS and a test chemical compound. The cells are then incubated for 2 hours, and the amount of the cytokine Tumor Necrosis Factor Alpha (TNF-α) is measured using an Enzyme Linked Immunoassay (ELISA). One such ELISA for detecting the levels of TNF-α is commercially available from R&D Systems. The amount of TNF-α production by the HPBMCs in each well is then compared to a control well to determine whether the chemical compound acts as an inhibitor of cytokine production.

LPS Induced Cytokine Synthesis in HPBMCS

Cryopreserved HPBMC (cat#CC-2702 Clonetics Corp)

LGM-3 media (cat#CC-3212 Clonetics Corp)

LPS stock 10 μg/ml (Cat. No. L 2630 serotype 0111:B4 Sigma)

Human TNF-α ELISA (R&D Systems)

DNase I (10 mg/ml stock)

Preparation of Cells.

LGM-3 media warmed to 37° C.

5 μl of DNase I stock added to 10 ml media.

Cells thawed rapidly and dispersed into above.

Centrifuge 200×g×10 min (room temperature.

Pellet up in 10 ml sterile PBS.

Centrifuge 200×g×10 min @ room temperature.

Pellet resuspended in 10 ml LGM-3 then diluted to 50 ml with LGM-3.

Perform cell count.

Adjust to 1×E06 cells/well.

Seed 1 ml/well of a 24 well plate.

Place plate in incubator to plate down for 1 hour.

Preparation of Incubation Media.

LGM-3 containing 100 ng/ml LPS (e.g. 50 ml media plus 0.5 ml LPS stock)

Aliquot into 2 ml aliquots and add 1000× inhibitor dilutions.

Incubation

When cells have plated down, aspirate media away and overlay with 1 ml relevant incubation media. Return plate to incubator for 2 hours or 24 hours. Remove supernatants after incubation to a labeled tube and either perform TNF (or other) ELISA immediately or freeze for later assay.

IC₅₀ values are calculated using the concentration of inhibitor that causes a 50% decrease as compared to a control.

Administration and Use

The compounds of the invention are useful among other indications in treating conditions associated with inflammation. Thus, the compounds of formula (I) or their pharmaceutically acceptable salts are used in the manufacture of a medicament for prophylactic or therapeutic treatment of mammals, including humans, in respect of conditions characterized by excessive production of cytokines and/or inappropriate or unregulated cytokine activity.

The compounds of the invention inhibit the production of cytokines such as TNF, IL-1, IL-6 and IL-8, cytokines that are important proinflammatory constituents in many different disease states and syndromes. Thus, inhibition of these cytokines has benefit in controlling and mitigating many diseases. The compounds of the invention are shown herein to inhibit a member of the MAP kinase family variously called p38 MAPK (or p38), CSBP, or SAPK-2. The activation of this protein has been shown to accompany exacerbation of the diseases in response to stress caused, for example, by treatment with lipopolysaccharides or cytokines such as TNF and IL-1. Inhibition of p38 activity, therefore, is predictive of the ability of a medicament to provide a beneficial effect in treating diseases such as Alzheimer's, coronary artery disease, congestive heart failure, cardiomyopathy, myocarditis, vasculitis, restenosis, such as occurs following coronary angioplasty, atherosclerosis, IBD, rheumatoid arthritis, rheumatoid spondylitis, osteoarthritis, gouty arthritis and other arthritic conditions, multiple sclerosis, acute respiratory distress syndrome (ARDS), asthma, chronic obstructive pulmonary disease (COPD), chronic pulmonary inflammatory disease, cystic fibrosis, silicosis, pulmonary sarcosis, sepsis, septic shock, endotoxic shock, Gram-negative sepsis, toxic shock syndrome, heart and brain failure (stroke) that are characterized by ischemia and reperfusion injury, surgical procedures, such as transplantation procedures and graft rejections, cardiopulmonary bypass, coronary artery bypass graft, CNS injuries, including open and closed head trauma, inflammatory eye conditions such as conjunctivitis and uveitis, acute renal failure, glomerulonephritis, inflammatory bowel diseases, such as Crohn's disease or ulcerative colitis, graft vs. host disease, bone fracture healing, bone resorption diseases like osteoporosis, soft tissue damage, type II diabetes, pyresis, psoriasis, cachexia, viral diseases such as those caused by HIV, CMV, and Herpes, and cerebral malaria.

Within the last several years, p38 has been shown to comprise a group of MAP kinases designated p38-α, p38-β, p38-γ and p38-δ. Jiang, Y., et al., J Biol Chem (1996) 271:17920-17926 reported characterization of p38-β as a 372-amino acid protein closely related to p38-α. In comparing the activity of p38-α with that of p38-δ, the authors state that while both are activated by proinflammatory cytokines and environmental stress, p38-β was preferentially activated by MAP kinase kinase-6 (MKK6) and preferentially activated transcription factor 2, thus suggesting that separate mechanisms for action may be associated with these forms.

Kumar, S., et al., Biochem Biophys Res Comm (1997) 235:533-538 and Stein, B., et al., J Biol Chem (1997) 272:19509-19517 reported a second isoform of p38-β, p38-β2, containing 364 amino acids with 73% identity to p38-α. All of these reports show evidence that p38-β is activated by proinflammatory cytokines and environmental stress, although the second reported p38-β isoform, p38-β2, appears to be preferentially expressed in the CNS, heart and skeletal muscle compared to the more ubiquitous tissue expression of p38-α. Furthermore, activated transcription factor-2 (ATF-2) was observed to be a better substrate for p38-β2 than for p38-α, thus suggesting that separate mechanisms of action may be associated with these forms. The physiological role of p38-β1 has been called into question by the latter two reports since it cannot be found in human tissue and does not exhibit appreciable kinase activity with the substrates of p38-α.

The identification of p38-γ was reported by Li, Z., et al., Biochem Biophys Res Comm (1996) 228:334-340 and of p38-6 by Wang, X., et al., J Biol Chem (1997) 272:23668-23674 and by Kumar, S., et al., Biochem Biophys Res Comm (1997) 235:533-538. The data suggest that these two p38 isoforms (γ and δ) represent a unique subset of the MAPK family based on their tissue expression patterns, substrate utilization, response to direct and indirect stimuli, and susceptibility to kinase inhibitors.

The manner of administration and formulation of the compounds useful in the invention and their related compounds will depend on the nature of the condition, the severity of the condition, the particular subject to be treated, and the judgment of the practitioner; formulation will depend on mode of administration. As the compounds of the invention are small molecules, they are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of 5%-95% of the total formulation, but wide variation is permitted depending on the carrier. Suitable carriers include sucrose, pectin, magnesium stearate, lactose, peanut oil, olive oil, water, and the like.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically, for topical conditions such as psoriasis, or in formulation intended to penetrate the skin. These include lotions, creams, ointments and the like which can be formulated by known methods.

The compounds may also be administered by injection, including intravenous, intramuscular, subcutaneous or intraperitoneal injection. Typical formulations for such use are liquid formulations in isotonic vehicles such as Hank's solution or Ringer's solution.

Alternative formulations include nasal sprays, liposomal formulations, slow-release formulations, and the like, as are known in the art.

Any suitable formulation may be used. A compendium of art-known formulations is found in Remington's Pharmaceutical Sciences, latest edition, Mack Publishing Company, Easton, Pa. Reference to this manual is routine in the art.

The dosages of the compounds of the invention will depend on a number of factors which will vary from patient to patient. However, it is believed that generally, the daily oral dosage will utilize 0.001-100 mg/kg total body weight, preferably from 0.01-50 mg/kg and more preferably about 0.01 mg/kg-10 mg/kg. The dose regimen will vary, however, depending on the conditions being treated and the judgment of the practitioner.

It should be noted that the compounds of formula (I) can be administered as individual active ingredients, or as mixtures of several embodiments of this formula. In addition, the inhibitors of p38 kinase can be used as single therapeutic agents or in combination with other therapeutic agents. Drugs that could be usefully combined with these compounds include natural or synthetic corticosteroids, particularly prednisone and its derivatives, monoclonal antibodies targeting cells of the immune system, antibodies or soluble receptors or receptor fusion proteins targeting immune or non-immune cytokines, and small molecule inhibitors of cell division, protein synthesis, or mRNA transcription or translation, or inhibitors of immune cell differentiation or activation.

As implied above, although the compounds of the invention may be used in humans, they are also available for veterinary use in treating animal subjects.

The following examples are intended to illustrate but not to limit the invention, and to illustrate the use of the above Reaction Schemes.

### EXAMPLE 1

### Preparation of Naphthalene-2-carboxylic acid methyl-[2-(1S-phenyl-ethylamino)-pyridin-4-yl]-amide

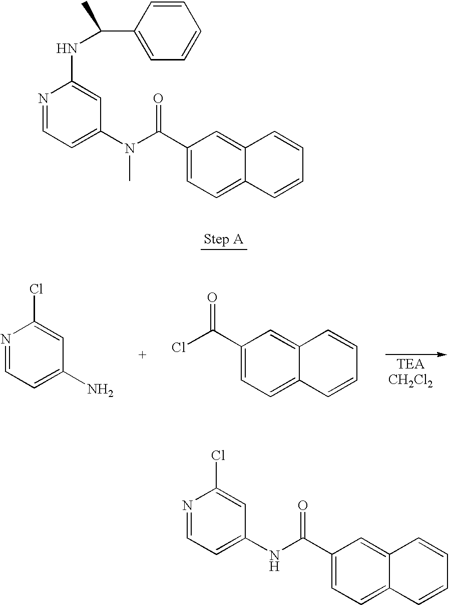

To a stirring solution of 4-amino-2-chloropyridine (3 g, 23.3 mmol) and TEA (3.25 mL, 23.3 mmol) in anhydrous CH₂Cl₂ (93 mL) at 0° C. was added 2-napththoyl chloride (4.9 g, 25.7 mmol), dropwise. The solution was stirred overnight, during which time the temperature was allowed to reach room temperature. The CH₂Cl₂ was removed, under reduce pressure, and the residue was redissolved in EtOAc (60 mL) and washed with water (3×40 mL), followed by brine. The formation of precipitate followed and was collected by filtration and placed under vacuum overnight. 2.5 g of the target compound were obtained (38%). M+H⁺ (283).

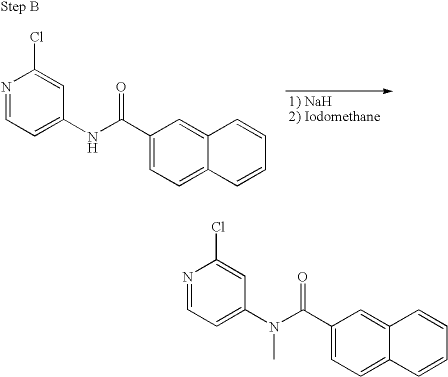

To a stirring solution of naphthalene-2-carboxylic acid (2-chloro-pyridin-4-yl)-amide (40 mg, 0.14 mmol) in DMF (0.56 mL) at 0° C. was added NaH (6 mg, 0.15 mmol). The slurry was stirred for 30 minutes, followed by addition of iodomethane (9 μL, 0.14 mmol). Stirring was continued overnight and the temperature was allowed to reach room temperature. The reaction was quenched with the addition of water and extracted with EtOAc, washed with water and brine and dried over Na₂SO₄ and concentrated under reduced pressure. The residue was purified by radial chromatography on silica gel eluting with 25% EtOAc/hexanes to yield 23.5 mg (57%). M+H⁺ (297).

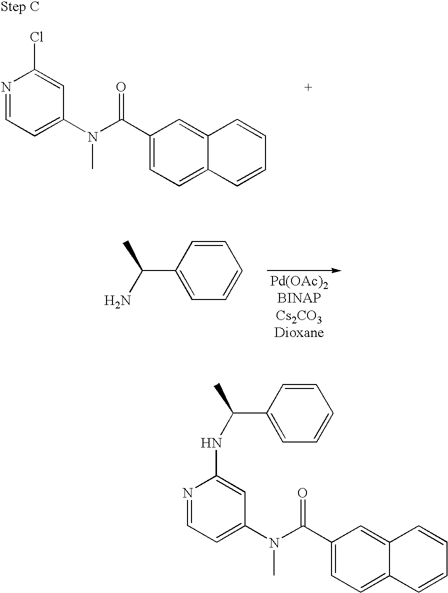

A reaction tube containing dioxane (0.2 mL) was charged with naphthalene-2-carboxylic acid (2-chloro-pyridin-4-yl)-amide (22 mg, 0.07 mmol), Pd(OAc)₂ (1 mg, 0.004 mmol) and BINAP (3.5 mg, 0.004 mmol) and prestirred at room temperature for 15 minutes. Then, Cs₂CO₃ (34 mg, 0.1 mmol) and α-methylbenzylamine (13 μL, 0.1 mmol) were added to the suspension and the tube was sealed and heated to 94° C. overnight. The reaction mixture was filtered and the dioxane stripped under reduced pressure. The residue was purified by preparative tlc on silica gel eluting with 30% EtOAc/hexanes to yield 1.8 mg (8%). M+H⁺ (382).

### EXAMPLE 2

### Preparation of Naphthalene-2-carboxylic acid ethyl-[2-(1S-phenyl-ethylamino)-pyridin-4-yl]-amide

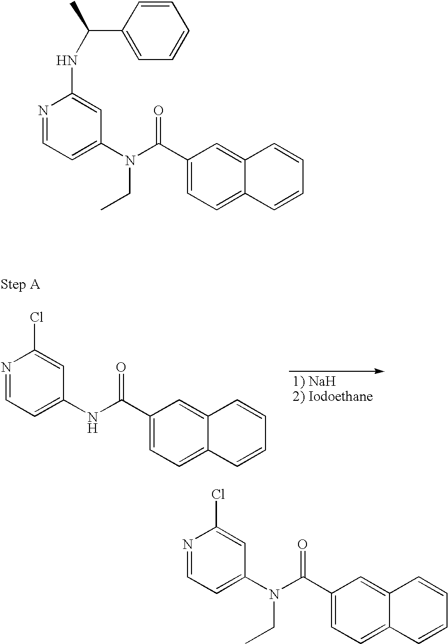

Prepared similarly to Example 1 (step B) with a 27% yield. M+H⁺ (311).

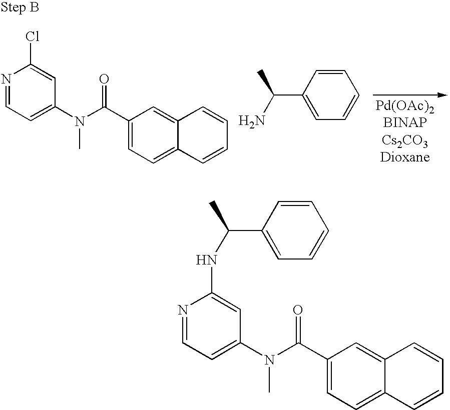

Prepared similarly to Example 1 (Step C) with a 71% yield. M+H⁺ (396).

### EXAMPLE 3

### Preparation of (4-Fluoro-benzyl)-[2-(1S-phenyl-ethylamino)-pyridin-4-yl]-carbamic acid benzyl ester

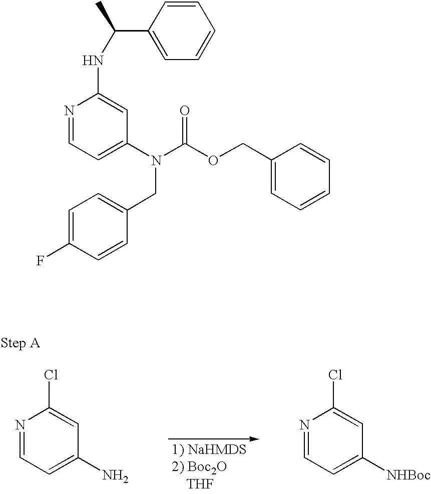

To a solution containing 4-amino-2-chloropyridine (3.05 g, 23.72 mmol) in THF (24 mL) was added sodium bis(trimethylysilyl)amide (47.45 mmol) and stirred at room temperature for 30 minutes. To this solution was added Boc₂O (23.72 mmol) and the gelatinous mixture was stirred overnight. The reaction was diluted with water and extracted with EtOAc. The combined organic phase was washed with water and brine and dried over Na₂SO₄ and concentrated to yield 4.17 g (77%). M+H⁺ (230).

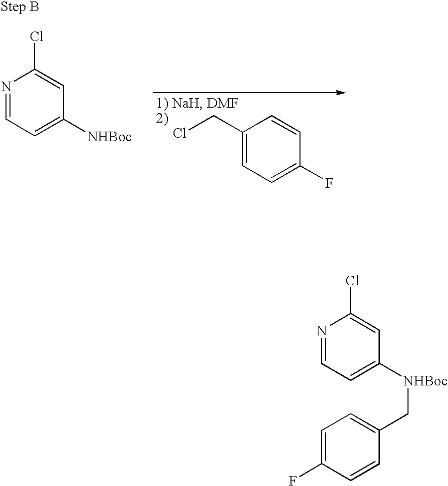

To a stirring solution of naphthalene-2-carboxylic acid (2-chloro-pyridin-4-yl)-amide (4.17 g, 18.25 mmol) in DMF (72 mL) was added NaH (0.8 g, 20.08 mmol). The slurry was stirred for one hour and cooled to 0° C., at which time 4-fluorobenzyl chloride (2.3 mL, 19.16 mmol) was added. The mixture continued stirring overnight and the temperature was allowed to reach room temperature. The reaction was quenched with the addition of water and extracted with EtOAc, washed with water and brine and dried over Na₂SO₄ and concentrated under reduced pressure. The residue was purified by flash chromatography on silica gel eluting with 25% EtOAc/hexanes to yield 4.57 g (74%). M+H⁺ (338).

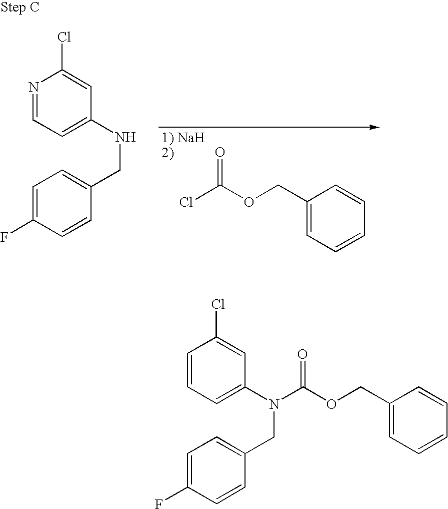

To a stirring solution of the substrate (278 mg, 1.17 mmol) in DMF (4.1 mL), at room temperature, was added NaH (94 mg, 2.35 mmol) and stirred for one hour. The solution was cooled to 0° C. and phenyl chloroformate (0.2 mL, 1.64 mmol) was added and stirring continued overnight, during which time the temperature of the mixture was allowed to reach room temperature. The reaction was quenched with water, extracted with EtOAc, washed with water and brine and dried over Na₂SO₄ and concentrated. The residue was purified by radial chromatography eluting with 30% EtOAc/hexanes to yield a colorless oil weighing 76 mg (10%). M+H⁺ (372).

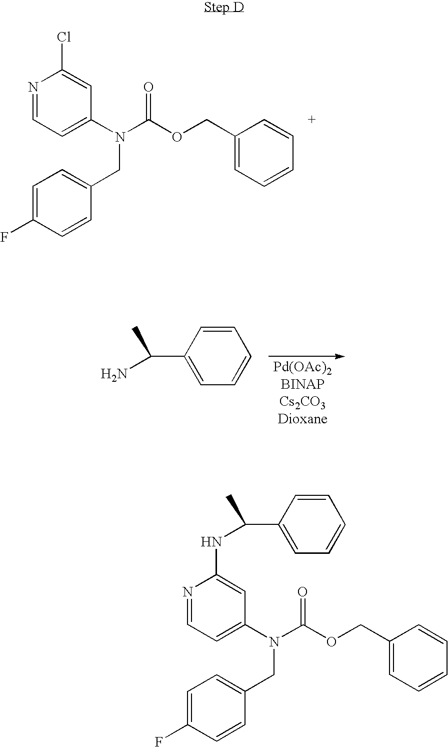

Prepared similarly to Example 1 (Step C) with a 11% yield. M+H⁺ (456).

### EXAMPLE 4

### Preparation of N-Methyl-N-[2-(1S-phenyl-ethylamino)-pyridin-4-yl]-benzamide

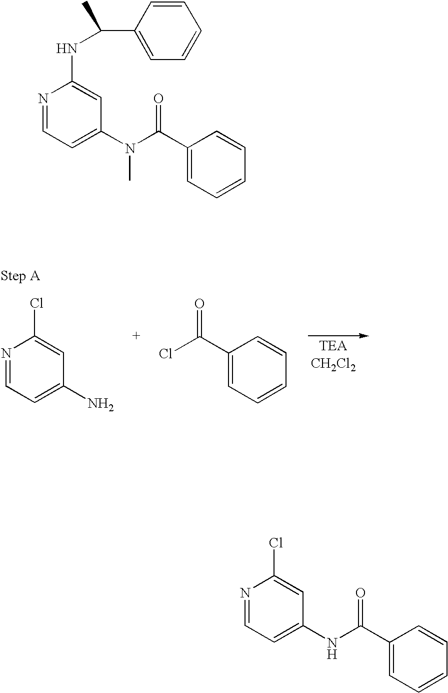

Charged round-bottom containing CH₂Cl₂ (31 mL) at 0° C. with 2-chloro-4-aminopyridine (1 g, 7.78 mmol) and TEA (1.08 mL, 7.78 mmol) and added benzoyl chloride (1 mL, 8.56 mmol). Stirring was continued overnight whereupon the temperature of the mixture was allowed to reach room temperature.

The transparent, yellow solution was diluted with CH₂Cl₂ (10 mL) and washed with water (2×30 mL) and brine, dried over Na₂SO₄ and concentrated. The residue was purified by preparative column chromatography on silica gel eluting with EtOAc/hexanes and yielding 1.06 g pink solid (59%). M+H⁺ (234).

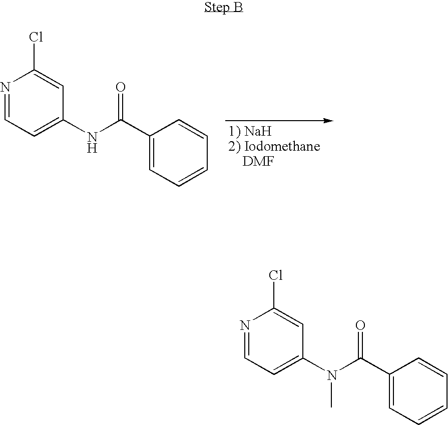

Prepared similarly to Example 1 (step B) with a 30% yield. M+H⁺ (247).

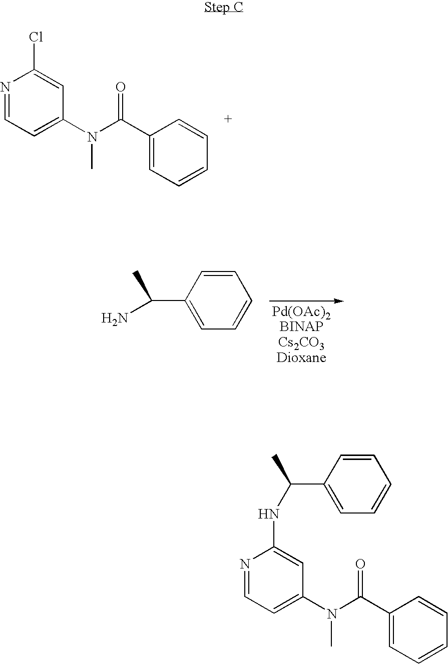

Prepared similarly to Example 1 (Step C) with a 72% yield. M+H⁺ (331).

### EXAMPLE 5

### Preparation of N-Ethyl-N-[2-(1S-phenyl-ethylamino)-pyridin-4-yl]-benzamide

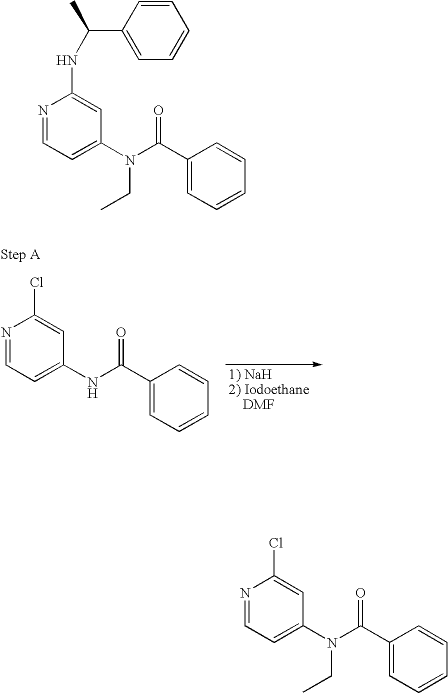

Prepared similarly to Example 3 (Step B) with an 18% yield. M+H⁺ (262).

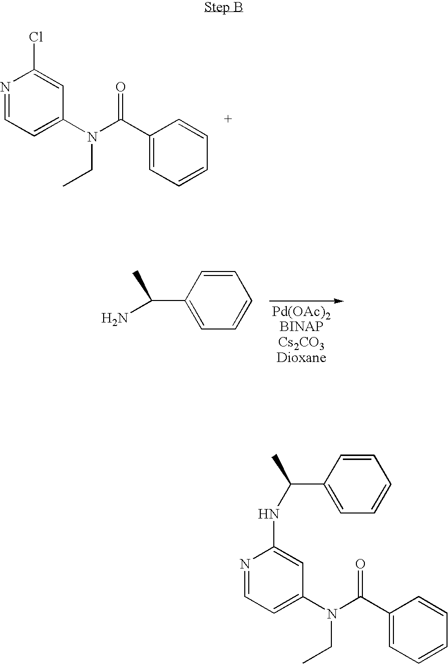

Prepared similarly to Example 1 (Step C) with an 11% yield. M+H⁺ (346).

### EXAMPLE 6

### Preparation of 2-Bromo-N-(4-fluoro-benzyl)-N-[2-(1S-phenyl-ethylamino)-pyridin-4-yl]-benzamide

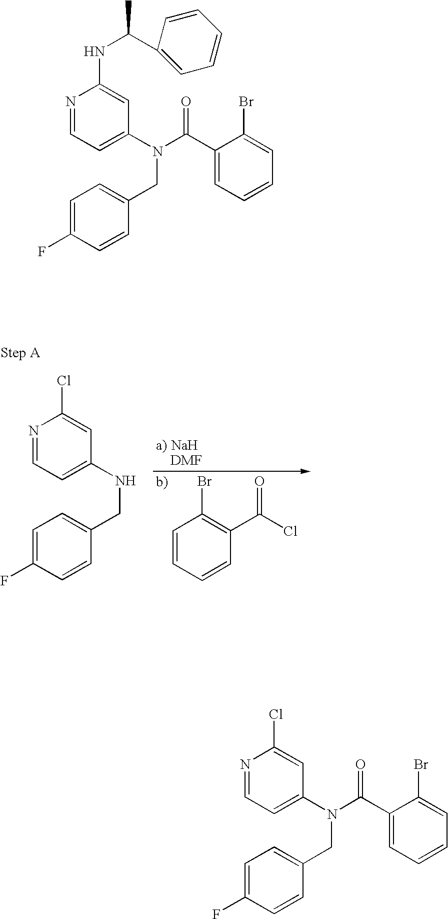

(2-Chloro-pyridin-4-yl)-(4-fluoro-benzyl)-amine (1.0 mmol) was dissolved in DMF (4 mL), and NaH(60% oil dispersion, 2 eq.) was added to the solution at room temperature. The reaction was allowed reaction to stir for 1 hr before adding the 2-bromobenzoyl chloride (1.5 eq.). The reaction was left to stir at room temperature overnight and was worked up by the addition of ethyl acetate and water (10 mL) to the reaction mixture. Following additional extraction with ethyl acetate, the combined organics were washed the water and brine, then dried over Na₂SO₄, and evaporated in vacuo. The material obtained was purified by using a gradient of 30% ethyl acetate/hexane. Final product was obtained in 55% yield. M+H⁺ (420).

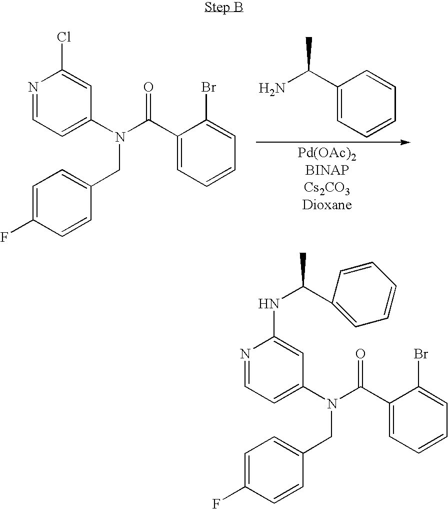

2-Bromo-N-(2-chloro-pyridin-4-yl)-N-(4-fluoro-benzyl)-benzamide (160 mg, 0.38 mmol) was dissolved in dioxane (1.0 mL), palladium acetate (4.3 mg, 0.019 mmol, 0.05 eq.), BINAP (17.8 mg, 0.029 mmol, 0.075 eq.) was added at room temperature to the solution and left to stir for 15 min. Cessium Carbonate (174 mg, 0.5345 mmol, 1.4 eq.) and α-methylbenzyl amine (64.8 mg, 0.535 mmol, 1.4 eq.) were then added to the reaction mixture. The reaction mixture was heated at 100° C. overnight. The reaction was worked up by diluting the reaction mixture with water (10 mL) and added ethyl acetate (10 mL). The organic layer was collected and the water layer was extracted with ethyl acetate (10 mL). The combined organics were washed with brine (20 mL), dried via Na₂SO₄ and evaporated in vacuo. The crude was dissolved in DMF and purified by preparative HPLC to yield the title compound as its TFA salt (20% yield). M+H⁺ (505).

### EXAMPLE 7

### Preparation of N-(4-Fluoro-benzyl)-2-methyl-N-[2-(1S-phenyl-ethylamino)-pyridin-4-yl]-benzamide

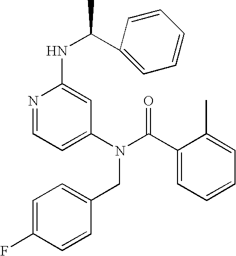

The title compound was prepared as in Example 6 from (2-Chloro-pyridin-4-yl)-(4-fluoro-benzyl)-amine and utilizing o-toluoyl chloride in place of 2-bromobenzoyl chloride. M+H⁺ (440.5).

### EXAMPLE 8

### Preparation of 3-Chloro-N-(4-fluoro-benzyl)-N-[2-(1S-phenyl-ethylamino)-pyridin-4-yl]-benzamide

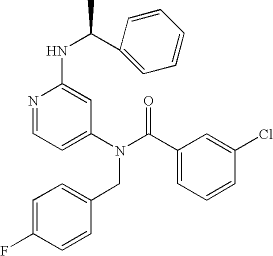

The title compound was prepared as in Example 6 from (2-Chloro-pyridin-4-yl)-(4-fluoro-benzyl)-amine and utilizing 3-chlorobenzoyl chloride in place of 2-bromobenzoyl chloride. M+H⁺ (460.95).

### EXAMPLE 9

### Preparation of 2-Fluoro-N-(4-fluoro-benzyl)-N-[2-(1S-phenyl-ethylamino)-pyridin-4-yl]-benzamide

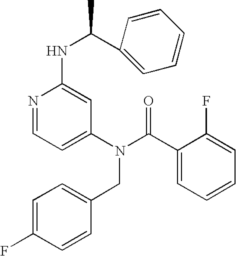

The title compound was prepared as in Example 6 from (2-Chloro-pyridin-4-yl)-(4-fluoro-benzyl)-amine and utilizing 2-fluorobenzoyl chloride in place of 2-bromobenzoyl chloride. M+H⁺ (444.495).

### EXAMPLE 10

### Preparation of 4-Chloro-N-(4-fluoro-benzyl)-N-[2-(1S-phenyl-ethylamino)-pyridin-4-yl]-benzamide

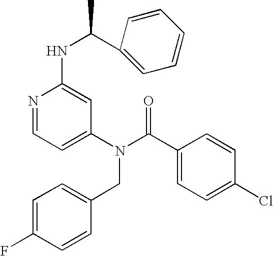

The title compound was prepared as in Example 6 from (2-Chloro-pyridin-4-yl)-(4-fluoro-benzyl)-amine and utilizing 4-chlorobenzoyl chloride in place of 2-bromobenzoyl chloride. M+H⁺ (460.95).

### EXAMPLE 11

### Preparation of Quinoxaline-2-carboxylic acid (4-fluoro-benzyl)-[2-(1S-phenyl-ethylamino)-pyridin-4-yl]-amide

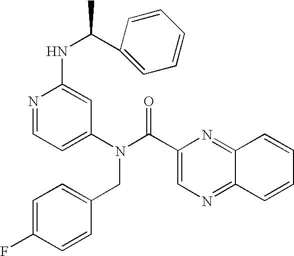

The title compound was prepared as in Example 6 from (2-Chloro-pyridin-4-yl)-(4-fluoro-benzyl)-amine and utilizing quinoxaline-2-carbonyl chloride in place of 2-bromobenzoyl chloride. M+H⁺ (478.541).

### EXAMPLE 12

### Preparation of 1-Bromo-naphthalene-2-carboxylic acid (4-fluoro-benzyl)-[2-(1S-phenyl-ethylamino)-pyridin-4-yl]-amide

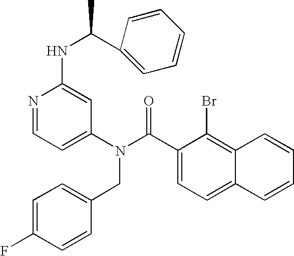

The title compound was prepared as in Example 1 utilizing 1-bromo-naphthalene-2-carbonyl chloride in place of naphthalene-2-carbonyl chloride. M+H⁺ (475.4).

### EXAMPLE 13

### Preparation of N-Ethyl-2-naphthalen-1-yl-N-[2-(1S-phenyl-ethylamino)-pyridin-4-yl]-acetamide

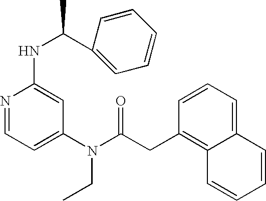

The title compound was prepared as in Example 3 where in Step B iodoethane is utilized in place of 4-fluorobenzylbromide, and in Step C naphthalen-1-yl-acetyl chloride is used in place of phenyl chloroformate. M+H⁺ (324.20+H⁺).

### EXAMPLE 14

### Preparation of Quinoline-3-carboxylic acid ethyl-[2-(1S-phenyl-ethylamino)-pyridin-4-yl]-amide

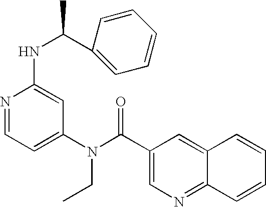

The title compound was prepared as in Example 1 where in Step A quinoline-3-carbonyl chloride is utilized in place of 2-naphthoyl chloride, and in Step B iodoethane is used in place of iodomethane. M+H⁺ (396.49+H⁺). 30% yield.

### EXAMPLE 15

### Preparation of 6-Methoxy-naphthalene-2-carboxylic acid ethyl-[2-(1S-phenyl-ethylamino)-pyridin-4-yl]-amide

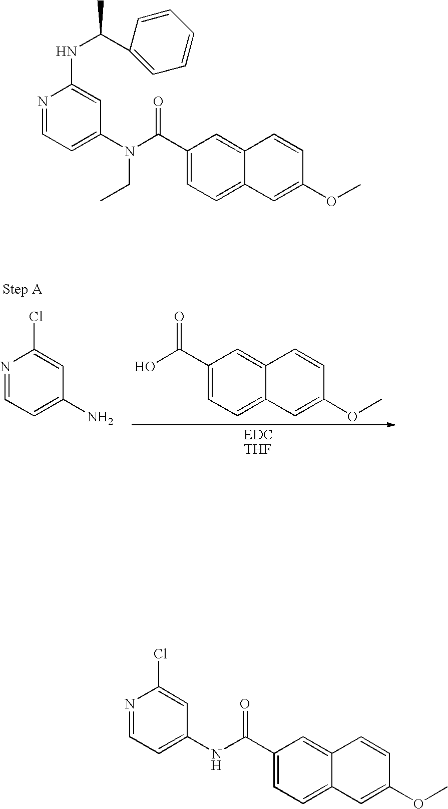

EDC (2 eq.) and the carboxylic acid (1.1 eq.) were stirred in THF (4×8 mmol) for 1 hr at room temperature at which time the DMAP (2 eq.) and 2-chloro-4-aminopyridine (1.0 g, 8.0 mmol) were added to the solution. The reaction was left to stir at room temperature overnight. Workup was carried out by diluting with water and dichloromethane. After further extraction, the combined organics were dried over Na₂SO₄, filtered, and concentrated. The crude material was purified by flash chromatography with a gradient of 10%-40% of EtOAc/Hexane. 40% yield. M+H⁺ (312.21).

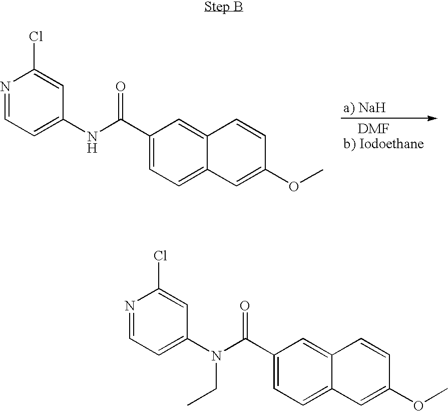

The reaction was carried out as in Example 1, Step B using iodoethane in place of iodomethane. M+H⁺ (312).

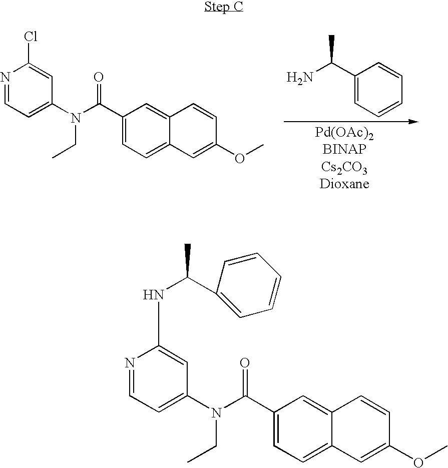

The reaction was carried out as in Example 1, Step C. M+H⁺ (340).

### EXAMPLE 16

### Preparation of N-(4-Fluoro-benzyl)-3-phenyl-N-[2-(1S-phenyl-ethylamino)-pyridin-4-yl]-propionamide

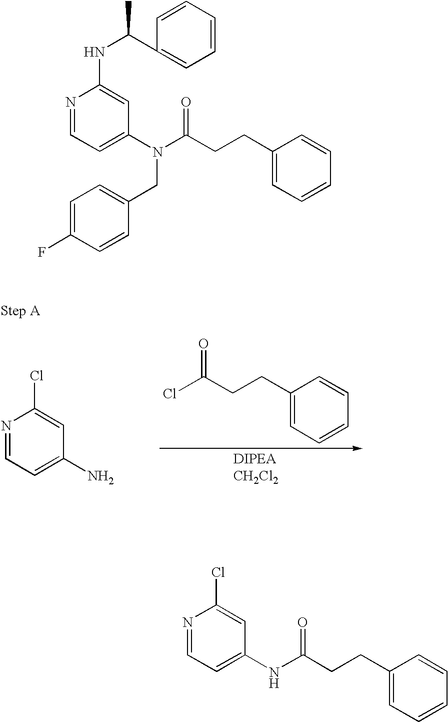

4-Amino-2-chloropyridine (0.663 g) was dissolved in 20 mL of anhydrous CH₂Cl₂. Under N₂ protection, to this solution was added 1.1 eq of DIPEA and 1.05 eq of hydrocinnamoyl chloride in one portion. The resulting solution was stirred at room temperature overnight. Extraction between H₂O and CH₂Cl₂. Separated organic layer was dried over anhydrous Na₂SO₄ and concentrated in vacuo. Silica Gel column separation (2% MeOH/CH₂Cl₂) afforded 1.058 g of product. (Yield: 81%, MH⁺: 261).

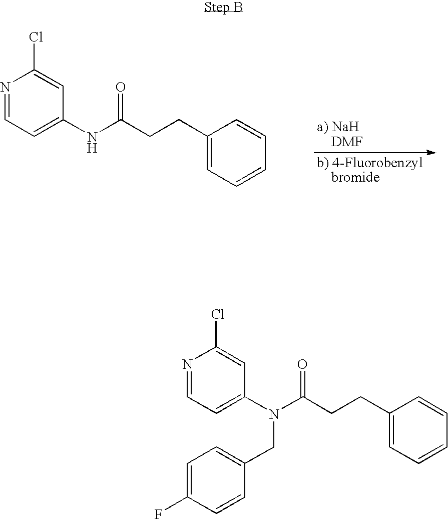

1.058 g of N-(2-chloro-pyridin-4-yl)-3-phenyl-propionamide was dissolved in 20 mL anhydrous DMF. Under N₂ protection, at 0° C., to this solution was added 1 eq of NaH (162.3 mg, 4.047 mmol). The reaction mixture was stirred at 0° C. for 15 min before the addition of 1.1 eq of 4-fluorobenzyl bromide. The reaction mixture was slowly warmed up to room temperature for 10 min and continued stirring for additional 2 hours. Solvent was removed under reduced pressure. Residue was redissolved in CH₂Cl₂ and washed with H₂O, then brine. The organic layer was dried over anhydrous Na₂SO₄ and concentrated in vacuo. Silica Gel column separation (1-2% MeOH/CH₂Cl₂) afforded 0.9 g product. (Yield: 60%, MH⁺: 369).

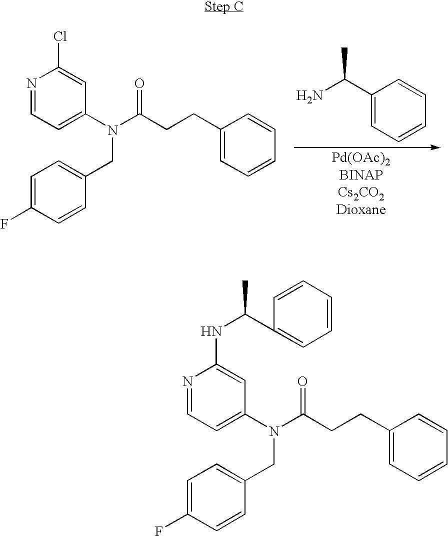

0.4125 g of N-(2-chloro-pyridin-4-yl)-N-(4-fluoro-benzyl)-3-phenyl-propionamide (1.1184 mmol) was dissolved in 8 mL anhydrous 1,4-dioxane. Under N₂ protection, to this solution was added 5 mol % of Pd₂(OAc)₂ (0.05592 mmol, 12.5 mg), 7.5 mmol % of BINAP (0.0783 mmol, 48.75 mg), 1.5 eq of amine, and 1.4 eq of anhydrous Cs2CO₃. The reaction mixture was then heated up to 100° C. overnight. Solvent was removed under reduced pressure. Residue was redissolved in CH₂Cl₂ and washed with H₂O, brine. Organic layer was dried over anhydrous Na₂SO₄ and concentrated in vacuo. Silica Gel column separation (0-4% MeOH/CH₂Cl₂) afforded 238 mg of product. (Yield: 47%, MH⁺: 454)

### EXAMPLE 17

### Preparation of N-(4-Fluoro-benzyl)-3-phenyl-N-[2-(1S-phenyl-ethylamino)-pyridin-4-yl]-acrylamide

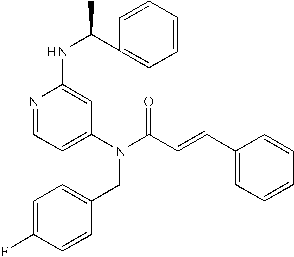

Performed as in Example 16 using cinnamoyl chloride in place of hydrocinnamoyl chloride (Yield: 43%, MH⁺: 452).

### EXAMPLE 18

### Preparation of 3,4-Dihydro-1H-isoquinoline-2-carboxylic acid (4-fluoro-benzyl)-[2-(1S-phenyl-ethylamino)-pyridin-4-yl]-amide

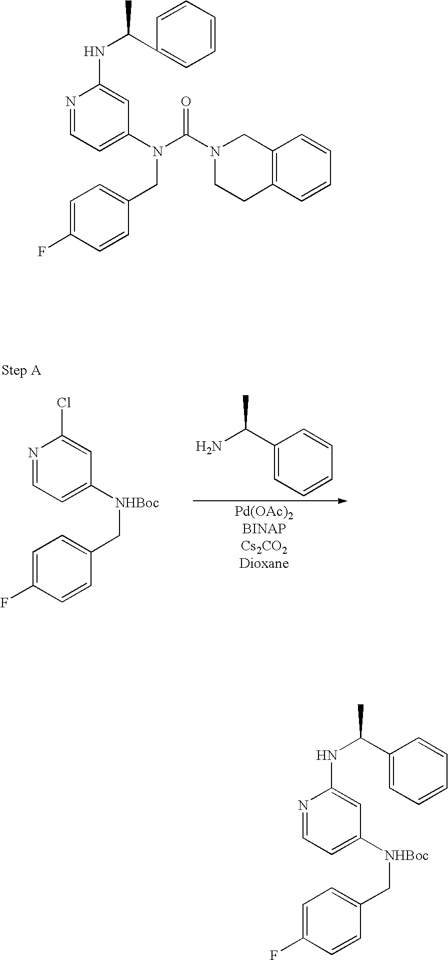

(2-Chloro-pyridin-4-yl)-(4-fluoro-benzyl)-carbamic acid tert-butyl ester (3.7713 g, 11.2 mmol) was dissolved in 45 mL anhydrous 1,4-dioxane. Under N₂ protection, was added 5 mol % of Pd₂(OAc)₂ (0.56 mmol, 12.5 mg), 7.5 mmol % of BINAP (0.84 mmol, 48.75 mg), 1.5 eq. of (S)-(−)-α-methylbenzylamine, and then 1.4 eq. of anhydrous Cs₂CO₃. The reaction mixture was then heated up to 100° C. overnight. Dioxane was removed under reduced pressure. Residue was redissolved in CH₂Cl₂ and washed with H₂O, brine. Organic layer was dried over anhydrous Na₂SO₄ and concentrated in vacuo. Silica Gel column separation (0-4% MeOH/CH₂Cl₂) afforded 1.46 g of product. (Yield: 31%, MH⁺: 422).

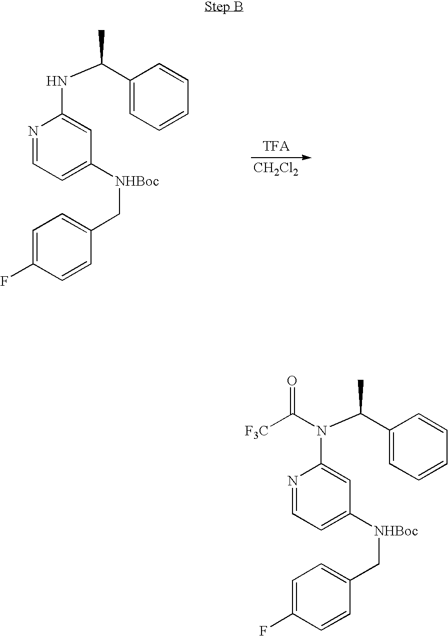

(4-Fluoro-benzyl)-[2-(1S-phenyl-ethylamino)-pyridin-4-yl]-carbamic acid tert-butyl ester (1.19 g, 2.823 mmol) was dissolved in 20 mL of anhydrous DMF. At 0° C., under N₂ protection, to this solution was added 1.1 eq. of NaH. The resulting slurry was allowed to stir at 0° C. for 15 min during which time the color changed to yellowish. 1 eq. of TFAA was then added afterwards. After 1 hour, the solvent was removed under reduced pressure. After extraction between CH₂Cl₂ and H₂O, the organic layer was washed with H₂O, brine, dried over anhydrous Na₂SO₄ and concentrated in vacuo.

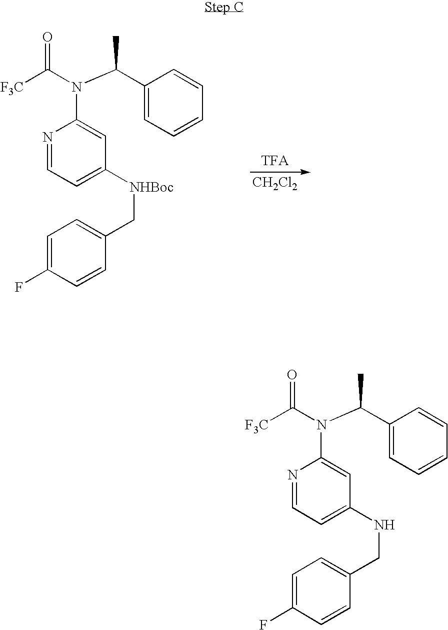

The crude product of Step B was dissolved in 1:1 mixture of TFA and CH₂Cl₂ (20 mL) and stirred at room temperature for half an hour. Satd. NaHCO₃ solution was added to neutralize the excess of TFA. After extraction between CH₂Cl₂ and H₂O, the organic layer was washed with H₂O, brine, and concentrated in vacuo. Silica Gel column separation (0-4% MeOH/CH₂Cl₂) afforded 0.695 g of product. (Yield: 59% for step 4&5, MH⁺: 418).

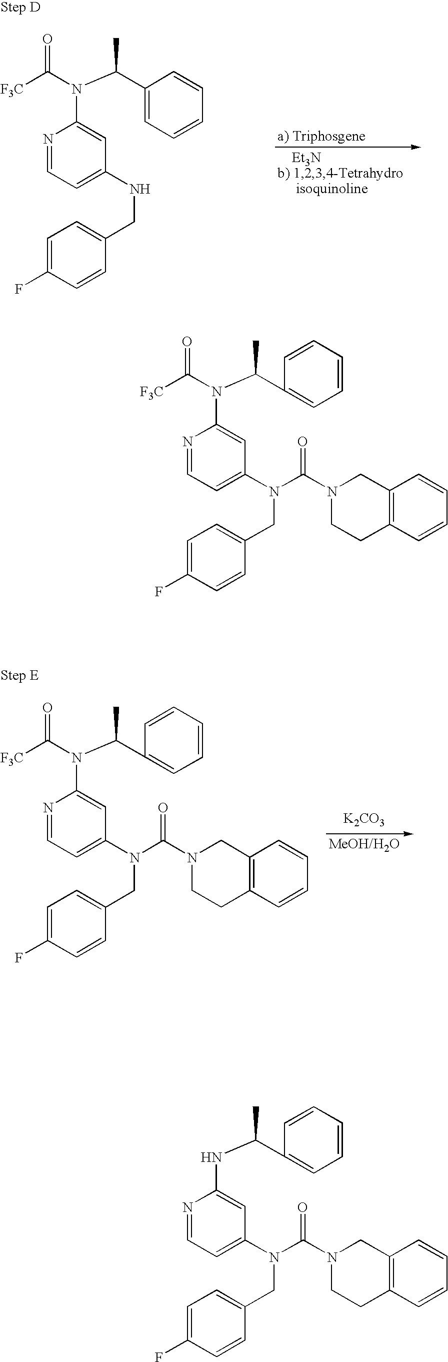

46 mg (0.08 mmol) of product of Step D was dissolved in 6 mL of MeOH, followed by the addition of 5 eq. of K₂CO₃ in 4 mL of H₂O. The reaction mixture was stirred at room temperature for 4 hours. MeOH was removed under reduced pressure and residue was redissolved in CH₂Cl₂. Extraction between CH₂Cl₂ and H₂O. The organic layer was dried over anhydrous Na₂SO₄ and concentrated in vacuo. After preparative TLC separation (3% MeOH/CH₂Cl₂), 31 mg of product was obtained. (Yield: 81%, MH⁺: 481)

### EXAMPLE 19

### Preparation of 3,4-Dihydro-1H-isoquinoline-2-carboxylic acid methyl-[2-(1S-phenyl-ethylamino)-pyridin-4-yl]-amide

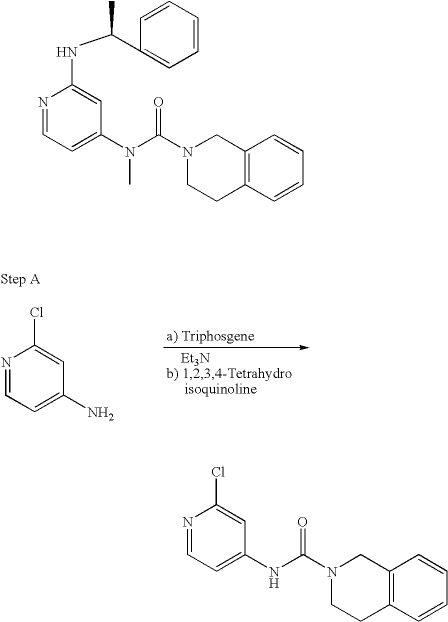

2-Chloro-pyridin-4-ylamine (3.432 g, 25.89 mmol) was dissolved in 100 mL of anhydrous 1,2-dichloroethane followed by the addition of 3 eq. of Et₃N (10.9 mL, 77.67 mmol). Under N₂ protection, at 0° C., to this solution was added triphosgene (2.56 g, 8.63 mmol). After stirring at 0° C. for 1 hour, 1.1 eq. of 1,2,3,4-tetrahydroisoquinoline was added. The resulting mixture was stirred at room temperature for another 2 hours. Solvent was removed under reduced pressure. Residue was extracted between CH₂Cl₂ and H₂O. The organic layer was dried over anhydrous Na₂SO₄ and concentrated in vacuo. Silica Gel column separation (0-4% MeOH/CH₂Cl₂) afforded 3.95 g of product. (Yield: 53%, MH⁺: 288).

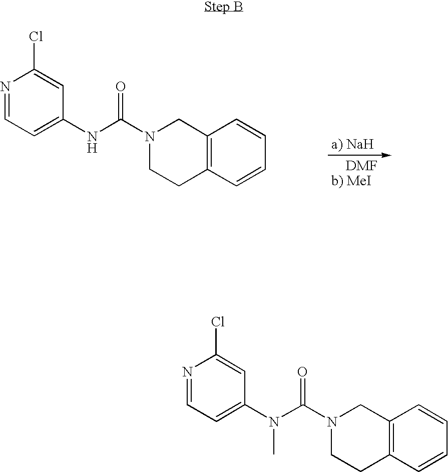

3,4-Dihydro-1H-isoquinoline-2-carboxylic acid (2-chloro-pyridin-4-yl)-amide (0.224 g, 0.78 mmol) was dissolved in 8 mL of anhydrous DMF. Under N₂ protection, at 0° C., was added 1.1 eq. of NaH (60% suspension in mineral oil, 34.3 mg, 0.86 mmol). The slurry was stirred at 0° C. for half an hour before the addition of 1.1 eq. of methyl iodide (0.122 g, 0.86 mmol). The reaction mixture was allowed to stir at room temperature for 2 hours. Solvent was removed under reduced pressure. Residue was extracted between CH₂Cl₂ and H₂O. The organic layer was dried over anhydrous Na₂SO₄ and concentrated in vacuo. Silica Gel column separation (0-2% MeOH/CH₂Cl₂) afforded 0.205 g of product. (Yield: 87%, MH⁺: 302).

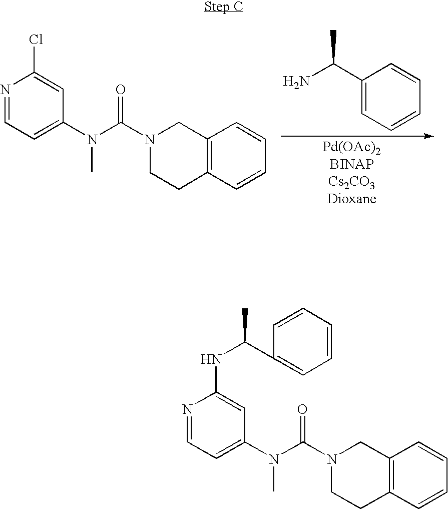

3,4-Dihydro-1H-isoquinoline-2-carboxylic acid (2-chloro-pyridin-4-yl)-methyl-amide (0.156 g, 0.517 mmol) was dissolved in 4 mL of anhydrous 1,4-dioxane. Under N₂ protection, to this solution, was added 5 mol % of Pd₂(OAc)₂ (0.026 mmol, 5.89 mg), 7.5 mmol % of BINAP (0.039 mmol, 24.2 mg), 1.5 eq. of (S)-(−)-α-methylbenzylamine, and then 1.4 eq. of anhydrous Cs₂CO₃. The reaction mixture was then heated up to 100° C. overnight. Dioxane was removed under reduced pressure. Residue was redissolved in CH₂Cl₂ and washed with H₂O, brine. Organic layer was dried over anhydrous Na₂SO₄ and concentrated in vacuo. Silica Gel column separation (0-4% MeOH/CH₂Cl₂) afforded 54 mg of product. (Yield: 27%, MH⁺: 387).

### EXAMPLE 20

### Preparation of Naphthalene-2-carboxylic acid ethyl-[2-(1S-phenyl-ethylamino)-pyrimidin-4-yl]-amide

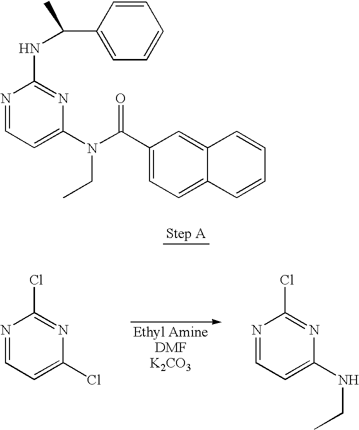

A solution containing 2,4-dichloropyrimidine (2.91 g, 19.53 mmol) and K₂CO₃ (4.05 g, 29.3 mmol) in DMF (78 mL) was cooled to −60° C. To this stirring slurry was added ethylamine (19.53 mmol) and stirring was continued overnight while the temperature was allowed to reach room temperature. The reaction mixture was diluted with water (75 mL) and extracted with EtOAc. The combined organic layer was washed first with water, then with brine and dried over Na₂SO₄ and concentrated. The residue was purified by preparative column chromatography on silica gel to yield 1.38 g (45%) of the target compound.

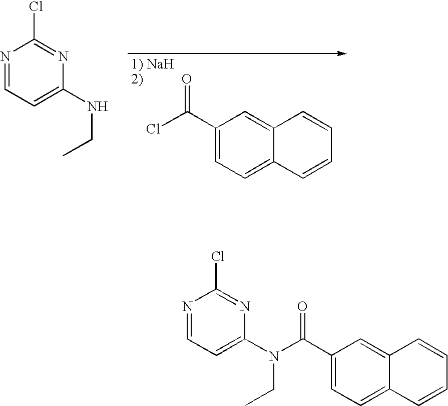

To a stirring solution of (2-Chloro-pyrimidin-4-yl)-ethyl-amine (0.75 g, 4.76 mmol) in DMF (19 mL) at room temperature was added NaH (0.38 g, 9.42 mmol) and stirred for 30 minutes. The solution was cooled to 0° C. and 2-naphthoyl chloride (0.99 g, 5.23 mmol) was added in one portion and stirring was continued overnight while the temperature was allowed to reach room temperature. Water was added to the reaction mixture and the product extracted with EtOAc. The combined organic layers were washed with water, followed by brine, dried over Na₂SO₄ and concentrated. The residue was purified by preparative column chromatography on silica gel to yield 0.71 g (48%) of the desired product. M+H⁺ (312).

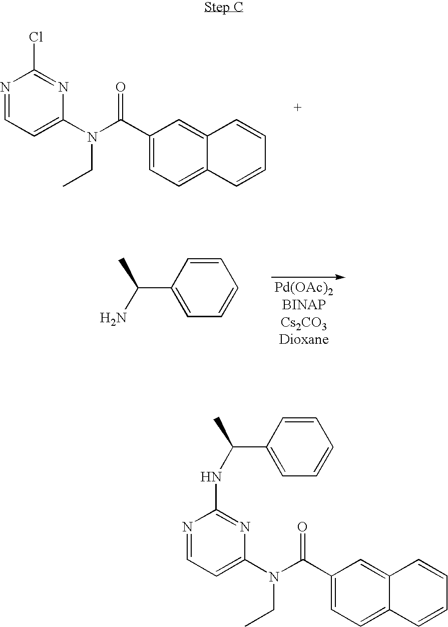

Prepared using similar conditions as in Example 1 (Step C), with a 74% yield of the target compound. M+H⁺ (398).

### EXAMPLE 21

### Preparation of Naphthalene-2-carboxylic acid ethyl-(2-isopropylamino-pyrimidin-4-yl)-amide

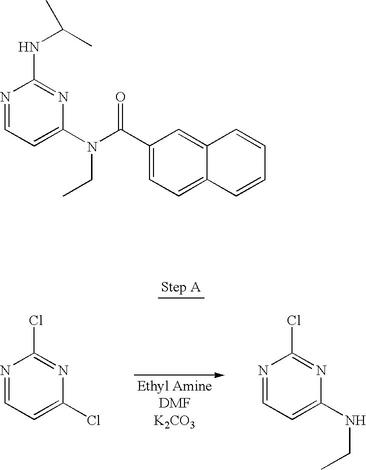

Prepared as in Example 20 (Step A).

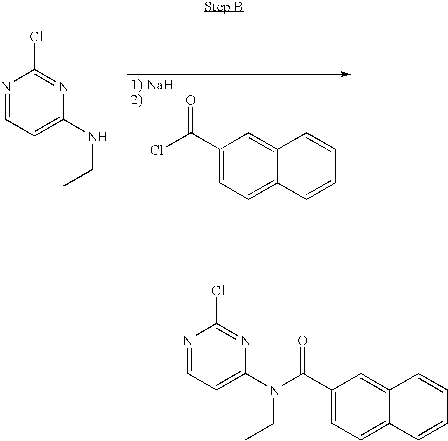

Prepared similarly to Example 20 (Step B), resulting in a 48% yield.

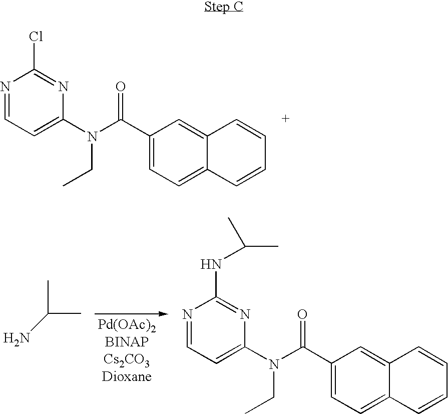

Prepared as in Example 1 (Step C), using isopropylamine and naphthalene-2-carboxylic acid (2-chloro-pyrimidin-4-yl)-ethyl-amide. The residue was purified by radial chromatography on silica gel, eluting with 40% EtOAc/hexanes to yield 14 mg (10%). M+H⁺ (335).

### EXAMPLE 22

### Preparation of Naphthalene-2-carboxylic acid [2-(1S-phenyl-ethylamino)-pyrimidin-4-yl]-piperidin-4-yl-amide

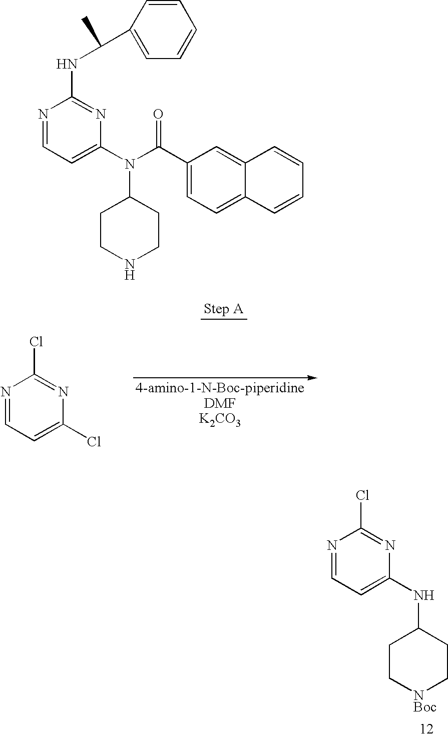

Prepared similarly to Example 20 (Step A), using 4-amino-1-N-Boc-piperidine to arrive at the target compound.

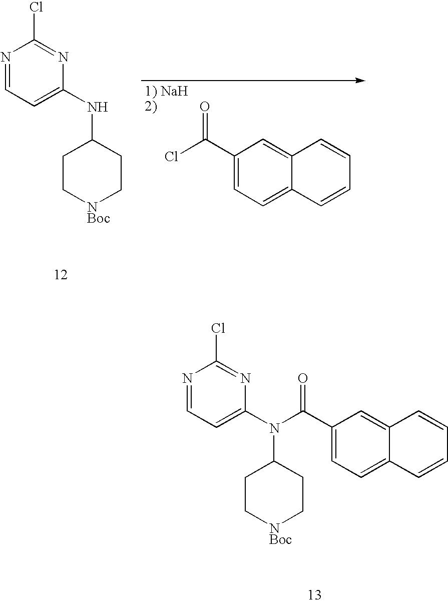

Prepared using similar conditions as seen in Example 20 (Step B), to yield the target compound (5%). M+H⁺ (467).

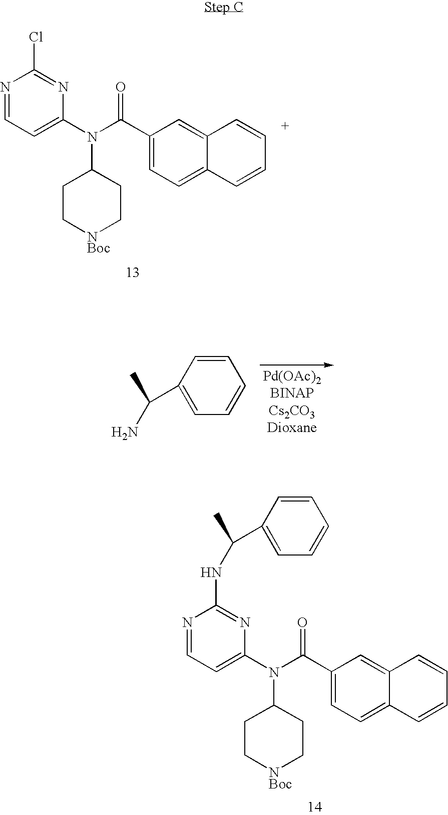

Prepared using conditions similar to Example 1 (Step C), to yield 6 mg HCl salt (19%) of the target compound. M+H⁺ (551).

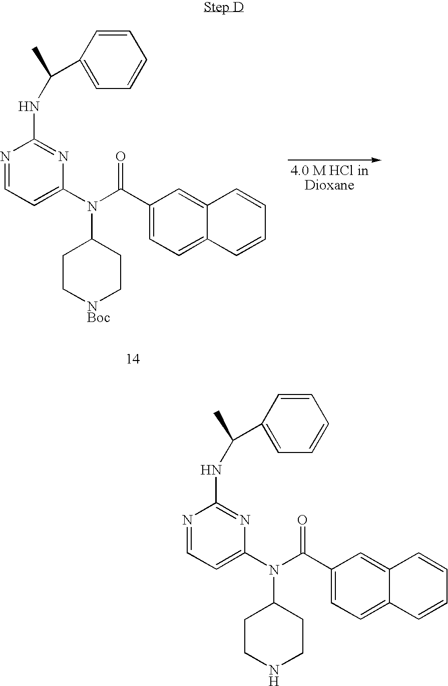

Dissolved the protected amine in excess 4.0 M HCl in dioxane overnight at room temperature. The solvent was removed under reduced pressure and the material lyophilized overnight to yield 6 mg of the hydrochloride salt of the target compound (7%). M+H⁺ (452).

### EXAMPLE 23

### Preparation of naphthalene-2-carboxylic acid [2-(1S-phenyl-ethylamino)-pyrimidin-4-yl]-piperidin-4-ylmethyl-amide

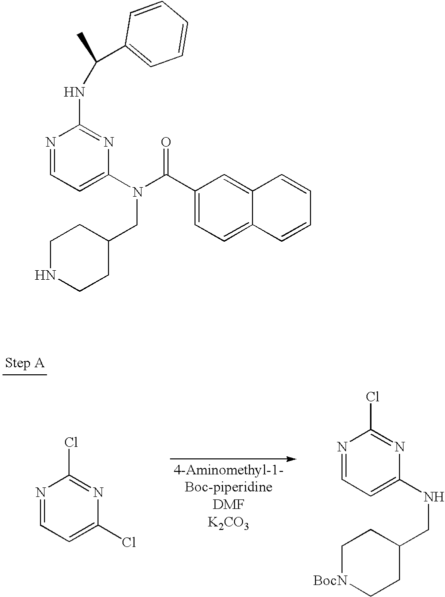

Prepared similarly to Example 20 (Step A), using 4-aminomethyl-1-Boc-piperidine to arrive at the target compound, (98%). M+H⁺ (327).

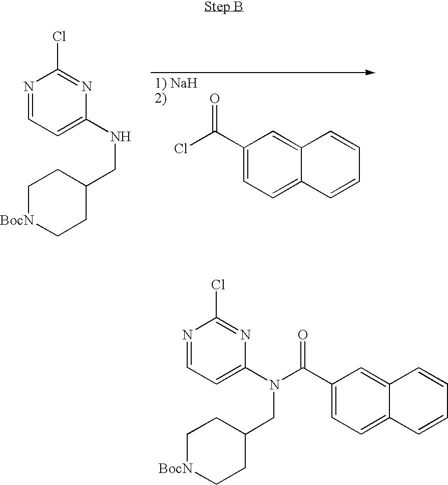

Prepared using similar conditions as seen in Example 20 (Step B) to arrive at the target compound. M+H⁺ (481).

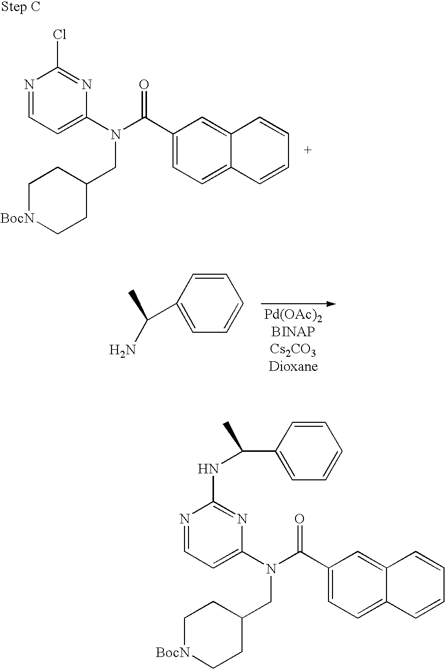

Prepared using conditions similar to Example 1 (Step C) for a yield of 31%. M+H⁺ (565).

### EXAMPLE 24

### Preparation of naphthalene-2-carboxylic acid (2-isopropylamino-pyrimidin-4-yl)-piperidin-4-ylmethyl-amide

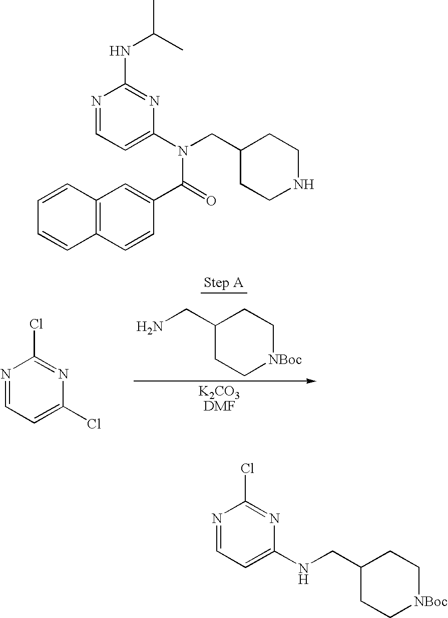

To 40 mL of DMF was added 2,4-dichloro-pyrimidine (4.41 g, 20.61 mmol), 1.1 eq. of potassium carbonate (3.13 g, 22.67 mmol), and 4-Aminomethyl-piperidine-1-carboxylic acid tert-butyl ester (4.42, 20.61 mmol). The reaction mixture was stirred at room temperature overnight. DMF was removed under reduced pressure. The residue was redissolved in CH₂Cl₂ and washed with H₂O and brine. The organic layer was dried over anhydrous Na₂SO₄ and concentrated in vacuo. 4.65 g of product (18.5 mmol) was obtained by Silica Gel column separation (0-4% MeOH/CH₂Cl₂). (Yield: 69%, MH⁺: 327).

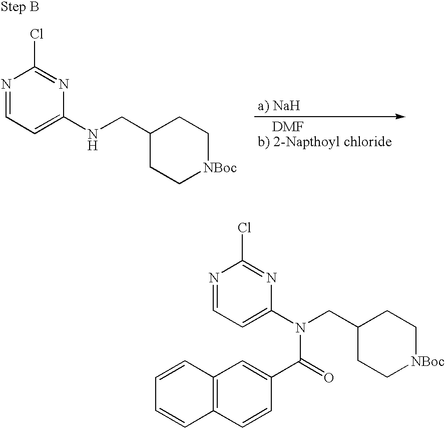

At 0° C., under N₂ protection, to 16 mL of anhydrous DMF was added 4-[(2-chloro-pyrimidin-4-ylamino)-methyl]-piperidine-1-carboxylic acid tert-butyl ester (0.523 g, 1.6 mmol) followed by the addition of 1.5 eq. of NaH (60% suspension in mineral oil, 0.096 g, 2.4 mmol). The resulting slurry was stirred at 0° C. for half an hour before warm up to room temperature and stirred for another hour. Cooled back to 0° C., to this solution was added 1.05 eq. of 2-naphthoyl chloride (0.32 g, 1.68 mmol). The reaction mixture was then allowed to stir at room temperature overnight. Solvent was removed under reduced pressure; residue was extracted between CH₂Cl₂ and H₂O. The organic layer was dried over anhydrous Na₂SO₄ and concentrated in vacuo. Silica Gel column separation (0-4% MeOH/CH₂Cl₂) afforded 0.64 g of product. (Yield: 83%, MH⁺: 482).

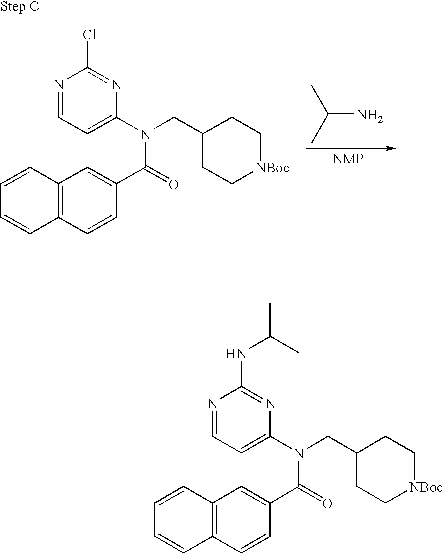

In a sealed tube, was added 4-{[(2-chloro-pyrimidin-4-yl)-(naphthalene-2-carbonyl)-amino]-methyl}-piperidine-1-carboxylic acid tert-butyl ester (0.096 g, 0.20 mmol), isopropyl amine (0.047 g, 0.8 mmol), and 2 mL of N-methylpyrrolidinone (NMP). The sealed tube was heated up to 120° C. for 1 hour. 0.093 g of product was obtained by reverse phase HPLC separation as a TFA salt. (Yield: 82%, MH⁺: 504).

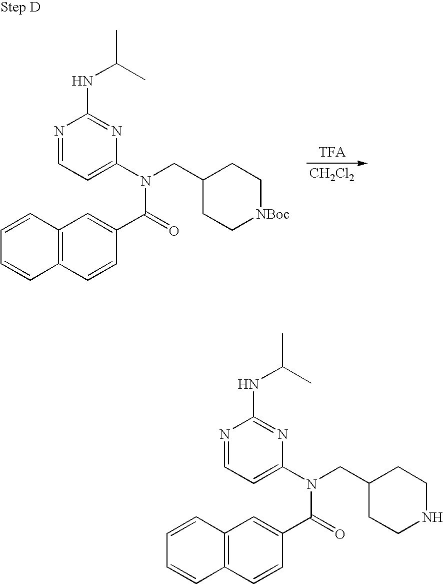

93 mg of 4-{[(2-isopropylamino-pyrimidin-4-yl)-(naphthalene-2-carbonyl)-amino]-methyl}-piperidine-1-carboxylic acid tert-butyl ester was treated with 10 mL of 1:1 mixture of TFA and CH₂Cl₂. The reaction mixture was stirred at room temperature for half an hour. Excess TFA and solvent were removed under reduced pressure. Residue was redissolved in 2 mL of DMF and subjected to reverse phase HPLC separation. 57 mg of product was obtained as a TFA salt. (Yield: 86%, MH⁺: 404).

### EXAMPLE 25

### Preparation of 2,3-Dihydro-benzofuran-5-carboxylic acid (2-amino-ethyl)-[2-(1S-phenyl-ethylamino)-pyrimidin-4-yl]-amide

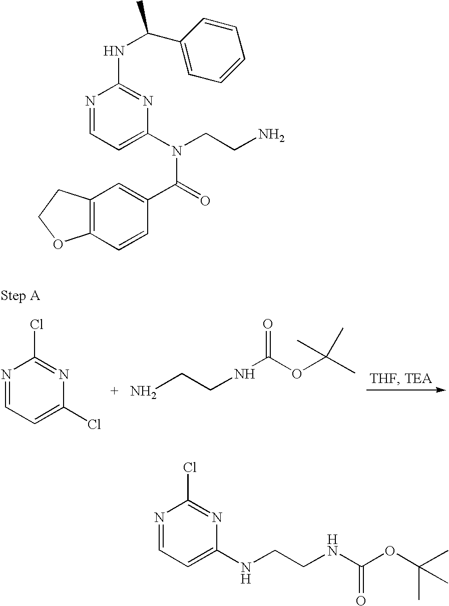

2,4-Dichloropyrimidine (2 g, 13.42 mmol) was dissolved in anhydrous THF (20 mL), then TEA (3 eq.) was added to the reaction mixture. The reaction mixture was cooled to 0° C., then the amine (2 eq.) was added slowly to the reaction. The mixture was left to stir from 0° C. to room temperature gradually overnight. The reaction was worked up with water and ethyl acetate, washed with brine, and dried with sodium sulfate. The resulting crude was purified by silica gel purification using gradient of ethyl acetate and hexane (10% ethyl acetate to 60% in 40 min.). White solids produced. 30% yield. Mass (273+H⁺¹).

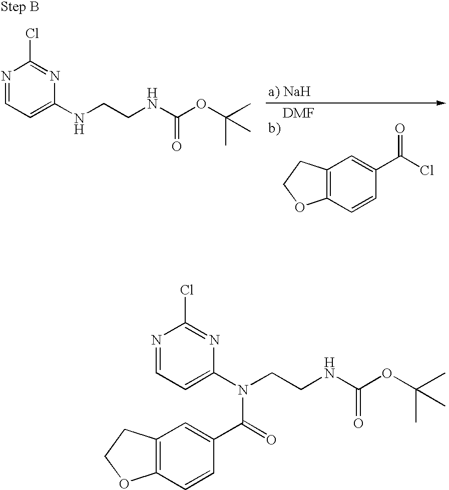

[2-(2-Chloro-pyrimidin-4-ylamino)-ethyl]-carbamic acid tert-butyl ester (1.3 g, 4.8 mmol) was dissolved in anhydrous DMF (10 mL), at room temperature. NaH (60% oil disp., 0.286 g, 1.5 eq.) was added to reaction mixture. The reaction was left to stir at room temperature for 30 min, then the acid chloride (1 g, 1.2 eq.) was added all at once. Reaction was let stir at room temperature overnight. The reaction was worked up with water and ethylacetate, dried via sodium sulfate and stripped. Crude was purified by silica gel chromatography using 10% to 50% ethyl acetate/hexane gradient in 40 min. (40% yield). LCMS mass (418+H⁺¹).

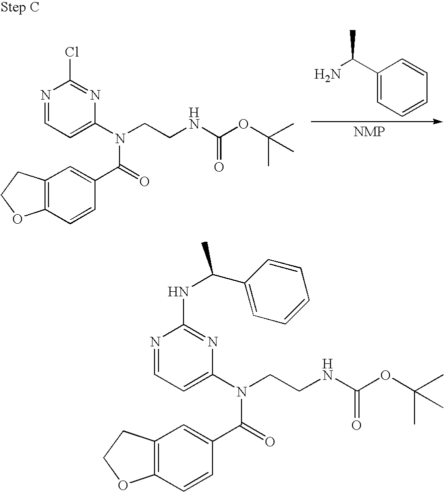

In a sealed tube (2-{(2,3-Dihydro-benzofuran-5-carbonyl)-[2-(1S-phenyl-ethylamino)-pyrimidin-4-yl]-amino}-ethyl)-carbamic acid tert-butyl ester (250 mg, 0.6 mmol) was dissolved in NMP (2 mL), added the benzylamine (3 eq.), the tube was sealed, and reaction was heated at 140° C. for 30 min. The reaction mixture was filtered, and purified by preparative HPLC to yield the TFA salt. (33% yield). LCMS (503+H⁺¹).

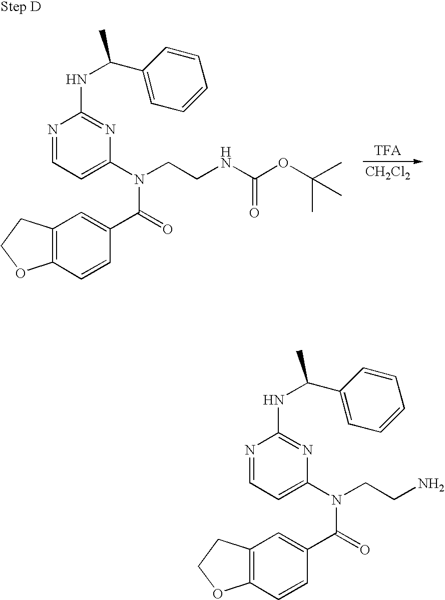

(2-{(2,3-Dihydro-benzofuran-5-carbonyl)-[2-(1S-phenyl-ethylamino)-pyrimidin-4-yl]-amino}-ethyl)-carbamic acid tert-butyl ester (100 mg, 0.20 mmol) was dissolved in 3 mL of DCM, then added excess TFA, let stir at room temperature for 1 h then stripped of solvent. Resulting oil was purified by prep HPLC and lyophilized. 25% yield. LCMS (403+H⁺¹).

Compounds 37-42 in Table 1 were prepared in a similar manner:

### EXAMPLE 26

### Preparation of Naphthalene-2-carboxylic acid ethyl-[2-(trans-4-hydroxy-cyclohexylamino)-pyrimidin-4-yl]-amide

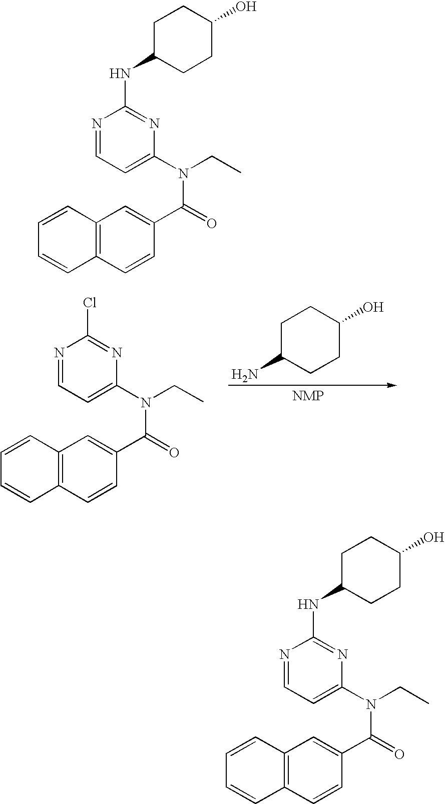

In a sealed tube, was added naphthalene-2-carboxylic acid (2-chloro-pyrimidin-4-yl)-ethyl-amide (0.35 g, 1.21 mmol), trans-4-Amino-cyclohexanol (0.56 g, 4.84 mmol), and 4 mL of N-methylpyrrolidinone (NMP). The sealed tube was heated at 120° C. for 1 hour. 0.175 g of product was obtained by reverse phase HPLC separation as its TFA salt. (Yield: 37%, MH⁺: 390).

### EXAMPLE 27 PIPERIDINYL

### Preparation of Naphthalene-2-carboxylic acid [2-(1S-phenyl-ethylamino)-pyrimidin-4-yl]-amide

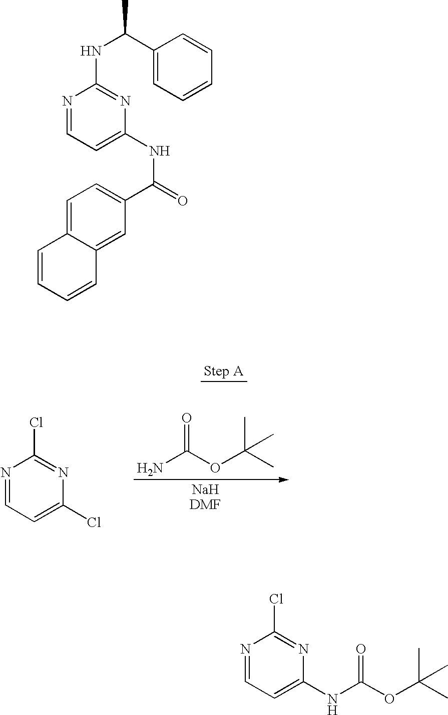

Under N₂ protection, to a solution of 2,4-dichloropyrimidine (7.582 g, 50.385 mmol) and tert-butyl carbamate (6.023 g, 50.385 mmol) in 180 mL of anhydrous DMF was added solid NaH (60% suspension in mineral oil, 4.434 g, 112.85 mmol) drop wise over 3 hours. The resulting slurry was kept under stirring for 16 hours at room temperature. Satd. NH₄Cl solution was added to quench the reaction followed by extraction CH₂Cl₂. The combined organic layer was dried over anhydrous Na₂SO₄ and concentrated in vacuo. Silica Gel column separation (0-2% MeOH/CH₂Cl₂) afforded 3 g of product. (Yield: 26%, MH⁺: 230).

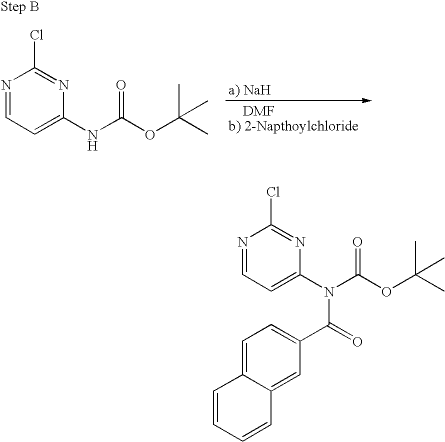

(2-Chloro-pyrimidin-4-yl)-carbamic acid tert-butyl ester (0.324 g, 1.411 mmol) was dissolved in 14 mL anhydrous DMF. At 0° C., under N₂ protection, to this solution was added 1.5 eq. of NaH (60% suspension in mineral oil, 85 mg). The resulting slurry was stirred for 15 min before warmed up to room temperature and stirred for another half an hour. 2-naphthoyl chloride (1 eq.) was added at 0° C. and the reaction mixture was stirred at room temperature for 4 hours. DMF was removed under reduced pressure. The residue was extracted between CH₂Cl₂ and H₂O. The organic layer was dried over anhydrous Na₂SO₄ and concentrated in vacuo.

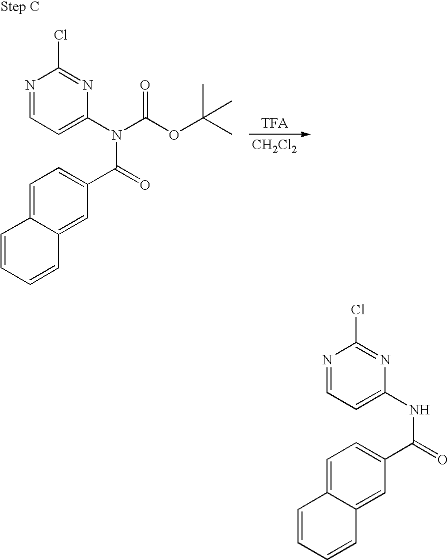

The crude product of step 2 was dissolved in 10 mL 1:1 mixture of TFA/CH₂Cl₂ and stirred at room temperature overnight. TFA and CH₂Cl₂ were removed under reduced pressure. Residue was first neutralized with Satd. NaHCO₃ solution and then extracted with CH₂Cl₂. Organic layer was dried over anhydrous Na₂SO₄ and concentrated in vacuo. Silica Gel column separation (0-2% MeOH/CH₂Cl₂) afforded 114 mg of product. (Yield: 28% for steps 2&3, MH⁺: 284).

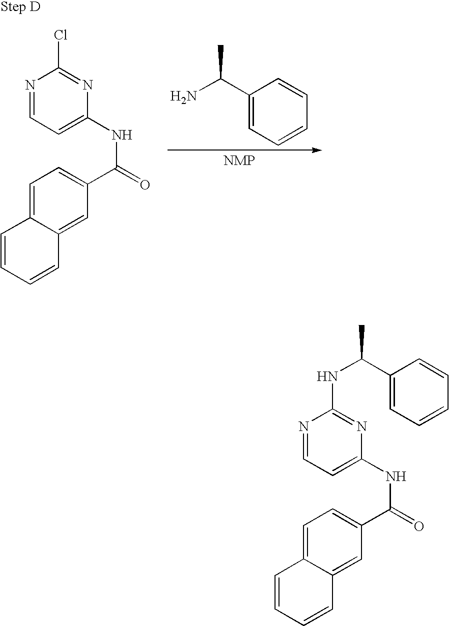

Performed as in Example 24, Step C. M+H⁺ (369).

### EXAMPLE 28

### Preparation of Naphthalene-2-carboxylic acid (4-methylsulfanyl-benzyl)-[2-(1S-phenyl-ethylamino)-pyrimidin-4-yl]-amide

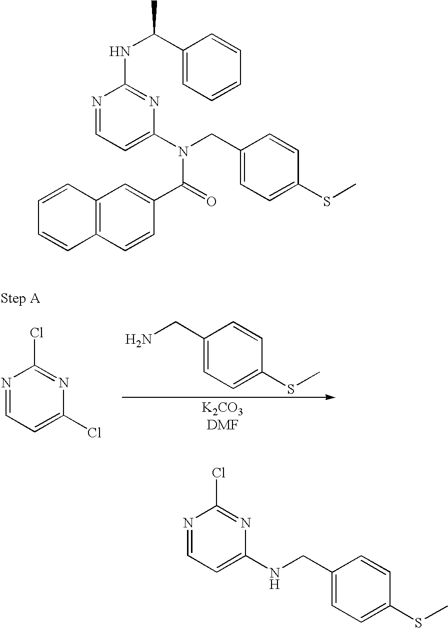

To 10 mL of DMF was added 2,4-dichloro-pyrimidine (1.44 g, 9.65 mmol), 1.1 eq. of potassium carbonate (1.47 g, 10.62 mmol), and 4-methylsulfanyl-benzylamine (1.48 g, 9.65 mmol). The reaction mixture was stirred at room temperature overnight. DMF was removed under reduced pressure. The residue was redissolved in CH₂Cl₂ and washed with H₂O and brine. The organic layer was dried over anhydrous Na₂SO₄ and concentrated in vacuo. 1.853 g of product (16.3 mmol) was obtained by Silica Gel column separation (0-4% MeOH/CH₂Cl₂). (Yield: 72%, MH⁺: 265).

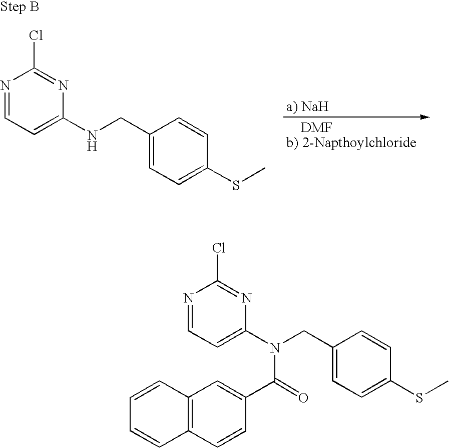

At 0° C., under N₂ protection, to 20 mL of anhydrous DMF was added (2-chloro-pyrimidin-4-yl)-(4-methylsulfanyl-benzyl)-amine (1.853 g, 6.97 mmol) followed by the addition of 1.5 eq. of NaH (60% suspension in mineral oil, 0.42 g, 10.46 mmol). The resulting slurry was stirred at 0° C. for half an hour before warm up to room temperature and stirred for another hour. Cooled back to 0° C., to this solution was added 1.5 eq. of 2-naphthoyl chloride. The reaction mixture was then allowed to stir at room temperature overnight. Solvent was removed under reduced pressure; residue was extracted between CH₂Cl₂ and H₂O. The organic layer was dried over anhydrous Na₂SO₄ and concentrated in vacuo. Silica Gel column separation (0-4% MeOH/CH₂Cl₂) afforded 2.49 g of product. (Yield: 85%, MH⁺: 420).

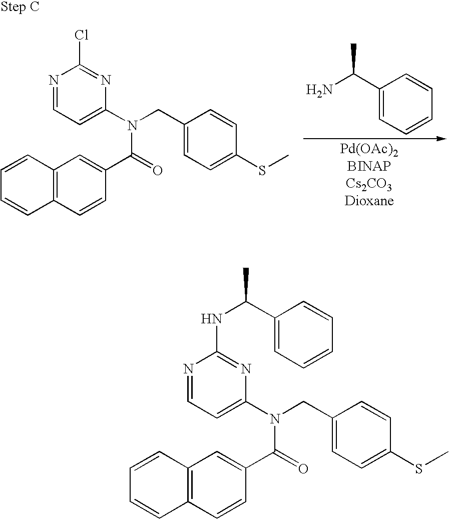

Naphthalene-2-carboxylic acid (2-chloro-pyrimidin-4-yl)-(4-methylsulfanyl-benzyl)-amide (0.515 g, 1.23 mmol) was dissolved in 6 mL of anhydrous 1,4-dioxane. Under N₂ protection, to this solution, was added 5 mol % of Pd₂(OAc)₂ (0.06 mmol, 13.8 mg), 7.5 mmol % of BINAP (0.092 mmol, 59.1 mg), 1.5 eq. of (S)-(−)-α-methylbenzylamine (0.223 g, 1.841 mmol), and then 1.4 eq. of anhydrous Cs₂CO₃ (0.56 g, 1.72 mmol). The reaction mixture was then heated up to 100° C. overnight. Dioxane was removed under reduced pressure. Residue was redissolved in CH₂Cl₂ and washed with H₂O, brine. Organic layer was dried over anhydrous Na₂SO₄ and concentrated in vacuo. Silica Gel column separation (0-4% MeOH/CH₂Cl₂) afforded 353 mg of product. (Yield: 57%, MH⁺: 504).

### EXAMPLE 29

### Preparation of Naphthalene-2-carboxylic acid (4-methanesulfinyl-benzyl)-[2-(1S-phenyl-ethylamino)-pyrimidin-4-yl]-amide

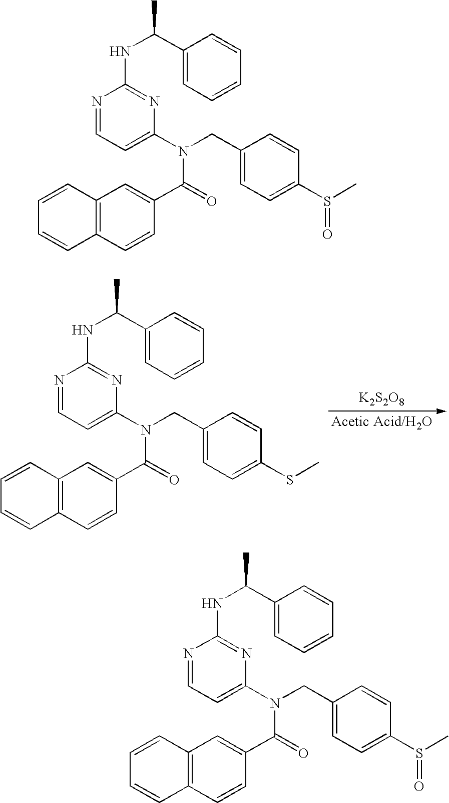

To a solution of naphthalene-2-carboxylic acid (4-methylsulfanyl-benzyl)-[2-(1S-phenyl-ethylamino)-pyrimidin-4-yl]-amide (88 mg, 0.210 mmol) in 1.71 mL of acetic acid was added a solution of K₂S₂O₈ (65 mg, 0.24 mmol) in 1.71 mL of H₂O. The resulting slurry was stirred at room temperature overnight. 12 mL of 10% NaOH was poured into the reaction flask. Extraction was carried out between CH₂Cl₂ and H₂O. The organic layer was dried over anhydrous Na₂SO₄ and concentrated in vacuo. Reverse phase HPLC separation afforded 98 mg of product as a TFA salt. (Yield: 90%, MH⁺: 520).

### EXAMPLE 30

### Preparation of Naphthalene-2-carboxylic acid (4-methanesulfonyl-benzyl)-[2-(1S-phenyl-ethylamino)-pyrimidin-4-yl]-amide

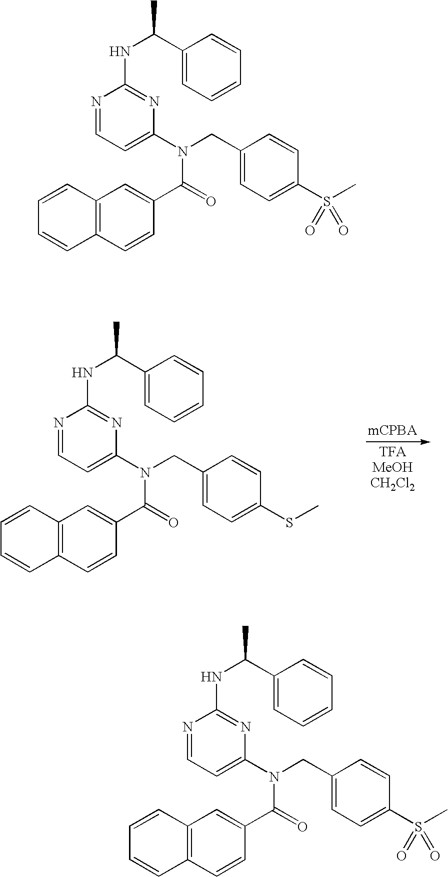

To a 0° C. solution of naphthalene-2-carboxylic acid (4-methylsulfanyl-benzyl)-[2-(1S-phenyl-ethylamino)-pyrimidin-4-yl]-amide (83.5 mg, 0.199 mmol) in 2 mL of MeOH was added TFA (0.025 mL, 0.215 mmol), then m-chloroperoxybenzoic acid (70 mg, 0.296 mmol) in 3 mL of CH₂Cl₂ dropwise. After the reaction mixture was stirred at 0° C. for 1 hour, the solvent was evaporated in vacuo. The residue was partitioned between CH₂Cl₂ and H₂O. The aqueous phase was made basic by the addition of 2N NaOH. The organic layer was separated, dried over anhydrous Na₂SO₄ and concentrated in vacuo. Reverse phase HPLC separation afforded 100 mg of product as a TFA salt. (Yield: 94%, MH⁺: 563).

### EXAMPLE 31

### Preparation of 2,3-Dihydro-benzofuran-5-carboxylic acid (2-hydroxy-ethyl)-[2-(1S-phenyl-ethylamino)-pyrimidin-4-yl]-amide

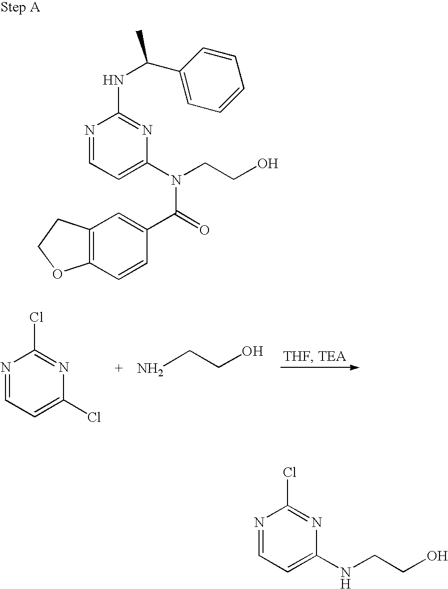

Performed as in Example 25, Step A.

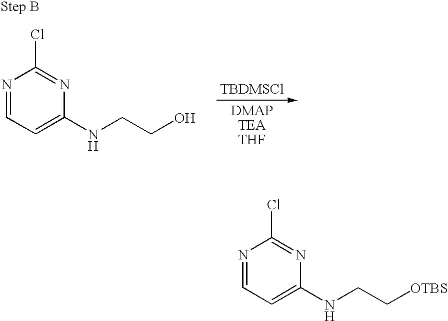

[2-(tert-Butyl-dimethyl-silanyloxy)-ethyl]-(2-chloro-pyrimidin-4-yl)-amine (2 g, 12 mmol) was dissolved in THF (50 mL), under a N₂ atmosphere. At 0° C., the DMAP (0.5 eq.), TEA (10 eq.), and TBDMSCl (3 eq.) were all added respectively. Reaction was left to stir overnight at room temperature. The reaction was worked up with water/ethyl acetate. Dried via sodium sulfate, and concentrated. The crude material was purified by silica gel chromatography, using a gradient of hexane/ethyl acetate (64% yield). LCMS (288+H⁺¹).

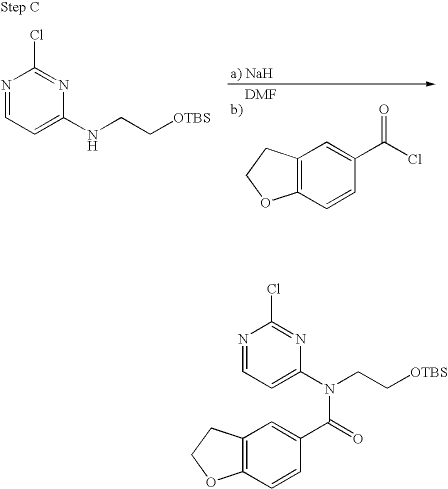

Performed as in Example 25, Step B.

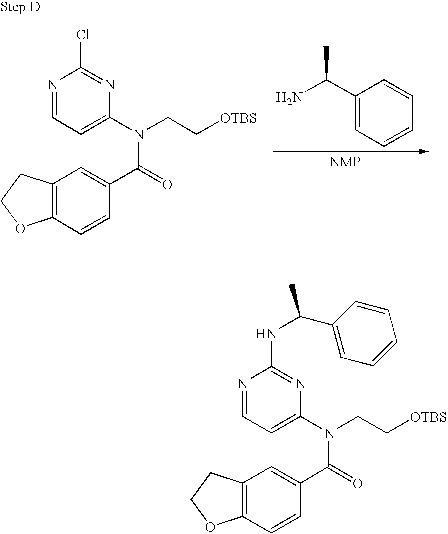

Performed as in Example 25, Step C.

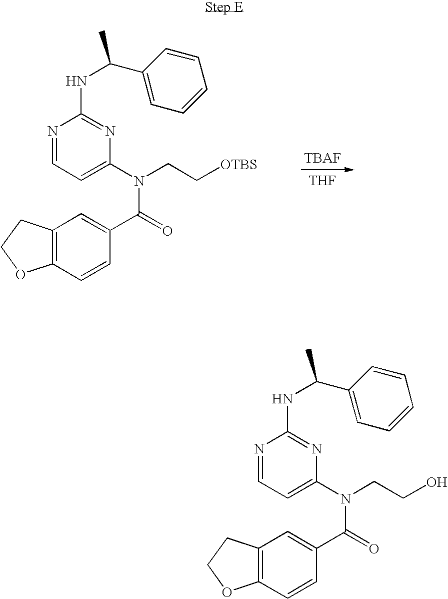

2,3-Dihydro-benzofuran-5-carboxylic acid [2-(tert-butyl-dimethyl-silanyloxy)-ethyl]-[2-(1S-phenyl-ethylamino)-pyrimidin-4-yl]-amide was dissolved in THF, and TBAF (4 eq.) was added. Reaction was left to stir for 2 h. The solvent was removed and the material was purified by preparative HPLC (39% yield). LCMS(404+H⁺¹).

### EXAMPLE 32

### Preparation of 2,3-Dihydro-benzofuran-5-carboxylic acid (2-hydroxy-ethyl)-(2-isopropylamino-pyrimidin-4-yl)-amide

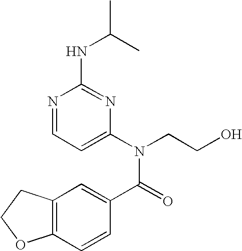

Performed as in Example 31.

### EXAMPLE 33

### Preparation of 2,3-Dihydro-benzofuran-5-carboxylic acid [2-(trans-4-hydroxy-cyclohexylamino)-pyrimidin-4-yl]-(2-hydroxy-ethyl)-amide

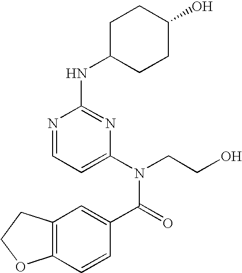

Performed as in Example 31.

### EXAMPLE 34

### Preparation of 2,3-Dihydro-benzofuran-5-carboxylic acid (2-tert-butylamino-ethyl)-[2-(1S-phenyl-ethylamino)-pyrimidin-4-yl]-amide

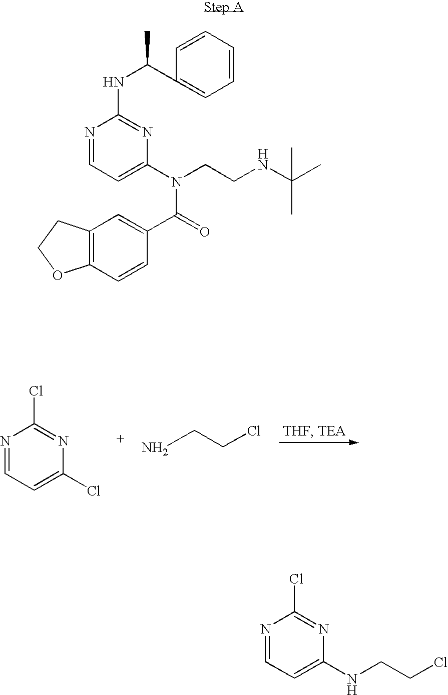

Performed as in Example 25, Step A.

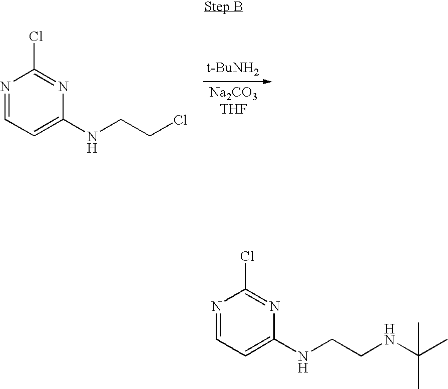

(2-Chloro-ethyl)-(2-chloro-pyrimidin-4-yl)-amine (200 mg, 1 mmol) was dissolved in THF (3 mL), and 0.8 mL water and catalytic amount of Na₂CO₃ were added. Then the tert-butyl amine (3 mL) was added. Reaction was sealed and heated at 100° C. for 4 h. The reaction was worked up with water/ethyl acetate, dried via Na₂SO₄, and concentrated. The crude material was carried to next step without purification (67% yield). LCMS (228+H⁺¹).

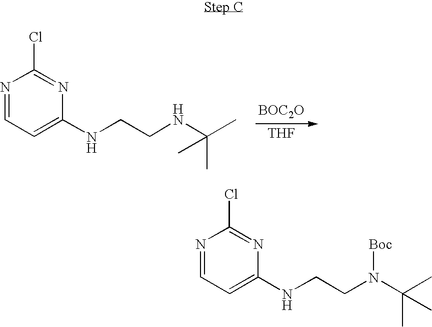

N-tert-Butyl-N′-(2-chloro-pyrimidin-4-yl)-ethane-1,2-diamine was dissolved in THF (10 mL) and excess of boc-anhydride was added. Reaction was left to stir overnight at room temperature. The reaction was worked up with water/ethyl acetate, dried with Na₂SO₄, and concentrated. The crude material was purified by silica gel chromatography (50% yield). LCMS (328+H⁺¹).

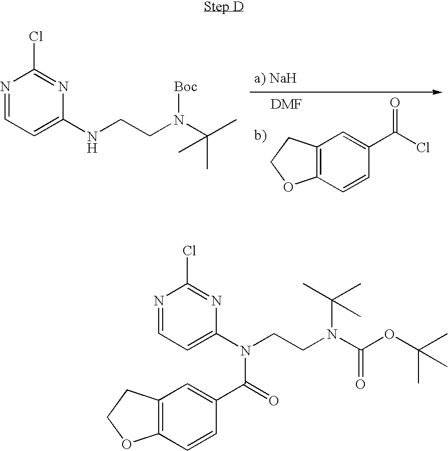

Performed as in Example 25, Step B.

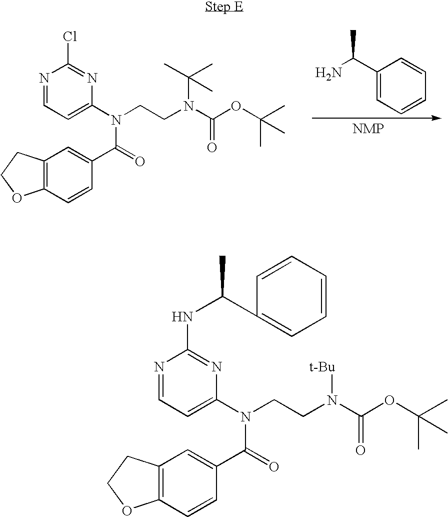

Performed as in Example 25, Step C.

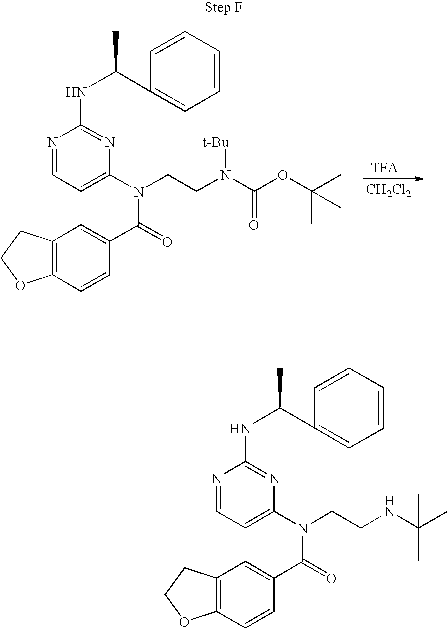

Performed as in Example 25, Step D.

### EXAMPLE 35

### Preparation of 2,3-Dihydro-benzofuran-5-carboxylic acid (2-methylamino-ethyl)-[2-(1S-phenyl-ethylamino)-pyrimidin-4-yl]-amide

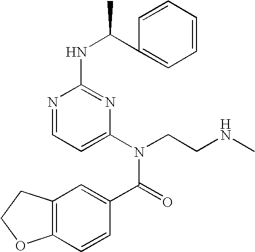

Performed similarly to Example 34.

### EXAMPLE 36

### Preparation of 2,3-Dihydro-benzofuran-5-carboxylic acid (2-isopropylamino-ethyl)-[2-(1S-phenyl-ethylamino)-pyrimidin-4-yl]-amide

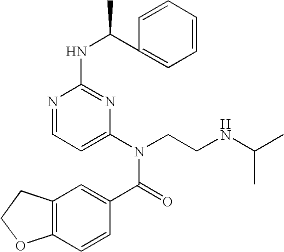

Performed similarly to Example 34.

### EXAMPLE 37

### Preparation of 2,3-Dihydro-benzofuran-5-carboxylic acid (2-isopropylamino-pyrimidin-4-yl)-(3-methyl-azetidin-3-yl)-amide

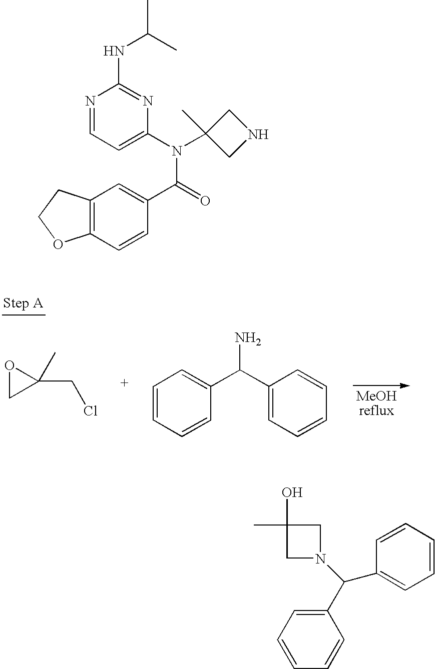

A round-bottom flask was charged with 2-(chloromethyl)-2-methyloxirane (3 g, 28.15 mmol) and aminodiphenylmethane (4.85 mL, 28.15 mmol) in MeOH (34 mL) and stirred at room temperature for 3 days. At this time, the round-bottom flask was equipped with a condenser and the contents of the flask brought to reflux for an additional 3 days. The MeOH was removed under reduced pressure and the solids washed with acetone and vacuum dried overnight to yield 5.49 g (white solid) of the hydrochloride salt of the target compound, (77%). M+H⁺ (254).

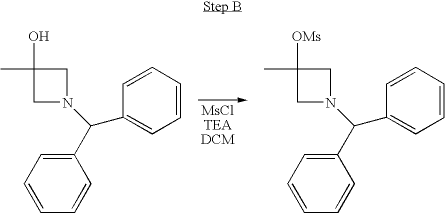

To a suspension of the alcohol (1 g, 3.9 mmol) and TEA (0.71 mL, 5.13 mmol) in DCM at 0° C. was added, dropwise, methanesulfonyl chloride (0.39 mL, 5.13 mmol). Stirring was continued overnight while the temperature of the reaction mixture was allowed to come to room temperature. The reaction mixture was then washed with water and dried over Na₂SO₄ and concentrated to yield 0.82 g of the target compound, (64%). The pale, yellow oil was pure enough to be taken to the next step without further purification.

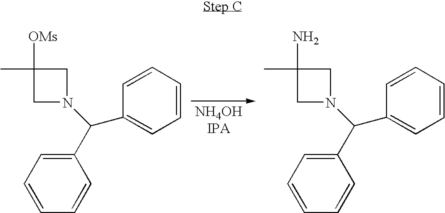

A sealed reaction tube was charged with the mesylate (0.47 g, 1.44 mmol), NH₄OH (1.5 mL) and isopropyl alcohol (2.5 mL) and heated to 70° C. for 3 h. The reaction mixture was then cooled and washed with DCM and the aqueous layer lyophilized overnight to yield 213 mg of a white solid, (58%). M+H⁺ (253).

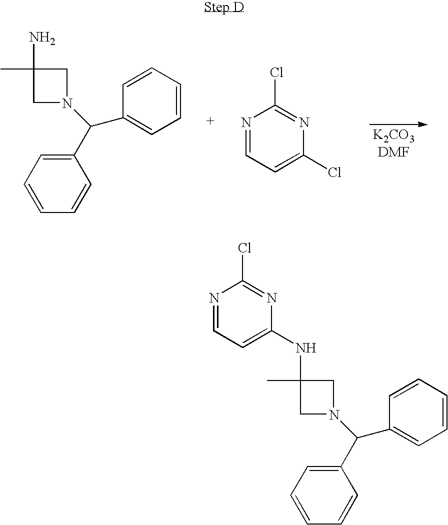

To a solution of the amine (0.34 g, 1.36 mmol) and K₂CO₃ (0.28 g, 2.04 mmol) in DMF at room temperature, was added 2,4-dichloropyrimidine (0.20 g, 1.36 mmol) and stirring continued overnight. The mixture was filtered and diluted with EtOAc and washed with water to remove DMF. Following a final wash with brine, the organic phase was dried over Na₂SO₄ and concentrated to yield 0.17 g of an colorless oil. The oil was purified by radial chromatography on silica gel (40% EtOAc/hexanes) to yield 0.052 g of product (10%). M+H⁺ (365)

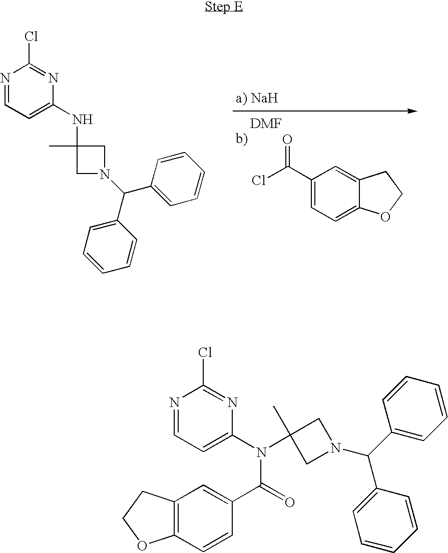

To a stirring solution of (1-Benzhydryl-3-methyl-azetidin-3-yl)-(2-chloro-pyrimidin-4-yl)-amine (0.052 g, 0.14 mmol) in DMF (0.56 mL) at room temperature was added NaH (11 mg, 0.28 mmol) and stirred for 30 minutes. The solution was cooled to 0° C. and 1,2-dihydrobenzo[B]furan-5-carbonyl chloride (0.031 g, 0.16 mmol) was added in one portion and stirring was continued overnight while the temperature was allowed to reach room temperature. Water was added to the reaction mixture and the product extracted with EtOAc (3×1 mL). The combined organic layers were washed with water, followed by brine, dried over Na₂SO₄ and concentrated. The residue was purified by radial chromatography on silica gel, eluting with 30% EtOAc/hexanes, to yield 0.04 g (56%) of the target compound. M+H⁺ (512).

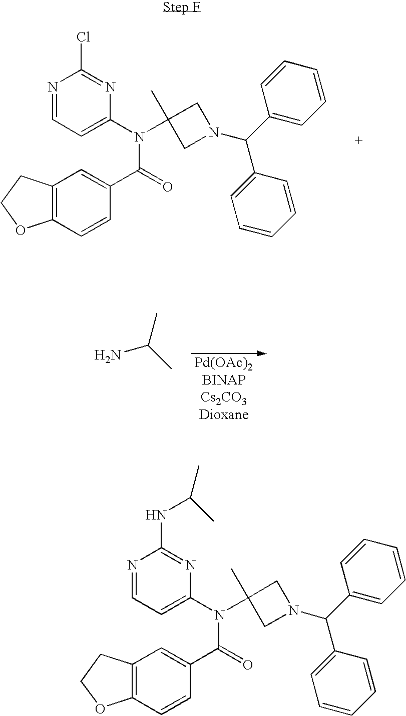

A reaction tube containing dioxane (0.56 mL) was charged with 2,3-Dihydro-benzofuran-5-carboxylic acid (1-benzhydryl-3-methyl-azetidin-3-yl)-(2-chloro-pyrimidin-4-yl)-amide (72 mg, 0.07 mmol), Pd(OAc)₂ (1.6 mg, 0.007 mmol) and BINAP (6.5 mg, 0.01 mmol) and prestirred at room temperature for 15 minutes. Then, Cs₂CO₃ (64 mg, 0.19 mmol) and α-methylbenzylamine (20 μL, 0.21 mmol) were added to the suspension and the tube was sealed and heated to 85° C. overnight. The reaction mixture was filtered and the dioxane removed under reduced pressure. The residue was purified by radial chromatography on silica gel, eluting with 30% EtOAc/hexanes, to yield 13 mg of the desired product, (17%). M+H⁺ (534).

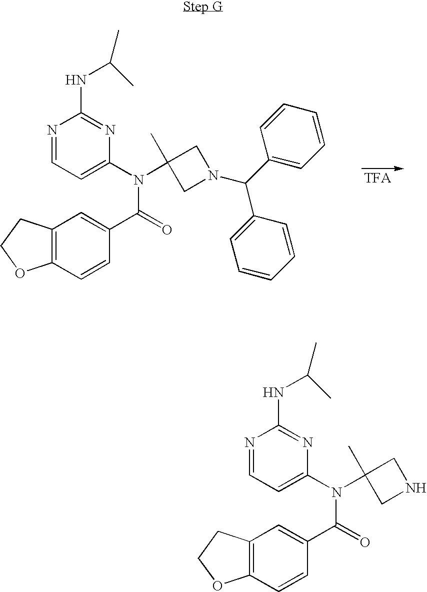

A reaction tube was charged with 2,3-Dihydro-benzofuran-5-carboxylic acid (1-benzhydryl-3-methyl-azetidin-3-yl)-(2-isopropylamino-pyrimidin-4-yl)-amide (10 mg, 0.018 mmol) and trifluoroacetic acid (1 mL) and heated to 72° C. overnight. The TFA was stripped under reduced pressure and the residue neutralized with saturated K₂CO₃(aq.) and purified by preparative thin layer chromatography, eluting with 100% EtOAc, to yield 0.8 mg of the free-base. The HCl salt was formed and lyophilized to yield 1 mg (12%) of the desired product. M+H⁺ (368).

### EXAMPLE 38

### Preparation 2,3-Dihydro-benzofuran-5-carboxylic acid (2-isopropylamino-pyrimidin-4-yl)-(1-isopropyl-piperidin-4-ylmethyl)-amide

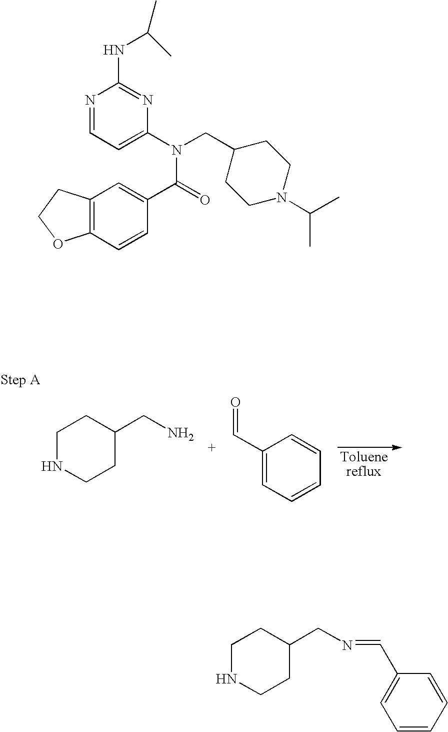

A round-bottom flask, equipped with a Dean-Starke trap, was charged with 4-aminomethylpiperidine (5 g, 43.7 mmol), benzaldehyde (4.45 mL, 43.7 mmol) and toluene (176 mL) and brought to reflux for 3 h. By this time, approximately 1 mL of water had collected in the trap and the reaction flask was removed from the heat source. The solvent was removed under reduced pressure to reveal 8.9 g of the imine as a pale, yellow oil.

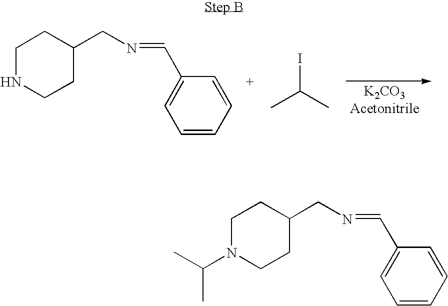

A reaction tube was charged with benzylidene-piperidin-4-ylmethyl-amine (320 mg, 1.58 mmol), iodopropane (0.19 mL, 1.9 mmol), K₂CO₃ (240 mg, 1.73 mmol) and acetonitrile (6 mL) and heated to 45° C. overnight. The mixture was then filtered and the solvent stripped under reduced pressure and place on a vacuum line overnight to yield 236 mg of benzylidene-(1-isopropyl-piperidin-4-ylmethyl)-amine.

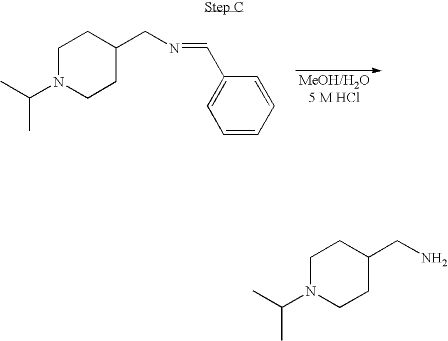

A round-bottom flask containing a mixture of 6.5 mL MeOH and 1.5 mL H₂O was charged with benzylidene-(1-isopropyl-piperidin-4-ylmethyl)-amine (237 mg, 0.97 mmol) and 1.2 mL 5 M HCl and stirred at room temperature for 2 h. The MeOH was stripped from the mixture under reduced pressure and the aqueous layer washed with Et₂O twice and then neutralized with 2 N NaOH and the product extracted with EtOAc. The combined organic layer was dried over Na₂SO₄ and concentrated to yield 76 mg of C-(1-Isopropyl-piperidin-4-yl)-methylamine (51%) as a orange oil. The desired product was sufficiently pure to continue with the next step.

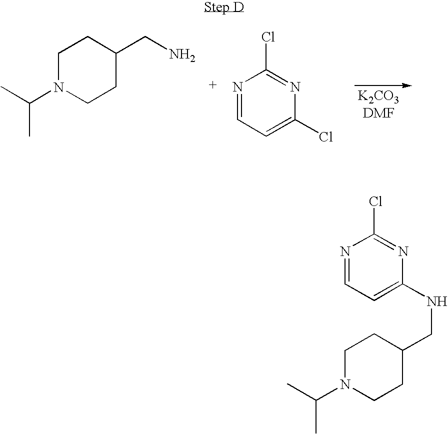

Prepared using conditions similar to Example 18 (Step D), starting with compound C-(1-Isopropyl-piperidin-4-yl)-methylamine and 2,4-dichloropyrimidine to yield the target compound, (45%). M+H⁺ (269).

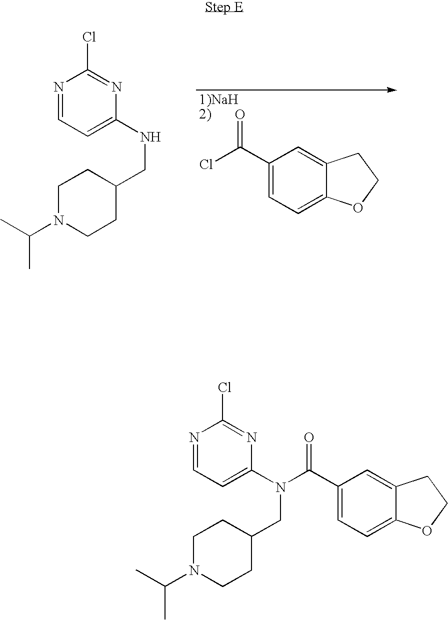

Prepared using conditions similar to Example 18 (Step E) to yield the target compound, (15%). M+H⁺ (415).

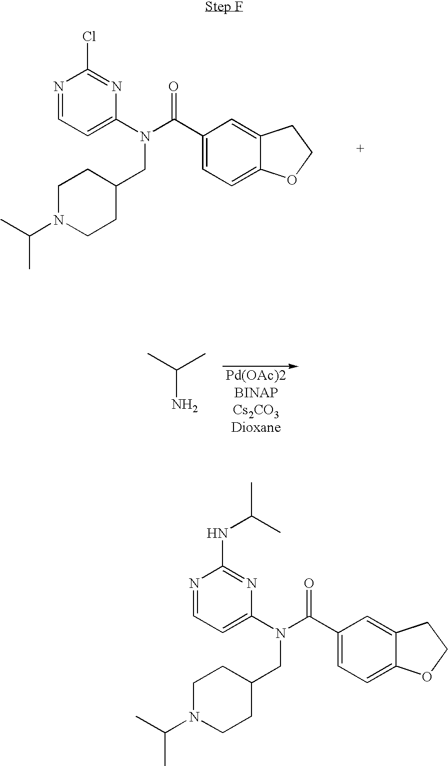

Prepared using conditions similar to Example 18 (Step F) to yield the target compound, (35%). M+H⁺ (438).

### EXAMPLE 39

### Preparation 2,3-Dihydro-benzofuran-5-carboxylic acid (1-cyclopentyl-piperidin-4-ylmethyl)-(2-isopropylamino-pyrimidin-4-yl)-amide

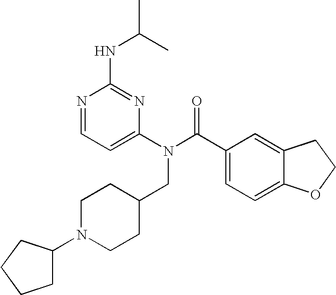

Prepared similarly to Example 38, but substituted cyclopentyl iodide for iodopropane in Step B.

### EXAMPLE 40

### Preparation 2,3-Dihydro-benzofuran-5-carboxylic acid (1-cyclopentyl-piperidin-4-ylmethyl)-[2-(1S-phenyl-ethylamino)-pyrimidin-4-yl]-amide

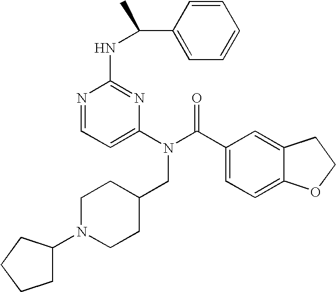

Prepared similarly to Example 38, but substituted cyclopentyl iodide for iodopropane in Step B and α-methylbenzylamine for isopropylamine in Step F.

### EXAMPLE 41

### Preparation of 2,3-Dihydro-benzofuran-5-carboxylic acid [2-(2-methoxy-cyclopentylamino)-pyrimidin-4-yl]-piperidin-4-ylmethyl-amide

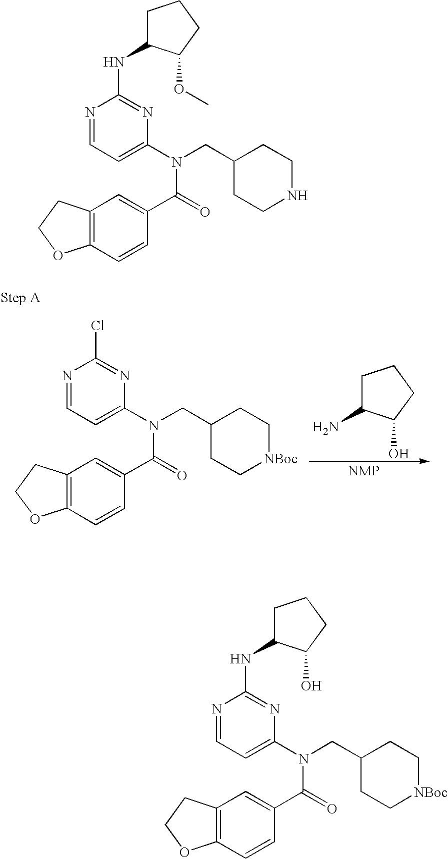

Prepared similarly to Example 24, but substituted trans-2-aminocyclopentanol hydrochloride for isoproplyamine in Step C.

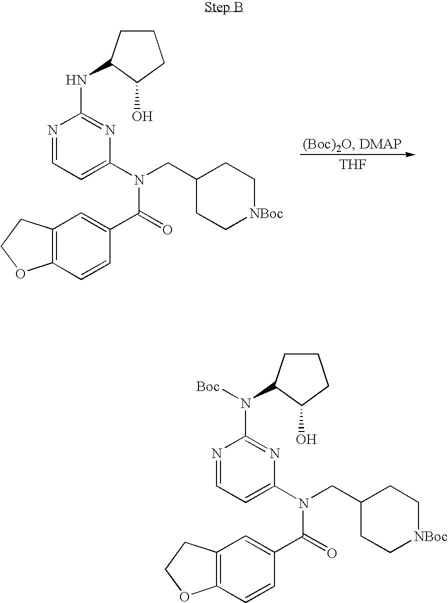

4-({(2,3-Dihydro-benzofuran-5-carbonyl)-[2-(2-hydroxy-cyclopentylamino)-pyrimidin-4-yl]-amino}-methyl)-piperidine-1-carboxylic acid tert-butyl ester (25 mg, 0.046 mMol) was dissolved in 1 mL of THF at room temperature, followed by the addition of di-tert-butyl dicarbonate (10 mg, 0.046 mMol) and a catalytical amount of DMAP. The mixture was stirred overnight at RT. The resulting mixture was partitioned between ethyl acetate and water. The ethyl acetate layer was dried over anhydrous Na₂SO₄ and concentrated. Silica gel column separation (10-50% ethyl acetate/hexane) afforded 10 mg of product, (Yield: 35%, M+H+: 638).

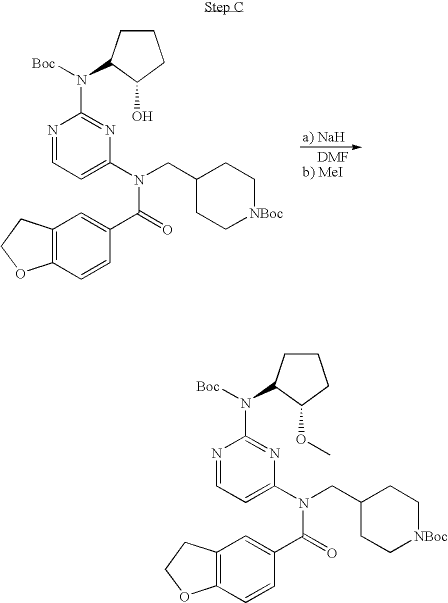

4-{[{2-[tert-Butoxycarbonyl-(2-hydroxy-cyclopentyl)-amino]-pyrimidin-4-yl}-(2,3-dihydro-benzofuran-5-carbonyl)-amino]-methyl}-piperidine-1-carboxylic acid tert-butyl ester was dissolved in DMF (1.0 mL). The solution was cooled to 0° C. and NaH (1 mg, 0.018 mMol) was added, followed by the addition of CH₃I (16 μL, 0.016 mMol). After 15 min the reaction was quenched with saturated NH4Cl, followed by extraction with ethyl acetate. The organic layer was dried over Na₂SO₄ and concentrated. Silica gel separation (10-50% ethyl acetate/hexane) afforded 8 mg of product, (Yield: 77%, M+H+: 652).

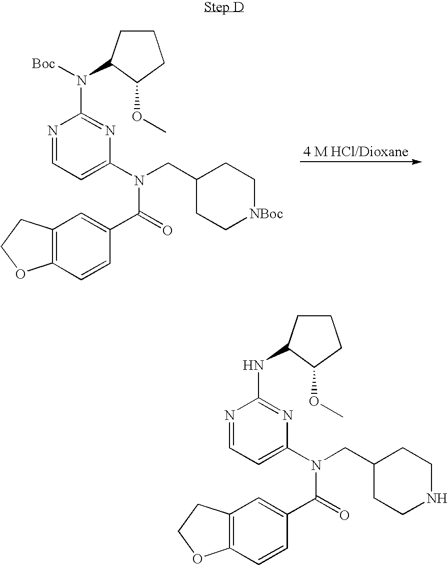

Prepared similarly to Example 24, but substituted 4 M hydrogen chloride in dioxane for 1:1 TFA/CH₂Cl₂. Obtained 5.5 mg of the desired product as an HCl salt, (Yield: 98%, M+H+: 452, R_{f}: 0.047 min, condition B).

### EXAMPLE 42

### Preparation of 2,3-Dihydro-benzofuran-5-carboxylic acid (4-amino-2,3-dihydroxy-butyl)-(2-isopropylamino-pyrimidin-4-yl)-amide

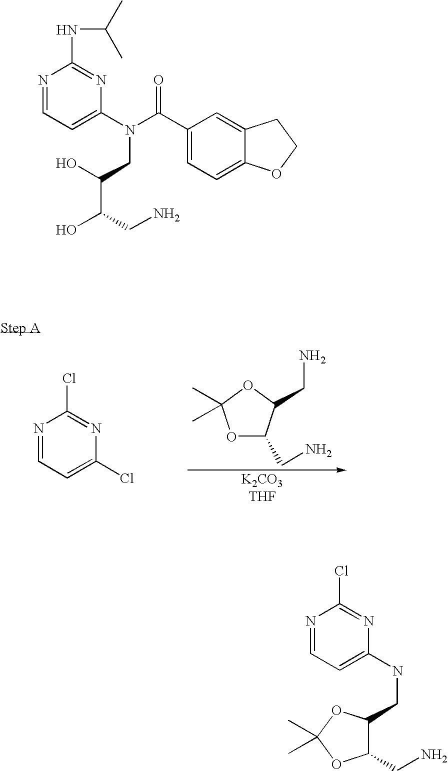

Prepared as in example 20, Step A using C-(5-Aminomethyl-2,2-dimethyl-[1,3]dioxolan-4-yl)-methylamine and THF in place of DMF. (Yield: 80%, MH⁺: 273).

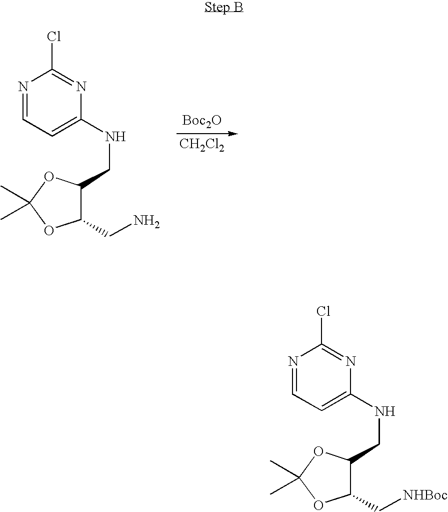

(5-Aminomethyl-2,2-dimethyl-[1,3]dioxolan-4-ylmethyl)-(2-chloro-pyrimidin-4-yl)-amine was dissolved in CH₂Cl₂ and di-tert-butyl dicarbonate (5 equiv.) was added. After stirring at RT for 2 h the reaction mixture was concentrated and the crude material was purified by silica gel chromatography (Yield: 78%, MH⁺: 372).

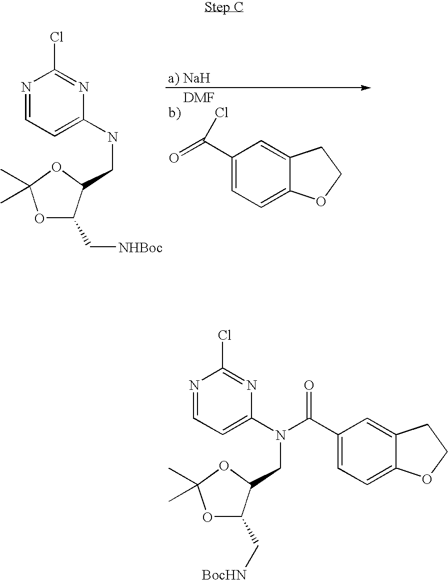

Prepared as in Example 20, Step B (Yield: 67%, MH⁺: 518).

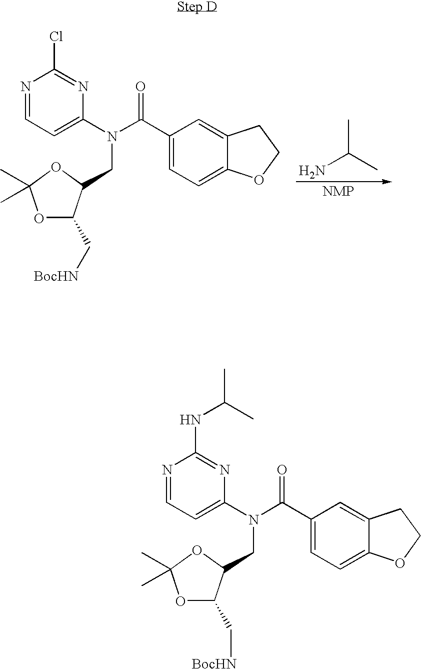

Performed as in Example 24, Step C (Yield: 40%, MH⁺: 540).

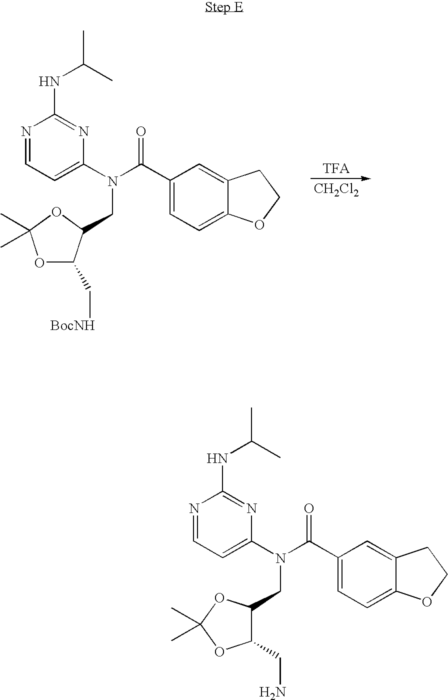

(5-{[(2,3-Dihydro-benzofuran-5-carbonyl)-(2-isopropylamino-pyrimidin-4-yl)-amino]-methyl}-2,2-dimethyl-[1,3]dioxolan-4-ylmethyl)-carbamic acid tert-butyl ester was dissolved in CH₂Cl₂ and to this stirring solution was added an excess of TFA at RT. After 1 h the solution was concentrated, redissolved in DMF and purified by preparative HPLC (Yield: 55%, MH+: 441, R_{f}: 0.940 min, condition B).

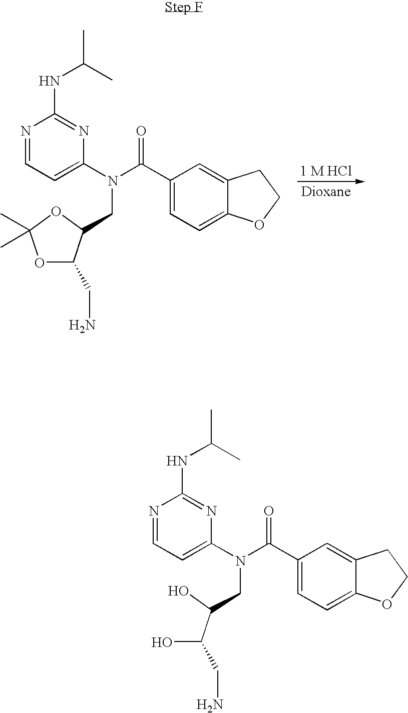

2,3-Dihydro-benzofuran-5-carboxylic acid (5-aminomethyl-2,2-dimethyl-[1,3]dioxolan-4-ylmethyl)-(2-isopropylamino-pyrimidin-4-yl)-amide was dissolved in CH₂Cl₂ and to this stirring solution was added an excess of 1M HCl at RT. After 1 h the solution was concentrated, redissolved in DMF and purified by preparative HPLC to arrive at the desired compound (Yield: 58%, MH+: 401, R_{f}: 0.853 min, condition B).

### EXAMPLE 43

### Preparation of 2,3-Dihydro-benzofuran-5-carboxylic acid (4-acetylamino-butyl)-[2-(2-methoxy-1-methyl-ethylamino)-pyrimidin-4-yl]-amide

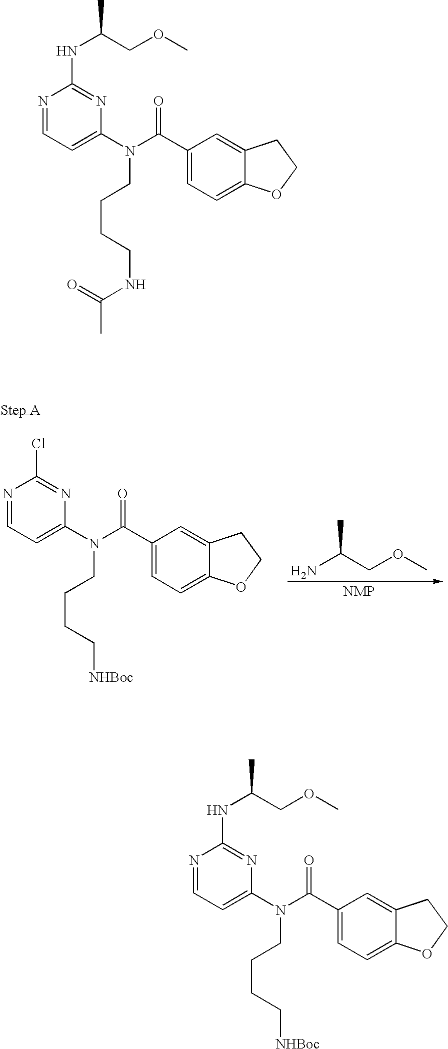

Prepared as in example 24, Step C using 2-methoxy-1-methyl-ethylamine in place of isopropylamine. (Yield: 45%, MH⁺: 499).

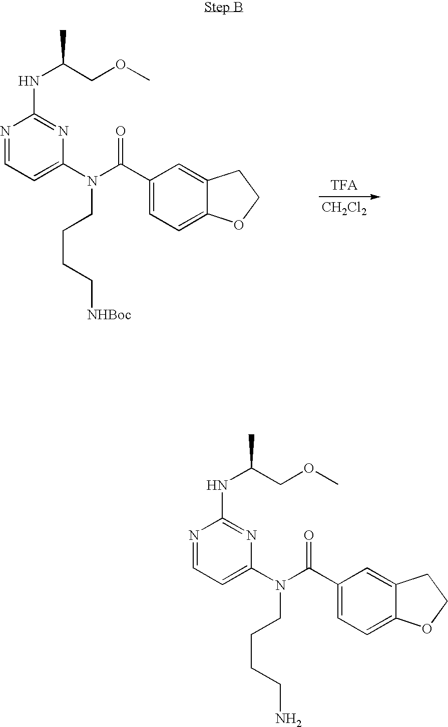

Performed as in Example 42, Step E (Yield: 65%, MH⁺: 425).

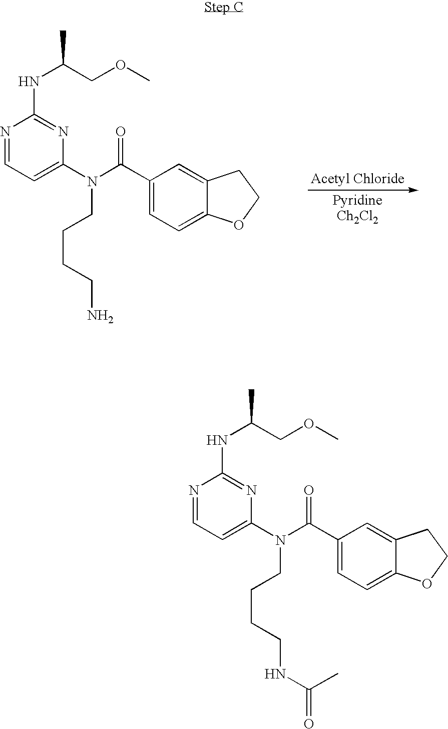

2,3-Dihydro-benzofuran-5-carboxylic acid (4-amino-butyl)-[2-(2-methoxy-1-methyl-ethylamino)-pyrimidin-4-yl]-amide was dissolved in CH₂Cl₂ and to this was added pyridine (6 equiv.) followed by acetyl chloride (1.2 equiv.). The reaction became cloudy and a precipitate formed. After 1 h the solvent was removed and the crude material was dissolved in DMF and purified by preparative HPLC (Yield: 26%, MH⁺: 441, R_{f}: 1.007 min, condition B).

### EXAMPLE 44

### Preparation of 2,3-Dihydro-benzofuran-5-carboxylic acid (4-amino-4-dimethylcarbamoyl-butyl)-(2-isopropylamino-pyrimidin-4-yl)-amide

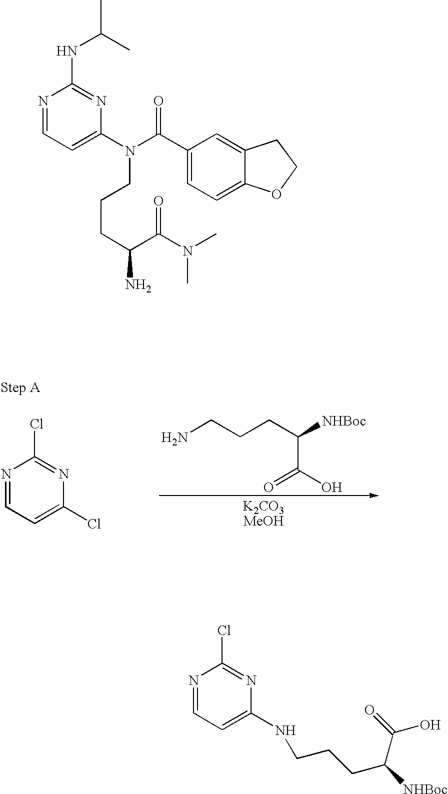

Prepared as in example 20, Step A using 5-Amino-2-tert-butoxycarbonylamino-pentanoic acid and MeOH in place of DMF. (Yield: 60%, MH⁺: 345).

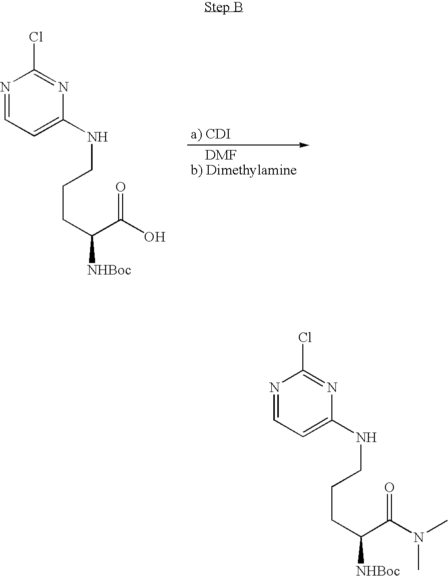

2-tert-Butoxycarbonylamino-5-(2-chloro-pyrimidin-4-ylamino)-pentanoic acid was dissolved in DMF, then CDI (2 equiv.) was added. The reaction mixture was heated at 70° C. for 3 h and then allowed to cool to RT whereupon dimethyl amine (3 equiv., 2M solution in THF) was added. After stirring for 1 h at RT the reaction was quenched with water and extracted with ethyl acetate. The organics were dried (Na₂SO₄), filtered, and concentrated. The crude material was purified by silica gel chromatography (Yield: 45%, MH⁺: 372).

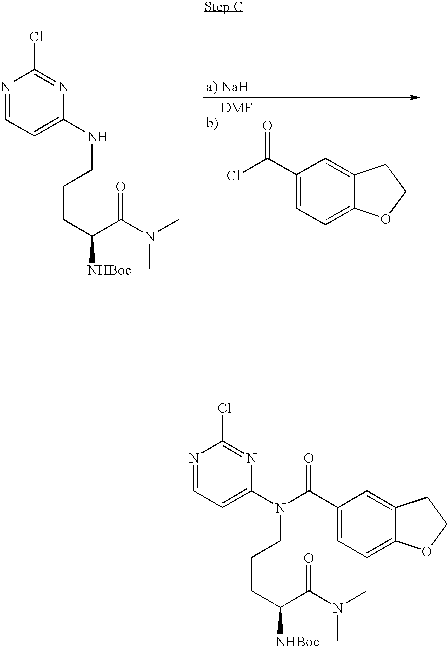

Prepared as in Example 20, Step B (Yield: 68%, MH⁺: 517).

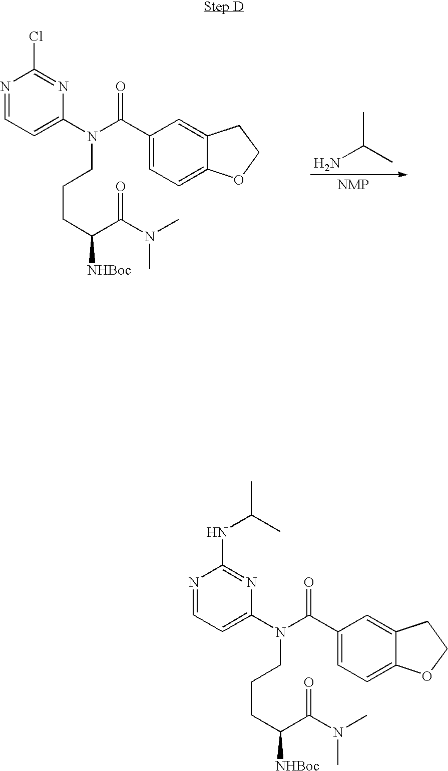

Performed as in Example 24, Step C (Yield: 57%, MH⁺: 540).

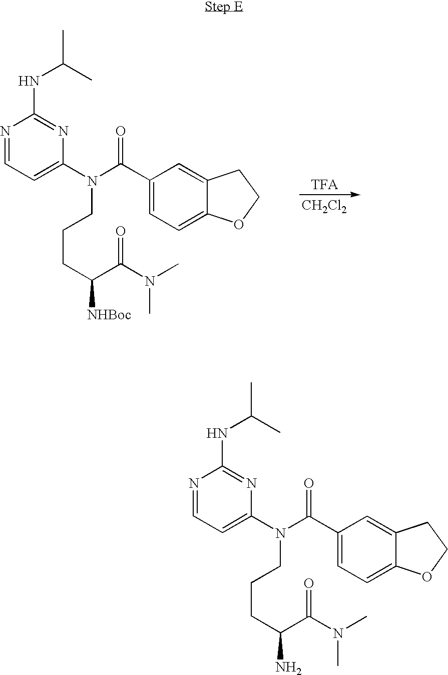

Performed as in Example 42, Step E (Yield: 78%, MH⁺: 440, R_{f}: 0.990 min, condition B).

### EXAMPLE 45

### Preparation of 2,3-Dihydro-benzofuran-5-carboxylic acid (2-guanidino-ethyl)-(2-isopropylamino-pyrimidin-4-yl)-amide

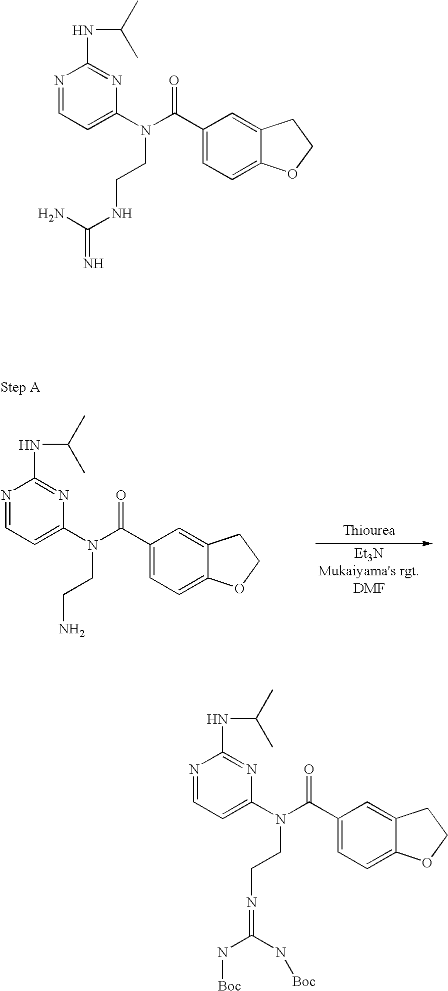

2,3-Dihydro-benzofuran-5-carboxylic acid (2-amino-ethyl)-(2-isopropylamino-pyrimidin-4-yl)-amide (0.322 mMol) was dissolved in DMF (2 mL) and thiourea (1.2 equiv.) was added, followed by triethylamine (2.2 equiv.). A suspension of Mukaiyama's reagent (1.2 equiv.) in DMF (1.0 mL) was added to the reaction mixture and stirring was continued overnight. Water and ethyl acetate were added. The organic layer was separated and the aqueous layer was extracted further with ethyl acetate. The combined organics were dried with sodium sulfate, filtered and concentrated. (Yield: 40%, MH⁺: 583).

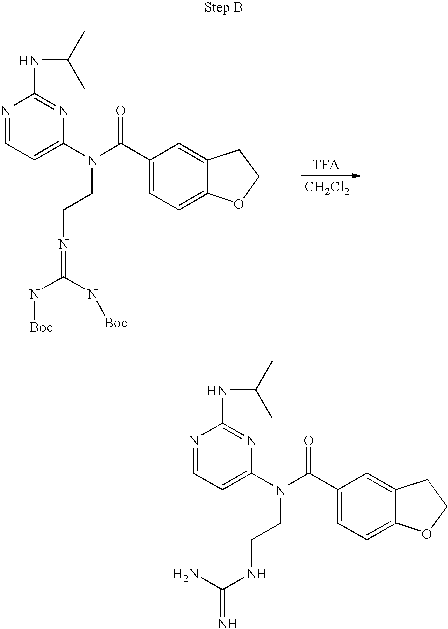

Performed as in Example 42, Step E (Yield: 23%, MH+: 383, R_{f}: 0.827 min, condition B).

### EXAMPLE 46

### Preparation of 2,3-Dihydro-benzofuran-5-carboxylic acid [2-(3-hydroxy-pyrrolidin-1-yl)-2-oxo-ethyl]-(2-isopropylamino-pyrimidin-4-yl)-amide

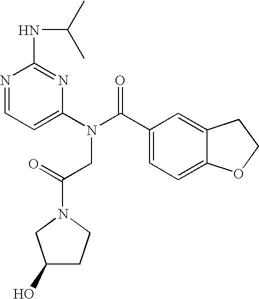

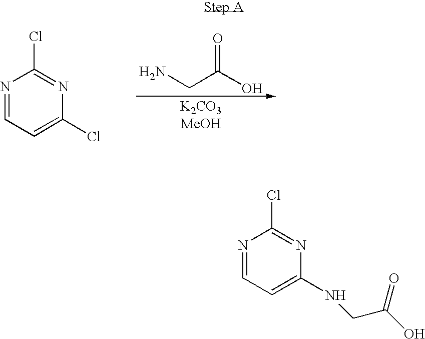

Prepared as in example 20, Step A using amino-acetic acid and MeOH in place of DMF. (Yield: 82%, MH⁺: 188).

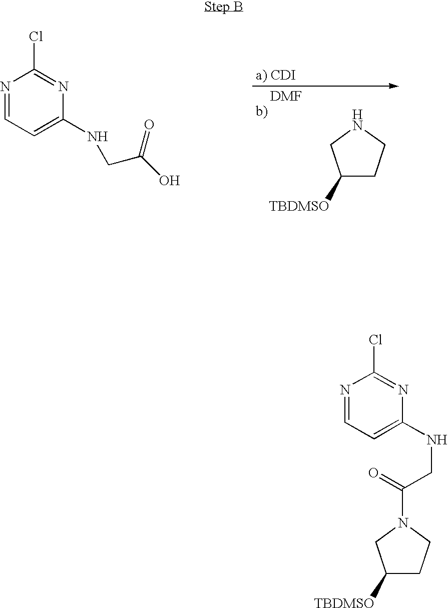

(2-Chloro-pyrimidin-4-ylamino)-acetic acid (1.45 mMol) was dissolved in DMF (50 mL), then CDI (2 equiv.) was added. The reaction mixture was heated at 70° C. for 3 h and then allowed to cool to RT whereupon 3-(tert-Butyl-dimethyl-silanyloxy)-pyrrolidine (3 equiv.) was added. After stirring for 1 h at RT the reaction was quenched with water and extracted with ethyl acetate. The organics were dried (Na₂SO₄), filtered, and concentrated. The crude material was purified by silica gel chromatography (Yield: 62%, MH⁺: 371).

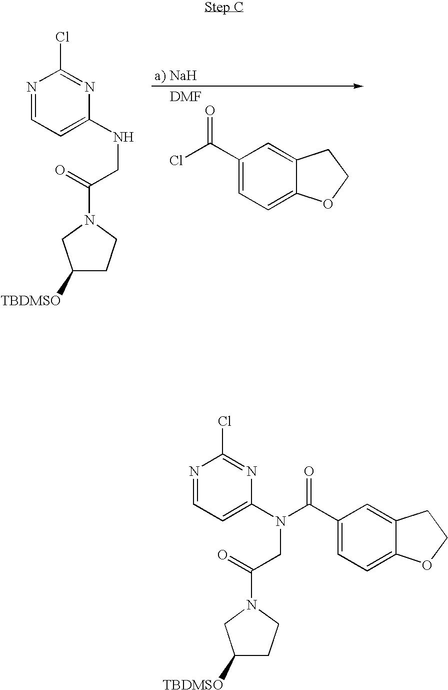

Prepared as in Example 20, Step B (Yield: 62%, MH⁺: 517).

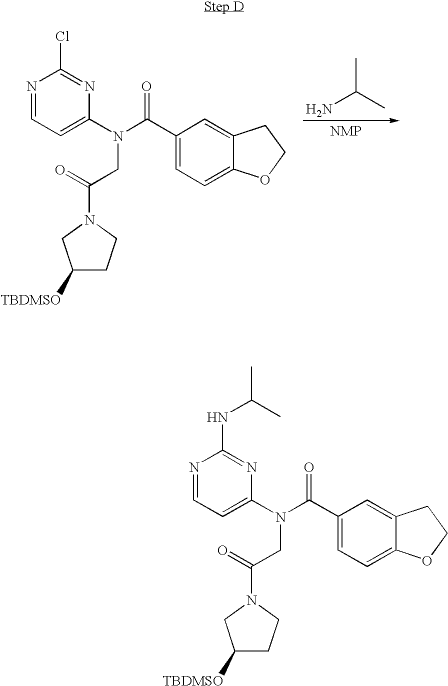

Performed as in Example 24, Step C (Yield: 64%, MH⁺: 539).

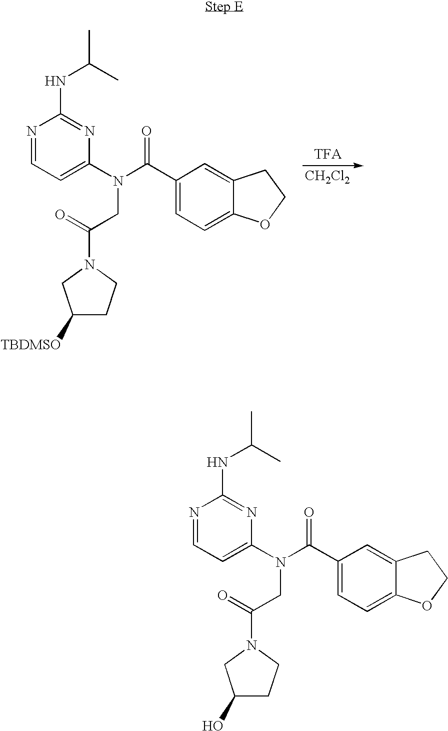

Performed as in Example 42, Step E (Yield: 88%, MH⁺: 425, R_{f}: 0.893 min, condition B).

### EXAMPLE 47

### Preparation of 2,3-Dihydro-benzofuran-5-carboxylic acid (2-guanidino-2-oxo-ethyl)-(2-isopropylamino-pyrimidin-4-yl)-amide

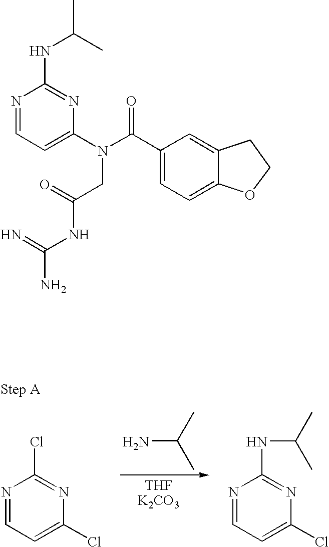

Prepared as in example 20, Step A using isopropylamine. (Yield: 25%, MH⁺: 188).

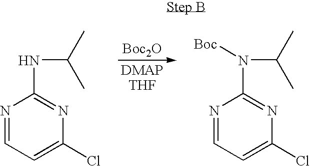

(4-Chloro-pyrimidin-2-yl)-isopropyl-amine (5.40 mMol) was dissolved in THF and then a catalytic amount of DMAP was added, followed by the addition of BOC₂O. The reaction mixture was stirred overnight at RT, whereupon it was quenched with water and extracted with ethyl acetate. The organics were dried (Na₂SO₄), filtered, and concentrated. The crude material was purified by silica gel chromatography (Yield: 94%, MH⁺: 271).

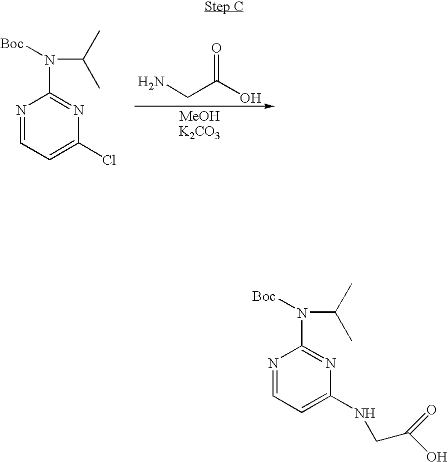

Prepared as in example 20, Step A using amino-acetic acid and MeOH in place of DMF. (Yield: 90%, MH⁺: 188).

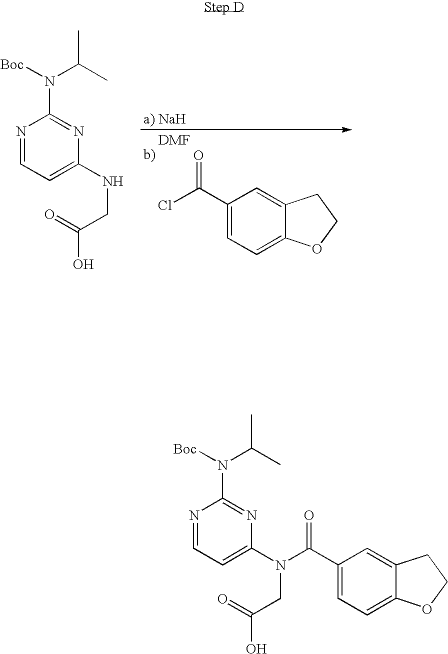

Prepared as in Example 20, Step B (Yield: 75%, MH⁺: 456).

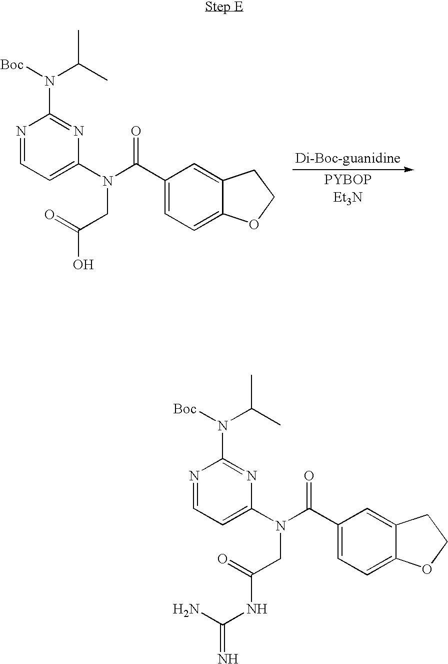

[[2-(tert-Butoxycarbonyl-isopropyl-amino)-pyrimidin-4-yl]-(2,3-dihydro-benzofuran-5-carbonyl)-amino]-acetic acid (0.22 mMol) was dissolved in DMF (5 mL) and to this was added PYBOP (1.5 equiv.), triethylamine (1.5 equiv.) and di-Boc-guanidine. After stirring at RT for 4 h, the reaction was quenched with water and extracted with ethyl acetate. The combined organics were dried (Na2SO4), filtered, and concentrated. (Yield: 54%, MH+: 697)

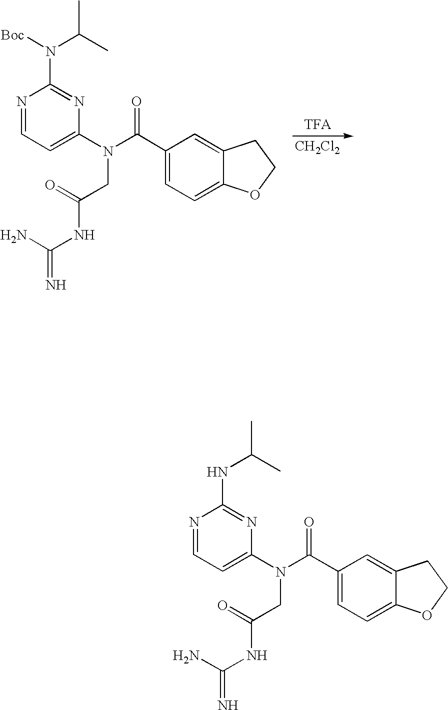

Performed as in Example 42, Step E (Yield: 30%, MH⁺: 397, R_{f}: 1.160 min, condition B).

### EXAMPLE 48

### Preparation of N-(4-Fluoro-benzyl)-2-(1-methyl-1H-indol-3-yl)-2-oxo-N-[2-(1-phenyl-ethylamino)-pyrimidin-4-yl]-acetamide

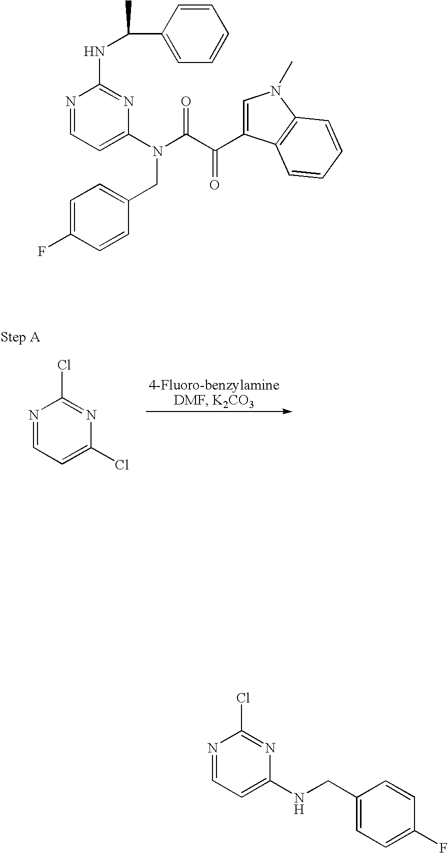

Prepared similar to Example 20 (Step A), using 4-fluorobenzylamine to arrive at the target compound.

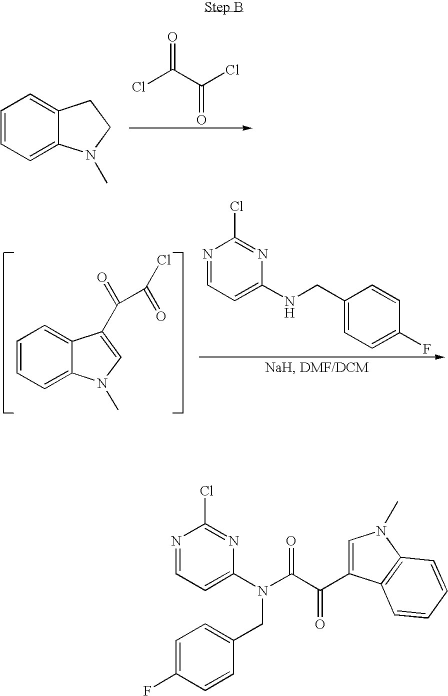

Methyl-1H-indole (0.1735 g, 1.2962 mMol) was dissolved in 13 mL of anhydrous DCM. Under nitrogen protection, at 0° C., to this solution was added 4 equiv. of 2 M of oxalyl chloride solution in DCM. The resulting mixture was stirred at 0° C. for 0.5 hour before warming to RT and stirring for 2 h. Excess oxalyl chloride was removed under reduced pressure and the residue was vacuum dried for another hour to get rid of any further trace amounts of oxalyl chloride. The (2-Chloro-pyrimidin-4-yl)-(4-fluoro-benzyl)-amine (1.30 mmol) was dissolved in 13 mL of anhydrous DMF. Under nitrogen protection, at 0° C., to this solution was added 1.5 equiv. of NaH (60% dispersion in mineral oil). After 1 hour, to this solution was added indole oxalyl chloride in 13 mL of anhydrous DCM. The resulting reaction mixture was stirred at 0° C. for 30 min before being allowed to warmed to RT and stir overnight. The solvent was then removed under reduced pressure and the residue was dissolved in DCM and washed with brine. The organic layer was dried over anhydrous Na₂SO₄ and concentrated in vacuo. Silica Gel chromatography separation (0-4% MeOH/DCM) then afforded 196.5 mg of product. (Yield: 46%)

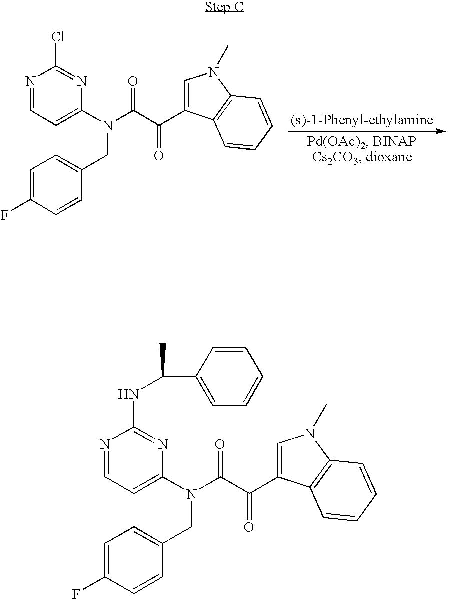

Prepared using conditions similar to Example 1 (step C), to yield 15 mg of product after silica gel chromatography separation (0-4% MeOH/DCM). (Yield: 37%, MH+: 508, R_{f}: 1.660 min, condition B).

Using procedures similar to those listed in the preceding Examples, the compounds listed in Table 1 were prepared. The Liquid Chromatography (LC) data was recorded on a Dionex P580 liquid chromatorgraph using a Dionex PDA-100 photodiode array detector with Mass Spectrometry (MS) data recorded using a Finnigan AQA MS detector. Two different LC conditions were used, Condition A (Phenomenex, 30×4.6 mm, 00A-4097-E0) and Condition B (Merck AGA Chromolith Flash, 25×4.6 mm, 1.51463.001). Additional data regarding the two LC conditions is provided below:

### LC/MS Method A

| Column | time (min) | % B | flow (mL/min) |
|---|---|---|---|
| Phenomenex | 0.00 | 5.0 | 2.00 |
| 30 × 4.6 mm | 5.00 | 95.0 | 2.00 |
| 00A-4097-E0 | 5.50 | 95.0 | 2.00 |
| | 6.00 | 5.0 | 2.00 |
| | 8.00 | 5.0 | 2.00 |
| | 8.10 | 5.0 | 0.05 |

Solvent A = water/0.1% TFA
Solvent B = acetonitrile/0.1% TFA

### LC/MS Method B

| Column | time (min) | % B | flow (mL/min) |
|---|---|---|---|
| Merck AGA Chromolith Flash | 0.00 | 5.0 | 3.00 |
| 25 × 4.6 mm | 2.50 | 95.0 | 3.00 |
| 1.51463.001 | 2.75 | 95.0 | 3.00 |
| | 3.00 | 5.0 | 3.00 |
| | 4.00 | 5.0 | 3.00 |
| | 4.10 | 5.0 | 0.05 |

Solvent A = water/0.1% TFA
Solvent B = acetonitrile/0.1% TFA

TABLE 1

| Example | | Column | Retention | Mass Observed |
|---|---|---|---|---|
| No. | Molecular Structure | Conditions | Time (min) | (M + H)+ |
| 49 | 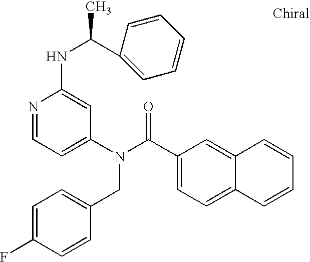 | A | 3.393 | 477 |
| 50 | 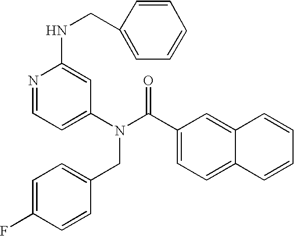 | A | 3.14 | 463 |
| 51 | 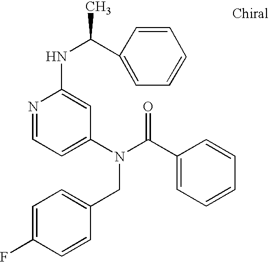 | A | 2.833 | 426 |
| 52 | 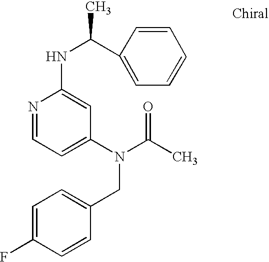 | A | 2.427 | 364 |
| 53 | 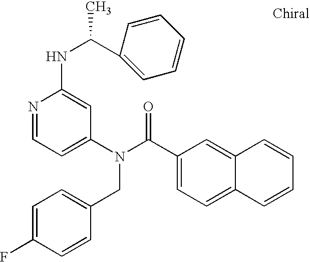 | A | 2.833 | 477 |
| 54 | 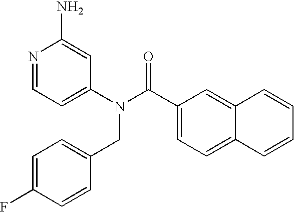 | A | 2.627 | 372 |
| 55 | 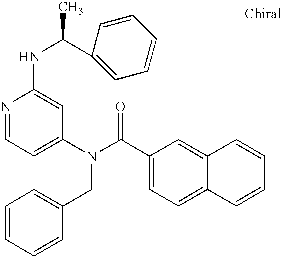 | A | 3.34 | 458 |
| 56 | 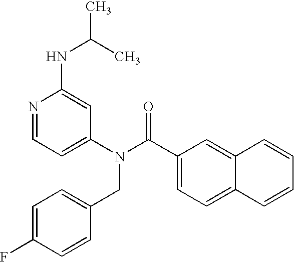 | A | 2.987 | 414 |
| 57 | 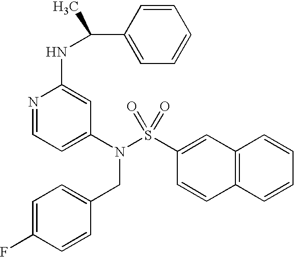 | A | 4.06 | 512 |
| 58 | 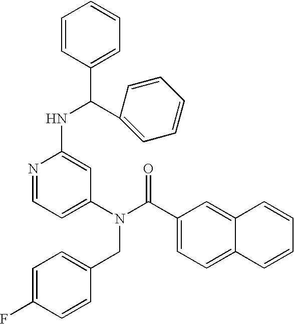 | A | 3.62 | 538 |
| 59 | 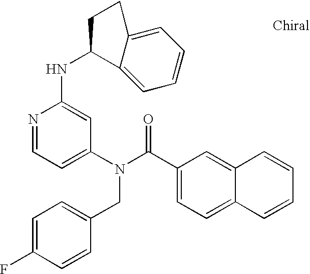 | A | 3.32 | 488 |
| 60 | 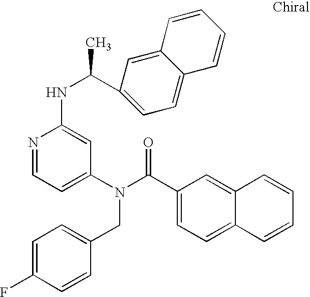 | A | 3.6 | 526 |
| 61 | 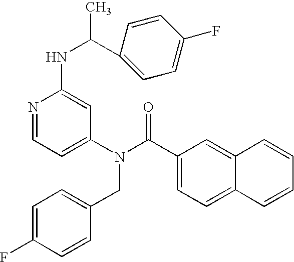 | A | 3.347 | 494 |
| 62 | 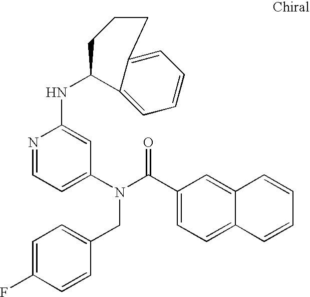 | A | 3.38 | 502 |
| 63 | 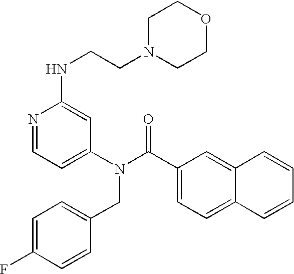 | A | 2.607 | 485 |
| 64 | 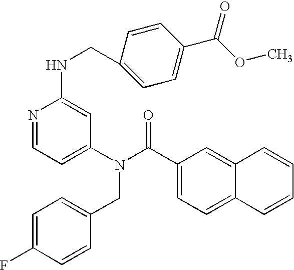 | A | 3.14 | 520 |
| 65 | 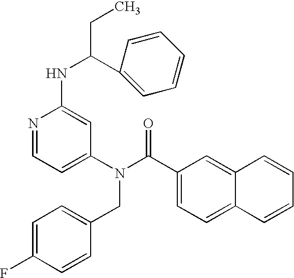 | A | 3.507 | 490 |
| 66 | 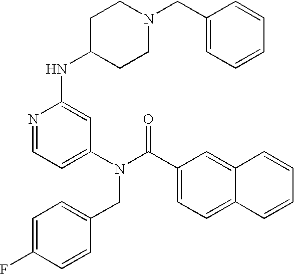 | A | 2.52 | 545 |
| 67 | 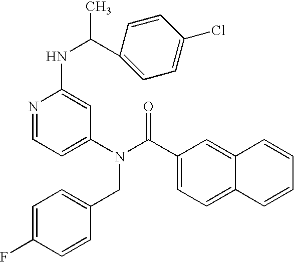 | A | 3.547 | 510 |
| 68 | 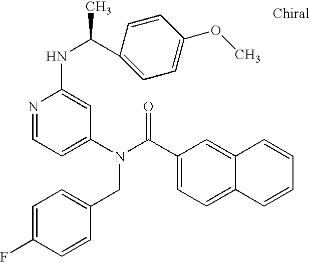 | A | 3.247 | 506 |
| 69 | 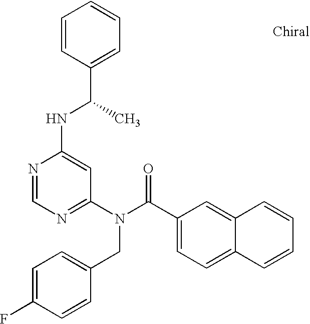 | A | 3.8 | 477 |
| 70 | 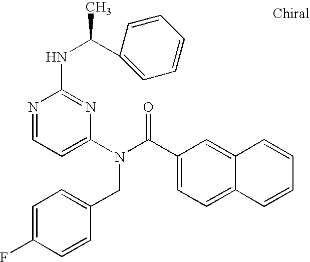 | A | 3.92 | 477 |
| 71 | 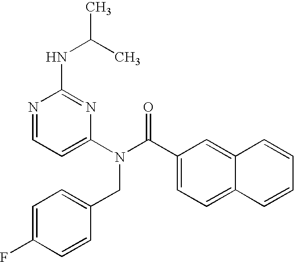 | A | 3.467 | 415 |
| 72 | 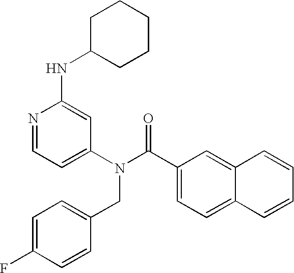 | A | 3.707 | 454 |
| 73 | 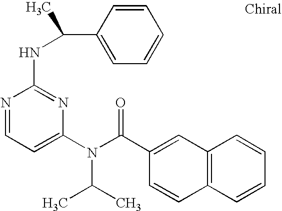 | A | 3.367 | 411 |
| 74 | 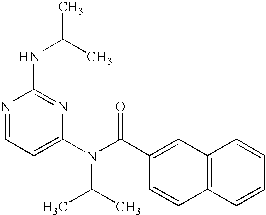 | A | 3.007 | 349 |
| 75 | 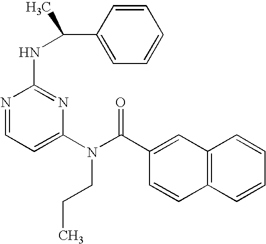 | A | 3.393 | 411 |
| 76 | 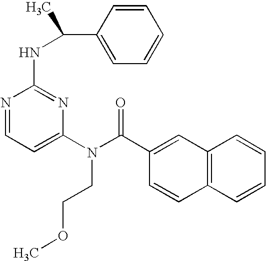 | A | 3.207 | 427 |
| 77 | 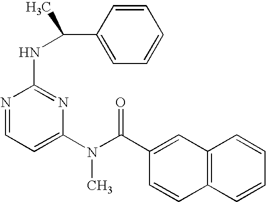 | A | 3.14 | 383 |
| 78 | 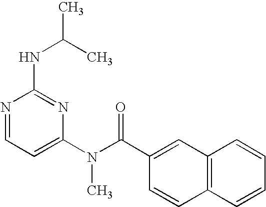 | A | 2.673 | 321 |
| 79 | 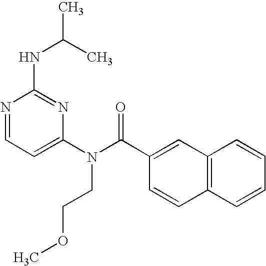 | A | 2.667 | 365 |
| 80 | 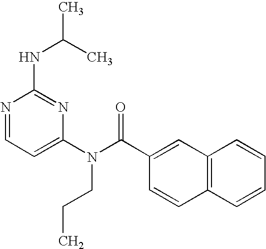 | A | 2.88 | 349 |
| 81 | 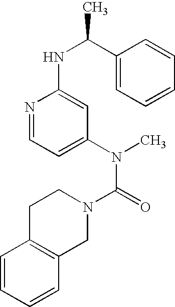 | A | 2.587 | 386 |
| 82 | 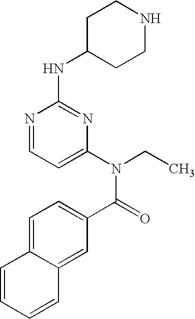 | A | 1.86 | 375 |
| 83 | 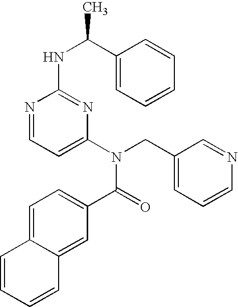 | A | 2.273 | 460 |
| 84 | 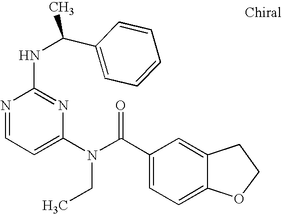 | A | 2.907 | 389 |
| 85 | 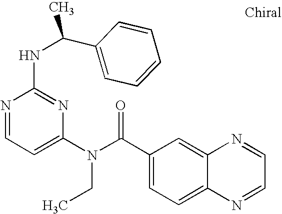 | A | 2.507 | 399 |
| 86 | 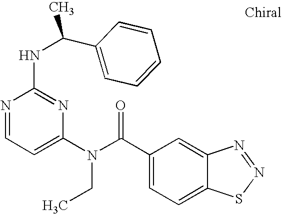 | A | 3.033 | 405 |
| 87 | 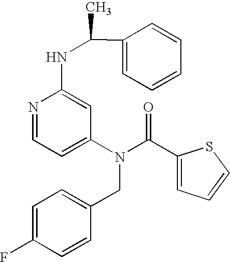 | A | 2.87 | 432 |
| 88 | 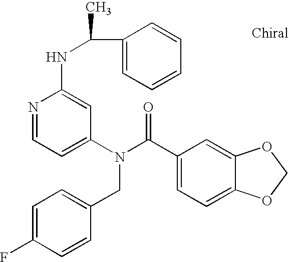 | A | 2.90 | 470 |
| 89 | 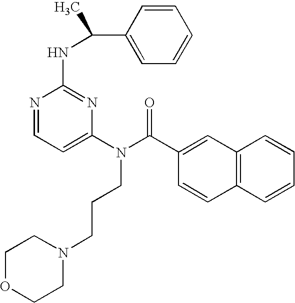 | A | 2.213 | 497 |
| 90 | 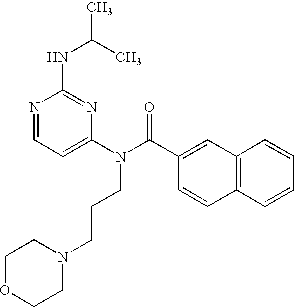 | A | 2.12 | 434 |
| 91 | 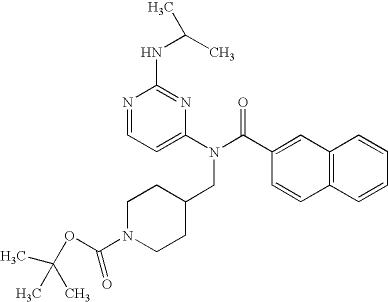 | A | 3.413 | 504 |
| 92 | 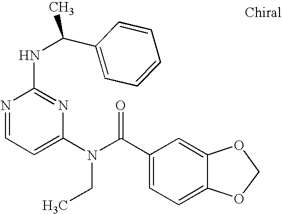 | A | 2.82 | 391 |
| 93 | 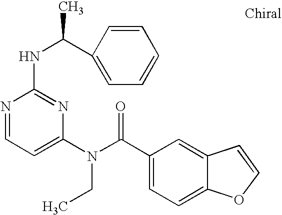 | A | 3.047 | 387 |
| 94 | 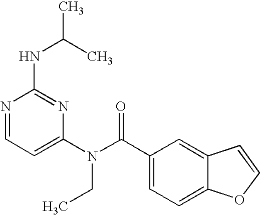 | A | 2.567 | 326 |
| 95 | 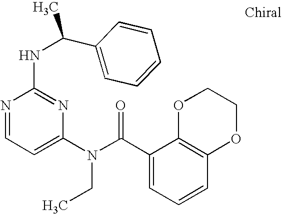 | A | 2.907 | 405 |
| 96 | 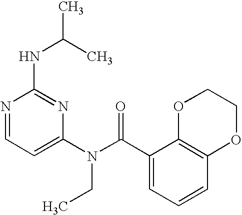 | A | 2.507 | 343 |
| 97 | 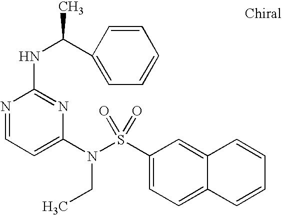 | A | 3.147 | 434 |
| 98 | 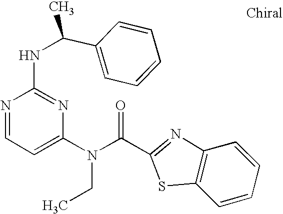 | A | 3.307 | 404 |
| 99 | 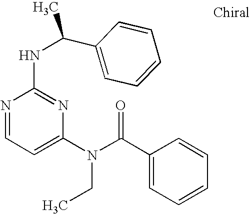 | A | 2.72 | 348 |
| 100 | 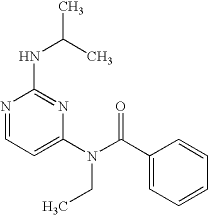 | A | 2.42 | 285 |
| 101 | 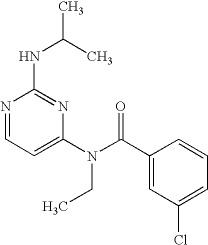 | A | 2.72 | 320 |
| 102 | 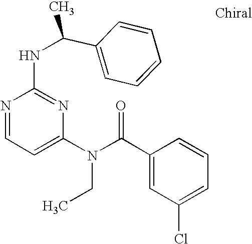 | A | 3 | 382 |
| 103 | 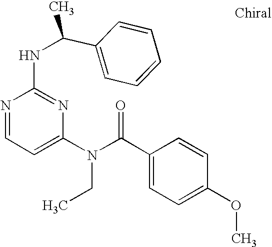 | A | 2.82 | 378 |
| 104 | 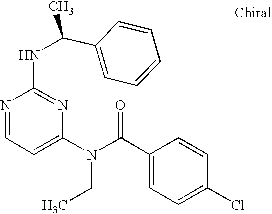 | A | 3.187 | 382 |
| 105 | 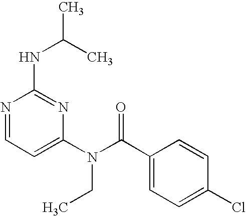 | A | 2.767 | 319 |
| 106 | 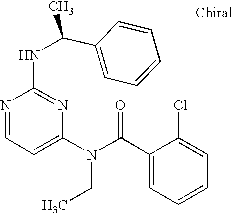 | A | 3.307 | 382 |
| 107 | 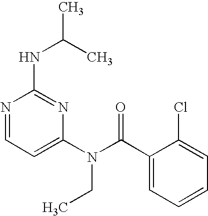 | A | 2.92 | 319 |
| 108 | 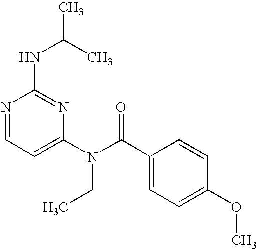 | A | 2.633 | 315 |
| 109 | 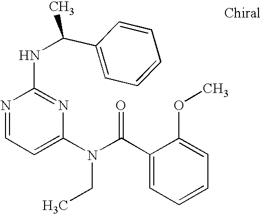 | A | 2.927 | 378 |
| 110 | 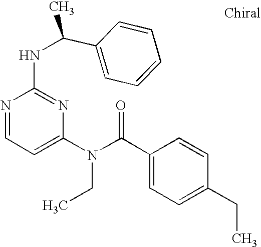 | A | 3.3 | 375 |
| 111 | 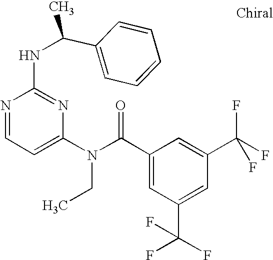 | A | 3.707 | 484 |
| 112 | 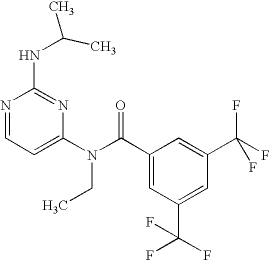 | A | 3.333 | 422 |
| 113 | 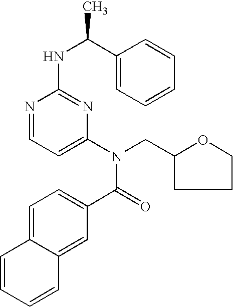 | A | 3.133 | 453 |
| 114 | 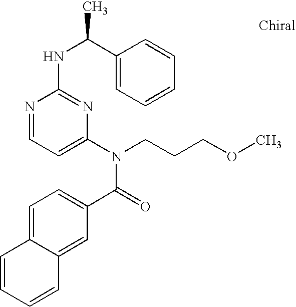 | A | 3.533 | 441 |
| 115 | 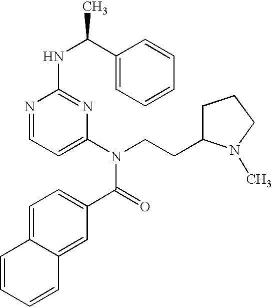 | A | 2.367 | 480 |
| 116 | 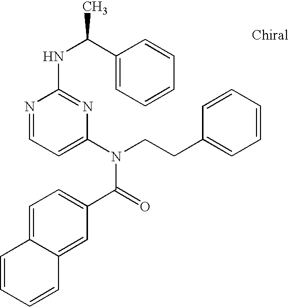 | A | 3.607 | 473 |
| 117 | 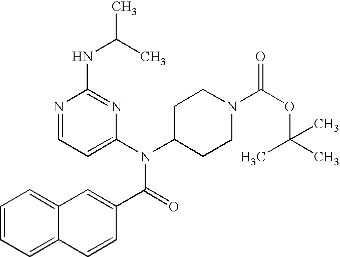 | A | 4.26 | 490 |
| 118 | 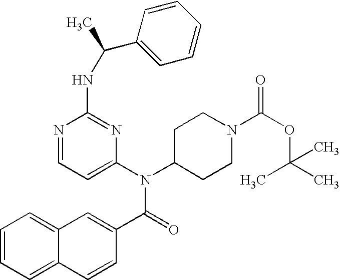 | A | 4.34 | 552 |
| 119 | 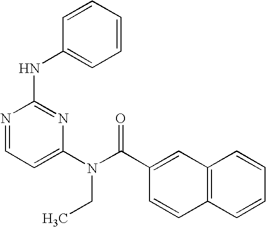 | A | 3.66 | 369 |
| 120 | 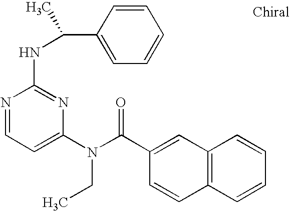 | A | 3.193 | 397 |
| 121 | 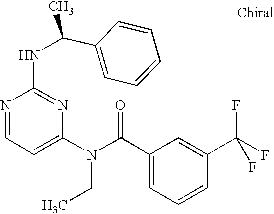 | A | 3.053 | 415 |
| 122 | 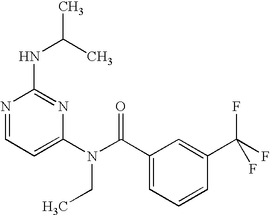 | A | 3.08 | 353 |
| 123 | 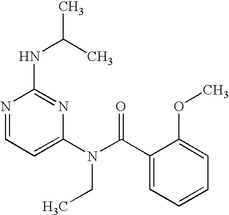 | A | 2.527 | 315 |
| 124 | 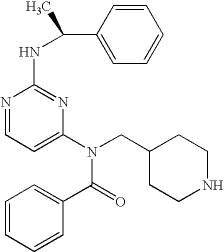 | A | 1.9 | 417 |
| 125 | 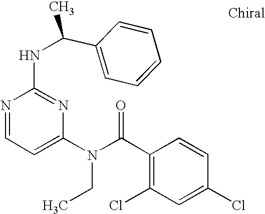 | A | 3.34 | 415 |
| 126 | 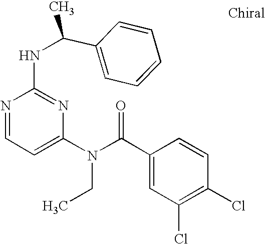 | A | 3.32 | 415 |
| 127 | 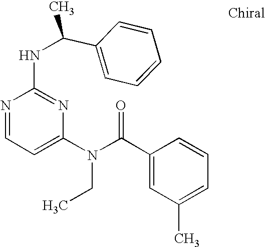 | A | 3.007 | 361 |
| 128 | 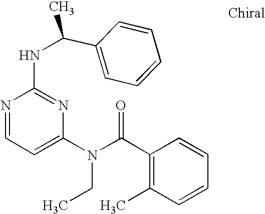 | A | 2.953 | 361 |
| 129 | 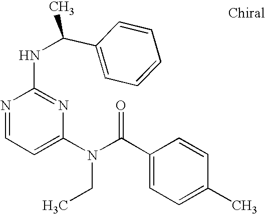 | A | 2.973 | 361 |
| 130 | 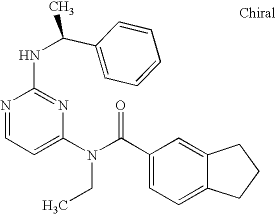 | A | 3.207 | 387 |
| 131 | 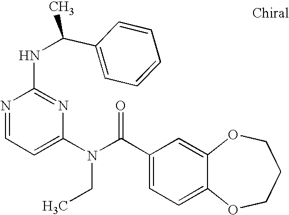 | A | 2.867 | 420 |
| 132 | 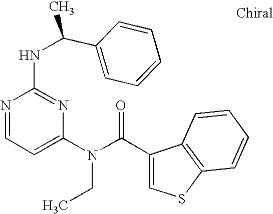 | A | 3.11 | 403 |
| 133 | 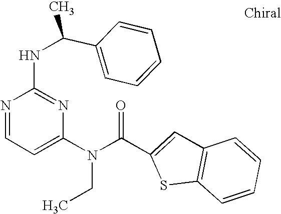 | A | 3.273 | 403 |
| 134 | 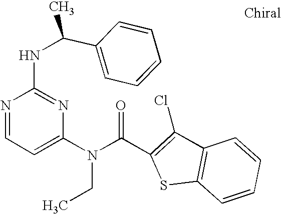 | A | 3.46 | 438 |
| 135 | 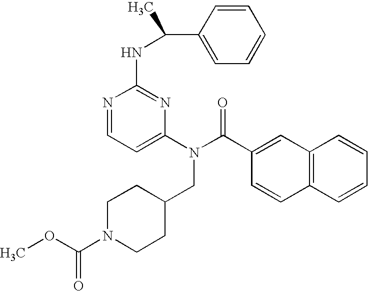 | A | 3.1 | 525 |
| 136 | 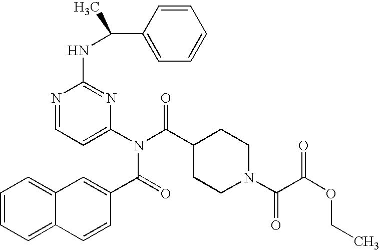 | A | 3.227 | 567 |
| 137 | 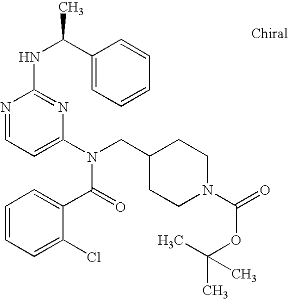 | A | 3.62 | 550 |
| 138 | 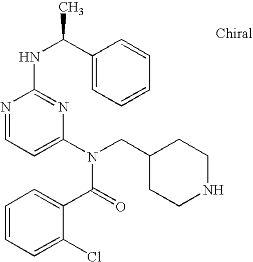 | A | 2.227 | 450 |
| 139 | 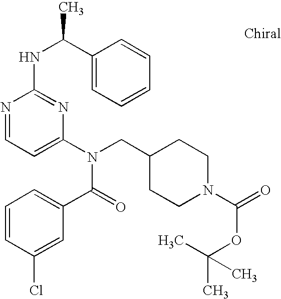 | A | 3.693 | 550 |
| 140 | 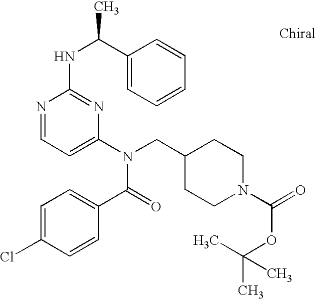 | A | 3.693 | 550 |
| 141 | 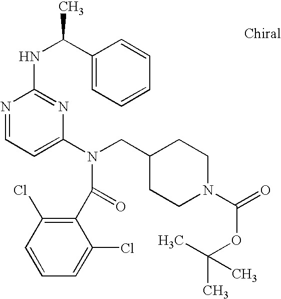 | A | 3.88 | 585 |
| 142 | 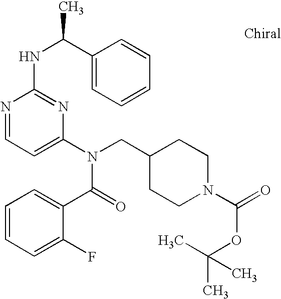 | A | 3.46 | 534 |
| 143 | 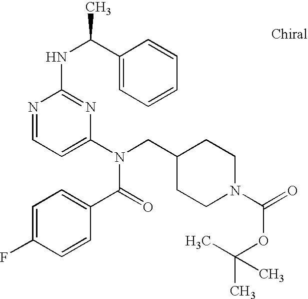 | A | 3.48 | 534 |
| 144 | 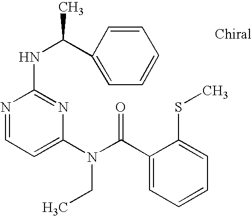 | A | 2.96 | 393 |
| 145 | 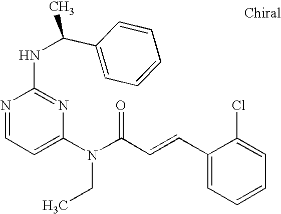 | A | 3.313 | 407 |
| 146 | 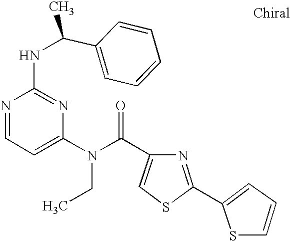 | A | 3.073 | 436 |
| 147 | 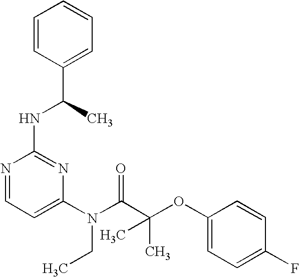 | A | 3.613 | 439 |
| 148 | 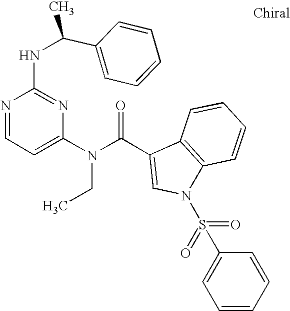 | A | 3.487 | 526 |
| 149 | 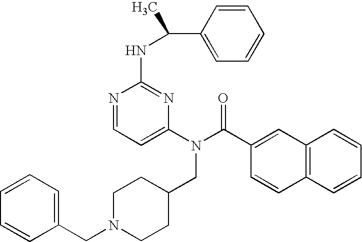 | A | 2.553 | 542 |
| 150 | 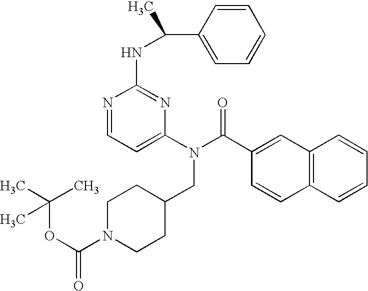 | A | 3.64 | 567 |
| 151 | 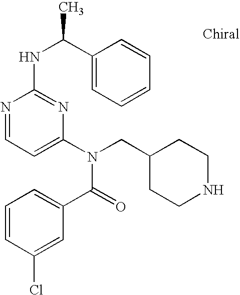 | A | 2.253 | 450 |
| 152 | 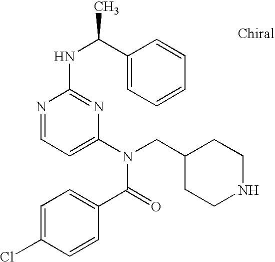 | A | 2.247 | 450 |
| 153 | 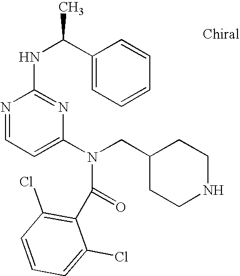 | A | 2.273 | 484 |
| 154 | 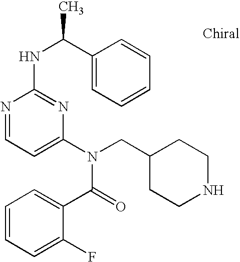 | A | 2.053 | 434 |
| 155 | 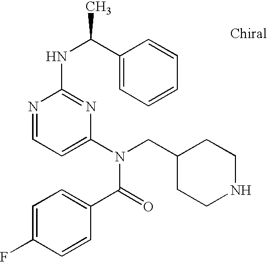 | A | 2.087 | 434 |
| 156 | 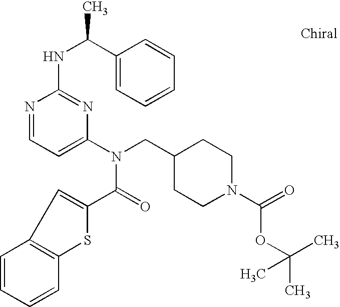 | A | 3.453 | 572 |
| 157 | 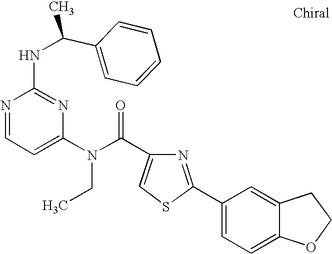 | A | 3.213 | 472 |
| 158 | 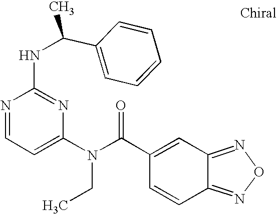 | A | 2.907 | 389 |
| 159 | 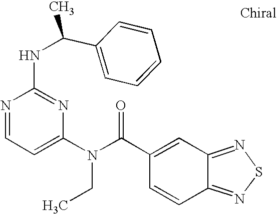 | A | 2.827 | 405 |
| 160 | 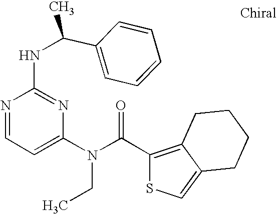 | A | 3.267 | 407 |
| 161 | 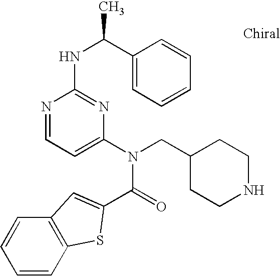 | A | 2.31 | 472 |
| 162 | 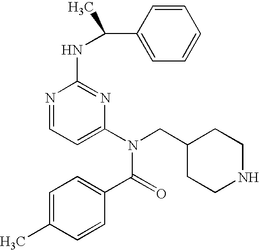 | A | 2.033 | 430 |
| 163 | 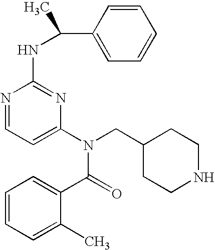 | A | 2.087 | 430 |
| 164 | 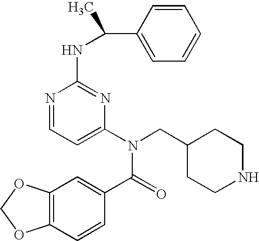 | A | 1.967 | 460 |
| 165 | 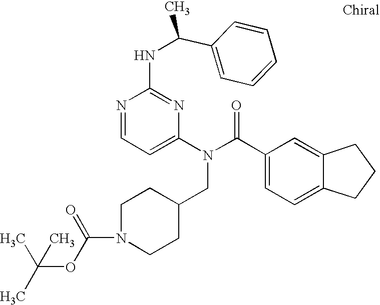 | A | 3.847 | 556 |
| 166 | 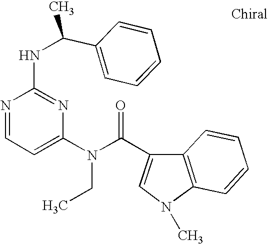 | A | 2.967 | 400 |
| 167 | 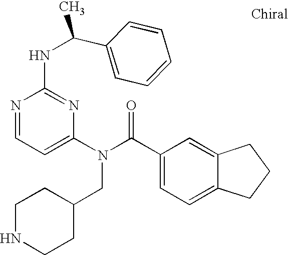 | A | 2.353 | 456 |
| 168 | 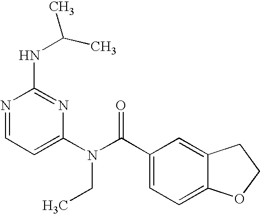 | A | 2.40 | 327 |
| 169 | 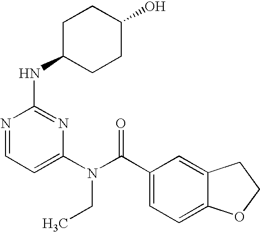 | A | 1.98 | 383 |
| 170 | 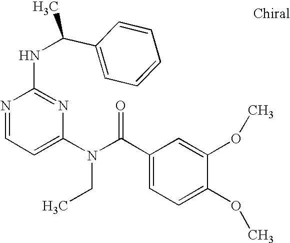 | A | 2.73 | 470 |
| 171 | 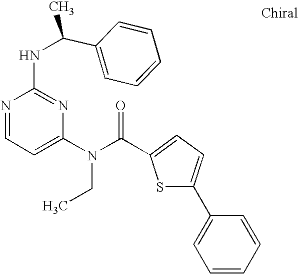 | A | 3.33 | 429 |
| 172 | 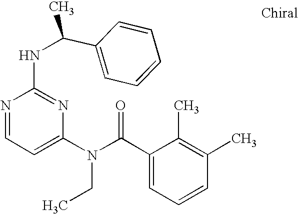 | A | 3.187 | 376 |
| 173 | 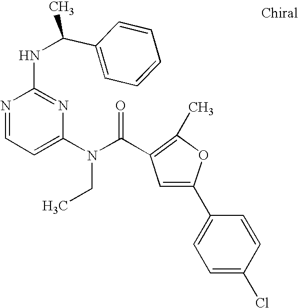 | A | 3.73 | 461 |
| 174 | 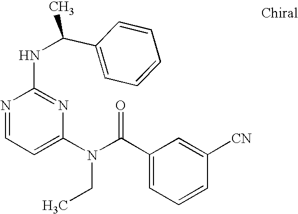 | A | 2.75 | 372 |
| 175 | 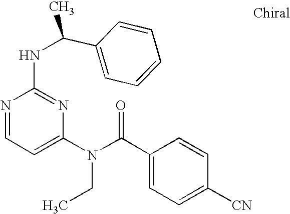 | A | 2.77 | 372 |
| 176 | 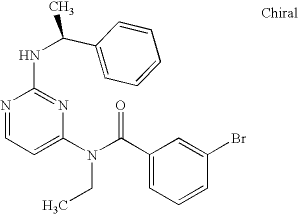 | A | 3.09 | 425 |
| 177 | 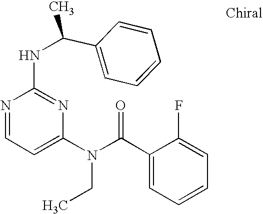 | A | 2.83 | 365 |
| 178 | 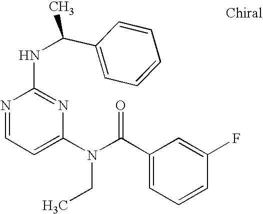 | A | 2.89 | 365 |
| 179 | 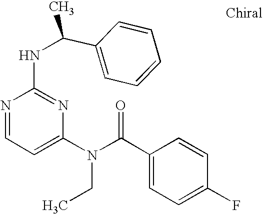 | A | 2.87 | 365 |
| 180 | 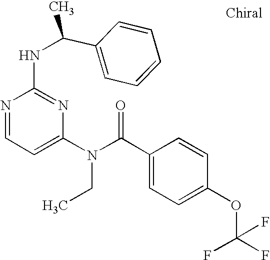 | A | 3.29 | 431 |
| 181 | 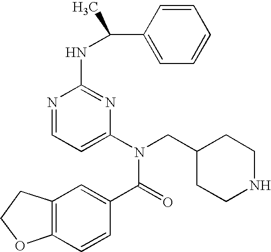 | A | 2.1 | 458 |
| 182 | 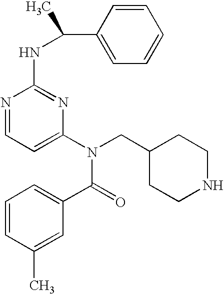 | A | 2.22 | 430 |
| 183 | 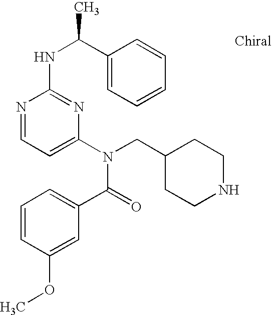 | A | 2.087 | 446 |
| 184 | 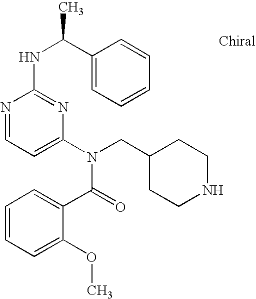 | A | 2.027 | 446 |
| 185 | 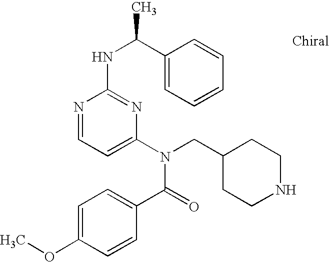 | A | 2.08 | 446 |
| 186 | 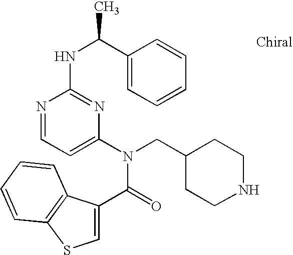 | A | 2.247 | 472 |
| 187 | 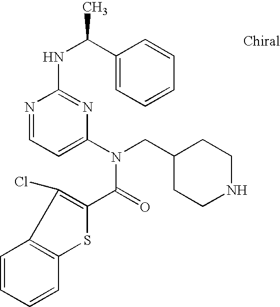 | A | 2.427 | 506 |
| 188 | 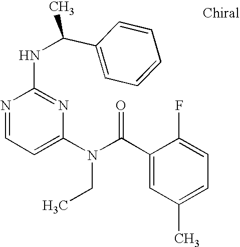 | A | 3.04 | 379 |
| 189 | 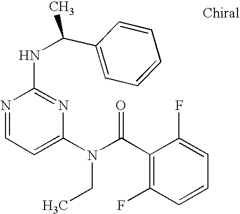 | A | 2.953 | 383 |
| 190 | 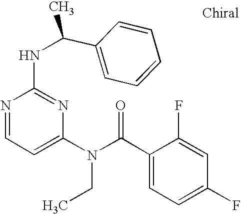 | A | 2.727 | 383 |
| 191 | 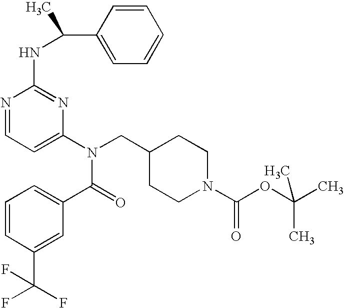 | A | 3.8 | 584 |
| 192 | 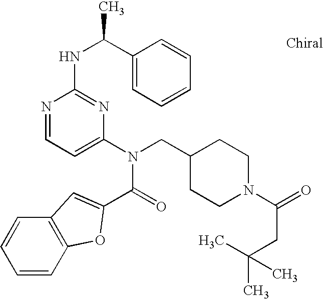 | A | 3.607 | 556 |
| 193 | 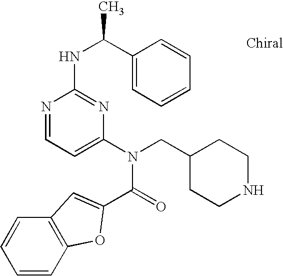 | A | 2.2 | 456 |
| 194 | 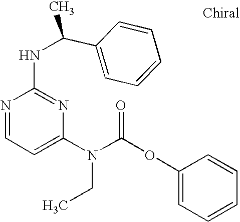 | A | 3.093 | 364 |
| 195 | 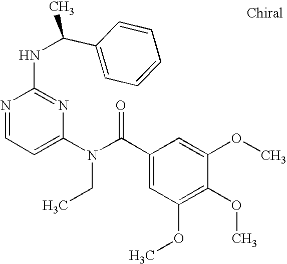 | A | 2.827 | 437 |
| 196 | 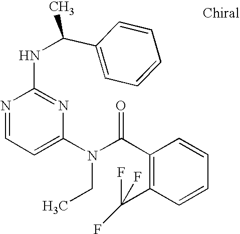 | A | 2.84 | 415 |
| 197 | 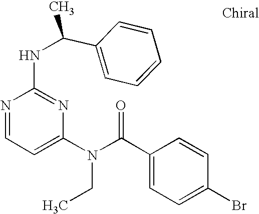 | A | 3.247 | 425 |
| 198 | 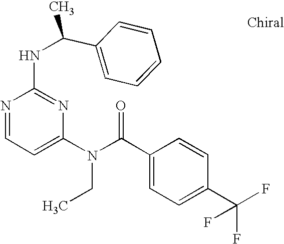 | A | 3.347 | 415 |
| 199 | 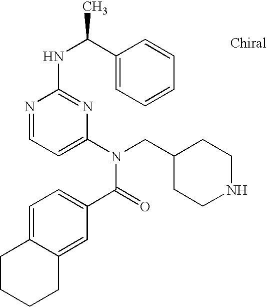 | A | 2.447 | 470 |
| 200 | 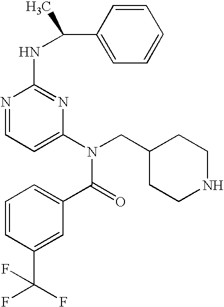 | A | 2.453 | 485 |
| 201 | 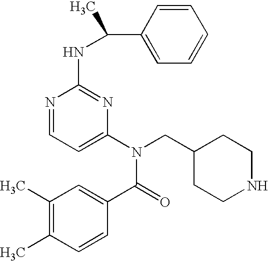 | A | 2.367 | 445 |
| 202 | 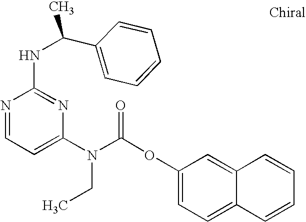 | A | 3.593 | 414 |
| 203 | 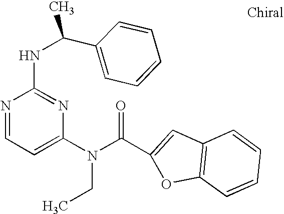 | A | 3.06 | 387 |
| 204 | 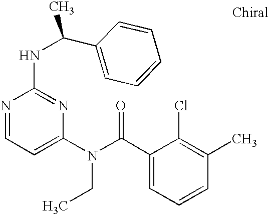 | A | 3.16 | 395 |
| 205 | 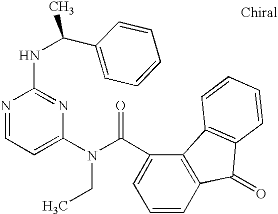 | A | 3.127 | 450 |
| 206 | 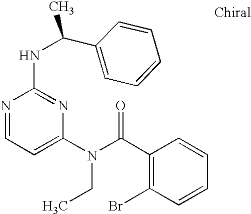 | A | 2.993 | 425 |
| 207 | 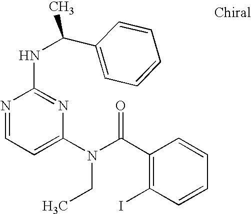 | A | 3.147 | 473 |
| 208 | 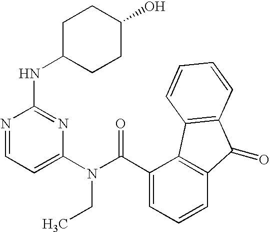 | A | 2.32 | 444 |
| 209 | 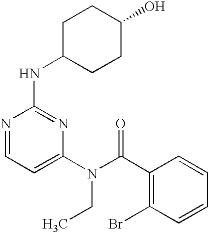 | A | 2.127 | 421 |
| 210 | 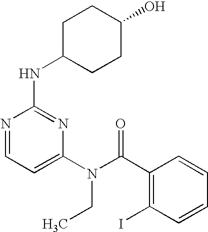 | A | 2.12 | 467 |
| 211 | 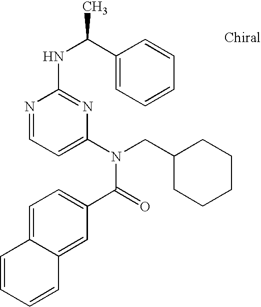 | A | 4.053 | 465 |
| 212 | 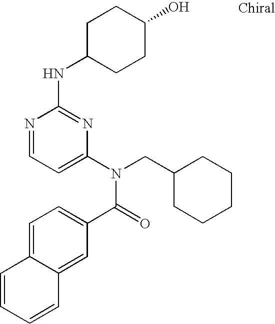 | A | 3.02 | 459 |
| 213 | 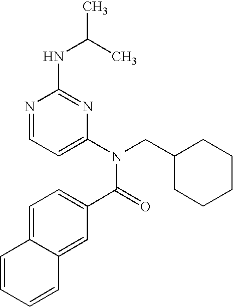 | A | 3.68 | 403 |
| 214 | 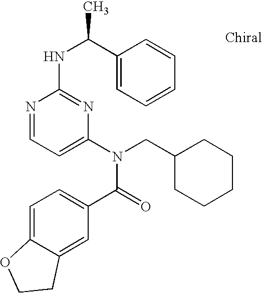 | A | 3.553 | 457 |
| 215 | 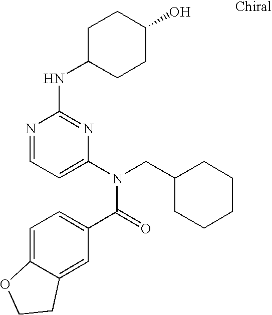 | A | 2.747 | 451 |
| 216 | 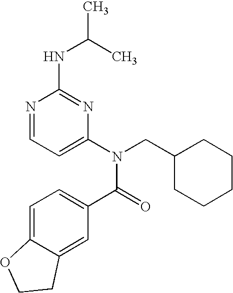 | A | 3.28 | 395 |
| 217 | 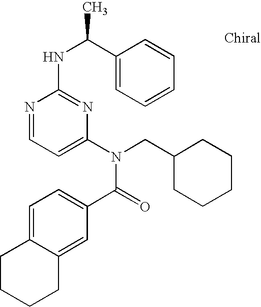 | A | 4.227 | 469 |
| 218 | 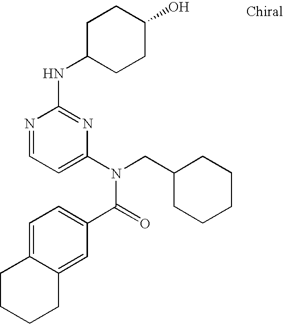 | A | 3.193 | 463 |
| 219 | 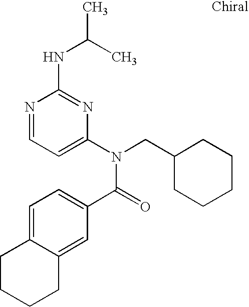 | A | 3.9 | 407 |
| 220 | 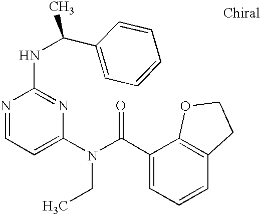 | A | 2.787 | 390 |
| 221 | 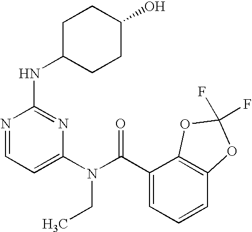 | A | 2.14 | 421 |
| 222 | 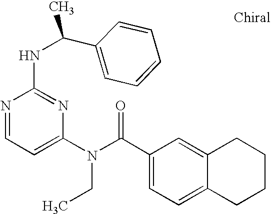 | A | 3.333 | 401 |
| 223 | 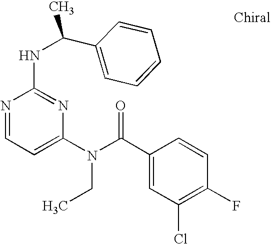 | A | 3.19 | 399 |
| 224 | 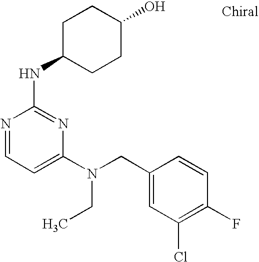 | A | 2.20 | 393 |
| 225 | 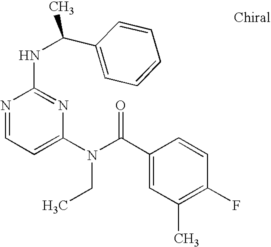 | A | 3.12 | 397 |
| 226 | 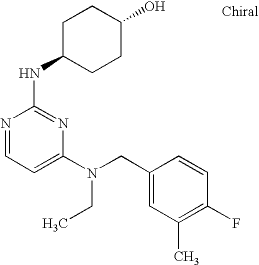 | A | 2.18 | 373 |
| 227 | 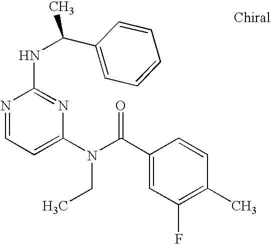 | A | 3.13 | 379 |
| 228 | 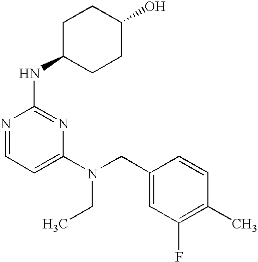 | A | 2.14 | 373 |
| 229 | 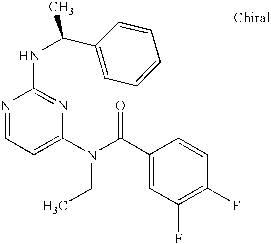 | A | 2.99 | 383 |
| 230 | 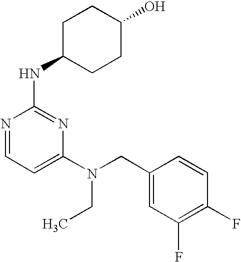 | A | 2.01 | 377 |
| 231 | 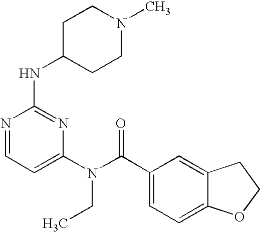 | A | 1.70 | 382 |
| 232 | 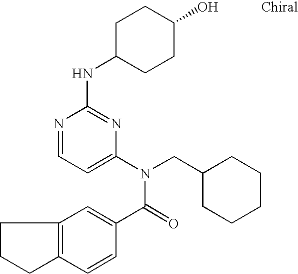 | A | 3.033 | 449 |
| 233 | 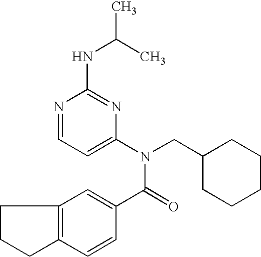 | A | 3.7 | 393 |
| 234 | 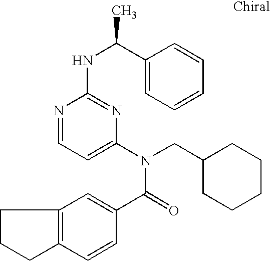 | A | 4.133 | 455 |
| 235 | 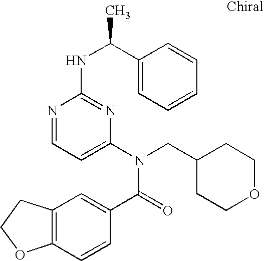 | A | 2.8 | 459 |
| 236 | 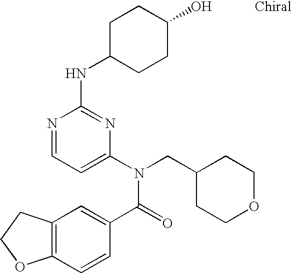 | A | 2.007 | 453 |
| 237 | 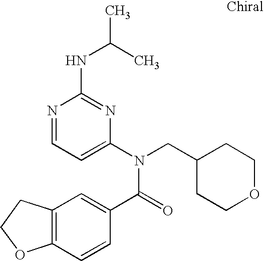 | A | 2.38 | 396 |
| 238 | 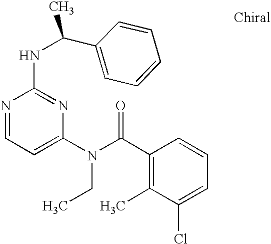 | A | 3.26 | 395 |
| 239 | 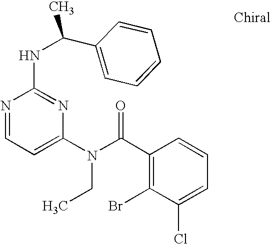 | A | 3.227 | 439 |
| 240 | 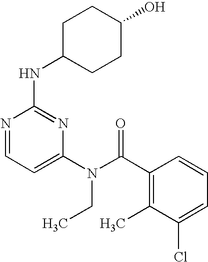 | A | 2.313 | 389 |
| 241 | 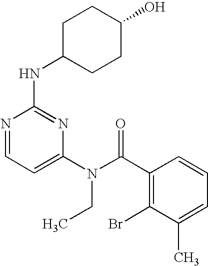 | A | 2.3 | 433 |
| 242 | 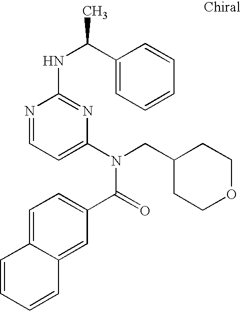 | A | 3.127 | 467 |
| 243 | 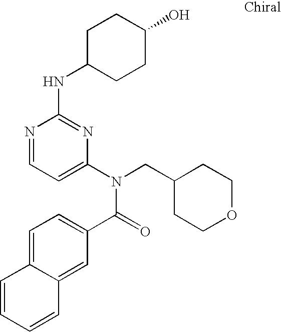 | A | 2.353 | 461 |
| 244 | 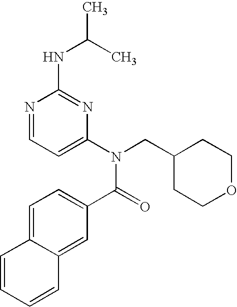 | A | 2.753 | 405 |
| 245 | 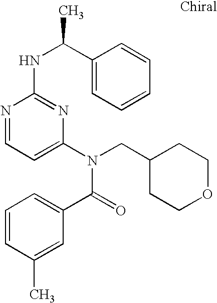 | A | 2.927 | 431 |
| 246 | 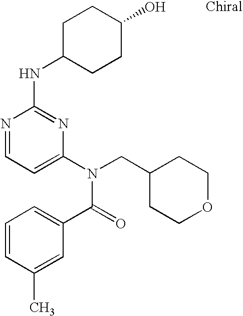 | A | 2.1 | 425 |
| 247 | 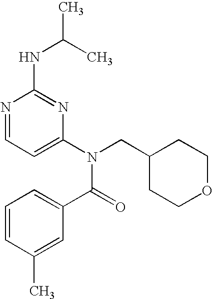 | A | 2.54 | 368 |
| 248 | 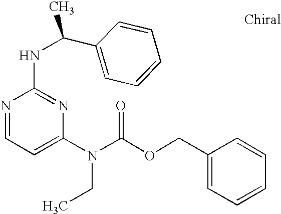 | A | 3.18 | 377 |
| 249 | 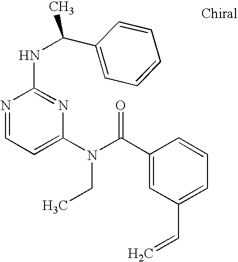 | A | 3.113 | 373 |
| 250 | 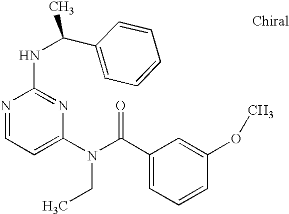 | A | 2.707 | 377 |
| 251 | 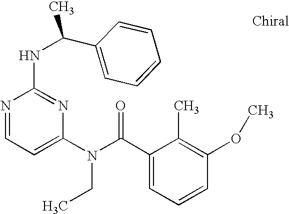 | A | 2.967 | 391 |
| 252 | 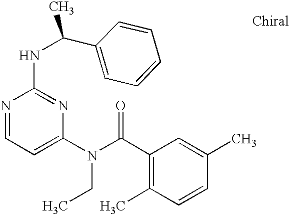 | A | 3.14 | 375 |
| 253 | 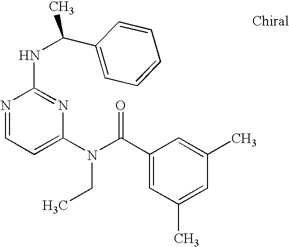 | A | 3.11 | 375 |
| 254 | 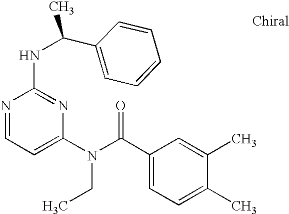 | A | 3.16 | 375 |
| 255 | 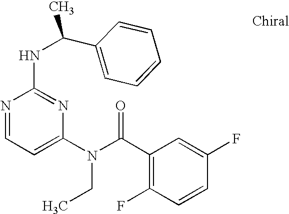 | A | 2.90 | 383 |
| 256 | 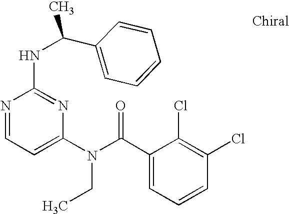 | A | 3.21 | 415 |
| 257 | 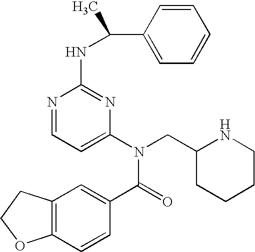 | A | 2.24 | 459 |
| 258 | 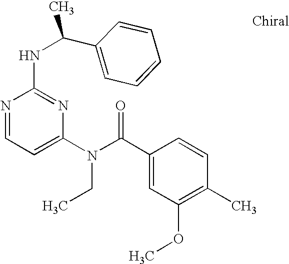 | A | 3.14 | 391 |
| 259 | 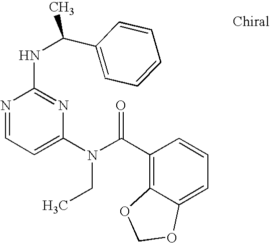 | A | 2.77 | 391 |
| 260 | 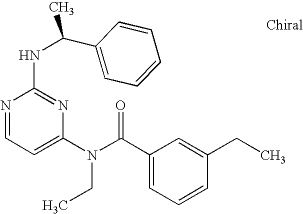 | B | 1.627 | 376 |
| 261 | 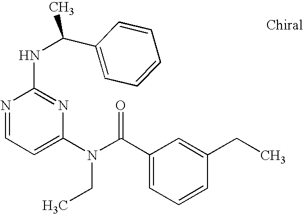 | A | 3.19 | 375 |
| 262 | 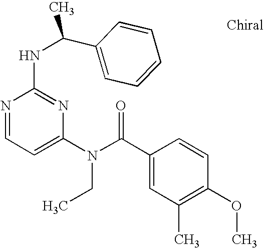 | A | 3.113 | 391 |
| 263 | 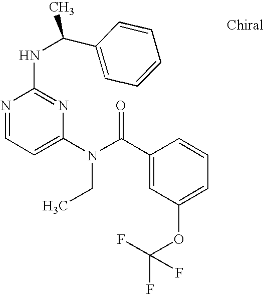 | A | 3.2 | 432 |
| 264 | 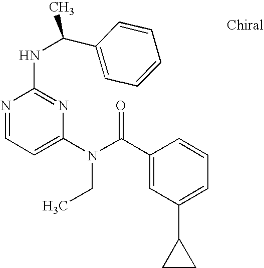 | A | 3.247 | 387 |
| 265 | 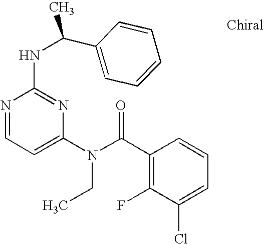 | A | 3.16 | 399 |
| 266 | 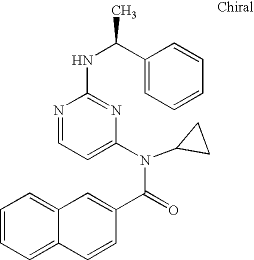 | A | 3.197 | 409 |
| 267 | 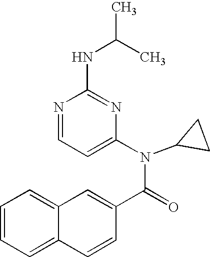 | A | 2.853 | 346 |
| 268 | 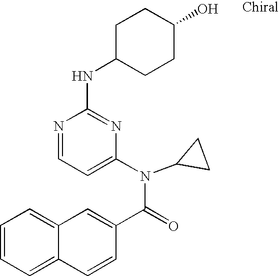 | A | 2.313 | 402 |
| 269 | 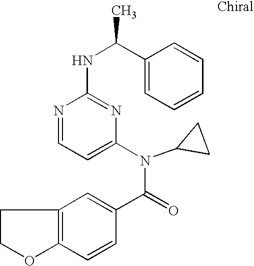 | A | 2.84 | 400 |
| 270 | 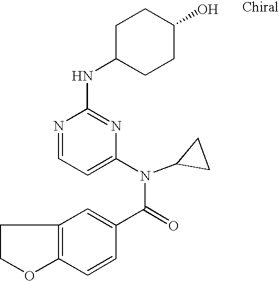 | A | 2.013 | 394 |
| 271 | 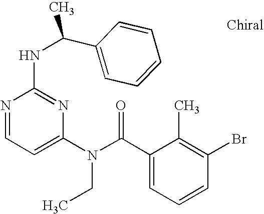 | A | 3.353 | 441 |
| 272 | 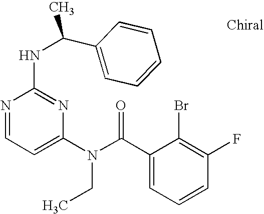 | A | 3.06 | 445 |
| 273 | 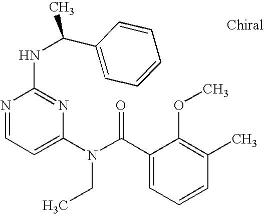 | A | 3.16 | 391 |
| 274 | 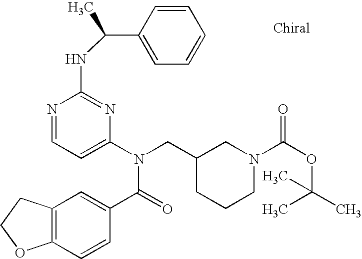 | A | 3.247 | 559 |
| 275 | 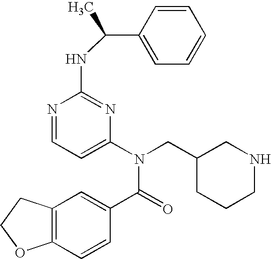 | A | 2.04 | 458 |
| 276 | 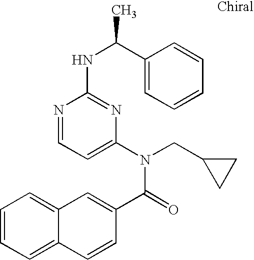 | A | 3.453 | 423 |
| 277 | 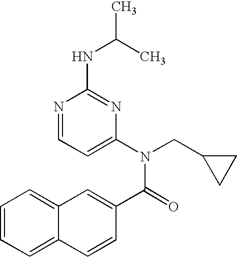 | A | 3.35 | 360 |
| 278 | 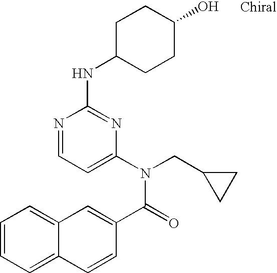 | A | 2.83 | 417 |
| 279 | 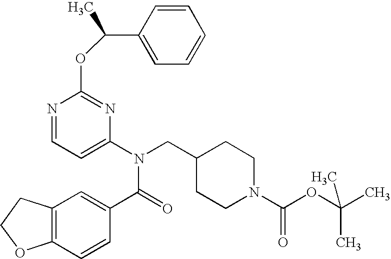 | A | 4.06 | 559 |
| 280 | 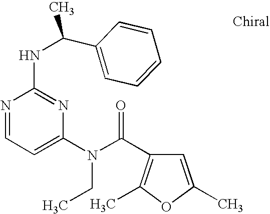 | A | 3.087 | 365 |
| 281 | 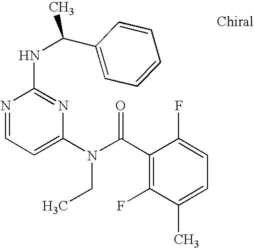 | A | 3.14 | 397 |
| 282 | 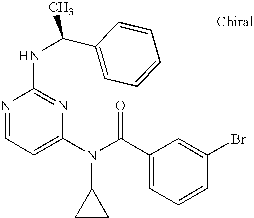 | A | 3.14 | 437 |
| 283 | 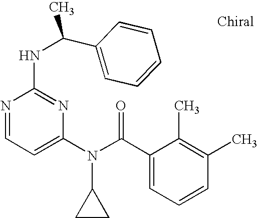 | A | 3.12 | 387 |
| 284 | 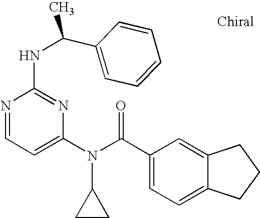 | A | 3.247 | 399 |
| 285 | 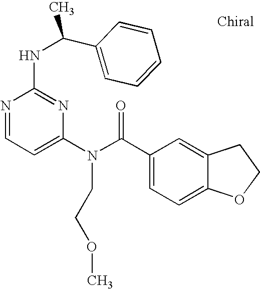 | A | 2.793 | 420 |
| 286 | 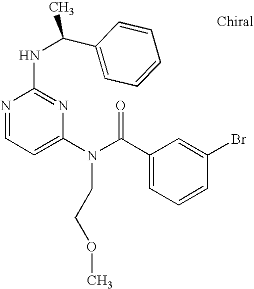 | A | 3.027 | 455 |
| 287 | 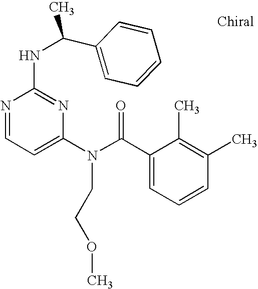 | A | 3.1 | 406 |
| 288 | 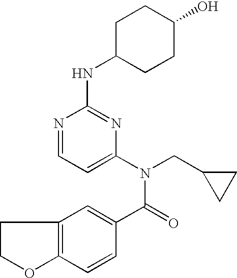 | A | 2.253 | 408 |
| 289 | 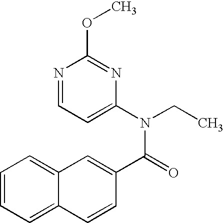 | A | 3.067 | 307 |
| 290 | 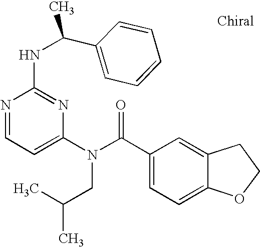 | A | 3.233 | 418 |
| 291 | 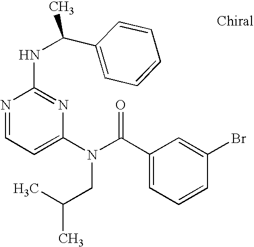 | A | 3.487 | 306 |
| 292 | 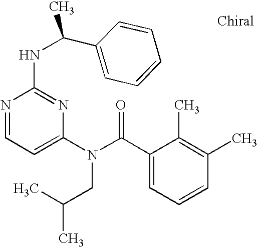 | A | 3.447 | 403 |
| 293 | 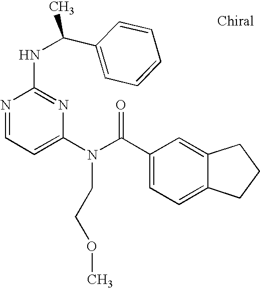 | A | 3.093 | 417 |
| 294 | 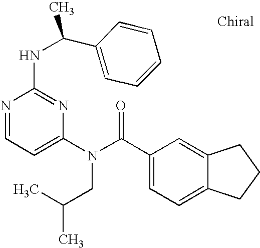 | A | 3.573 | 415 |
| 295 | 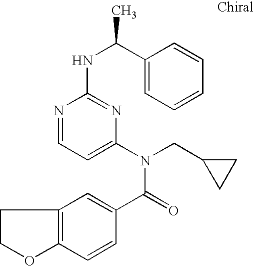 | A | 3.133 | 415 |
| 296 | 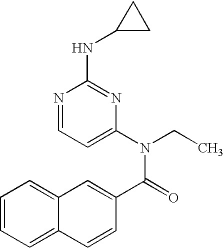 | A | 2.91 | 332 |
| 297 | 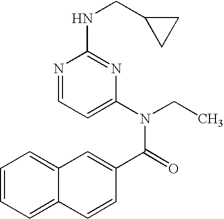 | A | 3.24 | 346 |
| 298 | 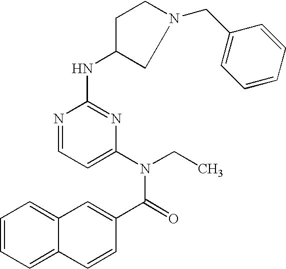 | A | 2.67 | 452 |
| 299 | 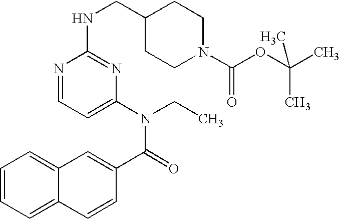 | A | 3.58 | 490 |
| 300 | 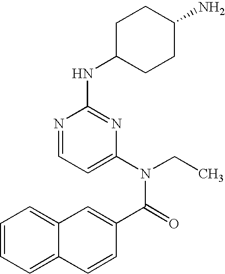 | A | 2.08 | 390 |
| 301 | 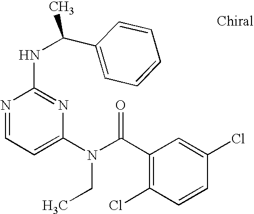 | A | 3.273 | 415 |
| 302 | 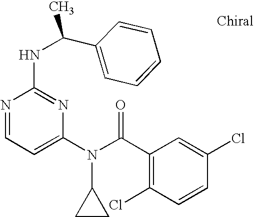 | A | 3.26 | 427 |
| 303 | 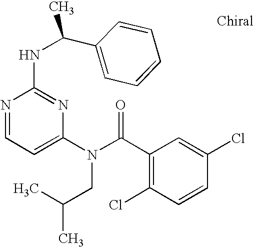 | A | 3.733 | 444 |
| 304 | 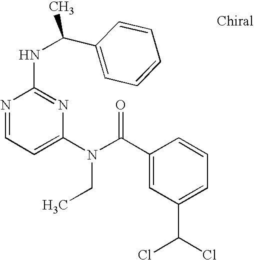 | A | 3.26 | 430 |
| 305 | 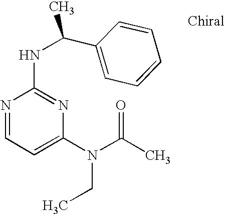 | A | 2.267 | 285 |
| 306 | 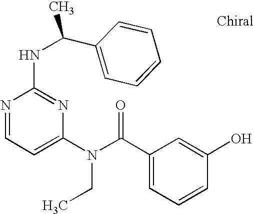 | A | 2.58 | 364 |
| 307 | 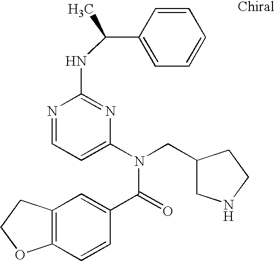 | A | 1.973 | 444 |
| 308 | 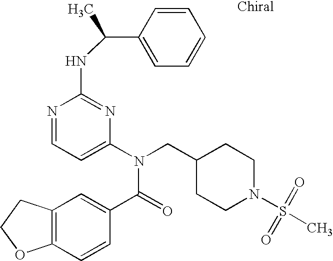 | B | 1.453 | 536 |
| 309 | 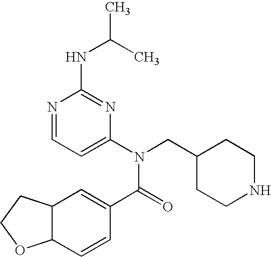 | B | 1.733 | 397 |
| 310 | 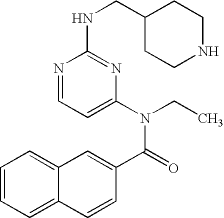 | B | 1.067 | 390 |
| 311 | 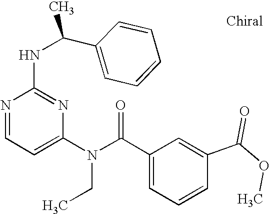 | A | 2.76 | 405 |
| 312 | 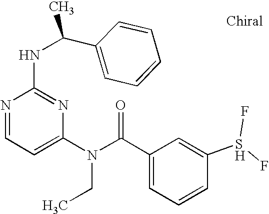 | A | 3.153 | 430 |
| 313 | 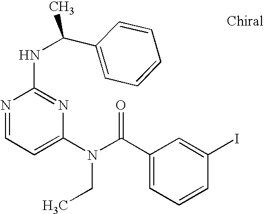 | A | 3.167 | 473 |
| 314 | 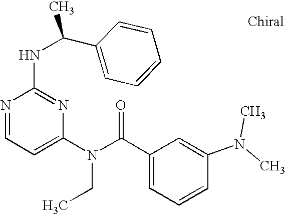 | A | 2.567 | 390 |
| 315 | 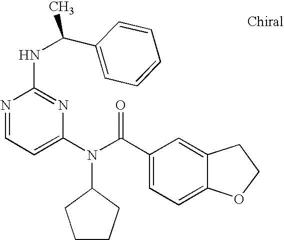 | A | 3.167 | 430 |
| 316 | 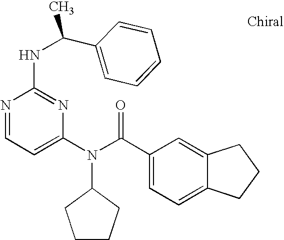 | A | 3.713 | 428 |
| 317 | 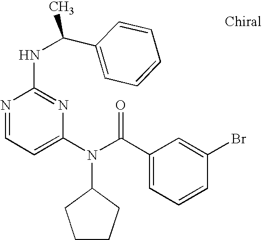 | A | 3.567 | 467 |
| 318 | 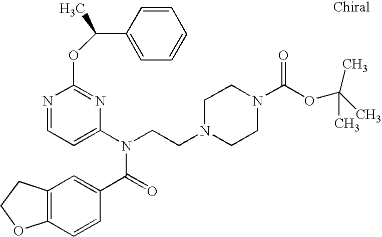 | A | 2.48 | 574 |
| 319 | 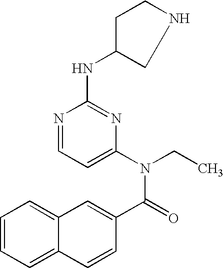 | B | 1.06 | 361 |
| 320 | 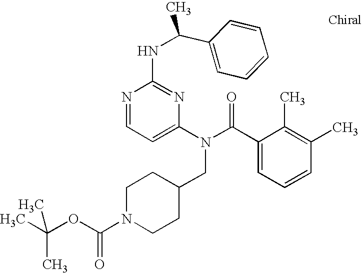 | B | 1.893 | 544 |
| 321 | 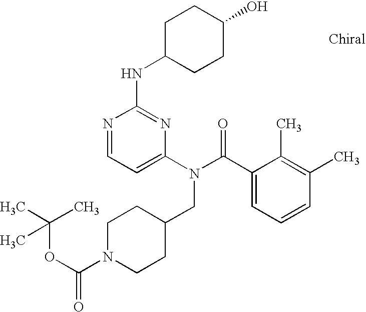 | B | 1.493 | 538 |
| 322 | 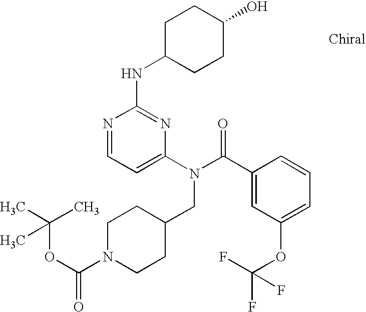 | B | 1.547 | 594 |
| 323 | 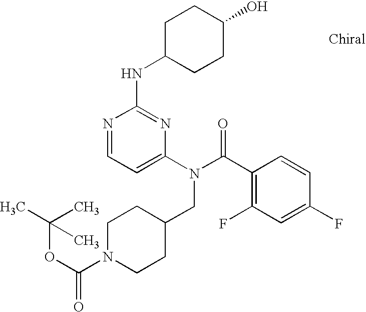 | B | 1.42 | 546 |
| 324 | 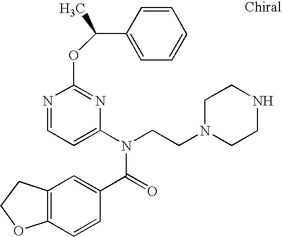 | A | 1.9 | 474 |
| 325 | 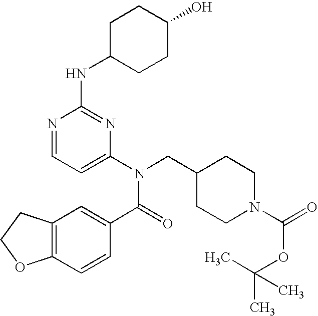 | B | 1.413 | 552 |
| 326 | 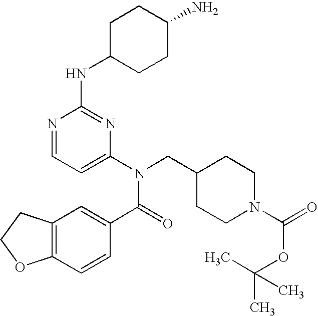 | B | 1.26 | 551 |
| 327 | 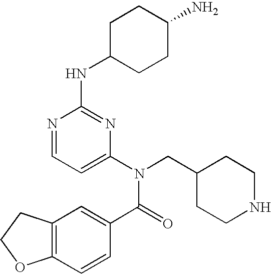 | B | 0.747 | 451 |
| 328 | 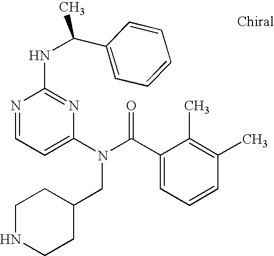 | B | 1.153 | 444 |
| 329 | 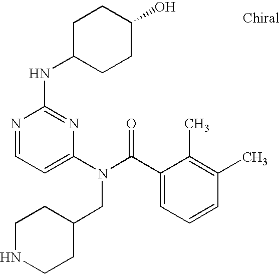 | B | 0.94 | 438 |
| 330 | 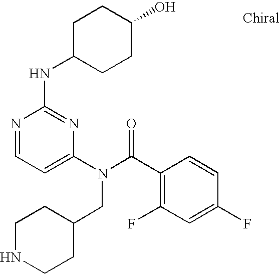 | B | 0.84 | 446 |
| 331 | 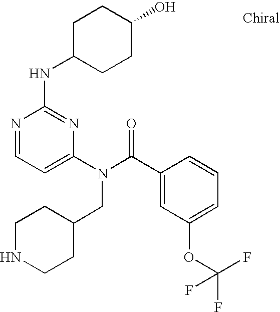 | B | 0.993 | 494 |
| 332 | 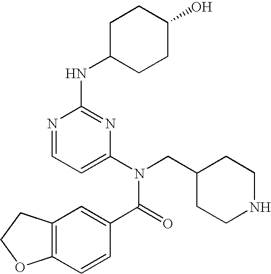 | B | 0.827 | 452 |
| 333 | 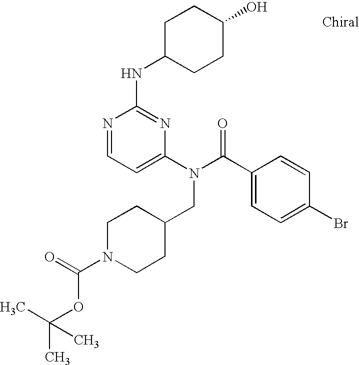 | B | 1.48 | 590 |
| 334 | 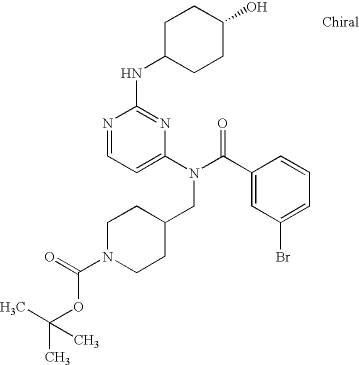 | B | 1.473 | 590 |
| 335 | 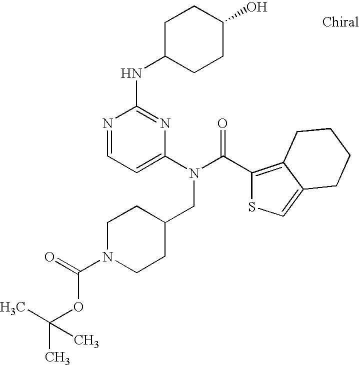 | B | 1.56 | 570 |
| 336 | 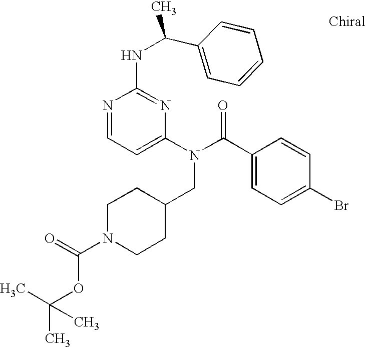 | B | 1.887 | 596 |
| 337 | 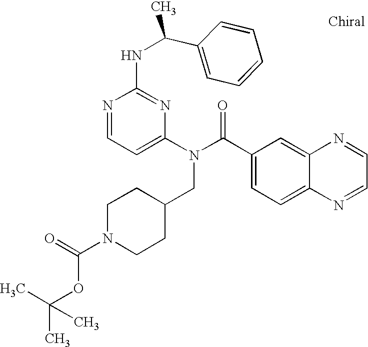 | B | 1.58 | 568 |
| 338 | 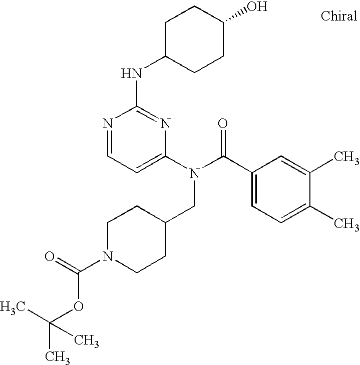 | B | 1.5 | 538 |
| 339 | 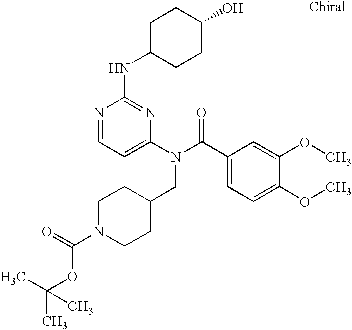 | B | 1.507 | 554 |
| 340 | 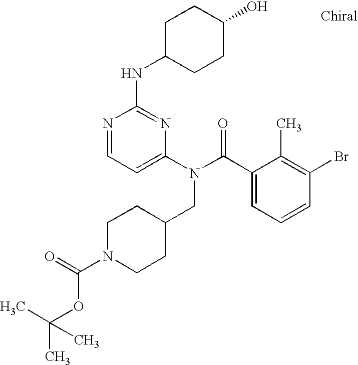 | B | 1.553 | 604 |
| 341 | 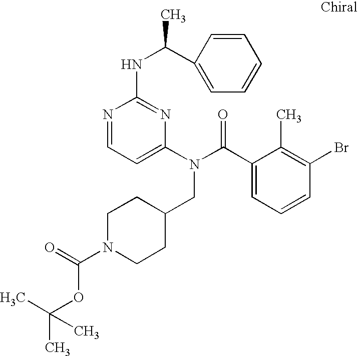 | B | 1.98 | 608 |
| 342 | 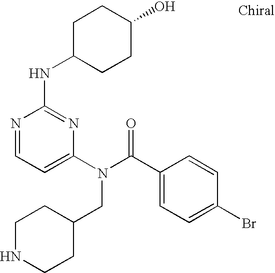 | B | 0.92 | 488 |
| 343 | 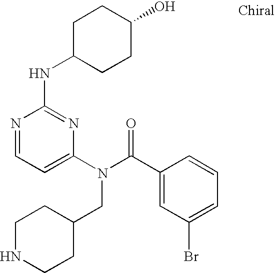 | B | 0.927 | 490 |
| 344 | 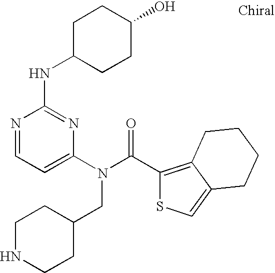 | B | 0.94 | 470 |
| 345 | 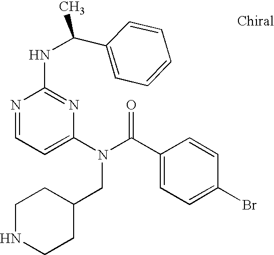 | B | 1.253 | 494 |
| 346 | 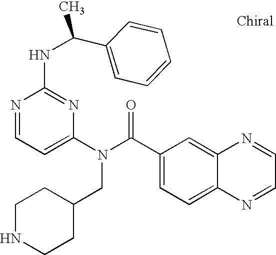 | B | 0.987 | 468 |
| 347 | 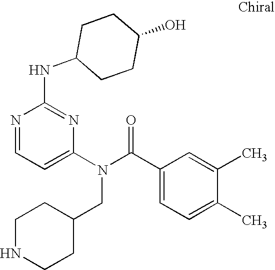 | B | 1.08 | 438 |
| 348 | 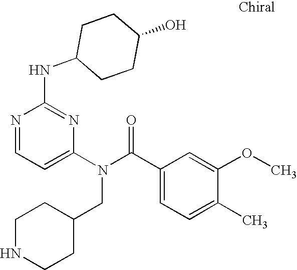 | B | 1.04 | 454 |
| 349 | 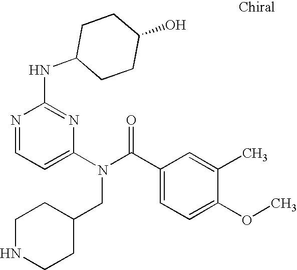 | B | 1.02 | 454 |
| 350 | 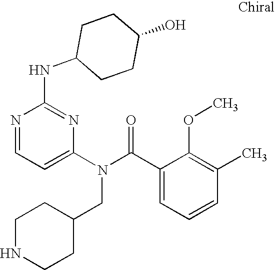 | B | 0.893 | 454 |
| 351 | 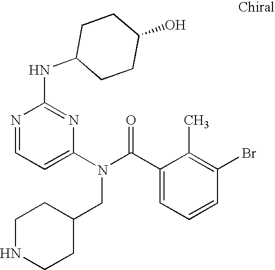 | B | 0.973 | 501 |
| 352 | 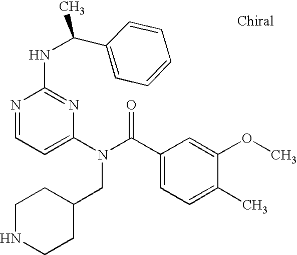 | B | 1.327 | 460 |
| 353 | 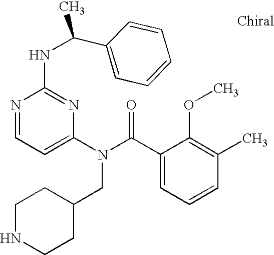 | B | 1.293 | 460 |
| 354 | 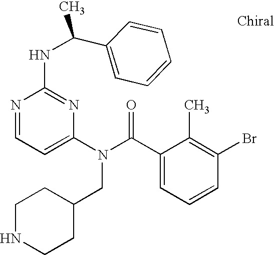 | B | 1.273 | 510 |

| Example | | Column | Retention | Mass Observed |
|---|---|---|---|---|
| No. | Molecular Structure | Conditions | Time (min) | (M + H)+ |
| 355 | 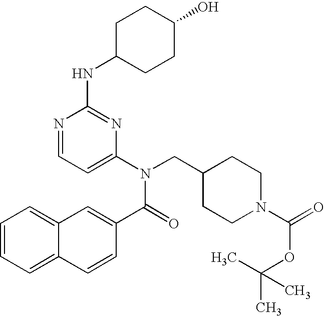 | B | 1.54 | 560 |
| 356 | 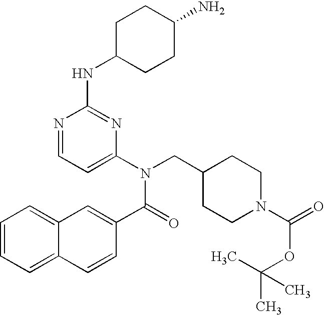 | B | 1.36 | 559 |
| 357 | 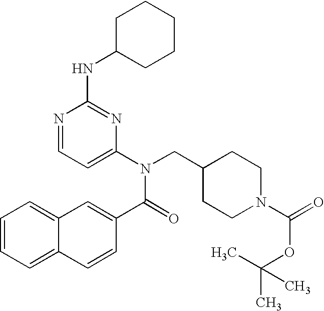 | B | 1.933 | 544 |
| 358 | 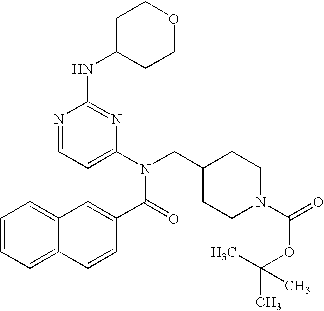 | B | 1.64 | 546 |
| 359 | 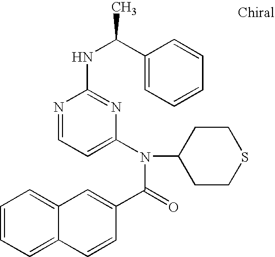 | B | 1.807 | 469 |
| 360 | 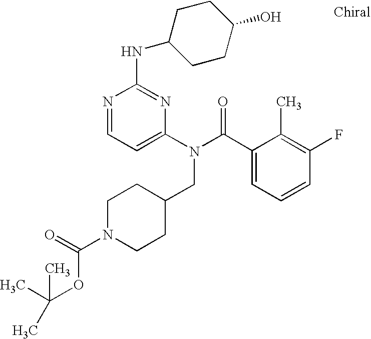 | B | 1.447 | 542 |
| 361 | 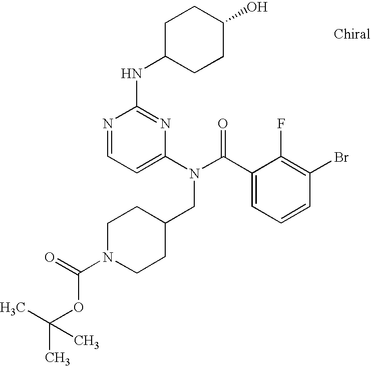 | B | 1.473 | 608 |
| 362 | 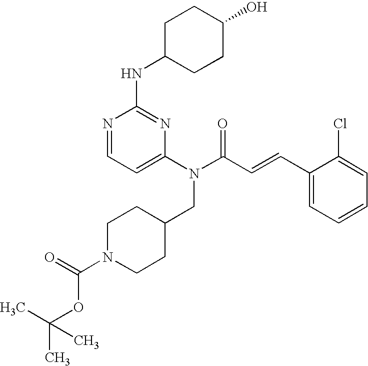 | B | 1.54 | 570 |
| 363 | 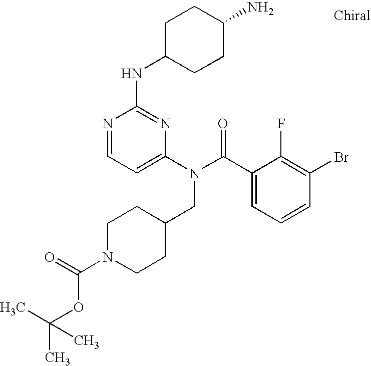 | B | 1.32 | 607 |
| 364 | 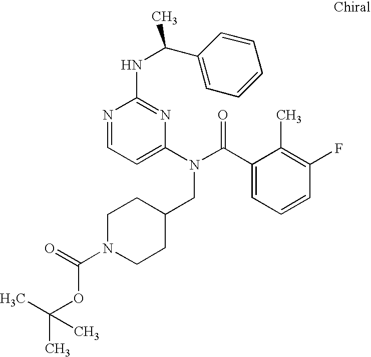 | B | 1.78 | 548 |
| 365 | 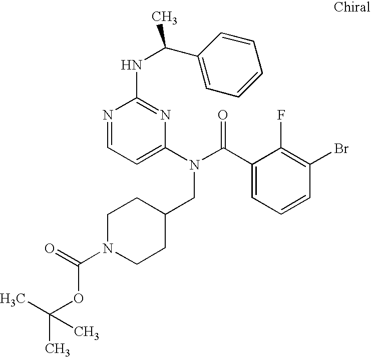 | B | 1.887 | 614 |
| 366 | 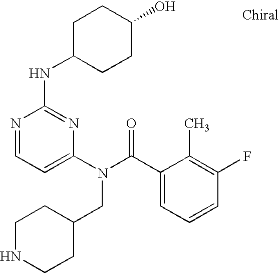 | B | 0.88 | 442 |
| 367 | 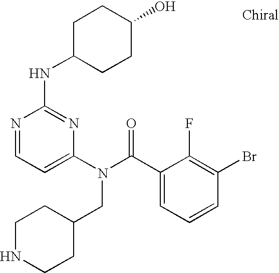 | B | 0.913 | 508 |
| 368 | 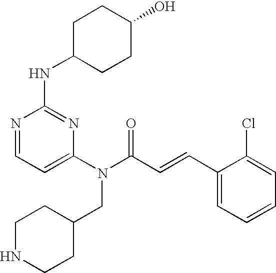 | B | 1.02 | 470 |
| 369 | 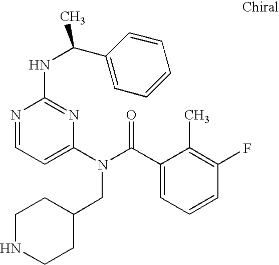 | B | 1.193 | 448 |
| 370 | 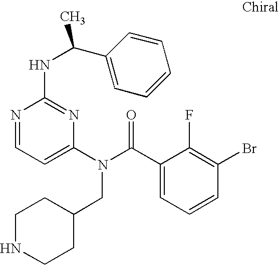 | B | 1.24 | 514 |
| 371 | 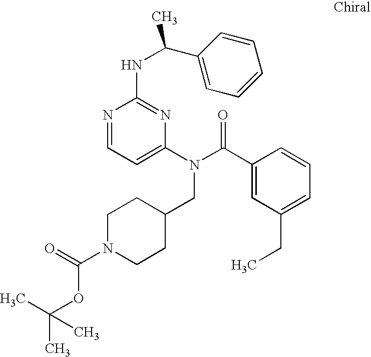 | B | 1.893 | 544 |
| 372 | 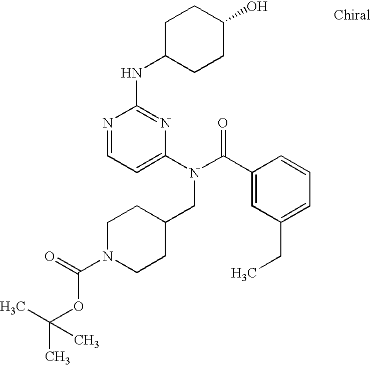 | B | 1.5 | 538 |
| 373 | 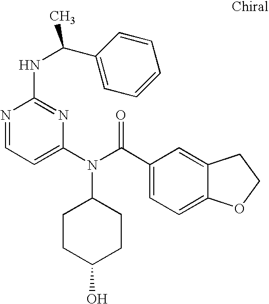 | B | 1.647 | 459 |
| 374 | 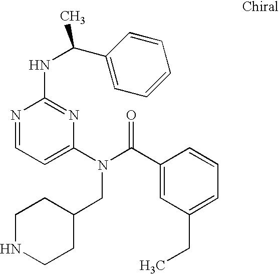 | B | 1.187 | 444 |
| 375 | 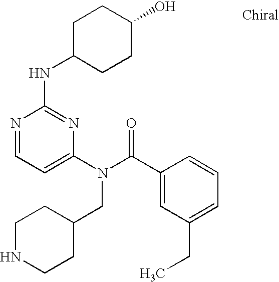 | B | 0.918 | 438 |
| 376 | 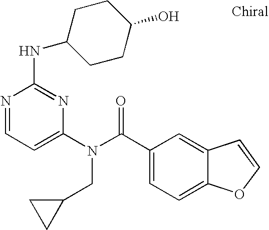 | B | 1.207 | 407 |
| 377 | 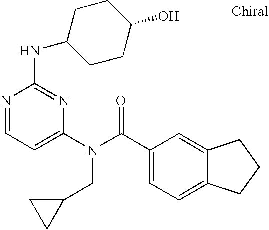 | B | 1.333 | 407 |
| 378 | 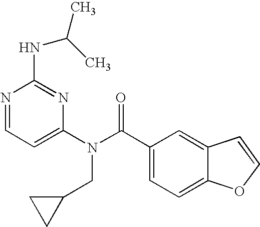 | B | 1.433 | 351 |
| 379 | 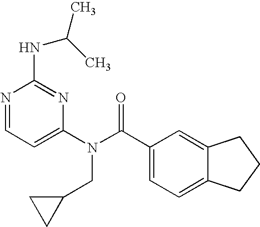 | B | 1.573 | 351 |
| 380 | 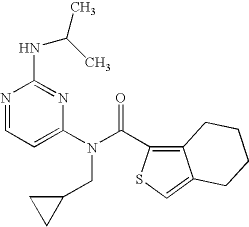 | B | 1.58 | 371 |
| 381 | 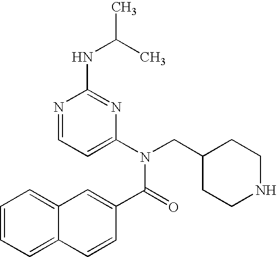 | B | 1.1 | 404 |
| 382 | 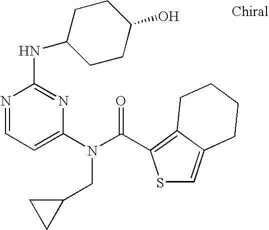 | B | 1.353 | 427 |
| 383 | 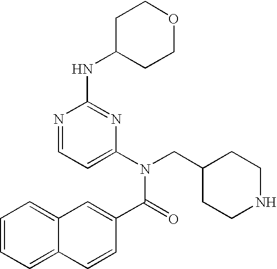 | B | 1 | 446 |
| 384 | 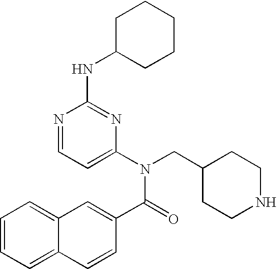 | B | 1.227 | 444 |
| 385 | 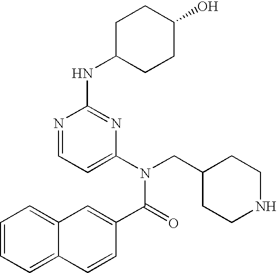 | B | 0.953 | 460 |
| 386 | 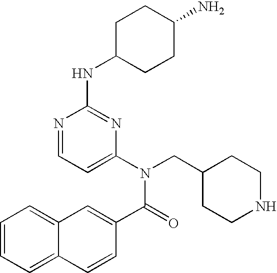 | B | 1.687 | 459 |
| 387 | 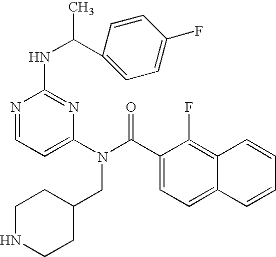 | B | 1.293 | 502 |
| 388 | 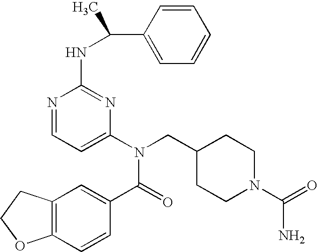 | B | 1.227 | 501 |
| 389 | 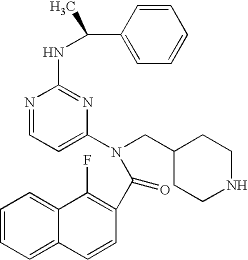 | B | 1.32 | 484 |
| 390 | 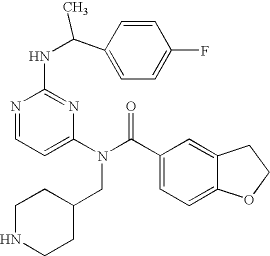 | B | 1.1 | 476 |
| 391 | 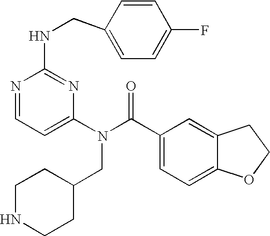 | B | 1.033 | 462 |
| 392 | 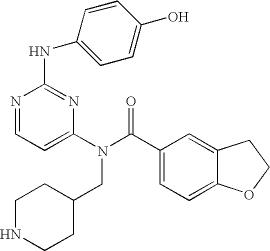 | B | 0.987 | 447 |
| 393 | 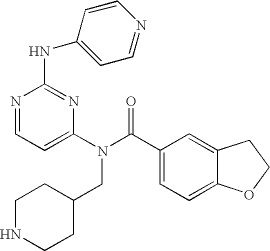 | B | 0.953 | 431 |
| 394 | 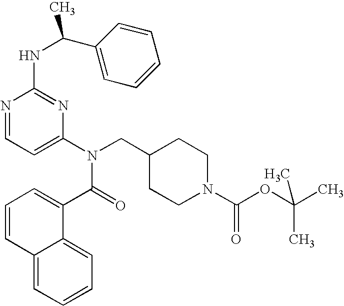 | B | 1.873 | 566 |
| 395 | 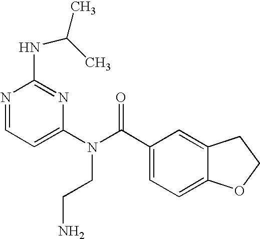 | B | 1.473 | 442 |
| 396 | 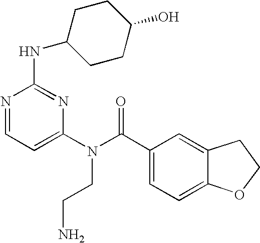 | B | 1.38 | 498 |
| 397 | 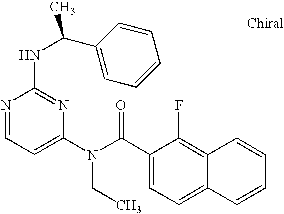 | B | 1.727 | 415 |
| 398 | 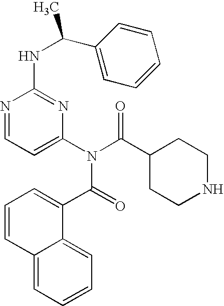 | B | 1.253 | 467 |
| 399 | 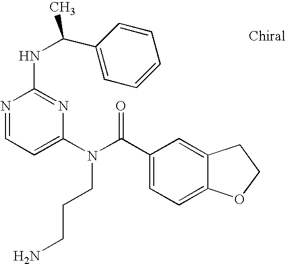 | B | 1.273 | 419 |
| 400 | 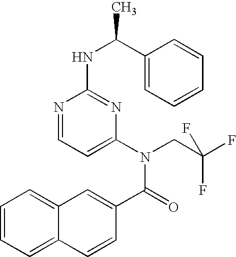 | B | 1.74 | 450 |
| 401 | 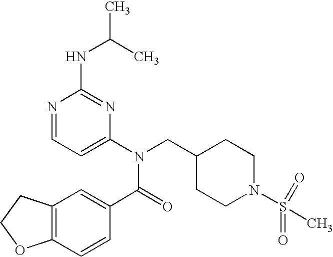 | B | 1.293 | 474 |
| 402 | 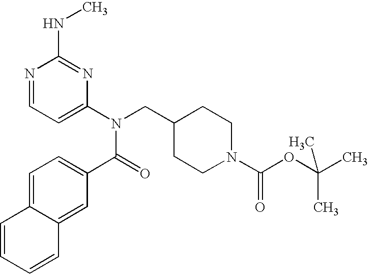 | B | 1.653 | 476 |
| 403 | 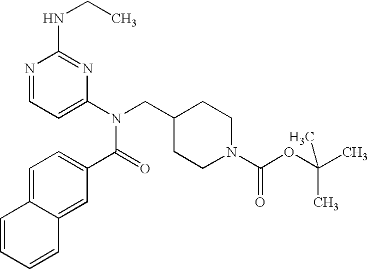 | B | 1.727 | 490 |
| 404 | 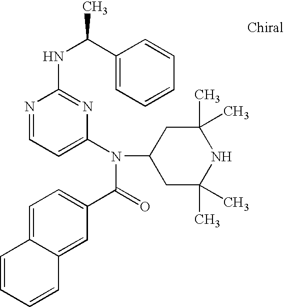 | B | 1.373 | 508 |
| 405 | 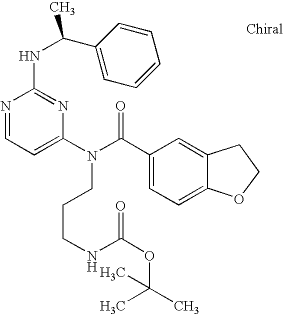 | B | 1.52 | 519 |
| 406 | 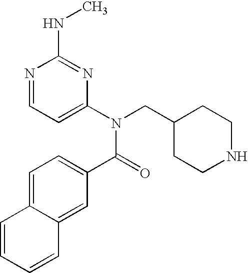 | B | 1.24 | 375 |
| 407 | 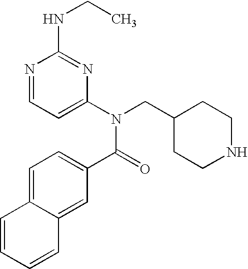 | B | 1.23 | 390 |
| 408 | 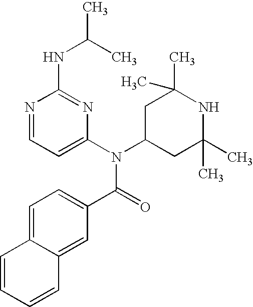 | B | 1.35 | 446 |
| 409 | 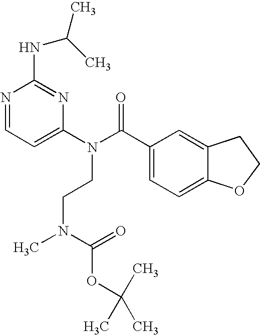 | B | 1.407 | 456 |
| 410 | 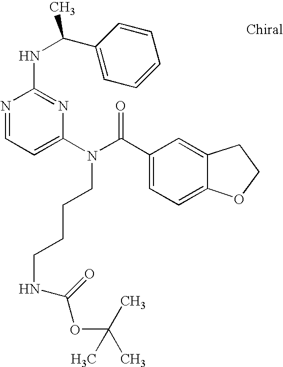 | B | 1.593 | 532 |
| 411 | 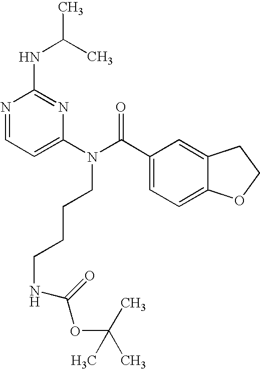 | B | 1.467 | 471 |
| 412 | 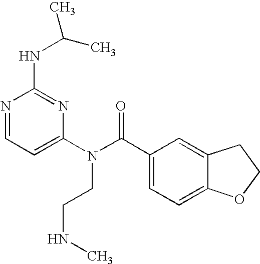 | B | 0.867 | 356 |
| 413 | 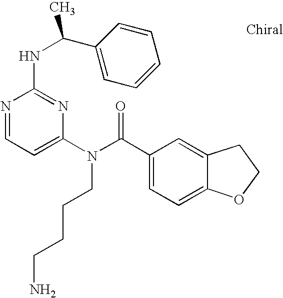 | B | 1.107 | 433 |
| 414 | 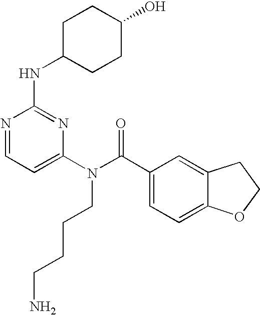 | B | 0.84 | 427 |
| 415 | 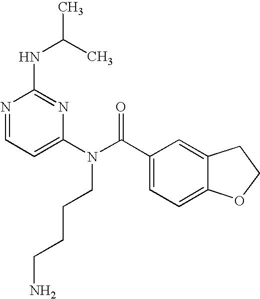 | B | 0.947 | 371 |
| 416 | 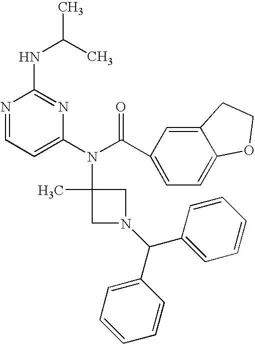 | B | 1.32 | 535 |
| 417 | 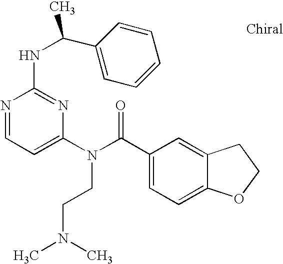 | B | 1.2 | 419 |
| 418 | 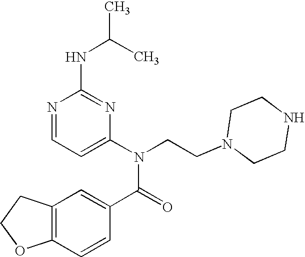 | B | 1.067 | 411 |
| 419 | 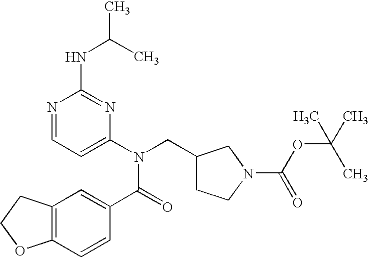 | B | 1.6 | 482 |
| 420 | 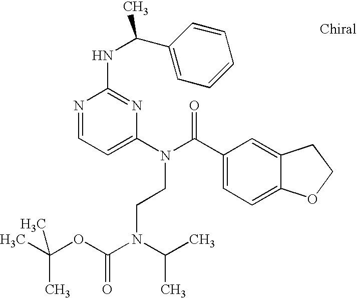 | B | 1.793 | 546 |
| 421 | 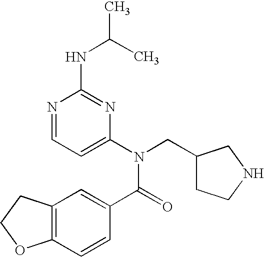 | B | 0.887 | 382 |
| 422 | 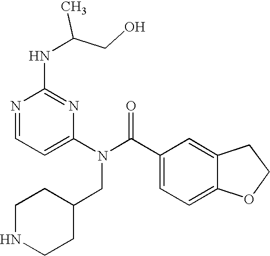 | B | 0.833 | 412 |
| 423 | 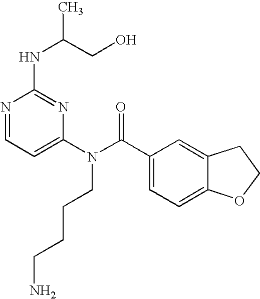 | B | 0.8 | 386 |
| 424 | 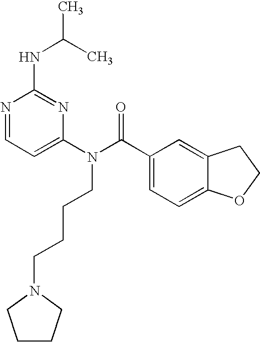 | B | 0.98 | 424 |
| 425 | 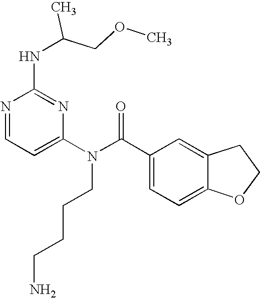 | B | 0.873 | 400 |
| 426 | 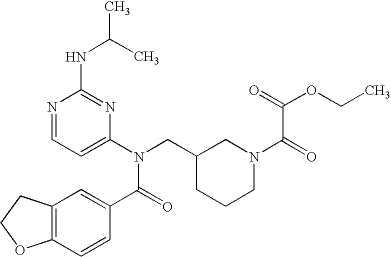 | B | 1.353 | 496 |
| 427 | 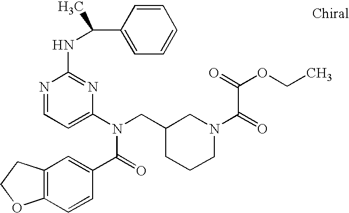 | B | 1.507 | 558 |
| 428 | 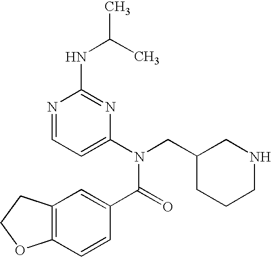 | B | 0.94 | 396 |
| 429 | 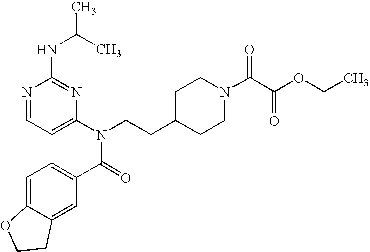 | B | 1.4 | 510 |
| 430 | 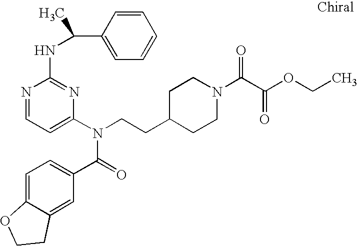 | B | 1.527 | 572 |
| 431 | 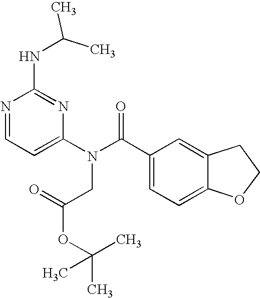 | B | 1.447 | 413 |
| 432 | 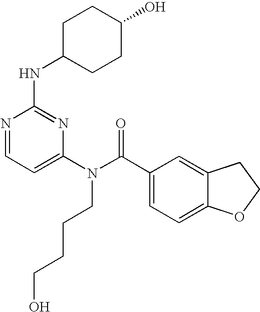 | B | 0.98 | 428 |
| 433 | 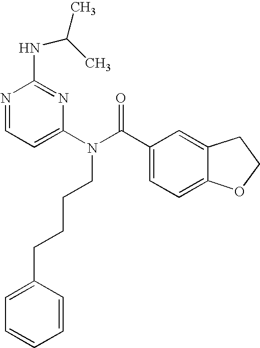 | B | 1.7 | 431 |
| 434 | 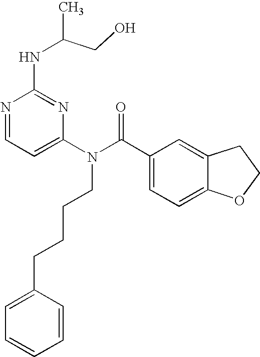 | B | 1.46 | 448 |
| 435 | 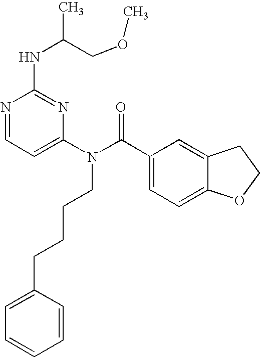 | B | 1.64 | 461 |
| 436 | 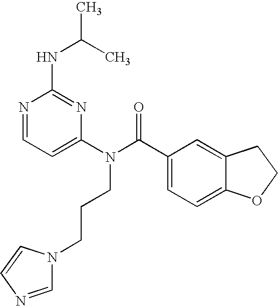 | B | 0.993 | 407 |
| 437 | 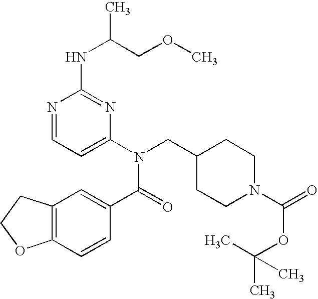 | B | 1.62 | 526 |
| 438 | 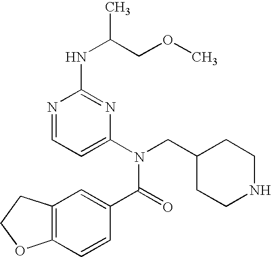 | B | 1.007 | 426 |
| 439 | 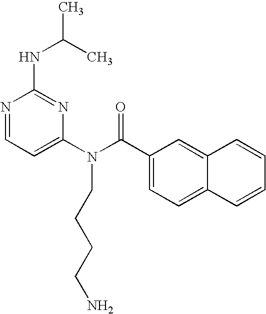 | B | 1.213 | 378 |
| 440 | 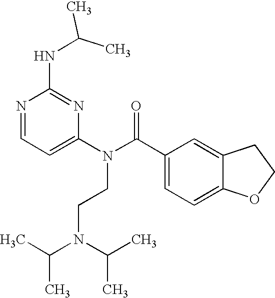 | B | 1.14 | 426 |
| 441 | 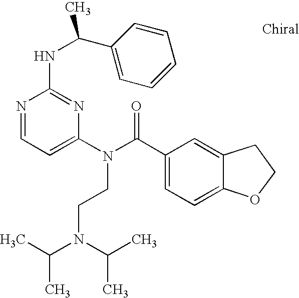 | B | 1.327 | 488 |
| 442 | 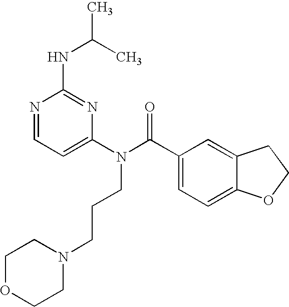 | B | 0.84 | 426 |
| 443 | 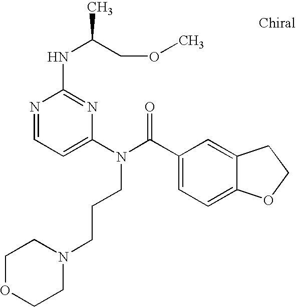 | B | 0.92 | 456 |
| 444 | 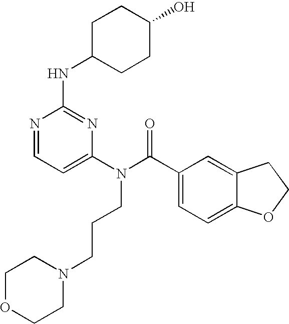 | B | 0.867 | 482 |
| 445 | 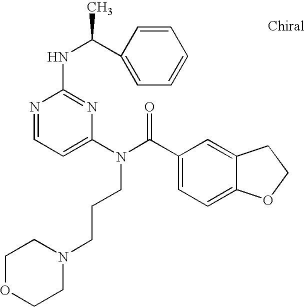 | B | 1.12 | 488 |
| 446 | 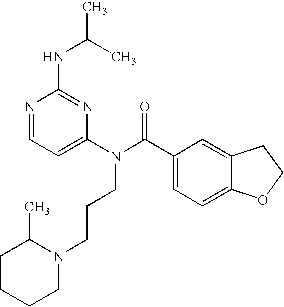 | B | 0.967 | 438 |
| 447 | 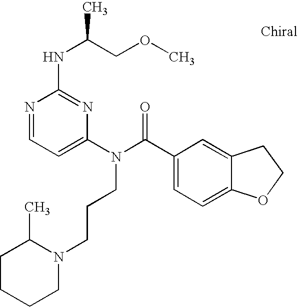 | B | 0.927 | 468 |
| 448 | 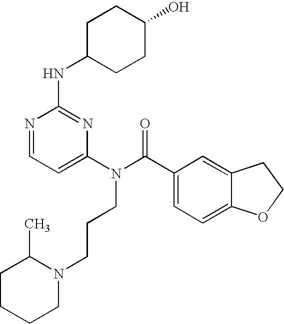 | B | 0.847 | 494 |
| 449 | 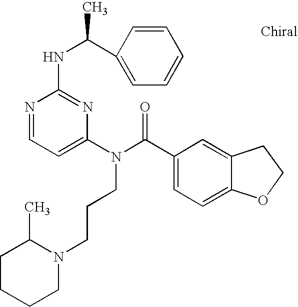 | B | 1.14 | 500 |
| 450 | 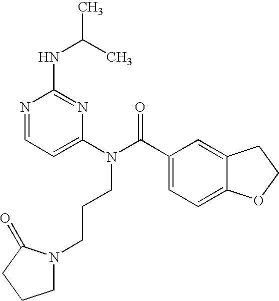 | B | 1.967 | 424 |
| 451 | 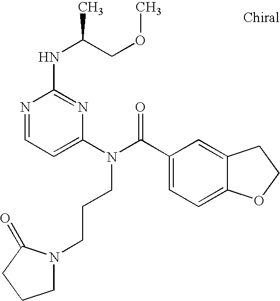 | B | 1.94 | 454 |
| 452 | 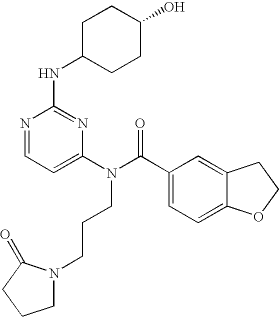 | B | 1.887 | 480 |
| 453 | 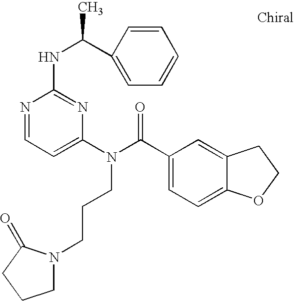 | B | 2.107 | 486 |
| 454 | 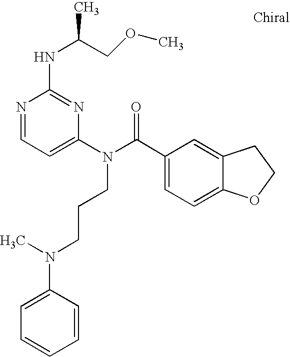 | B | 1.053 | 476 |
| 455 | 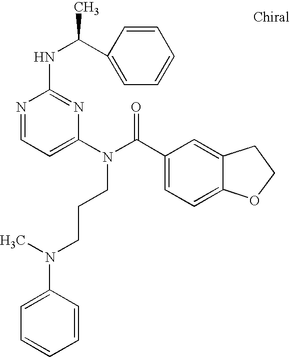 | B | 1.6 | 508 |
| 456 | 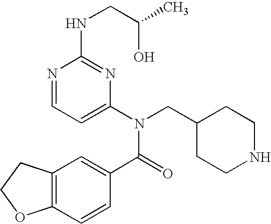 | B | 1.22 | 412 |
| 457 | 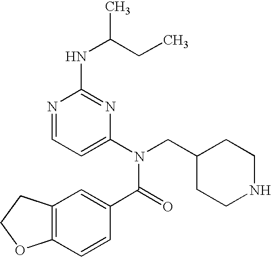 | B | 1.36 | 410 |
| 458 | 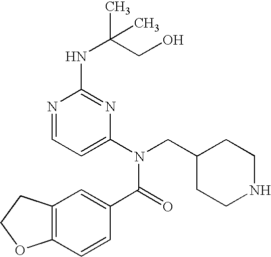 | B | 1.37 | 426 |
| 459 | 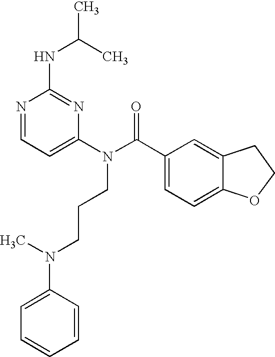 | B | 1.467 | 446 |
| 460 | 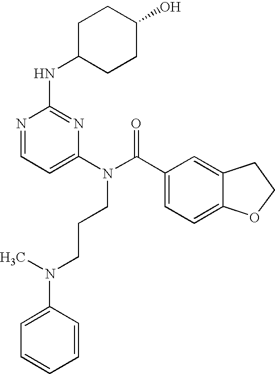 | B | 1.387 | 502 |
| 461 | 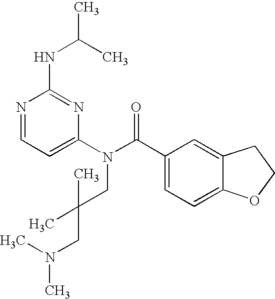 | B | 0.9 | 412 |
| 462 | 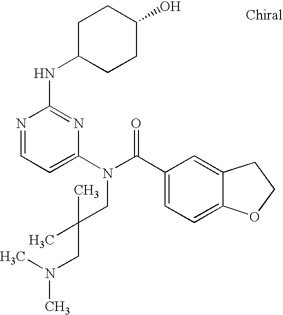 | B | 1.3 | 468 |
| 463 | 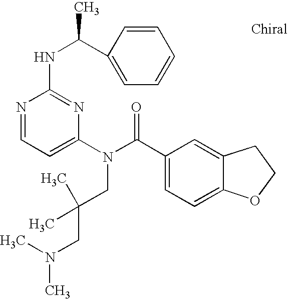 | B | 1.56 | 474 |
| 464 | 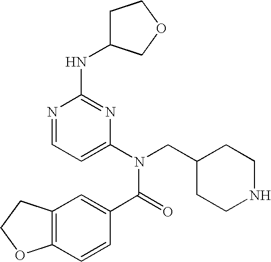 | B | 1.35 | 424 |
| 465 | 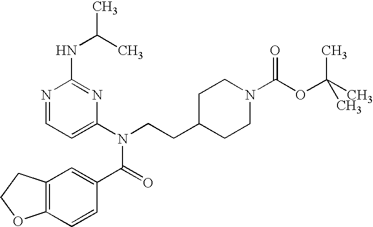 | B | 1.473 | 510 |
| 466 | 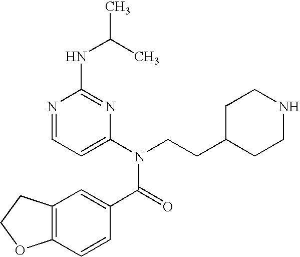 | B | 1.2 | 410 |
| 467 | 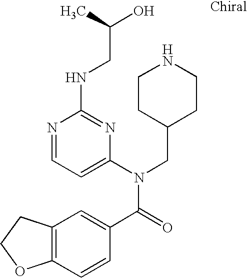 | B | 1.32 | 412 |
| 468 | 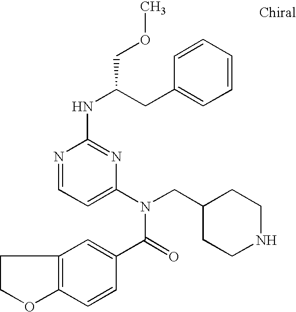 | B | 1.587 | 502 |
| 469 | 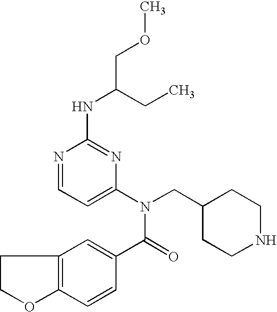 | B | 1.433 | 440 |
| 470 | 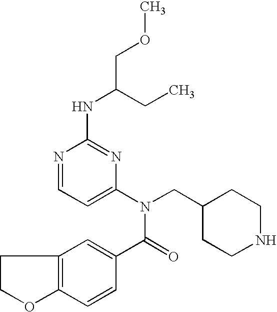 | B | 0.99 | 440 |
| 471 | 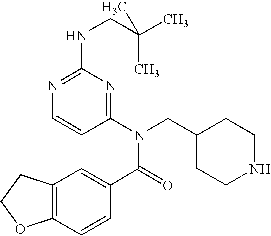 | B | 1.09 | 424 |
| 472 | 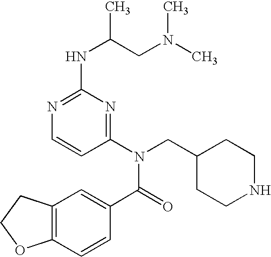 | B | 0.79 | 439 |
| 473 | 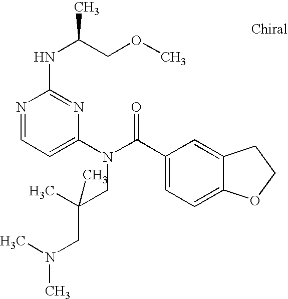 | B | 0.86 | 442 |
| 474 | 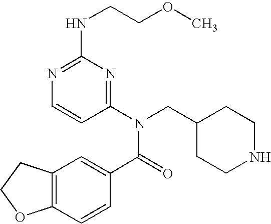 | B | 0.79 | 412 |
| 475 | 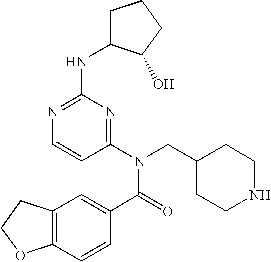 | B | 0.83 | 438 |
| 476 | 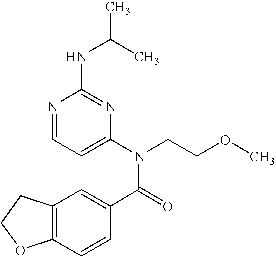 | B | 1.153 | 357 |
| 477 | 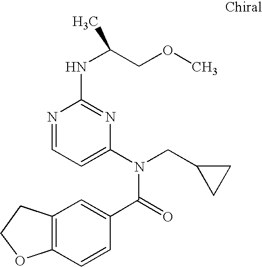 | B | 1.307 | 383 |
| 478 | 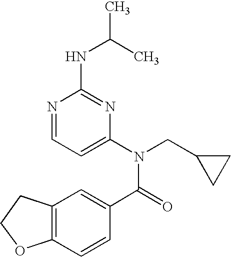 | B | 1.333 | 353 |
| 479 | 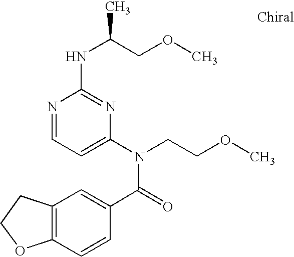 | B | 1.113 | 387 |
| 480 | 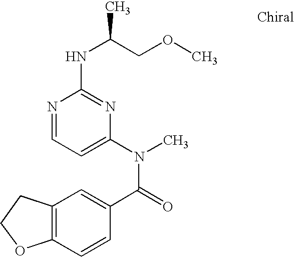 | B | 1.073 | 343 |
| 481 | 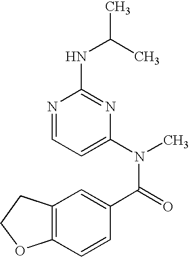 | B | 1.127 | 313 |
| 482 | 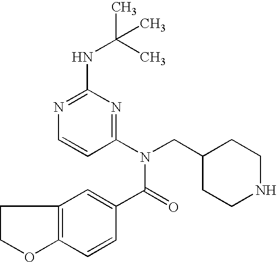 | B | 1 | 410 |
| 483 | 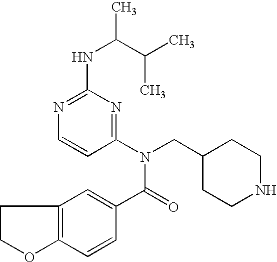 | B | 1.06 | 424 |
| 484 | 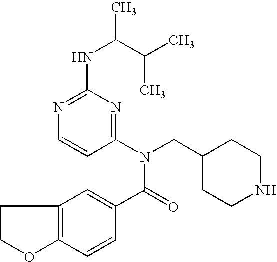 | B | 1.06 | 424 |
| 485 | 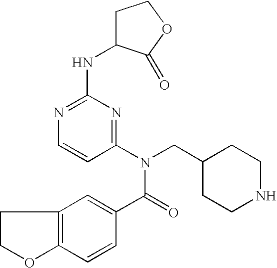 | B | 0.85 | 438 |
| 486 | 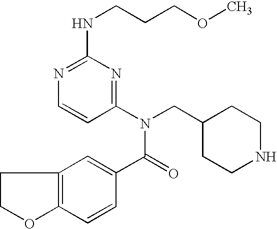 | B | 0.89 | 426 |
| 487 | 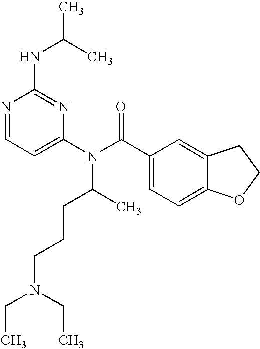 | B | 1.06 | 441 |
| 488 | 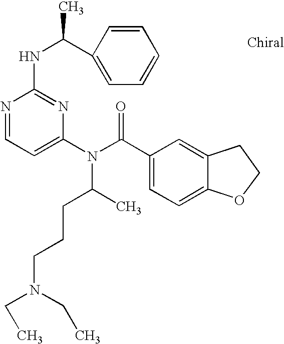 | B | 1.24 | 502 |
| 489 | 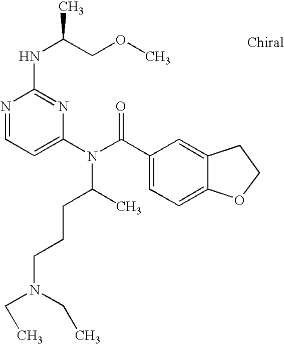 | B | 1.06 | 470 |
| 490 | 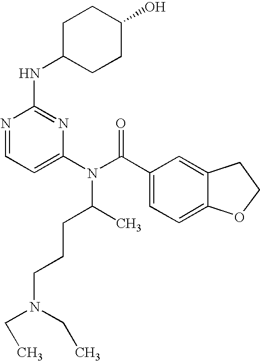 | B | 0.933 | 496 |
| 491 | 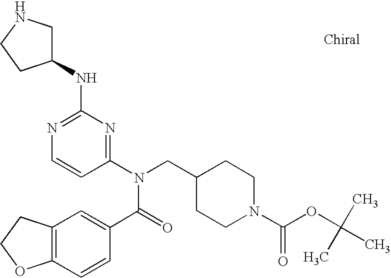 | B | 1.23 | 523 |
| 492 | 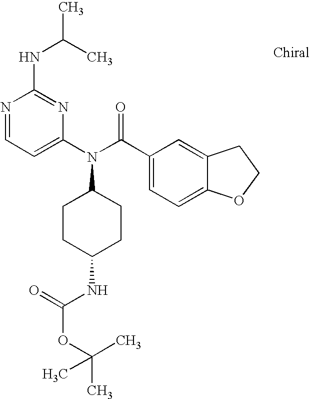 | B | 1.553 | 496 |
| 493 | 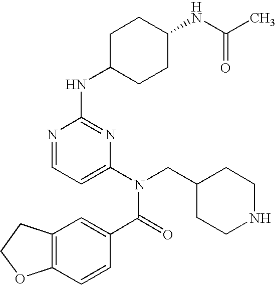 | B | 0.82 | 493 |
| 494 | 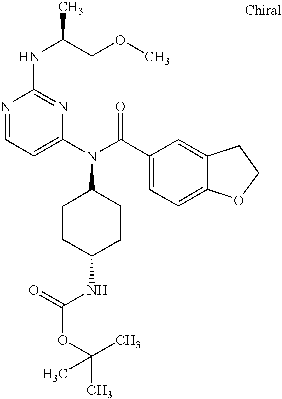 | B | 1.533 | 526 |
| 495 | 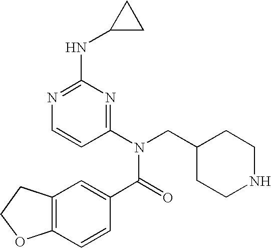 | B | 0.87 | 394 |
| 496 | 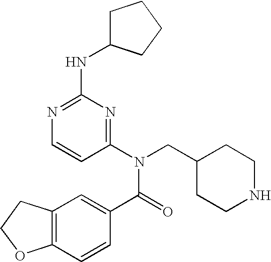 | B | 1.02 | 422 |
| 497 | 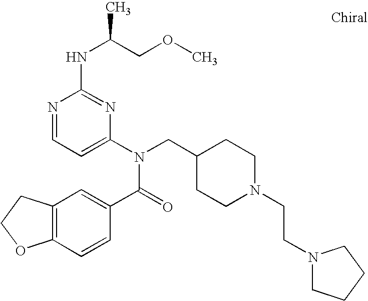 | B | 0.92 | 523 |
| 498 | 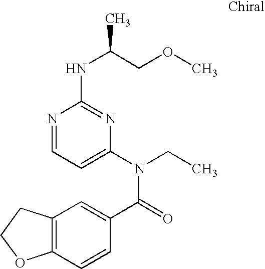 | B | 1.193 | 358 |
| 499 | 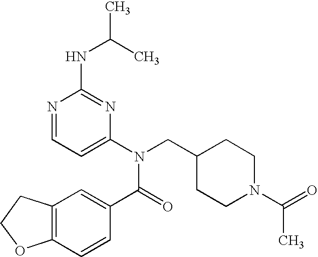 | B | 1.12 | 438 |
| 500 | 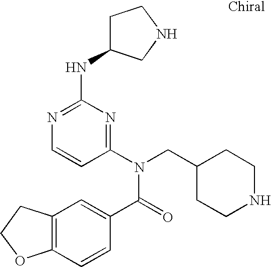 | B | 0.7 | 423 |
| 501 | 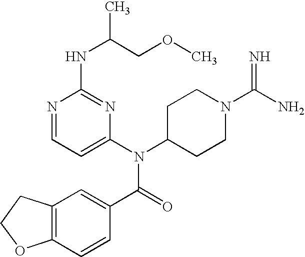 | B | 0.96 | 454 |
| 502 | 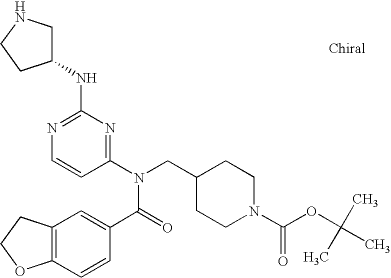 | B | 1.21 | 523 |
| 503 | 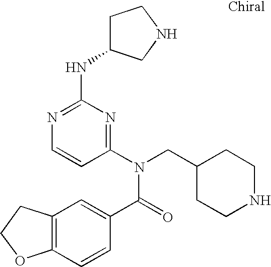 | B | 0.71 | 423 |
| 504 | 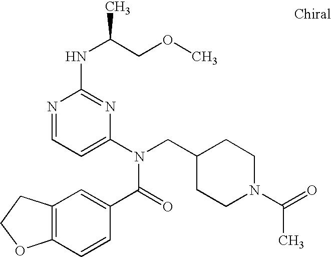 | B | 1.087 | 468 |
| 505 | 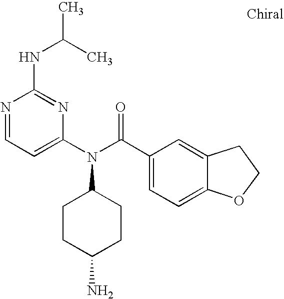 | B | 0.94 | 396 |
| 506 | 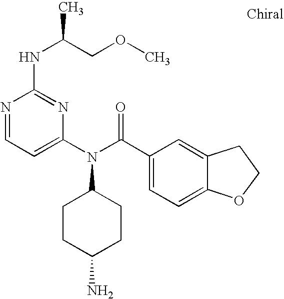 | B | 0.913 | 426 |
| 507 | 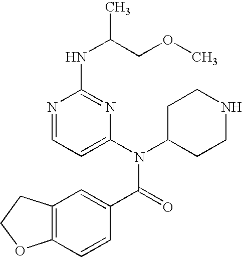 | B | 0.84 | 412 |
| 508 | 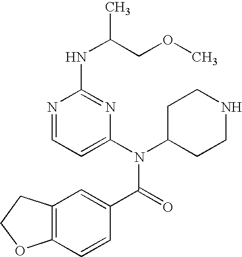 | B | 0.9 | 412 |
| 509 | 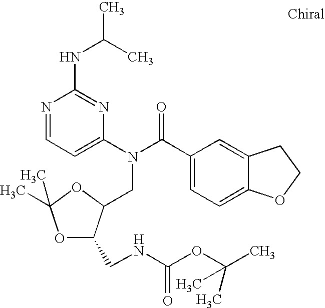 | B | 1.573 | 542 |
| 510 | 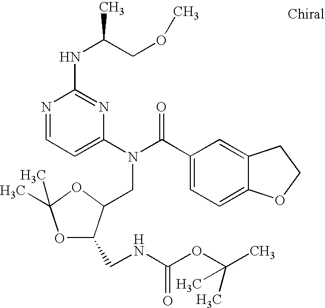 | B | 1.533 | 572 |
| 511 | 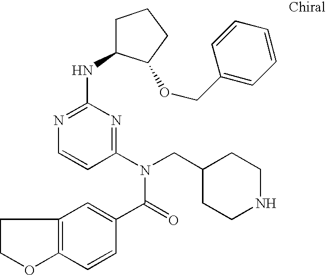 | B | 1.23 | 528 |
| 512 | 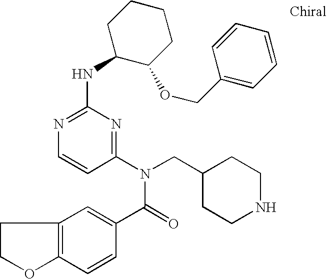 | B | 1.28 | 542 |
| 513 | 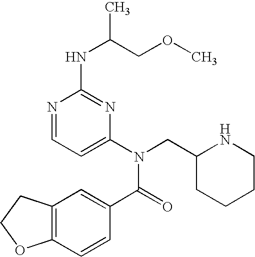 | B | 0.927 | 426 |
| 514 | 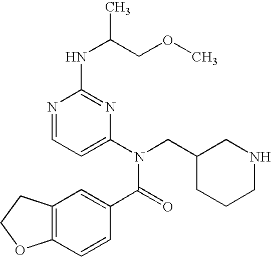 | B | 0.927 | 426 |
| 515 | 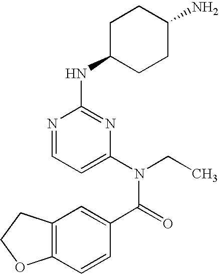 | B | 0.87 | 382 |
| 516 | 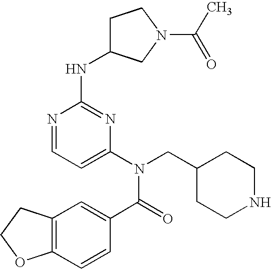 | B | 0.84 | 465 |
| 517 | 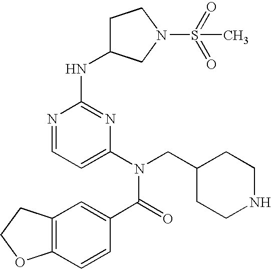 | B | 0.88 | 501 |
| 518 | 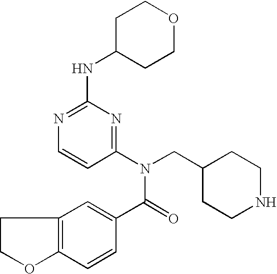 | B | 0.93 | 438 |
| 519 | 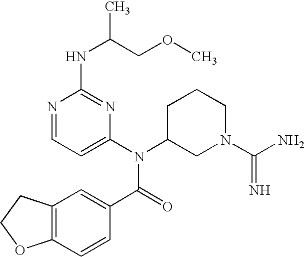 | B | 1.047 | 454 |
| 520 | 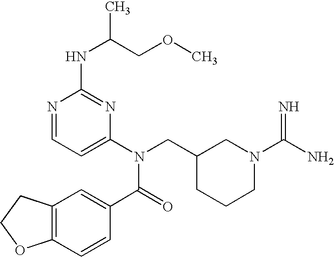 | B | 0.933 | 468 |
| 521 | 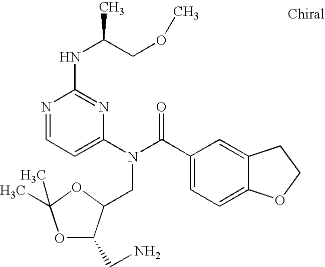 | B | 0.867 | 472 |
| 522 | 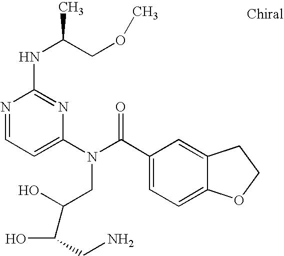 | B | 0.833 | 432 |
| 523 | 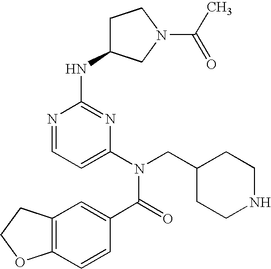 | B | 0.86 | 465 |
| 524 | 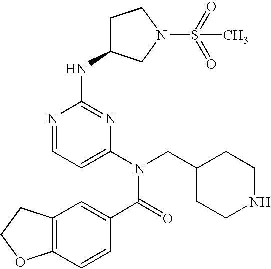 | B | 0.91 | 501 |
| 525 | 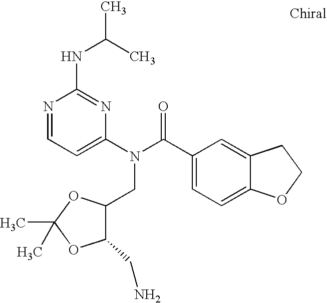 | B | 0.94 | 442 |
| 526 | 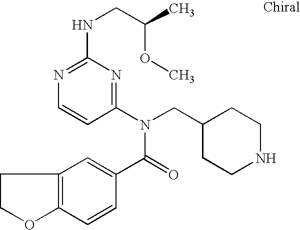 | B | 0.81 | 426 |
| 527 | 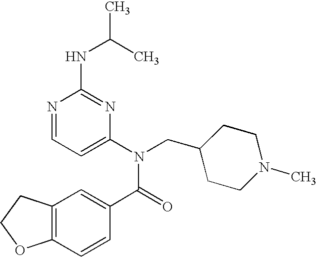 | B | 0.907 | 410 |
| 528 | 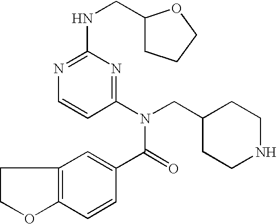 | B | 0.89 | 438 |
| 529 | 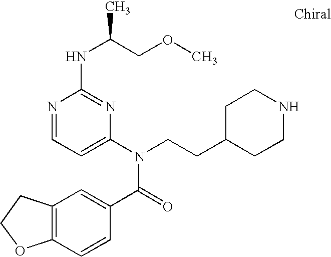 | B | 0.947 | 440 |
| 530 | 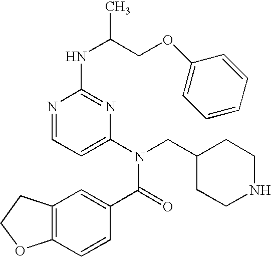 | B | 1.17 | 488 |
| 531 | 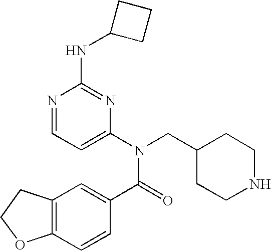 | B | 0.97 | 408 |
| 532 | 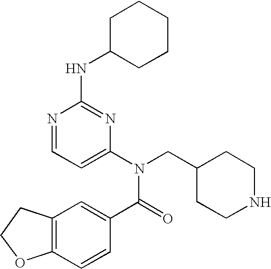 | B | 1.09 | 436 |
| 533 | 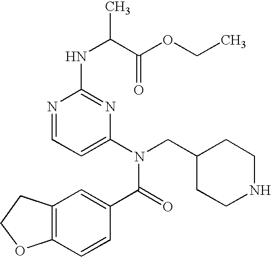 | B | 0.97 | 454 |
| 534 | 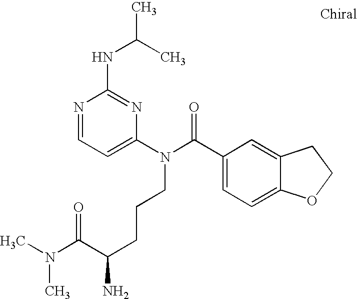 | B | 0.9 | 442 |
| 535 | 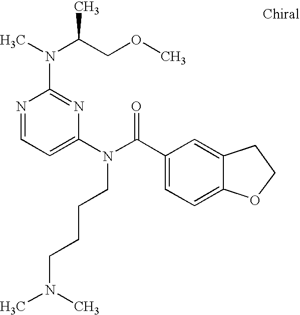 | B | 0.847 | 443 |
| 536 | 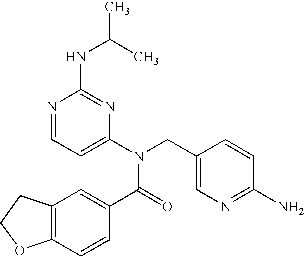 | B | 0.9 | 405 |
| 537 | 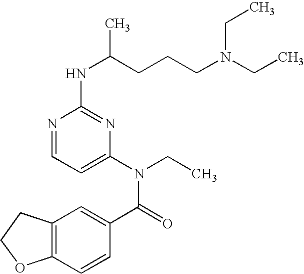 | B | 0.95 | 426 |
| 538 | 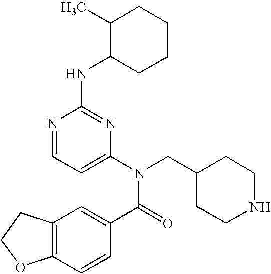 | B | 1.09 | 450 |
| 539 | 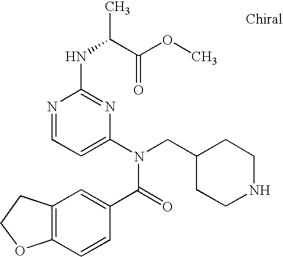 | B | 0.87 | 440 |
| 540 | 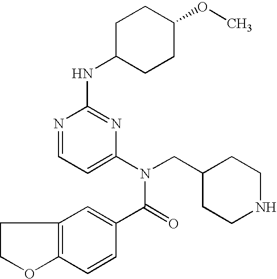 | B | 0.8 | 466 |
| 541 | 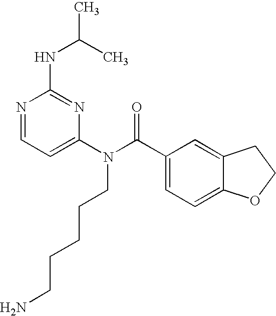 | B | 0.927 | 384 |
| 542 | 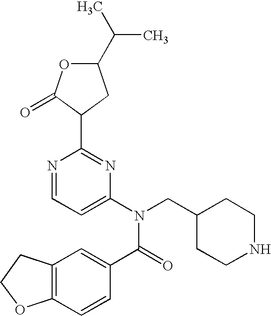 | B | 1.06 | 466 |
| 543 | 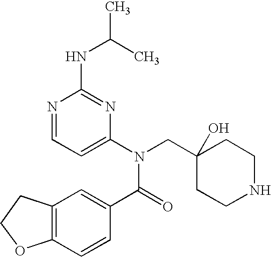 | B | 0.94 | 412 |
| 544 | 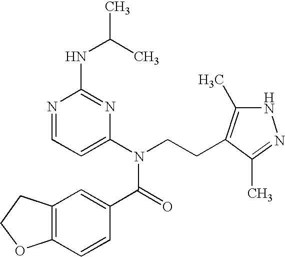 | B | 0.967 | 421 |
| 545 | 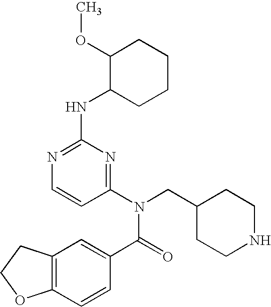 | B | 0.95 | 466 |
| 546 | 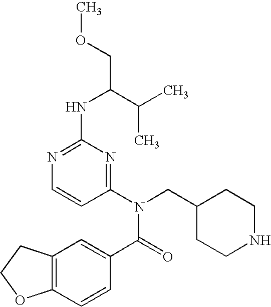 | B | 0.93 | 454 |
| 547 | 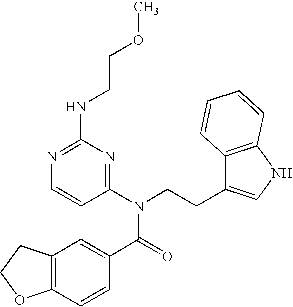 | B | 1.4 | 458 |
| 548 | 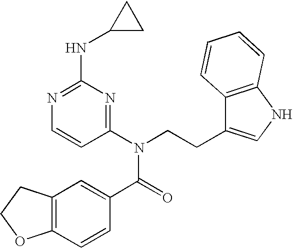 | B | 1.41 | 440 |
| 549 | 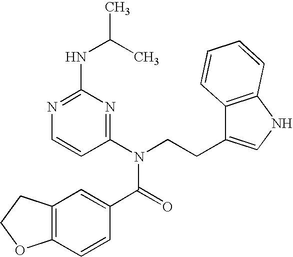 | B | 1.51 | 442 |
| 550 | 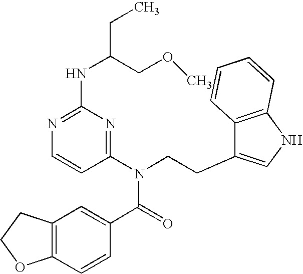 | B | 1.53 | 486 |
| 551 | 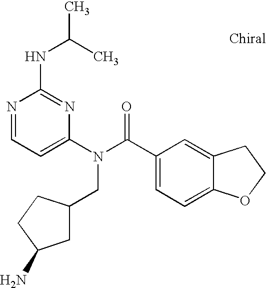 | B | 0.907 | 396 |
| 552 | 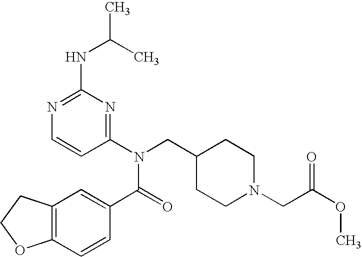 | B | 0.93 | 468 |
| 553 | 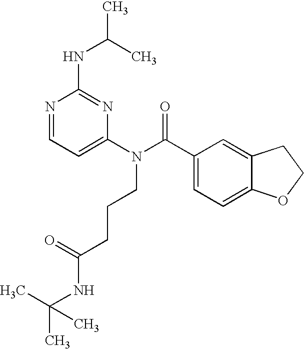 | B | 1.273 | 441 |
| 554 | 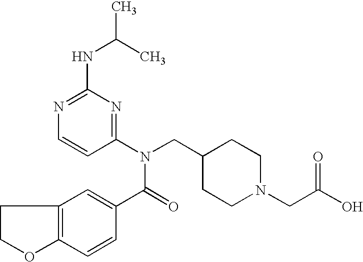 | B | 0.95 | 454 |
| 555 | 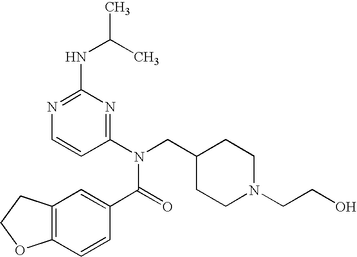 | B | 0.95 | 440 |
| 556 | 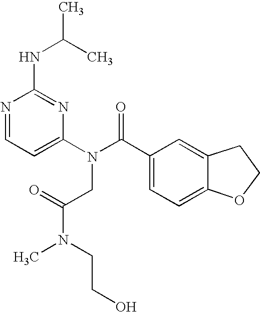 | B | 0.94 | 414 |
| 557 | 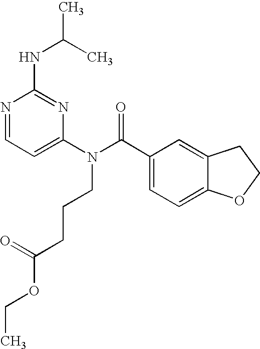 | B | 1.373 | 413 |
| 558 | 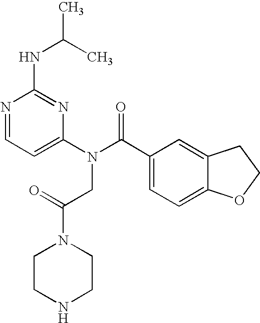 | B | 0.82 | 425 |
| 559 | 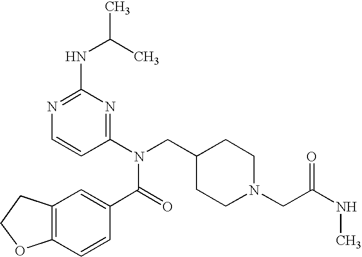 | B | 0.96 | 467 |
| 560 | 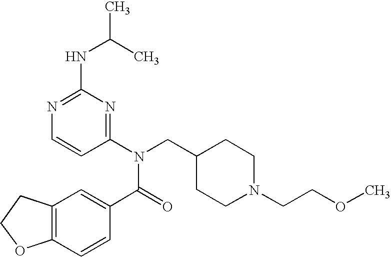 | B | 0.97 | 454 |
| 561 | 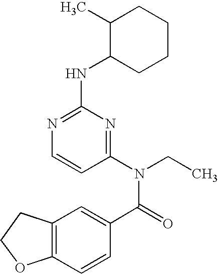 | B | 1.5 | 381 |
| 562 | 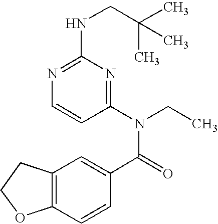 | B | 1.39 | 355 |
| 563 | 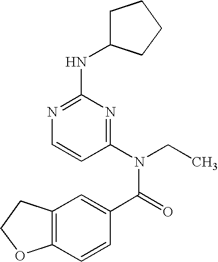 | B | 1.35 | 353 |
| 564 | 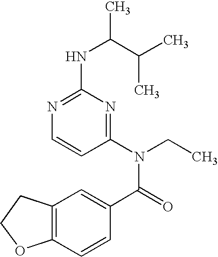 | B | 1.39 | 355 |
| 565 | 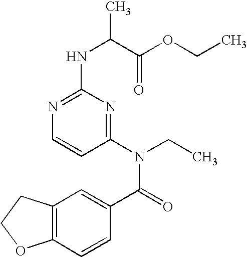 | B | 1.28 | 385 |
| 566 | 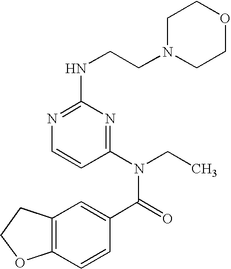 | B | 0.82 | 398 |
| 567 | 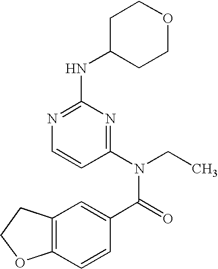 | B | 1.08 | 369 |
| 568 | 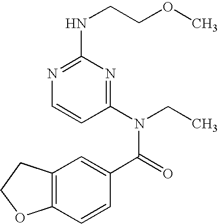 | B | 1.11 | 343 |
| 569 | 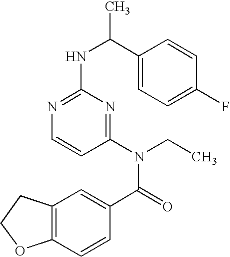 | B | 1.46 | 407 |
| 570 | 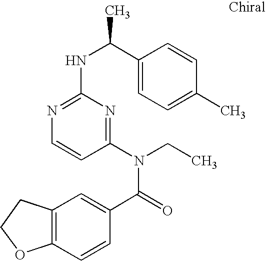 | B | 1.53 | 403 |
| 571 | 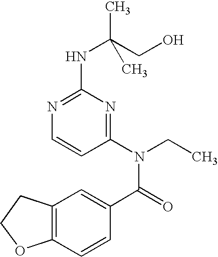 | B | 1.08 | 357 |
| 572 | 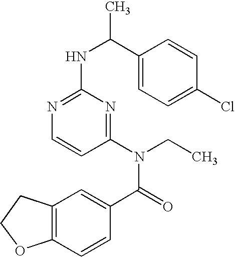 | B | 1.6 | 423 |
| 573 | 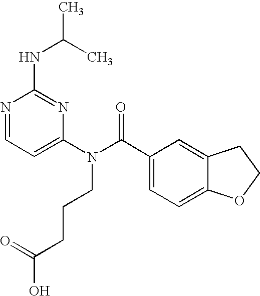 | B | 1.127 | 385 |
| 574 | 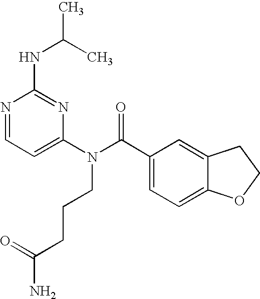 | B | 0.973 | 384 |
| 575 | 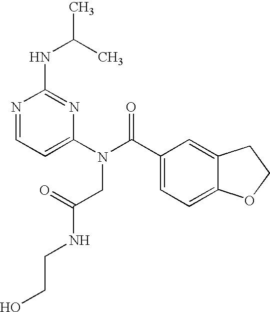 | B | 1 | 400 |
| 576 | 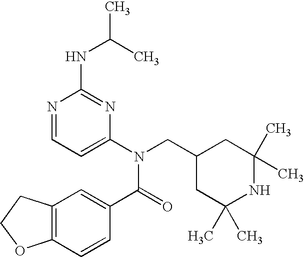 | B | 1.007 | 452 |
| 577 | 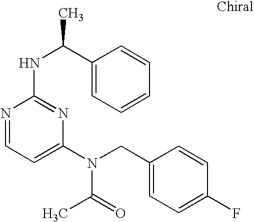 | B | 1.433 | 364 |
| 578 | 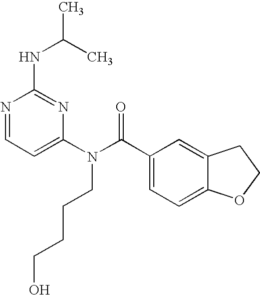 | B | 1.14 | 371 |
| 579 | 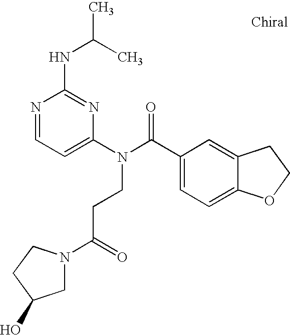 | B | 0.987 | 441 |
| 580 | 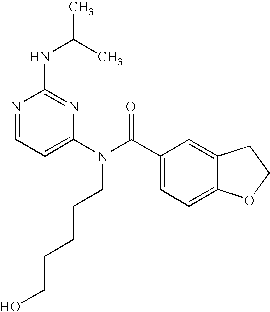 | B | 1.147 | 385 |
| 581 | 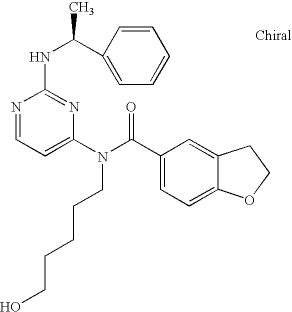 | B | 1.32 | 448 |
| 582 | 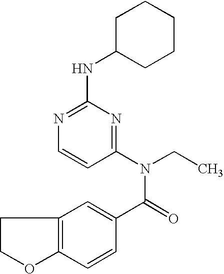 | B | 1.53 | 367 |
| 583 | 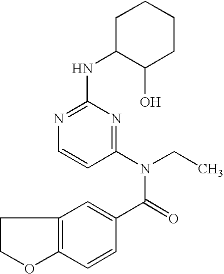 | B | 1.3 | 383 |
| 584 | 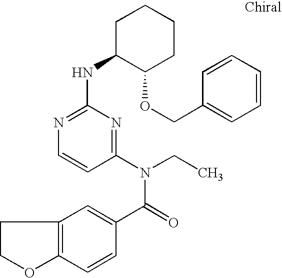 | B | 1.65 | 473 |
| 585 | 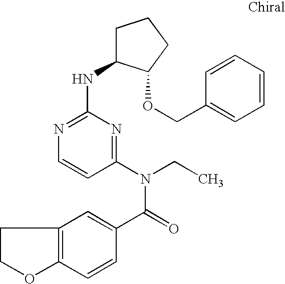 | B | 1.62 | 459 |
| 586 | 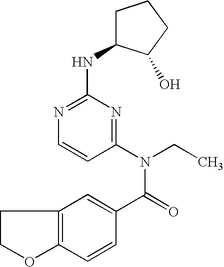 | B | 1.15 | 369 |
| 587 | 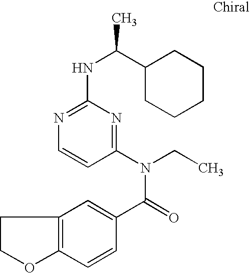 | B | 1.68 | 395 |
| 588 | 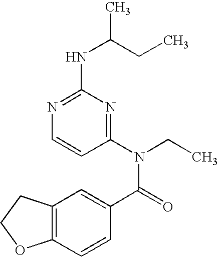 | B | 1.37 | 341 |
| 589 | 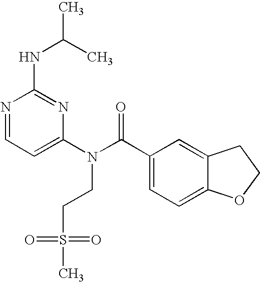 | B | 1.033 | 405 |
| 590 | 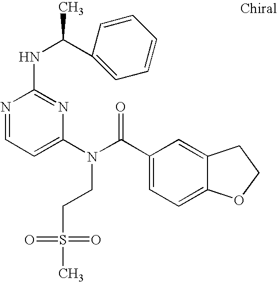 | B | 1.247 | 467 |
| 591 | 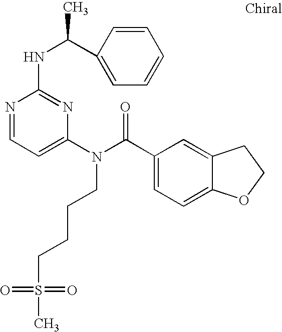 | B | 1.287 | 495 |

Compounds of Examples 20, 22, 23, 40, 70, 73, 76, 77, 83, 84, 93, 95, 102, 114, 118, 127, 130, 160, 167, 172, 181, 187, 188, 199, 220, 238, 261, 264, 276, 277, 278, 287, 293, 295, 300, 304, 307, 309, 377, 383, 388, 398, 404, 406, 413, 414, 415, 424, 425, 457, 470, 471, 474, 475, 483, 486, 495, 496, 534, 538, 541, 551 and 552 have an activity of <1 μM in the diluted whole blood assay.

### EXAMPLE 592

### Biological Activity

The compounds provided herein exhibit varying levels of activity towards p38a kinase. For example, compounds 2-39 in Table 1 and the compounds of Examples 20, 22, and 30 each exhibit an IC₅₀ value of 1 μM or less in the diluted Whole Blood Assay described below.

Assays for p38 α Kinase Inhibition

For each of the assay procedures described below, the TNF-α production correlates to the activity of p38-α kinase.

A. Human Whole Blood Assay for p38 Kinase Inhibition

Venous blood is collected from healthy male volunteers into a heparinized syringe and is used within 2 hours of collection. Test compounds are dissolved in 100% DMSO and 1 μl aliquots of drug concentrations ranging from 0 to 1 mM are dispensed into quadruplicate wells of a 24-well microtiter plate (Nunclon Delta SI, Applied Scientific, So. San Francisco, Calif.). Whole blood is added at a volume of 1 ml/well and the mixture is incubated for 15 minutes with constant shaking (Titer Plate Shaker, Lab-Line Instruments, Inc., Melrose Park, Ill.) at a humidified atmosphere of 5% CO₂ at 37° C. Whole blood is cultured either undiluted or at a final dilution of 1:10 with RPMI 1640 (Gibco 31800+NaHCO₃, Life Technologies, Rockville, Md. and Scios, Inc., Sunnyvale, Calif.). At the end of the incubation period, 10 μl of LPS (E. coli 0111:B4, Sigma Chemical Co., St. Louis, Mo.) is added to each well to a final concentration of 1 or 0.1 μg/ml for undiluted or 1:10 diluted whole blood, respectively. The incubation is continued for an additional 2 hours. The reaction is stopped by placing the microtiter plates in an ice bath and plasma or cell-free supernates are collected by centrifugation at 3000 rpm for 10 minutes at 4° C. The plasma samples are stored at −80° C. until assayed for TNF-α levels by ELISA, following the directions supplied by Quantikine Human TNF-α assay kit (R&D Systems, Minneapolis, Minn.).

IC₅₀ values are calculated using the concentration of inhibitor that causes a 50% decrease as compared to a control.

B. Enriched Mononuclear Cell Assay for p38 Kinase Inhibition

The enriched mononuclear cell assay, the protocol of which is set forth below, begins with cryopreserved Human Peripheral Blood Mononuclear Cells (HPBMCs) (Clonetics Corp.) that are rinsed and resuspended in a warm mixture of cell growth media. The resuspended cells are then counted and seeded at 1×10⁶ cells/well in a 24-well microtitre plate. The plates are then placed in an incubator for an hour to allow the cells to settle in each well.

After the cells have settled, the media is aspirated and new media containing 100 ng/ml of the cytokine stimulatory factor Lipopolysaccharide (LPS) and a test chemical compound is added to each well of the microtiter plate. Thus, each well contains HPBMCs, LPS and a test chemical compound. The cells are then incubated for 2 hours, and the amount of the cytokine Tumor Necrosis Factor Alpha (TNF-α) is measured using an Enzyme Linked Immunoassay (ELISA). One such ELISA for detecting the levels of TNF-α is commercially available from R&D Systems. The amount of TNF-α production by the HPBMCs in each well is then compared to a control well to determine whether the chemical compound acts as an inhibitor of cytokine production.

LPS Induced Cytokine Synthesis in HPBMCS

Cryopreserved HPBMC (cat#CC-2702 Clonetics Corp)

LGM-3 media (cat#CC-3212 Clonetics Corp)

LPS stock 10 μg/ml (Cat. No. L 2630 serotype 0111:B4 Sigma)

Human TNF-α ELISA (R&D Systems)

DNase I (10 mg/ml stock)

Preparation of Cells.

LGM-3 media warmed to 37° C.

5 μl of DNase I stock added to 10 ml media.

Cells thawed rapidly and dispersed into above.

Centrifuge 200×g×10 min @ room temperature.

Pellet up in 10 ml sterile PBS.

Centrifuge 200×g×10 min @ room temperature.

Pellet resuspended in 10 ml LGM-3 then diluted to 50 ml with LGM-3.

Perform cell count.

Adjust to 1×E06 cells/well.

Seed 1 ml/well of a 24 well plate.

Place plate in incubator to plate down for 1 hour.

Preparation of Incubation Media.

LGM-3 containing 100 ng/ml LPS (e.g. 50 ml media plus 0.5 ml LPS stock)

Aliquot into 2 ml aliquots and add 1000× inhibitor dilutions.

Incubation

When cells have plated down, aspirate media away and overlay with 1 ml relevant incubation media. Return plate to incubator for 2 hours or 24 hours. Remove supernatants after incubation to a labeled tube and either perform TNF (or other) ELISA immediately or freeze for later assay.

IC₅₀ values are calculated using the concentration of inhibitor that causes a 50% decrease as compared to a control.

What is claimed is:

1. A compound of Formula I 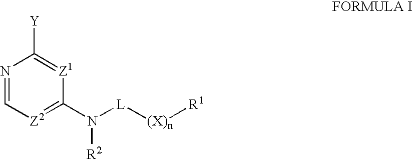
or a pharmaceutically acceptable salt or prodrug thereof, wherein
R¹ is C₁₋₁₀ alkyl, or a C3-12 cyclic hydrocarbyl and which may contain 0, 1, 2, or 3 heteroatoms and which may be optionally substituted by 1-4 groups selected from halo, R³, C₁₋₆ optionally substituted alkenyl, amidine, guanidine, R³CO, COOR³, CONR³₂, OR³, NR³R³, SR³, SO₂R³NHCOR³, CN, and NHCONR³₂, wherein R³ is H, C₁₋₆ alkyl or aryl each of which is optionally substituted with R, OR halo, NR₂, SR, SO₂R, CN, COOR, CONR₂ or CF₃, where each R is independently H or C₁-C₆ alkyl;
L is CO or SO₂;
each X is independently O, CO, CR₂, or NR, where R is lower alkyl and two R groups can be joined to form a 5-7 membered ring, provided that where X is NR or O it is not directly linked to another N or O, and that not more than two X groups are CO;
n=0, 1, 2, or 3;
R² is H, C₁-C₆ alkyl, C₁-C₆ alkenyl, C₁-C₆ heteroalkyl, heterocyclylalkyl, aryl, arylalkyl, heteroaryl, or heteroarylalkyl, each of which is optionally substituted with up to four groups selected from R, halo, CN, OR, ═O, C(NR)NR₂, NR₂, COR, COOR, CONR₂, SR, SOR, SO₂R, SO₂NR₂, NRCOOR, and COCOOR, wherein each R is independently H, alkyl, heteroalkyl, arylalkyl, or diarylalkyl, each of which may be substituted with hydroxy, amino, C₁-C₆ alkoxy, C₁-C₆-alkyl-COOR, C₁-C₆-alkyl-CONR₂ or halo, and wherein two R groups can cyclize to form a 3 to 8 membered ring, optionally including up to two heteroatoms selected from N, O and S;
Y is NR⁴R⁵ or OR⁵,
wherein R⁴ is H or C₁₋₆ alkyl which is optionally substituted with R, OR, NR₂, SR, SO₂R, halo, COOR, ═O, NRCOOR, COR, NRCOR, aryl, arylalkyl, arylalkoxy, or CONR₂, wherein each R is independently H or C₁-C₆ alkyl;
each R⁵ is independently H, a C₁₋₁₀ alkyl optionally substituted with a hydrocarbyl or heterocyclic ring or ring system which may contain 0, 1, 2, or 3 heteroatoms selected from O, N and S, and which is optionally substituted with R, OR, NR₂, SR, SO₂R, halo, COOR, ═O, NRCOOR, COR, NRCOR, aryl, arylalkyl, arylalkoxy, or CONR₂, wherein each R is independently H or C₁-C₆ alkyl; or a C₃₋₇ cycloalkyl, aryl, arylalkyl, heteroaryl, or a fused or unfused carbocyclic or heterocyclic ring, each of which is optionally substituted with up to four groups selected from R, OR, NR₂, SR, SO₂R, halo, COOR, ═O, and CONR₂, wherein each R is independently H or C₁-C₆ alkyl; and
one of Z¹ and Z² is CH, and the other is either CH or N.

2. The compound of claim 1, wherein n=0.

3. The compound of claim 2, wherein L is CO.

4. The compound of claim 3, wherein R¹ is a C₃-C₁₀ alkyl or a C₃-C₁₂ aromatic or partially aromatic group, each of which may contain 0 to 3 heteroatoms and which may be optionally substituted by 1-4 groups selected from halo, R³, C₁₋₆ optionally substituted alkenyl, amidine, guanidine, R³CO, COOR³, CONR³₂, OR³, NR³R³, SR³, SO₂R³NHCOR³, CN, and NHCONR³₂, wherein R³ is H, C₁₋₆ alkyl or aryl each of which is optionally substituted with R, OR halo, NR₂, SR, SO₂R, CN, COOR, CONR₂ or CF₃, where each R is independently H or C₁-C₆ alkyl.

5. The compound of claim 3, wherein R¹ is an aryl(C₂₋₆)alkenyl or a C₃₋₆ cyclic alkyl or aromatic ring or ring system which may contain 0, 1, 2, or 3 heteroatoms and which may be optionally substituted.

6. The compound of claim 3, wherein R¹ is bicyclic.

7. The compound of claim 1, wherein Z¹ and Z² are both CH.

8. The compound of claim 1, wherein either Z¹ or Z² is N.

9. The compound of claim 1, wherein n=1 and X is O.

10. The compound of claim 1, wherein Z¹ is N.

11. The compound of claim 1, wherein Z² is N.

12. The compound of claim 7, wherein n=0.

13. The compound of claim 8, wherein n=0.

14. The compound of claim 3, wherein R¹ is optionally substituted phenyl, thienyl, furanyl, or thiazolyl.

15. The compound of claim 6, wherein R¹ is selected from the group consisting of naphthyl, benzofuranyl, indanyl, 2,3-dihydrobenzofuranyl, benzothienyl, and 1,2,3,4-tetrahydronaphthyl, each of which is optionally substituted by 1-4 groups selected from halo, R³, C₁₋₆ optionally substituted alkenyl, amidine, guanidine, R³CO, COOR³, CONR³₂, OR³, NR³R³, SR³, SO₂R³NHCOR³, CN, and NHCONR³₂, wherein R³ is H, C₁₋₆ alkyl or aryl each of which is optionally substituted with R, OR halo, NR2, SR, SO2R, CN, or CF3, where each R is independently H or C₁-C₆ alkyl.

16. The compound of claim 6, wherein R¹ is selected from the group consisting of naphthyl, indanyl, and 2,3-dihydrobenzofuranyl, each of which may be optionally substituted by 1-4 groups selected from halo, R³, C₁₋₆ optionally substituted alkenyl, amidine, guanidine, R³CO, COOR³, CONR³₂, OR³, NR³R³, SR³, SO₂R³NHCOR³, CN, and NHCONR³₂, wherein R³ is H, C₁₋₆ alkyl or aryl each of which is optionally substituted with R, OR halo, NR2, SR, SO₂R, CN, or CF₃, where each R is independently H or C₁-C₆ alkyl.

17. The compound of claim 1, wherein Y is NH₂ or NR⁴R⁵.

18. The compound of claim 1, wherein Y is NHR⁵ or OR⁵, wherein R⁵ is C₁₋₁₀ alkyl, optionally substituted with a heterocyclic or hydrocarbyl ring.

19. The compound of claim 18, wherein said hydrocarbyl or heterocyclic ring is cyclopropyl, cyclobutyl, cyclopentyl, cyclohexyl, pyrrolidinyl, piperidinyl, morpholinyl, phenyl, pyridinyl, naphthalenyl, tetrahydronapthalenyl, indanyl, tetradrofuranyl, dihydro-furan-2-one, or tetrahydropyranyl.

20. The compound of claim 19, wherein R⁵ is C₁₋₁₀ alkyl substituted with a phenyl group.

21. The compound of claim 1, wherein said heterocyclic or hydrocarbyl ring or ring system is cyclopropyl, cyclobutyl, cyclopentyl, cyclohexyl, pyrrolidinyl, piperidinyl, morpholinyl, phenyl, pyridinyl, naphthalenyl, tetrahydronapthalenyl, indanyl, tetradrofuranyl, dihydro-furan-2-one, or tetrahydropyranyl.

22. The compound of claim 1, wherein R² is a nonaromatic group containing at least one N.

23. The compound of claim 6, wherein R² is 4-piperidinylmethyl, 3-pyrrolidyinylmethyl, or 4-aminobutyl.

24. The compound of claim 1, wherein Y is arylalkylamine.

25. The compound of claim 24, wherein Y is an optionally substituted phenylethylamine.

26. The compound of claim 25, wherein Y is an optionally substituted 1-phenylethylamine.

27. The compound of claim 25, wherein the substituted 1-phenylethylamine is of the S configuration.

28. The compound of claim 25, wherein the substituted 1-phenylethylamine is of the R configuration.

29. The compound of claim 1, wherein
R¹ is selected from the group consisting of: 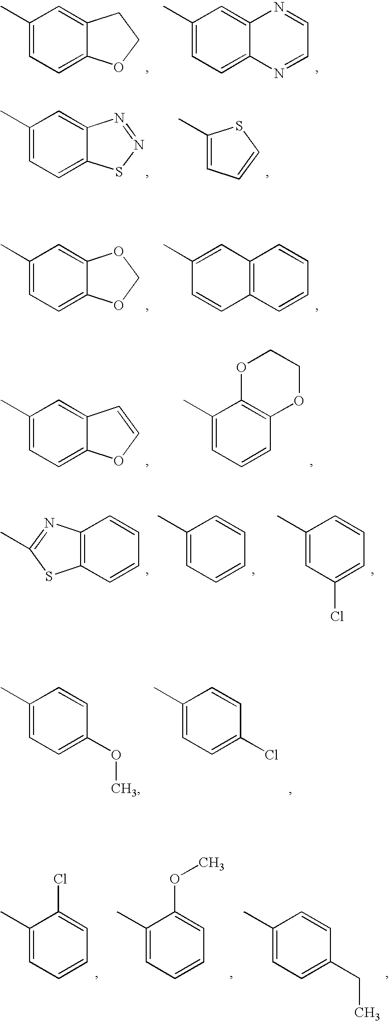 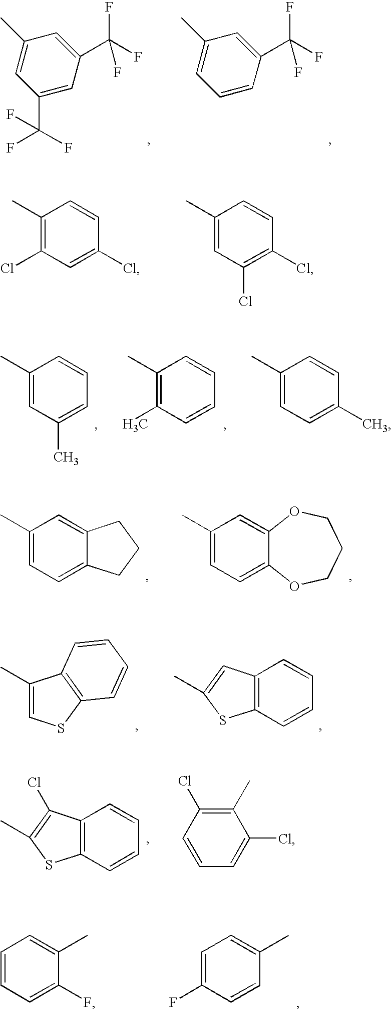 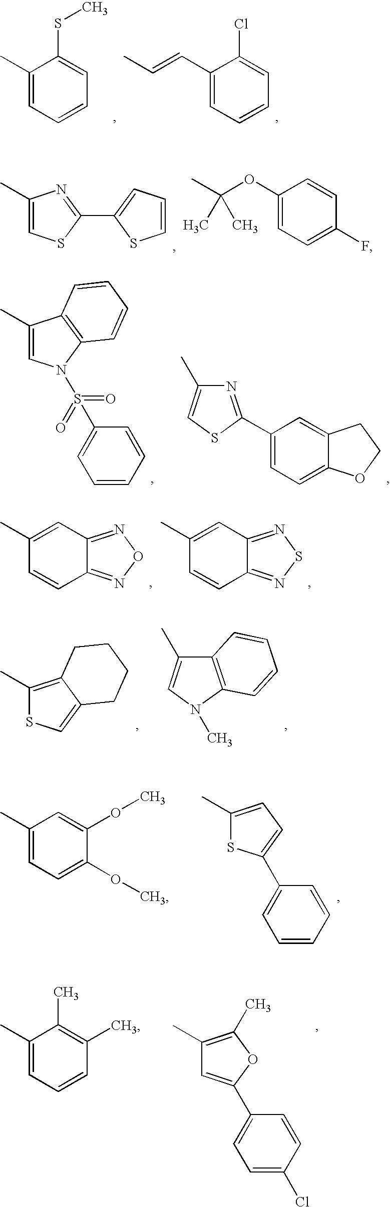 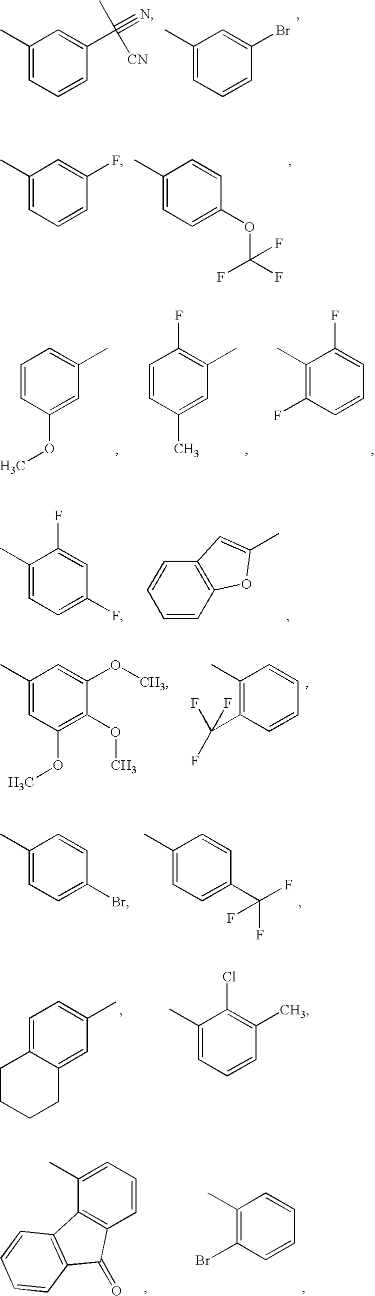 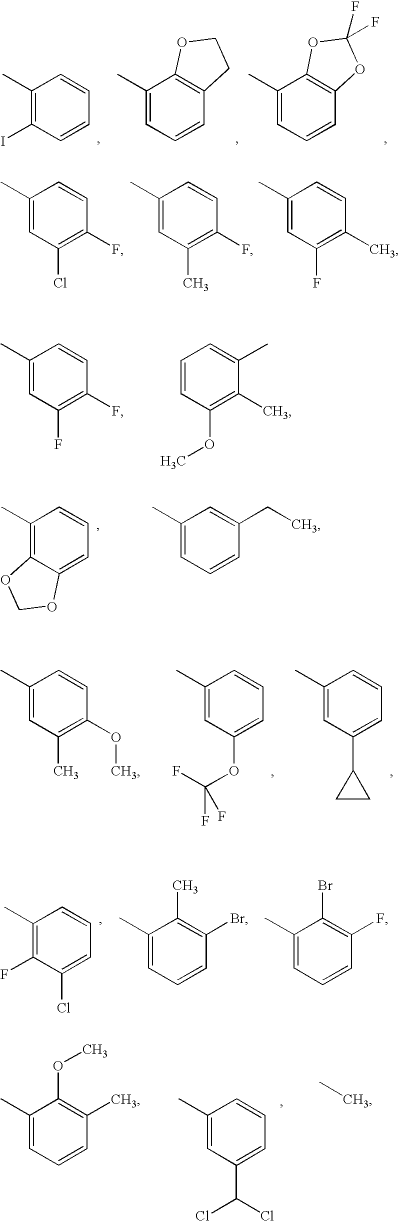 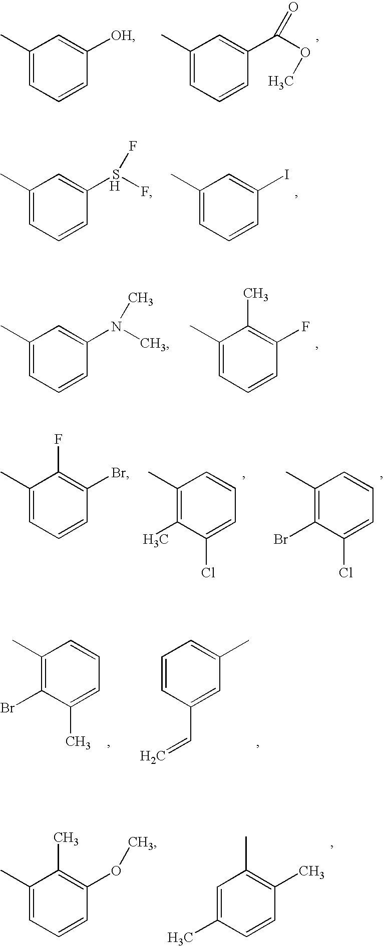 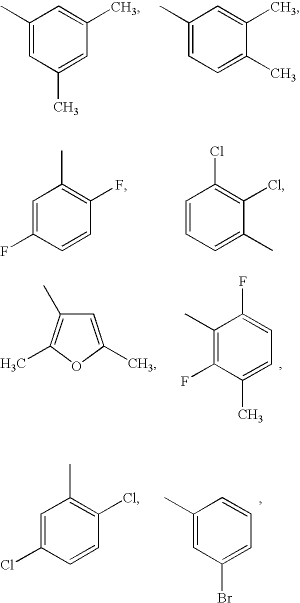 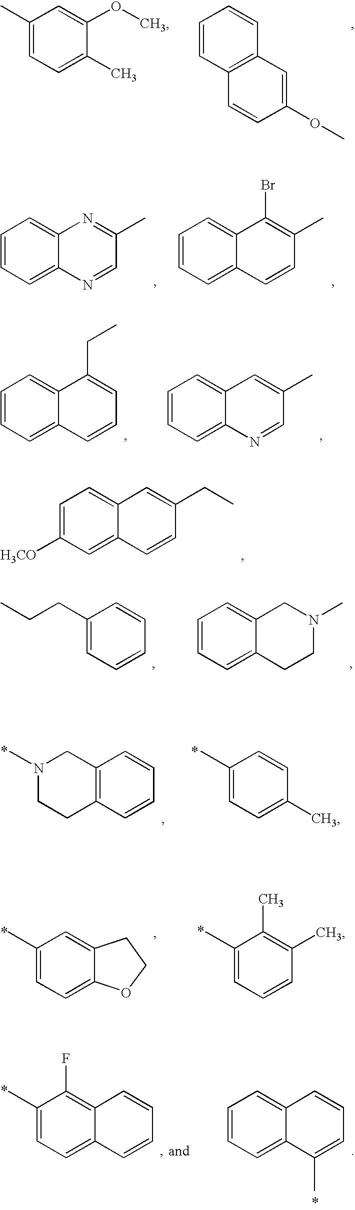
R² is selected from the group consisting of: 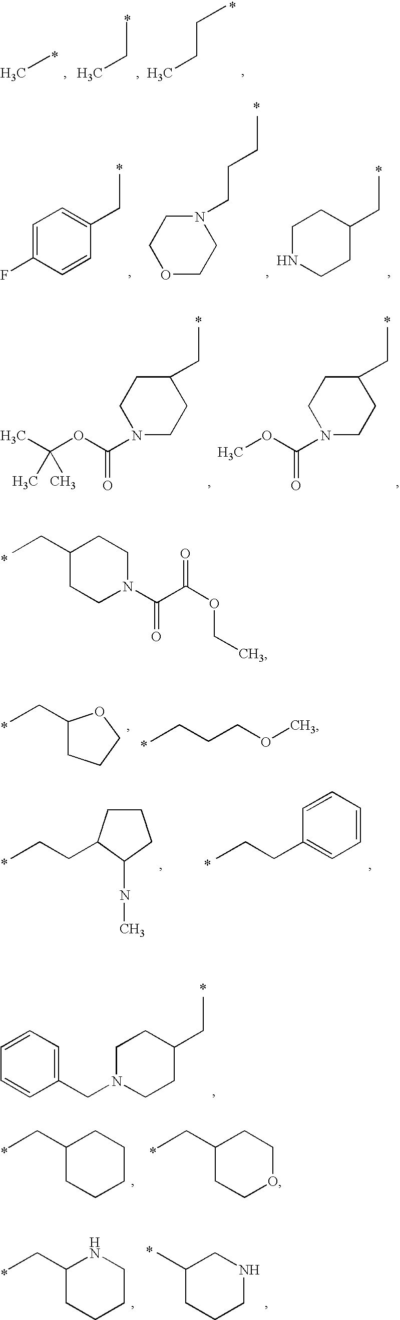 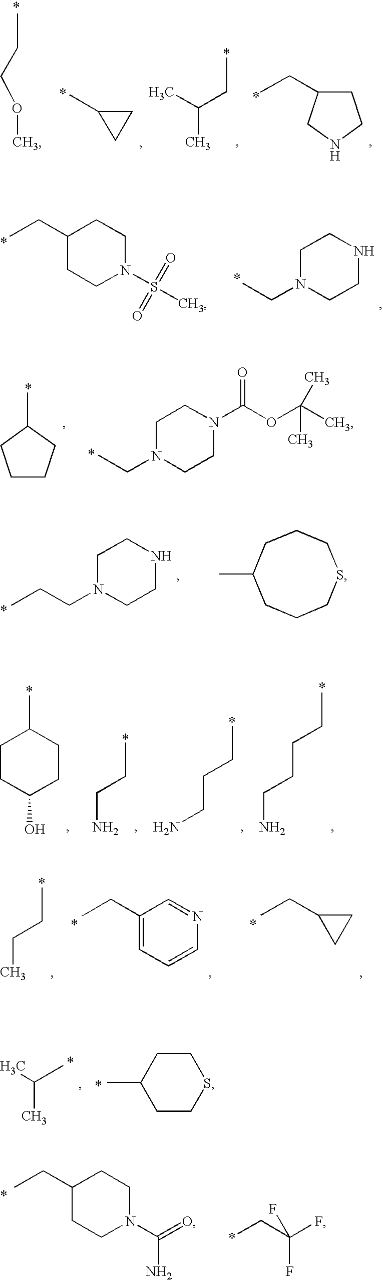 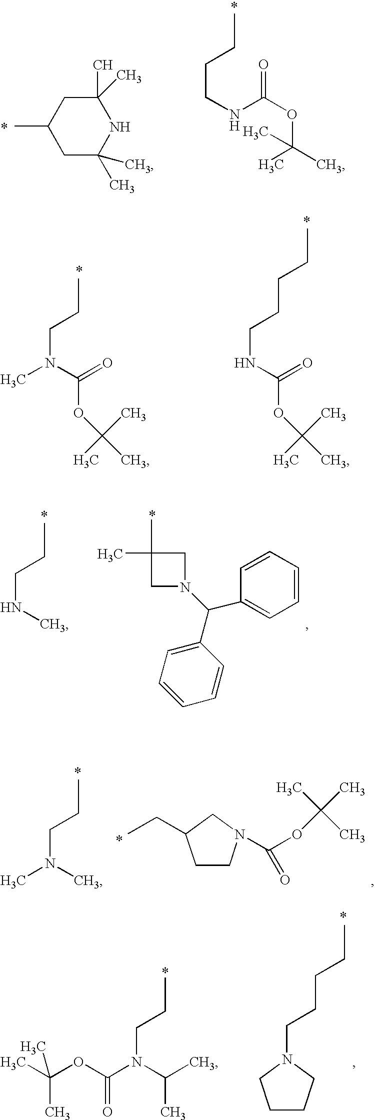 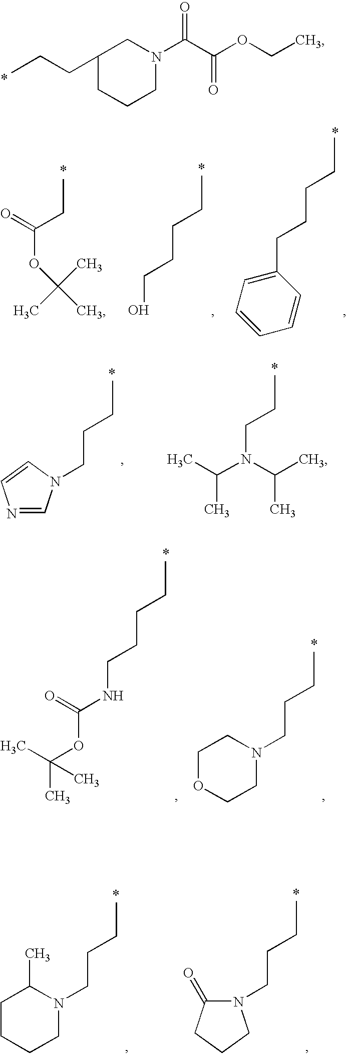 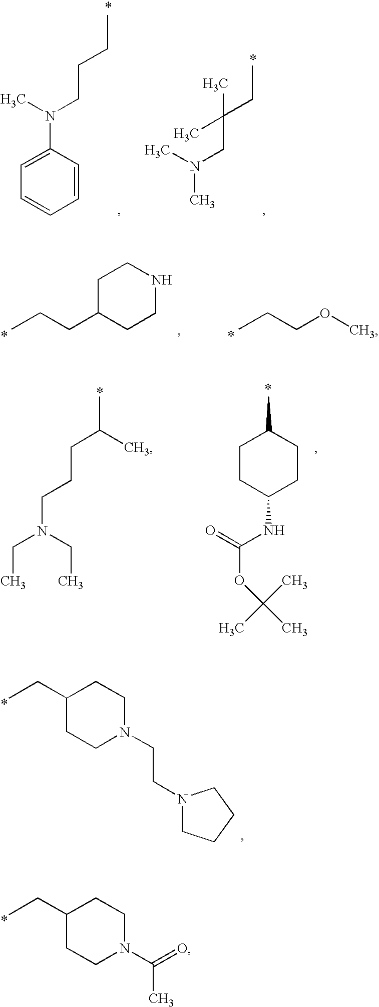 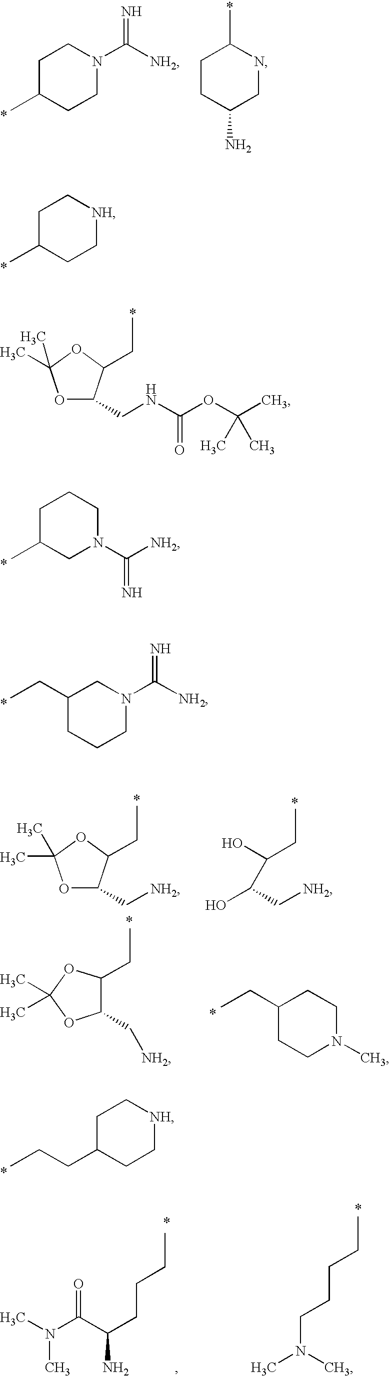 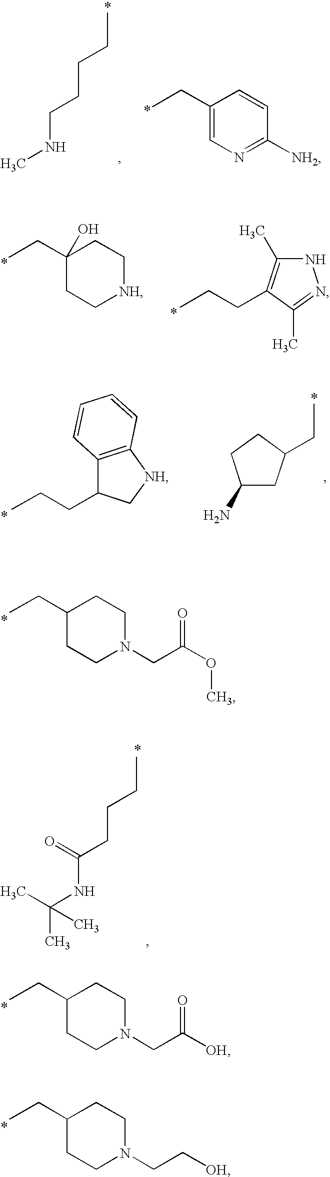 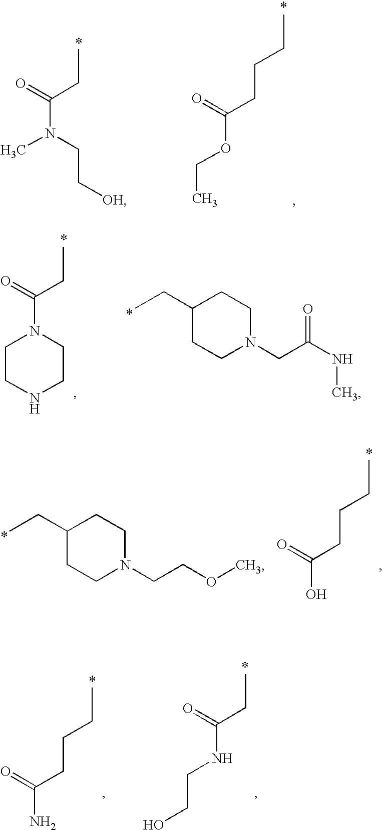 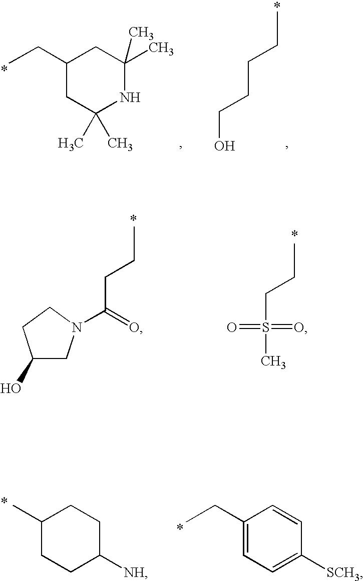 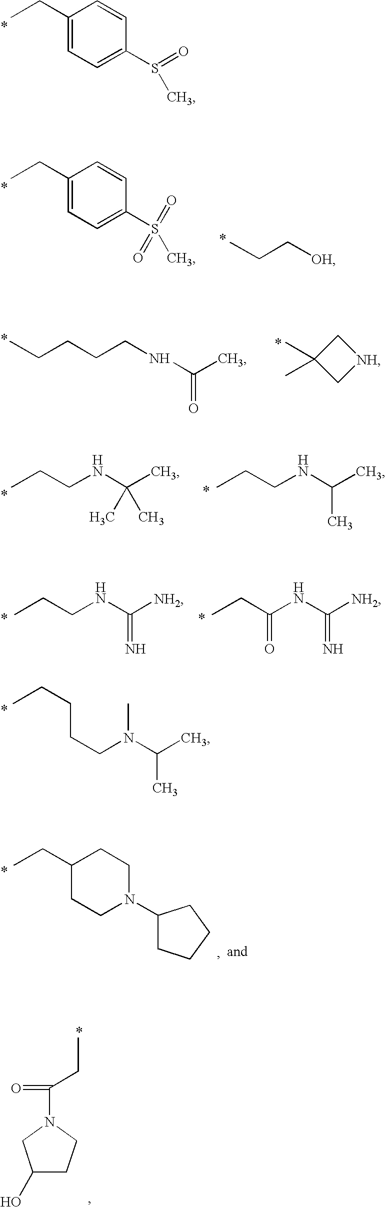
In a preferred embodiment, R² is 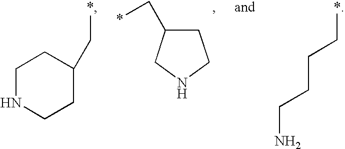
and Y is selected from the group consisting of:

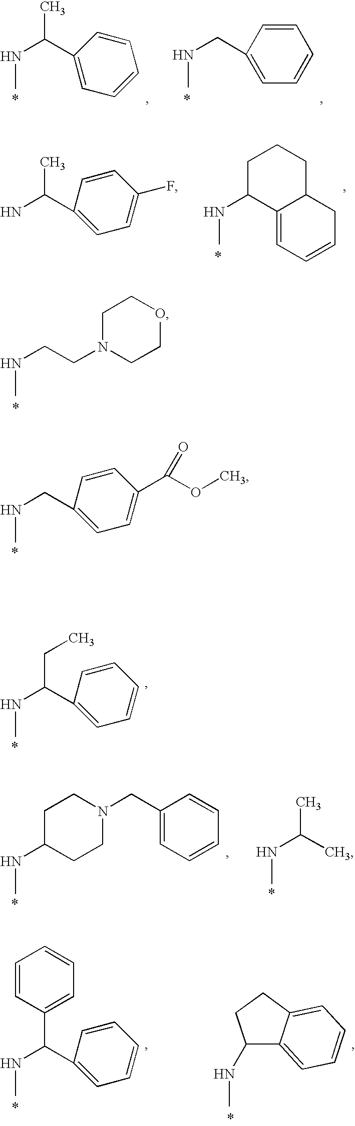

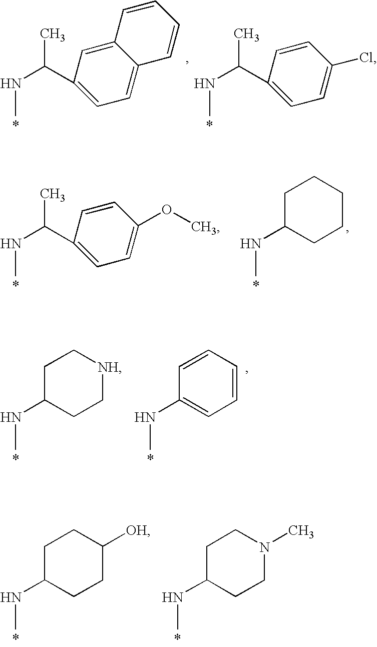

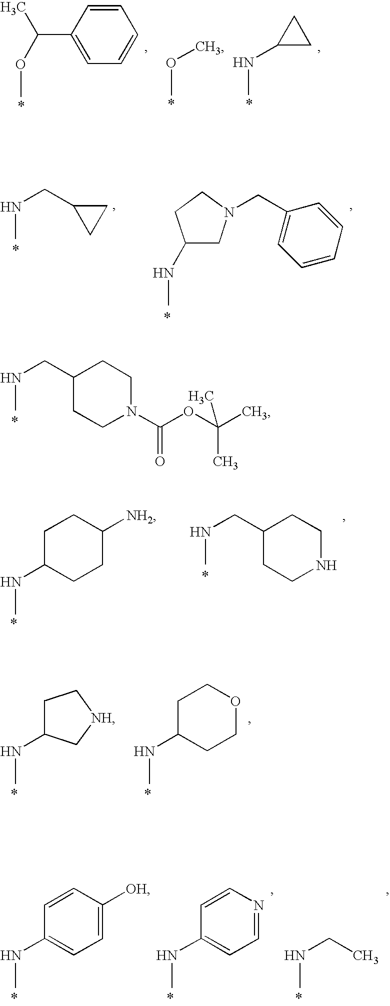

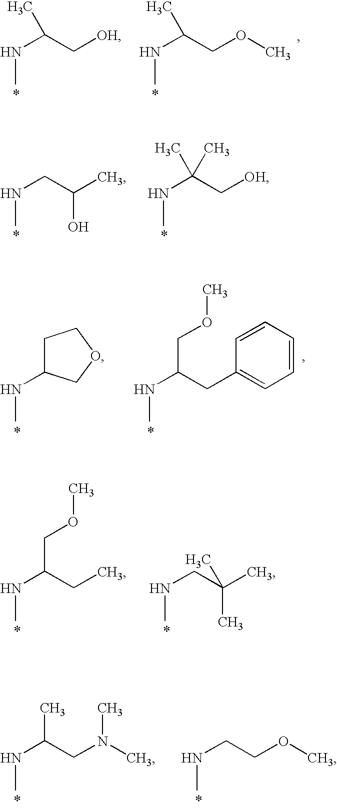

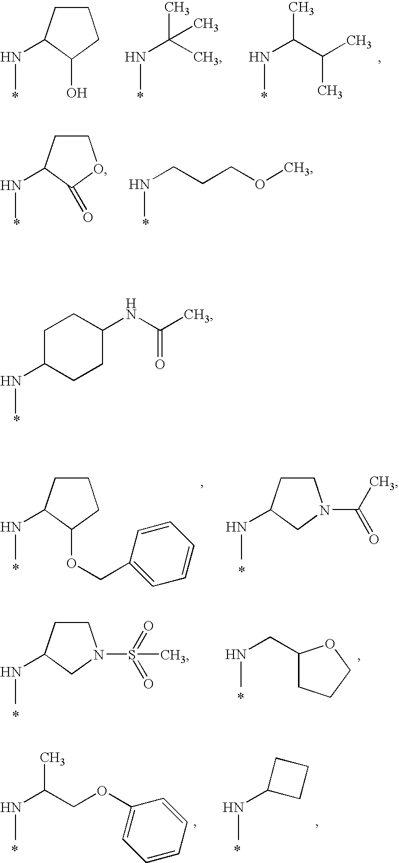

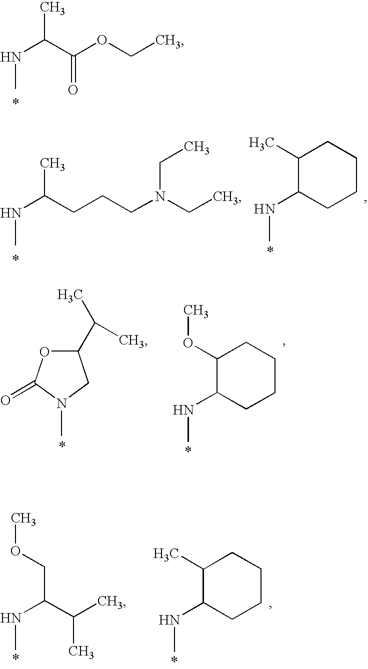

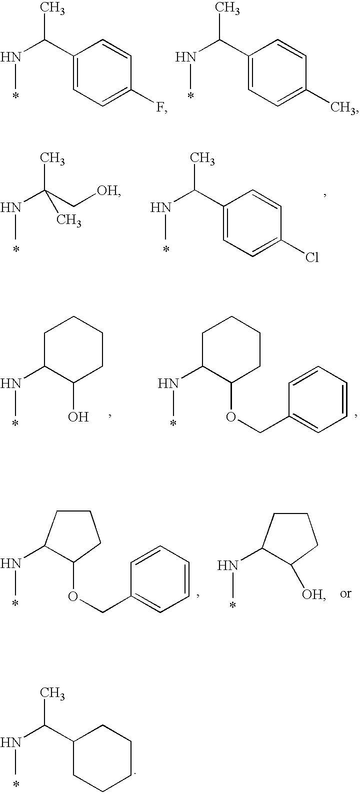

30. A pharmaceutical composition for treating conditions characterized by enhanced p38-α activity which composition comprises
a therapeutically effective amount of at least one compound of claim 1 and at least one pharmaceutically acceptable excipient.

31. The composition of claim 30 which further contains an additional therapeutic agent.

32. The composition of claim 31 wherein said additional therapeutic agent is a corticosteroid, a monoclonal antibody, or an inhibitor of cell division.

33. A method to treat a condition mediated by p38-α kinase comprising administering to a subject in need of such treatment a compound of claim 1, or a pharmaceutical composition thereof.

34. The method of claim 33 wherein said condition is a proinflammation response.

35. The method of claim 34 wherein said proinflammation response is multiple sclerosis, IBD, rheumatoid arthritis, rheumatoid spondylitis, osteoarthritis, gouty arthritis, other arthritic conditions, sepsis, septic shock, endotoxic shock, Gram-negative sepsis, toxic shock syndrome, asthma, adult respiratory distress syndrome, stroke, reperfusion injury, CNS injury, psoriasis, restenosis, cerebral malaria, chronic pulmonary inflammatory disease, chronic obstructive pulmonary disease, cystic fibrosis, silicosis, pulmonary sarcosis, bone fracture healing, a bone resorption disease, soft tissue damage, graft-versus-host reaction, Crohn's Disease, ulcerative colitis, Alzheimer's disease or pyresis.

36. The compound of claim 1 wherein the compound of formula (1) is selected from the group consisting of compounds made in Examples 1-591.
